(19) United States
(12) Patent Application Publication
(10) Pub. No.: US 2022/0267829 A1
(43) Pub. Date: Aug. 25, 2022

(54) Combinatorial Microarray Assay for Detecting and Genotyping SARS-CoV-2

(71) Applicant: University of Maryland, Baltimore, Baltimore, MD (US)

(72) Inventors: Frederick Henry Eggers, Sahuarita, AZ (US); Benjamin Alan Katchman, Tucson, AZ (US); Fushi Wen, Tucson, AZ (US); Candy Mavis Rivas, Tucson, AZ (US); Cory Scott Newland, Tucson, AZ (US); Michael Edward Hogan, Stony Brook, NY (US)

(73) Assignee: PathogenDx, Inc., Scottsdale, AR (US)

(21) Appl. No.: 17/677,600

(22) Filed: Feb. 22, 2022

Related U.S. Application Data
(63) Continuation-in-part of application No. 17/529,666, filed on Nov. 18, 2021.
Application No. 17/529,666 is a continuation-in-part of application No. 17/332,837, filed on May 27, 2021.
(60) Provisional application No. 63/147,613, filed on Feb. 9, 2021.

(51) Int. Cl. C12Q 1/6806; G01N 21/64

(52) U.S. Cl. CPC C12Q 1/6806; G01N 2800/26; G01N 2021/6441; G01N 21/6428

(57) ABSTRACT

Provided herein is a method for detecting the presence of clade variants in the COVID-19 virus in a human sample and/or an environmental sample. Samples are processed to obtain total RNA. The RNA is used as a template in a combined reverse transcription and amplification reaction to obtain fluorescent COVID-19 virus amplicons. These amplicons are hybridized on a microarray with nucleic acid probes having sequences that discriminate among the various clade variants. The microarray is imaged to detect the clade variant and each clade variant is distinguished from others by generating an intensity distribution profile from the image, which is unique to each of the clade variants. Also provided are methods for detecting and genotyping SARS-CoV-2.

### CROSS-REFERENCE TO RELATED APPLICATIONS

This continuation-in-part application claims the benefit of priority under 35 U.S.C. § 120 of pending continuation-in-part application U.S. Ser. No. 14/529,666, filed Nov. 18, 2021, which claims the benefit of priority under 35 U.S.C. § 120 of pending non-provisional application U.S. Ser. No. 17/332,837, filed May 27, 2021, which claims the benefit of priority under 35 U.S.C. § 119(e) of provisional application U.S. Ser. No. 63/147,613, filed Feb. 9, 2021, now abandoned, all of which are hereby incorporated by reference in their entireties.

### BACKGROUND OF THE INVENTION

### Field of the Invention

The present invention relates to the field of multiplex based viral pathogen detection and analysis. More particularly, the present invention relates to detecting the presence of clade variants of SARS-COVID-2 virus in patient and environmental samples.

### Description of the Related Art

The COVID-19 pandemic has increased awareness that viral infection can be an existential threat to health, public safety and the US economy. More fundamentally, there is a recognition that the viral risks are exceedingly dangerous and complex and require new approaches to diagnostics and screening.

The next pandemic wave is expected to have more pronounced flu-like symptoms (seasonal influenza A and/or B) coupled with the COVID-19, or COVID-19 variants that will coexist with the Coronavirus already responsible for the common cold. These complexities are expected to pose significant challenges to public health and the healthcare system in diagnosing multi-symptom conditions accurately and efficiently.

The COVID-19 pandemic has also led to the realization of an additional level of complexity that the realization that human health and environmental contamination are linked in a fundamental way that affects collection efficiency and increases risk to the healthcare workers (1, 2). Alternatives to nasopharyngeal collection methods such as for example, saliva collection are needed to enable scalability among millions of individuals.

Q-RT-PCR technology has dominated COVID-19 diagnostics and public health screening. Independent of the test developer, Q-RT-PCR has been shown to have an unusually high false negative rate (15% up to 30%). As of May 2020, the CDC has recorded 613, 041 COVID-19 tests. With a 15% false negative rate, approximately 91, 956 people would thus be falsely classified as free of infection. Meta-analysis has shown that the false negative rate for Q-RT-PCR is high below day 7 of infection when viral load is still low. This renders Q-RT-PCR ineffective as a tool for early detection of weak symptomatic carriers while also lessening its value in epidemiology.

As for other organisms, genetic variations in SARS-COVID-2 are grouped into clades. There are over 52, 600 complete and high-coverage genomes available on the Global Initiative on Sharing Avian Influenza Data (GISAID). Presently, WHO has identified 10, 022 SARS-COVID-2 genomes from 68 different countries and detected 65, 776 variants and 5, 775 distinct variants that comprised missense mutations, synonymous mutations, mutations in non-coding regions, non-coding deletions, in-frame deletions, non-coding insertions, stop-gained variants, frameshift deletions and in-frame insertions among others. Identifying these clade variants in population and environmental samples while a daunting task, is critical for global public health management directed to controlling the pandemic.

When first identified, it was widely assumed that COVID-19 would mutate slowly, based on a relatively stable genome that would experience minimal genetic drift as the pandemic spread. Unfortunately, perhaps as a function of environmental selection pressure (crowding) physical selection pressure (PPE) and therapeutic selection pressure (vaccination) the original Wuhan clade has evolved into a very large number of clade variants. Consequently, in the past 3 months there has been an international effort to discover and track the full range of clade variant evolution.

Next Generation Sequencing (NGS), primarily Targeted Resequencing of the CoV-2 Spike gene, has been instrumental in elucidating the patterns of genetic variation which define the growing set of clade variants of present international concern (UK, South Africa, Brazil, India, US California, US NY, US Southern) with others emerging at an expanding rate. Whereas NGS is without equal as a discovery tool in genetic epidemiology, it is not ideally suited for field-deployed, public health screening at population scale due to complexities associated with purchasing and managing the kits supply chain, setting up and training personnel, especially when compared to Q-RT-PCR, which is the present standard for nucleic acid based COVID-19 screening. Conversely, while Q-RT-PCR (especially TaqMan) is now the clear standard in COVID-19 testing laboratories for simple positive/negative screening, its suitability for screening clade variants is limited. Deploying TaqMan for COVID-19 clade Identification requires running about 10-15 TaqMan kits on each sample to generate sequence content equivalent to Spike targeted NGS, thereby negating the benefits of costs and logistics with Q-RT-PCR.

Thus, there is a need in the art for superior tools to not only administer and stabilize sample collection for respiratory viruses from millions of samples in parallel obtained from diverse locations including, clinic, home, work, school and in transportation hubs, but also to detect and identify clade variants in the population at the highest levels of sensitivity and specificity. The present invention fulfills this longstanding need and desire in the art.

### SUMMARY OF THE INVENTION

The present invention is directed to a method for detecting severe acute respiratory syndrome coronavirus 2 (SARS-CoV-2) in a subject. In the method a sample is obtained from the subject and total RNA is isolated therefrom. In a single assay a combined reverse transcription and asymmetric PCR amplification reaction is performed on the total RNA using a plurality of fluorescently labeled primer pairs comprising an unlabeled primer and a fluorescently labeled primer that are selective for target sequences within a Spike gene in the SARS-CoV-2 virus to generate a plurality of fluorescent labeled SARS-CoV-2 amplicons. The plurality of fluorescently labeled SARS-CoV-2 amplicons are hybridized to a plurality of nucleic acid probes comprising universal probes, wild type probes and mutant probes, each having a sequence that specifically base-pairs with one of the target sequences in the fluorescently labeled SARS-CoV-2 amplicons and at least one control probe to which the fluorescently labeled SARS-CoV-2 amplicons do not hybridize and where each of the nucleic acid probes is attached to specific positions on a solid microarray support. The microarray is washed at least once. The microarray is imaged to detect fluorescent signals above a threshold for all the nucleic acid probes upon hybridization to the fluorescently labeled SARS-CoV-2 amplicons.

The present invention is directed to a related method for detecting severe acute respiratory syndrome coronavirus 2 (SARS-CoV-2) in a subject where the Spike gene is genotyped at each target sequence. In the related method further comprises measuring the fluorescent signal from hybridization of the fluorescently labeled SARS-CoV-2 amplicons to the wild type probes and from the hybridation of the fluorescently labeled SARS-CoV-2 amplicons to the mutant probes is measured at each of the target sequences. A relative size of the fluorescent signal from hybridization to the mutant probes vs. the fluorescent signal from hybridization to the wild type probes is analyzed directly to produce a hybridization pattern of wild type vs. mutant genotyping among all the target sites in SARS-CoV-2. The hybridization pattern is compared to a known pattern of wild type vs. mutant genotype variation among known SARS-CoV-2 variants to identify the SARS-CoV-2 in the sample as a known variant of concern or a known variant of interest or a combination thereof or as an unknown variant.

The present invention also is directed to a method for detecting, genotyping and identifying a variant of severe acute respiratory syndrome coronavirus 2 (SARS-CoV-2) a subject. In the method a sample is obtained from the subject and total RNA is isolated therefrom. In a single assay a combined reverse transcription and asymmetric PCR amplification reaction is performed on the total RNA using a set of fluorescently labeled primer pairs, each comprising an unlabeled primer and a fluorescently labeled primer, that are selective for sequences within a Spike gene in the SARS-CoV-2 virus to generate a plurality of fluorescent labeled SARS-CoV-2 amplicons. The plurality of fluorescently labeled SARS-CoV-2 amplicons are hybridized to a plurality of nucleic acid probes comprising a set of universal probes, wild type probes and mutant probes, each having a sequence that specifically base-pairs with one of the target sequences in the fluorescently labeled SARS-CoV-2 amplicons and at least one control probe to which the fluorescently labeled SARS-CoV-2 amplicons do not hybridize and where each of the nucleic acid probes attached to a specific position on a solid microarray support. The microarray is washed at least once. The microarray is imaged to detect fluorescent signals above threshold for all the nucleic acid probes produced upon hybridization to the fluorescently labeled SARS-CoV-2 amplicons. At least N fluorescent signals above the threshold from hybridization of the fluorescently labeled SARS-CoV-2 amplicons to the universal probes are measured thereby detecting the SARS-CoV-2 in the sample. The Spike gene is genotyped at each target sequence. During genotyping the fluorescent signals from hybridization of the fluorescently labeled SARS-CoV-2 amplicons to the wild type probes and from the hybridation of the fluorescently labeled SARS-CoV-2 amplicons are compared to the mutant probes at each position on the microarray. A relative size of the fluorescent signal from hybridization to the mutant probes vs. the fluorescent signal from hybridization to the wild type probes is directly analyzed to produce a hybridization pattern of wild type vs. mutant genotyping at each target sequence in SARS-CoV-2. A variant of SARS-CoV-2 is identified as a known variant of concern or a known variant of interest or a combination thereof or as an unknown variant by comparing the hybridization pattern to a known pattern of wild type vs. mutant genotype variation among known SARS-CoV-2 variants.

Other and further aspects, features, and advantages of the present invention will be apparent from the following description of the presently preferred embodiments of the invention. These embodiments are given for the purpose of disclosure.

### BRIEF DESCRIPTION OF THE FIGURES

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows Clade Chip target site performance data for the Spike 69-70 deletion.

FIG. 2 shows Clade Chip target sites performance data normalized to a universal probe for the Spike D80A mutation.

FIG. 3 shows Clade Chip target sites performance data normalized to a universal probe for the Spike D138Y mutation.

FIG. 4 shows Clade Chip target sites performance data normalized to a universal probe for the Spike W152C mutation.

FIG. 5 shows Clade Chip target sites performance data normalized to a universal probe for the Spike N440K mutation.

FIG. 6 shows Clade Chip target sites performance data normalized to a universal probe for the Spike L452R mutation.

FIG. 7 shows Clade Chip target sites performance data normalized to a universal probe for the Spike E484K mutation.

FIG. 8 shows Clade Chip target sites performance data normalized to a universal probe for the Spike N501Y mutation.

FIG. 9 shows Clade Chip target sites performance data normalized to a universal probe for the Spike D614G mutation.

FIG. 10 shows Clade Chip target sites performance data normalized to a universal probe for the Spike P681H mutation.

FIG. 11 shows Clade Chip target sites performance data normalized to a universal probe for the Spike A701V mutation.

FIGS. 12A-12B shows the results of DETECTX-Cv analysis using a multiplex of Amplimers 2, 3, 6, 8. FIG. 12A shows the multiplex analysis data normalized to Universal probe. FIG. 12B shows the multiplex analysis data normalized to Wild-type probe.

FIGS. 13A-13B shows the results of DETECTX-Cv analysis using a multiplex of Amplimers 2, 3, 5, 6, 8. FIG. 13A shows the multiplex analysis data normalized to Universal probe. FIG. 13B shows the multiplex analysis data normalized to Wild-type probe.

FIGS. 14A-14Y shows analytical LoD data for a series of synthetic G-block fragments, used as “synthetic clade variant standards”, corresponding to domains 2-8. FIG. 14A shows DETECTX-Cv analysis for the indicated variants corresponding to the Brazil region. FIG. 14B shows DETECTX-Cv analysis for the indicated variants corresponding to the Brazil region. FIG. 14C shows DETECTX-Cv analysis for the indicated variants corresponding to the Brazil region. FIG. 14D shows DETECTX-Cv analysis for the indicated variants corresponding to the Brazil region. FIG. 14E shows DETECTX-Cv analysis for the indicated variants corresponding to the Brazil region. FIG. 14F shows DETECTX-Cv analysis for the indicated variants corresponding to the Brazil region. FIG. 14G shows DETECTX-Cv analysis for the indicated variants corresponding to the California region. FIG. 14H shows DETECTX-Cv analysis for the indicated variants corresponding to the California region. FIG. 14I shows DETECTX-Cv analysis for the indicated variants corresponding to the California region. FIG. 14J shows DETECTX-Cv analysis for the indicated variants corresponding to the California region. FIG. 14K shows DETECTX-Cv analysis for the indicated variants corresponding to the Indian region. FIG. 14L shows DETECTX-Cv analysis for the indicated variants corresponding to the Indian region. FIG. 14M shows DETECTX-Cv analysis for the indicated variants corresponding to the South Africa region. FIG. 14N shows DETECTX-Cv analysis for the indicated variants corresponding to the South Africa region. FIG. 14O shows DETECTX-Cv analysis for the indicated variants corresponding to the South Africa region. FIG. 14P shows DETECTX-Cv analysis for the indicated variants corresponding to the South Africa region. FIG. 14Q shows DETECTX-Cv analysis for the indicated variants corresponding to the South Africa region. FIG. 14R shows DETECTX-Cv analysis for the indicated variants corresponding to the South Africa region. FIG. 14S shows DETECTX-Cv analysis for the indicated variants corresponding to the South Africa region. FIG. 14T shows DETECTX-Cv analysis for the indicated variants corresponding to the South Africa region. FIG. 14U shows DETECTX-Cv analysis for the indicated variants corresponding to the UK region. FIG. 14V shows DETECTX-Cv analysis for the indicated variants corresponding to the UK region. FIG. 14W shows DETECTX-Cv analysis for the indicated variants corresponding to the UK region. FIG. 14X shows DETECTX-Cv analysis for the indicated variants corresponding to the UK region. FIG. 14Y shows DETECTX-Cv analysis for the indicated variants corresponding to the UK region.

FIGS. 15A-15E shows DETECTX-Cv analysis using synthetic Clade Variant standards. FIG. 15A shows the analysis using synthetic Clade Variant standard corresponding to Brazil. FIG. 15B shows the analysis using synthetic Clade Variant standard corresponding to California 452 (CA 452). FIG. 15C shows the analysis using synthetic Clade Variant standard corresponding to India. FIG. 15D shows the analysis using synthetic Clade Variant standard corresponding to South Africa. FIG. 15E shows the analysis using synthetic Clade Variant standard corresponding to United Kingdom.

FIG. 16 shows LoD range finding DETECTX-Cv analysis for clinical samples processed using Zymo bead capture.

FIG. 17 shows LoD range finding DETECTX-Cv analysis for clinical negative saliva samples processed using Zymo bead capture.

FIGS. 18A-18E shows representative DETECTX-Cv analysis of synthetic Clade variant standards. FIG. 18A shows a histogram analysis for the South Africa synthetic cocktail, D80A-, E484K, N501Y, A701V. FIG. 18B shows a histogram analysis for the California synthetic cocktail, W152C, L452R. FIG. 18C shows a histogram analysis for the India synthetic cocktail, N440K. FIG. 18D shows a histogram analysis for the Brazil P.1 synthetic cocktail, D138Y, E484K, N501Y. FIG. 18E shows a histogram analysis for the UK (B.1.1.7) synthetic cocktail, 69-70 deletion, N501Y, P681H.

FIGS. 19A-19K show representative data for DETECTX-Cv analysis of clinical positive samples performed at TriCore. FIG. 19A shows a histogram analysis for a sample comprising Wuhan/European progenitor variants. FIG. 19B shows a histogram analysis for a sample comprising California variants, W152C AND L452R. FIG. 19C shows a histogram analysis for a sample comprising California variants, W152C and L452R. FIG. 19D shows a histogram analysis for a sample comprising UK variants, 69-70 deletion, N501Y and P681H. FIG. 19E shows a histogram analysis for a sample comprising UK variants, 69-70 deletion, N501Y and P681H. FIG. 19F shows a histogram analysis fora sample comprising, 69-70 deletion, and P681H. FIG. 19G shows a histogram analysis for a sample comprising variant P681H. FIG. 19H shows a histogram analysis for a sample comprising variant P681H. FIG. 19I shows a histogram analysis for a sample comprising California variants, W152C and L452R. FIG. 19J shows a histogram analysis for a sample that did not pass QA/QC. FIG. 19K shows a histogram analysis for a sample that did not pass QA/QC.

FIGS. 20A-20J show representative data for DETECTX-Cv analysis of clinical positive samples performed at PathogenDx. FIG. 20A shows a histogram analysis for a sample comprising California variants W152C and L452R. FIG. 20B shows a histogram analysis for a sample comprising likely California variant L452R. FIG. 20C shows a histogram analysis for a sample comprising California variants, W152C and L452R. FIG. 20D shows a histogram analysis for a sample that did not pass QA/QC. FIG. 20E shows a histogram analysis for a sample comprising California variants W152C and L452R. FIG. 20F shows a histogram analysis for a sample comprising California variant, W152C and L452R. FIG. 20G shows a histogram analysis for a sample comprising California variants, W152C and L452R. FIG. 20H shows a histogram analysis for a sample comprising variant P681H. FIG. 20I shows a histogram analysis for a sample comprising variant P681H. FIG. 20J shows a histogram analysis for a sample comprising Wuhan/European progenitor variants.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or”.

As used herein, “comprise” and its variations, such as “comprises” and “comprising” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

In one embodiment of the present invention there is provided a method for detecting clade variants in a Coronavirus disease 2019 virus (COVID-19) in a sample, comprising obtaining the sample; harvesting viruses from the sample; isolating a total RNA from the harvested viruses; performing a combined reverse transcription and first amplification reaction on the total RNA using at least one first primer pair selective for all COVID-19 viruses to generate COVID-19 virus cDNA amplicons; performing a second amplification using the COVID-19 virus cDNA amplicons as template and at least one fluorescent labeled second primer pair selective for a target nucleotide sequence in the COVID-19 virus cDNA to generate at least one fluorescent labeled COVID-19 virus amplicon; hybridizing the fluorescent labeled COVID-19 virus amplicons to a plurality of nucleic acid probes, each having a sequence corresponding to a sequence determinant that discriminates among the clade variants of the COVID-19 virus, where the nucleic acid probes are attached to a solid microarray support; washing the microarray at least once; and imaging the microarray to detect at least one fluorescent signal from the hybridized fluorescent labeled COVID-19 virus amplicons, thereby detecting the clade variants of the COVID-19 virus in the sample. In a related embodiment after the imaging step, a step is performed of generating an intensity distribution profile from the at least one fluorescent signal that is unique to one of the clade variants thereby detecting the clade variant of the COVID-19 virus in the sample.

A total RNA potentially comprising RNA from COVID-19 virus and other contaminating pathogens and human cells is isolated from the sample. Commercially available RNA isolation kits such as for example, a Quick-DNA/RNA Viral MagBead Kit from Zymo Research are used for this purpose. The total RNA thus isolated is used without further purification. Alternatively, intact virus may be captured with magnetic beads, using kits such as that from Ceres Nanosciences (e.g., CERES NANOTRAP technology), or by first precipitating the virus with polyethylene glycol (PEG), followed by lysis of the enriched virus by heating with a “PCR-Friendly” lysis solution such as 1% NP40 in Tris-EDTA buffer and then used without additional purification.

The COVID-19 virus RNA in the total RNA isolate is used as a template for amplifying a COVID-19 virus specific sequence. This comprises first performing a combined reverse transcriptase enzyme catalyzed reverse transcription reaction and a first amplification reaction using a first primer pair selective for the virus to generate COVID-19 virus cDNA amplicons. In this embodiment, the first primer pairs have forward (odd numbers) and reverse (even number) sequences shown in SEQ ID NO: 1 to SEQ ID NO: 8 (Table 1).). Commercially available reverse transcriptase enzyme and buffers are used in this step. Controls including, but not limited to a RNAse P control having first primer pair (forward primer SEQ ID NO: 130, reverse primer SEQ ID NO: 131) are also used herein (Table 1). The COVID-19 virus cDNA amplicons generated in the first amplification reaction are used as a template for a second amplification that employs at least one fluorescent labeled second primer pair selective for a target nucleotide sequence in the COVID-19 virus cDNA to generate at least one fluorescent labeled COVID-19 virus amplicon.

The fluorescent labeled COVID-19 virus amplicons hybridize to the nucleic acid probes, which are attached at specific positions on a microarray support, for example, a 3-dimensional lattice microarray support. After hybridization, the microarray is washed at least once to remove unhybridized amplicons. Washed microarrays are imaged to detect a fluorescent signal from the hybridized fluorescent labeled COVID-19 virus amplicons to detect the Clade variants of the COVID-19 virus in the sample.

TABLE 1
Primer sequences used for the first amplification reaction

| | Amplimer | | | Primer Sequence |
|---|---|---|---|---|
| SEQ ID NOS. | # | Target | Gene | (5′ to 3′) |
| SEQ ID NO: 1 | 1/2 | AA 00-103 | Spike | TTTAACAGAGTTGTT |
| | | | | ATTTCTAGTGATG |
| SEQ ID NO: 2 | 1/2 | AA 00-103 | Spike | TTTTCTAAAGTAGTA |
| | | | | CCAAAAATCCAGC |
| SEQ ID NO: 3 | 3/4 | AA 124-262 | Spike | TTTCCCTACTTATTG |
| | | | | TTAATAACGCTAC |
| SEQ ID NO: 4 | 3/4 | AA 124-262 | Spike | TTTAGATAACCCAC |
| | | | | ATAATAAGCTGCAG |
| SEQ ID NO: 5 | 5/6 | AA 397-517 | Spike | TTTATCTCTGCTTTA |
| | | | | CTAATGTCTATGC |
| SEQ ID NO: 6 | 5/6 | AA 397-517 | Spike | TTTACAAACAGTTG |
| | | | | CTGGTGCATGTAGA |
| SEQ ID NO: 7 | 7/8 | AA 601-726 | Spike | TTTTGGTGTCAGTG |
| | | | | TTATAACACCAGGA |
| SEQ ID NO: 8 | 7/8 | AA 601-726 | Spike | TTTTGTCTTGGTCAT |
| | | | | AGACACTGGTAGA |
| SEQ ID NO: 264 | N:9 | (AA 167-174) | Nucleocapsid | TTTTGCCAAAAGGC |
| | | | | TTCTACGCAGAA |
| SEQ ID NO: 265 | N:9 | (AA 254-262) | Nucleocapsid | TTGTTTTTGCCGAG |
| | | | | GCTTCTTAGAAG |
| SEQ ID: 130 | — | RNAse P | RNAse P | TTTACTTCAGCATG |
| | | control | | GCGGTGTTTGCAGA |
| SEQ ID: 131 | — | RNAse P | RNAse P | TTTTGATAGCAACA |
| | | control | | ACTGAATAGCCAAG |

Further to this embodiment, prior to the harvesting step, the method comprises mixing the sample with an RNA stabilizer. A representative RNA stabilizer is a chemical stabilizer that protects the RNA from degradation during storage and transportation.

In both embodiments at least one of the fluorescent labeled second primer pair is selective for a panel of target nucleotide sequences within a target region of a gene in the COVID-19 virus; and the nucleic acid probes are specific to the target region of the gene, whereby the at least one fluorescent labeled COVID-19 virus amplicon generated is hybridized to the nucleic acid probe thereby discriminating the clade variants of the COVID-19 virus in the sample. Further to this embodiment the imaging and generating steps comprise detecting the at least one fluorescent signal from the hybridized at least one fluorescent labeled COVID-19 virus amplicons associated with the panel of target nucleotide sequences within the target region of the gene; and generating an intensity distribution profile unique to each of the clade variants, whereby each of the clade variants is distinguishable from others. Particularly, the gene may be a Spike gene.

In a non-limiting example, the target region may be in the Spike gene and/or Nucleoprotein gene in the COVID-19 virus and the fluorescent labeled second primer pairs may have forward (odd numbers) and reverse (even number) sequences shown in SEQ ID NO: 9 to SEQ ID NO: 29 (Tables 2 and 11) and the paired fluorescent labeled second primer sequences shown in Table 27. Controls including, but not limited to a RNAse P control having primer pair (forward primer SEQ ID NO: 132, reverse primer SEQ ID NO: 133) are also used herein (Table 2).

Any fluorescent label may be used in the fluorescent labeled second primer pairs including, but not limited to, a CY3, a CY5, SYBR Green, a DYLIGHT™ DY647, a ALEXA FLUOR 647, a DYLIGHT™ DY547 and a ALEXA FLUOR 550.

TABLE 2
Fluorescent labeled primer sequences used for
amplification reactions

| | Amplimer | | | Primer Sequence |
|---|---|---|---|---|
| SEQ ID NOS. | # | Target | Gene | (5′ to 3′) |
| SEQ ID NO: 9 | 2 | AA64- | Spike | ACCTTTCTTTTCCAATGT |
| | | 80 | | TACTTGGTTC |
| SEQ ID NO: 10 | 2 | AA64- | Spike | Cy3-TTTTATGTTAGA |
| | | 80 | | CTTCTCAGTGGAAGCA |
| SEQ ID NO: 11 | 3 | AA126- | Spike | TTTCTTATTGTTAATAAC |
| | | 157 | | GCTACTAATG |
| SEQ ID NO: 12 | 3 | AA126- | Spike | Cy3-TTTCATTCGCACT |
| | | 157 | | AGAATA AACTCTGAA |
| SEQ ID NO: 13 | 5 | AA408- | Spike | TGTAATTAGAGGTGATG |
| | | 456 | | AAGTCAGA |
| SEQ ID NO: 14 | 5 | AA408- | Spike | Cy3-TTTAAAGGTTTGA |
| | | 456 | | GATTAGACTTCCTAA |
| SEQ ID NO: 15 | 6 | AA475- | Spike | TTTTATTTCAACTGAAAT |
| | | 505 | | YTATCAGGCC |
| SEQ ID NO: 16 | 6 | AA475- | Spike | Cy3-TTTAAAGTACTAC |
| | | 505 | | TACTCT GTATGGTTG |
| SEQ ID NO: 17 | 8 | AA677- | Spike | TTTTATATGCGCTAGTTA |
| | | 707 | | TCAGACTCAG |
| SEQ ID NO: 18 | 8 | AA677- | Spike | Cy3-TTTTGGTATGGC |
| | | 707 | | AATAGA GTTATTAGAG |
| SEQ ID NO: 19 | 1 | AA11- | Spike | TTTTTTTCTTGTTTTATTG |
| | | 33 | | CCACTAGTC |
| SEQ ID NO: 20 | 1 | AA11- | Spike | Cy3-TTTTTGTCAGGG |
| | | 33 | | TAATAAACACCACGTG |
| SEQ ID NO: 21 | 4 | AA213- | Spike | TTTTAAGCACACGCCTA |
| | | 260 | | TTAATTTAGTG |
| SEQ ID NO: 22 | 4 | AA213- | Spike | Cy3-TTTCCACATAAT |
| | | 260 | | AAGCTGCAGCACCAGC |
| SEQ ID NO: 23 | 7 | AA603- | Spike | TTTAGTGTTATAACACCA |
| | | 618 | | GGAACAAATA |
| SEQ ID NO: 24 | 7 | AA603- | Spike | Cy3-TTTTGCATGAAT |
| | | 618 | | AGCAACAGGGACTTCT |
| SEQ ID NO: 137 | 1 | AA15-- | Spike | TTTAGAGTTGTTATTTCT |
| | | 11 | | AGTGATGTTC |
| SEQ ID NO: 138 | 2 | AA98- | Spike | Cy3- |
| | | 105 | | TTTAATCCAGCCTCTTAT |
| | | | | TATGTTAGAC |
| SEQ ID NO: 139 | 3 | AA161- | Spike | Cy3- |
| | | 169 | | TTTCAAAAGTGCAATTAT |
| | | | | TCGCACTAGA |
| SEQ ID NO: 140 | 6 | AA471- | Spike | TTTCTTTTGAGAGAGATA |
| | | 463 | | TTTCAACTGA |
| SEQ ID NO: 141 | 8 | AA666- | Spike | TTTATTGGTGCAGGTAT |
| | | 673 | | ATGCGCTAG |
| SEQ ID NO: 142 | N:9 | AA167- | Nucleoprotein | TTTGCCAAAAGGCTTCT |
| | | 175 | | ACGCAGAAG |
| SEQ ID NO: 143 | N:9 | AA253- | Nucleoprotein | Cy3- |
| | | 261 | | TTTTTTGCCGAGGCTTC |
| | | | | TTAGAAGCC |
| SEQ ID NO: 132 | - | RNAse | RNAse P | TTTGTTTGCAGATTTGG |
| | | P | | ACCTGCGAGCG |
| | | control | | |
| SEQ ID NO: 133 | - | RNAse | | Cy3-TTTAAGGTGAG |
| | | P | RNAse P | CGGCTGTCTCCACAAGT |
| | | control | | |

In all embodiments the clade variant of the COVID-19 virus is identified as a variant of concern, a variant of interest, or a Wuhan variant, or a combination thereof. Particularly, the clade variants of the COVID-19 virus may be Denmark, UK (B.1.1.7), South African (B.1.351), Brazil/Japan (P1), Brazil(B1.1.28), California USA, L452R (1.429), India (N440K), or Wuhan, or a combination thereof. The COVID-19 virus is a Severe Acute Respiratory Syndrome Coronavirus 2 (SARS-CoV 2) or a mutated form thereof. A combination of these variants also may be detected simultaneously.

Also in all embodiments the first primer pair may comprise the nucleotide sequences of SEQ ID NO: 1 and SEQ ID NO: 2, or SEQ ID NO: 3 and SEQ ID NO: 4, or SEQ ID NO: 5 and SEQ ID NO: 6, or SEQ ID NO: 7 and SEQ ID NO: 8, or SEQ ID. NO: 264 or SEQ ID NO: 265, or a combination thereof. Sequences of the first primer pairs for the Spike gene and Nucleocapsid gene are shown in Table 1.

In addition in all embodiments the fluorescent labeled second primer pair may comprise the nucleotide sequences of SEQ ID NO: 9 and SEQ ID NO: 10, or SEQ ID NO: 11 and SEQ ID NO: 12, or SEQ ID NO: 13 and SEQ ID NO: 14, or SEQ ID NO: 15 and SEQ ID NO: 16, or SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 24, SEQ ID NO: 137 and SEQ ID NO: 20, or SEQ ID NO: 9 and SEQ ID NO: 138, or SEQ ID NO: 11 and SEQ ID NO: 139, or SEQ ID NO: 140 and SEQ ID NO: 16, or SEQ ID NO: 141 and SEQ ID NO: 18, or SEQ ID NO: 142 and SEQ ID NO: 143 or a combination thereof. Sequences of the fluorescent labeled second primer pairs are shown in Tables 2, 11 and 27.

Furthermore, in all embodiments the nucleic acid probes may comprise at least one nucleotide sequence selected from the group consisting of SEQ ID NOS: 30-129 and/or 144-263. The nucleic acid probes may have a sequence corresponding to a sequence determinant that discriminates among the Clade variants of the COVID-19 virus. The nucleic acid probes are specific to the target region of the gene in the COVID-19 virus as discussed supra. This enables hybridization of the one fluorescent labeled COVID-19 virus amplicon generated to the Spike gene-specific nucleic acid probe thereby discriminating the Clade variants of the COVID-19 virus in the sample. In a non-limiting example, the target region is in a Spike gene or nucleoprotein gene in the COVID-19 virus and the nucleic acid probes have a sequence shown in SEQ ID NO: 30 to SEQ ID: 129 (Tables 3, 10 and 14) or SEQ ID NO: 144 to SEQ ID NO: 263 (Tables 28A and 28B). Controls including, but not limited to, a RNAse P control nucleic acid probe (SEQ ID NO: 68 and SEQ ID NO: 69) and a negative control nucleic acid probe (SEQ ID NO: 70) are also used herein (Table 3).

TABLE 3
Nucleic acid probe sequences used for hybridization

| | Amplimer | | | |
|---|---|---|---|---|
| SEQ ID NOS. | # | Target | Gene | Probe Sequence (5′ to 3′) |
| SEQ ID NO: 31 | 2 | AA69-70 HV | S* | TTTTTCCCATGCTATACATGTCTCT* |
| | | | | GTTTTTT |
| SEQ ID NO: 32 | 2 | AA69-70 | S | TTTTTTTTTCCATGCTATCTCTGGG |
| | | DEL | | ATTTTTT |
| SEQ ID NO: 33 | 2 | AA D80A | S | TTTTTCAGAGGTTTGMTAACCCTG |
| | | | | TCTTTTTT |
| SEQ ID NO: 34 | 2 | AA D80_ | S | TTTTTTTGGTTTGATAACCCTGCTT |
| | | | | TTTTT |
| SEQ ID NO: 35 | 2 | AA 80A | S | TTTTTTTGGTTTGCTAACCCTGCTT |
| | | | | TTTTT |
| SEQ ID NO: 36 | 3 | AA D138Y | S | TTTTATTTTGTAATKATCCATTTTT |
| | | | | GTTTT |
| SEQ ID NO: 37 | 3 | AA D138 | S | TTTTCTTTGTAATGATCCATTTTC |
| | | | | TTTTT |
| SEQ ID NO: 38 | 3 | AA 138Y | S | TTTTTTTTTGTAATTATCCATTTTCT |
| | | | | TTTT |
| SEQ ID NO: 39 | 3 | AA W1520 | S | TTTTTAGTTGKATGGAAAGTGAGT |
| | | | | TCTTTT |
| SEQ ID NO: 40 | 3 | AA W152_ | S | TTTCTCTAAAAGTTGGATGGAAAC |
| | | | | TCTTCT |
| SEQ ID NO: 41 | 3 | AA 152C | S | TTCTTCAAAGTTGTATGGAAAGC |
| | | | | CTTCTT |
| SEQ ID NO: 42 | 5 | AA | S | TTTTTAATTCTAAMAAKCTTGATTC |
| | | 439K+N440K | | TAATTTT |
| SEQ ID NO: 43 | 5 | AA | S | TTTTTAATTCTAACAATCTTGATTT |
| | | N439_+N440 | | CTTTT |
| SEQ ID NO: 44 | 5 | AA | S | TTTTTTATTCTAACAAGCTTGATTT |
| | | N439_+_440K | | TTTTT |
| SEQ ID NO: 45 | 5 | AA_ | S | TTTTCTATTCTAAAAATCTTGATTT |
| | | 439K+N440_ | | CTTTT |
| SEQ ID NO: 46 | 5 | AA L452R | S | TTTCTATAATTACCTGTATAGATTG |
| | | | | TCTTT |
| SEQ ID NO: 47 | 5 | AA L452 | S | TTTTTTTAATTACCTGTATAGATTT |
| | | | | CTTTT |
| SEQ ID NO: 48 | 5 | AA 452R | S | TTTTTCATAATTACTGGTATAGATC |
| | | | | TTTTT |
| SEQ ID NO: 49 | 6 | AA S477_ | S | TTTTTTCGCCGGTAGCACACCTCT |
| | | | | TTTTTT |
| SEQ ID NO: 50 | 6 | AA_477N | S | TTTTCTTCCGGTAACACACCTTTTT |
| | | | | TTTTT |
| SEQ ID NO: 51 | 6 | AA | S | TTTTTTAATGGTGTTRAAGGTTTTA |
| | | V483A+E484K | | ATTTTTT |
| SEQ ID NO: 52 | 6 | AA | S | TTTTTTCTGGTGTTGAAGGTTTTA |
| | | V483_+E484 | | CTTTTT |
| SEQ ID NO: 53 | 6 | AA | | TTTTTTTATGGTGTTAAAGGTTTTC |
| | | V483_+_484K | S | TTTTT |
| SEQ ID NO: 54 | 6 | AA | S | TTTTTTTATGGTGCTGAAGGTTCT |
| | | 483A+E484 | | TTTTTT |
| SEQ ID NO: 55 | 6 | AA N501Y | S | TTTTTTTTCCAACCCACTWATGGT |
| | | | | GTTTTTTTT |
| SEQ ID NO: 56 | 6 | AA N501_ | S | TTTTTTTACCCACTAATGGTGTCT |
| | | | | TTTTT |
| SEQ ID NO: 57 | 6 | AA N501Y | S | TTTTTTTTACCCACTTATGGTGTCT |
| | | | | TTTTT |
| SEQ ID NO: 58 | 8 | AA P681H | S | TTTTTTCAGACTAATTCTCMTCGG |
| | | | | CTTTTT |
| SEQ ID NO: 59 | 8 | AA P681_ | S | TTTTTTTCTAATTCTCCTCGGCGTT |
| | | | | TTTTT |
| SEQ ID NO: 60 | 8 | AA_681H | S | TTTTTTTTAATTCTCATCGGCGTT |
| | | | | TTTTT |
| SEQ ID NO: 61 | 8 | AA A701V | S | TTTTCACTTGGTGYAGAAAATTCA |
| | | | | GTTTTT |
| SEQ ID NO: 62 | 8 | AA A701_ | S | CTTCTTCTTGGTGCAGAAAATTA |
| | | | | TTCTTT |
| SEQ ID NO: 63 | 8 | AA_701V | S | TCTTCTTCTTGGTGTAGAAAATTAT |
| | | | | TCTTT |
| SEQ ID NO: 144 | 1 | AA L5F | S | TTTTCGTTTGTTTTTYTTGTTTTATT |
| | | | | GCTTTT |
| SEQ ID NO: 145 | 1 | AA L5_ | S | TTTTCGTTTGTTTTTCTTGTTTTAT |
| | | | | TTTTT |
| SEQ ID NO: 146 | 1 | AA _5F | S | TTTTCGTTTGTTTTTTTTGTTTTATT |
| | | | | TTTT |
| SEQ ID NO: 147 | 1 | AA _18F | S | TTTTCTTGTTAATTTTACAACCAT |
| | | | | TTTTT |
| SEQ ID NO: 148 | 1 | AA _19R | S | TTTTTCTTTAATCTTAGAACCAGAC |
| | | | | TTTTT |
| SEQ ID NO: 149 | 2 | AA | S | TTTTTCTCCATGCTATACATGTCCT |
| | | A67_.HV69- | | TTTTT |
| | | 70 | | |
| SEQ ID NO: 150 | 2 | AA A67_.69- | S | TTTTTTTTTCCATGCTATCTCTGTT |
| | | 70DEL | | TTTTT |
| SEQ ID NO: 151 | 2 | AA_67V.69- | S | TTTTTTTTTCCATGTTATCTCTGTT |
| | | 70DEL | | TTTTT |
| SEQ ID NO: 152 | 2 | AA_80G | S | TTTTTTGGTTTGGTAACCCTGCT |
| | | | | TTTTTT |
| SEQ ID NO: 153 | 2 | AA T95I | S | TTTCTTTTTGCTTCCAYTGAGAAG |
| | | | | TCTTTTTT |
| SEQ ID NO: 154 | 2 | AA T95_ | S | TTTTTTCCGCTTCCACTGAGAAGC |
| | | | | ATTTTT |
| SEQ ID NO: 155 | 2 | AA_95I | S | TTTTTCCGCTTCCATTGAGAAGC |
| | | | | ATTTTT |
| SEQ ID NO: 156 | 3 | AA Y144_ | S | TTTCTTTGGGTGTTTATTACCACA |
| | | | | AAAATTTT |
| SEQ ID NO: 157 | 3 | AA_ | S | TTTTTTTTTGGGTGTTTACCACAAA |
| | | 144DEL_ | | AACTTTT |
| SEQ ID NO: 158 | 3 | AA_ | S | TTTATTTTTGGGTGTTACTTATTAC |
| | | 144T.145in | | CACATTT |
| | | sS | | |
| SEQ ID NO: 159 | 3 | AA_143G_ | S | TTCTTTTGGATGTTTATTACCACAA |
| | | | | AAACTTT |
| SEQ ID NO: 160 | 3 | AA_ | S | TTCGTAATGATCCATTTTATTACCA |
| | | 141Y.142- | | CAAATTT |
| SEQ ID NO: 161 | 3 | 144DEL | S | TCTCTTCAAAAGTTTGATGGAAAT |
| | | AA 152L | | CTCTTT |
| SEQ ID NO: 162 | 3 | AA_152R | S | TTTCTTTACAAAAGTAGGATGGAT |
| | | | | TTCTTT |
| SEQ ID NO: 163 | 3 | AA_154K | S | TTTCTTTGTTGGATGAAAAGTGAT |
| | | | | CTTCTT |
| SEQ ID NO: 164 | 3 | AA F157_ | S | TTTTGAAAGTGAGTTCAGAGTTTA |
| | | | | CCTTTT |
| SEQ ID NO: 165 | 3 | AA_157del | S | TTCTTTGGAAAGTGGAGTTTATTC |
| | | | | TCTTTT |
| SEQ ID NO: 166 | 4 | AA 241-243 | S | TTTTTTTTCAAACTTTACTTGCTTT |
| | | LLA | | ACTCTTT |
| SEQ ID NO: 167 | 4 | AA_241- | S | TTTTTTTTCAAACTTTACATAGAAG |
| | | 243DEL | | CCTTTTT |
| SEQ ID NO: 168 | 4 | AA_R246_ | S | TTTCTACATAGAAGTTATTTGACT |
| | | | | CCCTTTT |
| SEQ ID NO: 169 | 4 | AA_ | S | TTTTCTGCTTTACATATGACTCCT |
| | | 246N.247- | | GGTTTTTT |
| | | 253DEL | | |
| SEQ ID NO: 170 | 4 | AA D253G | S | TTTCTACTCCTGGTGRTTCTTCTT |
| | | | | CATTTT |
| SEQ ID NO: 171 | 4 | AA D253_ | S | TTTTTTCCCTGGTGATTCTTCTTTC |
| | | | | TTTTT |
| SEQ ID NO: 172 | 4 | AA_253G | S | TTTTTCCCTGGTGGTTCTTCTTTT |
| | | | | TTTTT |
| SEQ ID NO: 173 | 5 | AA_452Q | S | TTTTTTAATTACCAGTATAGATCC |
| | | | | TTTTT |
| SEQ ID NO: 174 | 6 | AA T478K | S | TTTTTCGGTAGCAMACCTTGTAAT |
| | | | | GTTTTT |
| SEQ ID NO: 175 | 6 | AA T478_ | S | TTTTTTTGTAGCACACCTTGTATTT |
| | | | | TTTTT |
| SEQ ID NO: 176 | 6 | AA_478K | S | TTTTTTGTAGCAAACCTTGTATTT |
| | | | | TTTTT |
| SEQ ID NO: 177 | 6 | AA_484Q | S | TTTTTATGGTGTTCAAGGTTTTCT |
| | | | | TTTTT |
| SEQ ID NO: 178 | 6 | AA F490S | S | TTTTCTAATTGTTACTTTCCTTTAC |
| | | | | AATTTTT |
| SEQ ID NO: 179 | 6 | AA F490_ | S | TTTTTTTTGTTACTTTCCTTTACTT |
| | | | | TTTT |
| SEQ ID NO: 180 | 6 | AA_490S | S | TTTTTTTTGTTACTCTCCTTTACT |
| | | | | TTTTT |
| SEQ ID NO: 181 | 6 | AA S494P | S | TTTTTCTCCTTTACAAYTATATGGT |
| | | | | TTTTTTT |
| SEQ ID NO: 182 | 6 | AA S494_ | S | TTTTTCTCTTTACAATCATATGGTC |
| | | | | TTTTT |
| SEQ ID NO: 183 | 6 | AA_494P | S | TTTTTCTCTTTACAACCATATGGTC |
| | | | | TTTTT |
| SEQ ID NO: 184 | 6 | AA_501T | S | TTTTTTACCCACTACTGGTGTTTT |
| | | | | TTTTT |
| SEQ ID NO: 185 | 8 | AA Q677P/H | S | TTTTTTATCAGACTCMGACTAATT |
| | | | | CTCTTTTT |
| SEQ ID NO: 186 | 8 | AA Q677_ | S | TTTTTTCCAGACTCAGACTAATTTC |
| | | | | TTTTT |
| SEQ ID NO: 187 | 8 | AA_677P | S | TTTTTCTTCAGACTCCGACTAATC |
| | | | | TTTTTT |
| SEQ ID NO: 188 | 8 | AA_677H2 | S | TTTTTTCCAGACTCACACTAATTTC |
| | | | | TTTTT |
| SEQ ID NO: 189 | 8 | AA_677H1 | S | TTTTTTCCAGACTCATACTAATTTC |
| | | | | TTTTT |
| SEQ ID NO: 190 | 8 | AA 681R | S | TTTTTTTTAATTCTCGTCGGCGTT |
| | | | | TTTTT |
| SEQ ID NO: 191 | N:9 | AA S194L | N* | TTTTCCGCAACAGTTYAAGAAATT |
| | (AA183- | | | CATTTT |
| | 252) | | | |
| SEQ ID NO: 192 | N:9 | AA S194_ | N | TTTTTTTAACAGTTCAAGAAATTTT |
| | (AA183- | | | TTTTT |
| | 252) | | | |
| SEQ ID NO: 193 | N:9 | AA_194L | N | TTTTTTTAACAGTTTAAGAAATTTT |
| | (AA183- | | | TTTTT |
| | 252) | | | |
| SEQ ID NO: 194 | N:9 | AA S197L | N | TTTTCTCAAGAAATTYAACTCCAG |
| | (AA183- | | | GCTTTT |
| | 252) | | | |
| SEQ ID NO: 195 | N:9 | AA S197_ | N | TTTTTCTAAGAAATTCAACTCCATT |
| | (AA183- | | | TTTTT |
| | 252) | | | |
| SEQ ID NO: 196 | N:9 | AA_197L | N | TTTTTCTAAGAAATTTAACTCCATT |
| | (AA183- | | | TTTTT |
| | 252) | | | |
| SEQ ID NO: 197 | N:9 | AA P199L | N | TTTTTAAATTCAACTCYAGGCAGC |
| | (AA183- | | | ATCTTT |
| | 252) | | | |
| SEQ ID NO: 198 | N:9 | AA P199_ | N | TTTTTTTTTCAACTCCAGGCAGCT |
| | (AA183- | TTTTTT | | |
| | 252) | | | |
| SEQ ID NO: 199 | N:9 | AA_199L | N | TTTTTTTTTCAACTCTAGGCAGCTT |
| | (AA183- | | | TTTTT |
| | 252) | | | |
| SEQ ID NO: 200 | N:9 | AA S2011 | N | TTTTTACTCCAGGCAKCWSTADRS |
| | (AA183- | | | GATTTT |
| | 252) | | | |
| SEQ ID NO: 201 | N:9 | AA S201_ | N | TTTTTTTTCCAGGCAGCAGTADRT |
| | (AA183- | | | TTTTTT |
| | 252) | | | |
| SEQ ID NO: 202 | N:9 | AA _2011 | N | TTTTTTTTCCAGGCATCAGTAGGT |
| | (AA183- | | | TTTTTT |
| | 252) | | | |
| SEQ ID NO: 203 | N:9 | AA S202N | N | TTTTCCCAGGCAGCARTADRSGAA |
| | (AA183- | | | CCTTTT |
| | 252) | | | |
| SEQ ID NO: 204 | N:9 | AA S202_ | N | TTTTTTTAGGCAGCAGTADRSGAT |
| | (AA183- | | | TTTTTT |
| | 252) | | | |
| SEQ ID NO: 205 | N:9 | AA_202N | N | TTTTTTTAGGCAGCAATAGGGGAT |
| | (AA183- | | | TTTTTT |
| | 252) | | | |
| SEQ ID NO: 206 | N:9 | AA R203M/K | N | TTTTGCAGCWSTADRSGAACTTCT |
| | (AA183- | G204R | | CTTTTT |
| | 252) | | | |
| SEQ ID NO: 207 | N:9 | AA R203_ | N | TTTTTTTCAGCAGTAGGGGAACTC |
| | (AA183- | G204 | | TTTTTT |
| | 252) | | | |
| SEQ ID NO: 208 | N:9 | AA_203M | N | TTTTTTTCAGCAGTATGGGAACTC |
| | (AA183- | G204_ | | TTTTTT |
| | 252) | | | |
| SEQ ID NO: 209 | N:9 | AA_203K | N | TTTTTTTCAGCAGTAAACGAACTC |
| | (AA183- | G204R | | TTTTTT |
| | 252) | | | |
| SEQ ID NO: 210 | N:9 | AA_203K | N | TTTTTTTCAGCTCTAAACGAACTCT |
| | (AA183- | G204R (2) | | TTTTT |
| | 252) | | | |
| SEQ ID NO: 211 | N:9 | AA T2051 | N | TTTTCSTADRSGAAYTTCTCCTGC |
| | (AA183- | | | TATTTT |
| | 252) | | | |
| SEQ ID NO: 212 | N:9 | AA T205_ | N | TTTTTTTGGGGAACTTCTCCTGCC |
| | (AA183- | | | TTTTTT |
| | 252) | | | |
| SEQ ID NO: 213 | N:9 | AA_2051 | N | TTTTTTTGGGGAATTTCTCCTGCC |
| | (AA183- | | | TTTTTT |
| | 252) | | | |
| SEQ ID NO: 214 | N:9 | AA A208G | N | TTTTAAYTTCTCCTGCTAGAATGG |
| | (AA183- | R209del | | CTGTTT |
| | 252) | | | |
| SEQ ID NO: 215 | N:9 | AA A208G | N | TTTTACGAACTTCTCCTGGAATGG |
| | (AA183- | R209del (2) | | CTGTTT |
| | 252) | | | |
| SEQ ID NO: 216 | N:9 | AA A208_ | N | TTTTCTCTCCTGCTAGAATGGCTG |
| | (AA183- | R209 | | TTTTTT |
| | 252) | | | |
| SEQ ID NO: 217 | N:9 | AA 208G_ | N | TTTTTACTTCTCCTGGAATGGCTG |
| | (AA183- | 209del | | TTTTTT |
| | 252) | | | |
| SEQ ID NO: 218 | N:9 | AA G212V | N | TTTTTTGGCTGKCWATKGCKGTGA |
| | (AA183- | N213Y | | TTTTTT |
| | 252) | | | |
| SEQ ID NO: 219 | N:9 | AA G212_ | N | TTTTTTTAATGGCTGGCWATKGCT |
| | (AA183- | N213Y | | TTTTTT |
| | 252) | | | |
| SEQ ID NO: 220 | N:9 | AA_212V | N | TTTTTTTAATGGCTGTCAATGGCT |
| | (AA183- | N213_ | | TTTTTT |
| | 252) | | | |
| SEQ ID NO: 221 | N:9 | AA G212V | N | TTTTTTTTGGCTGKCAATKGCKGC |
| | (AA183- | N213_ | | TTTTTT |
| | 252) | | | |
| SEQ ID NO: 222 | N:9 | AA G212 | N | TTTTTTTTGGCTGGCTATGGCGGC |
| | (AA183- | 213Y | | TTTTTT |
| | 252) | | | |
| SEQ ID NO: 223 | N:9 | AA G2140 | N | TTTTTTGGCTGKCWATKGCKGTGA |
| | (AA183- | G215C | | TTTTTT |
| | 252) | | | |
| SEQ ID NO: 224 | N:9 | AA G214_ | N | TTTTTTTTGKCWATGGCKGTGATT |
| | (AA183- | G2150 | | TTTTTT |
| | 252) | | | |
| SEQ ID NO: 225 | N:9 | AA_214C | N | TTTTTTTTGGCAATTGCGGTGATT |
| | (AA183- | G215_ | | TTTTTT |
| | 252) | | | |
| SEQ ID NO: 226 | N:9 | AA G2140 | N | TTTTTTTCWATKGCGGTGATGCTT |
| | (AA183- | G215_ | | TTTTTT |
| | 252) | | | |
| SEQ ID NO: 227 | N:9 | AA G214_ | N | TTTTTTTCAATGGCTGTGATGCTTT |
| | (AA183- | _2150 | | TTTTT |
| | 252) | | | |
| SEQ ID NO: 228 | N:9 | AA M234I | N | TTTTTGAGCAAAATDTYTGGTAAA |
| | (AA183- | S235F | | GTTTTT |
| | 252) | | | |
| SEQ ID NO: 229 | N:9 | AA M234_ | N | TTTTTTTCAAAATGTCTGGTAAATT |
| | (AA183- | | | TTTTT |
| | 252) | S235_ | | |
| SEQ ID NO: 230 | N:9 | AA_2341 | N | TTTTTCTAGCAAAATTTCTGGTATC |
| | (AA183- | S235_ | | TTTTT |
| | 252) | | | |
| SEQ ID NO: 231 | N:9 | AA_2341 | N | TTTTTCTAGCAAAATATCTGGTATC |
| | (AA183- | S235_(2) | | TTTTT |
| | 252) | | | |
| SEQ ID NO: 232 | N:9 | AA M234_ | N | TTTTTCTCAAAATGTTTGGTAAATC |
| | (AA183- | _235F | | TTTTT |
| | 252) | | | |
| SEQ ID NO: 134 | — | RNAse P | — | TTTTTTTTCTGACCTGAAGGCTCT |
| | | | | GCGCGTTTTT |
| SEQ ID NO: 135 | — | RNAse P | — | TTTTTCTTGACCTGAAGGCTCTGC |
| | | | | TTTTTT |
| SEQ ID NO: 136 | — | Negative | — | TTTTTTCTACTACCTATGCTGATTC |
| | | Control | | ACTCTTTTT |

*S = Spike, N = Nucleoprotein

Further still in all embodiments the sample may comprise at least one of a nasopharyngeal swab, a nasal swab, a mouth swab, a mouthwash, an aerosol, or a swab from a hard surface. In one aspect the sample may be any sample obtained from a subject including, but not limited to, a nasopharyngeal swab, a nasal swab, a mouth swab, and a mouthwash (sample obtained by rinsing the subject's buccal cavity). A pooled sample obtained by combining two or more of these samples or by combining samples from multiple subjects also may be used. In another aspect, the sample is an environmental sample obtained from inanimate sources include, but are not limited to, an aerosol and a hard surface. The aerosol samples may be obtained using commercial air samplers such as for example a Coriolis Micro Air Sampler. A sample from a hard surface may be obtained using a swab. In both aspects, the viruses from samples obtained on swabs are dispersed in a liquid such as phosphate buffered saline. Aerosol samples are transferred into a volume of a liquid such as phosphate buffered saline.

In another embodiment of the present invention, there is provided a method for detecting Clade variants in the Coronavirus disease 2019 virus (COVID-19) in a sample, comprising obtaining the sample; harvesting viruses from the sample; isolating total RNA from the harvested viruses; performing a combined reverse transcription and asymmetric PCR amplification reaction on the total RNA using at least one fluorescent labeled primer pair comprising an unlabeled primer, and a fluorescently labeled primer, selective fora target sequence in all COVID-19 viruses to generate at least one fluorescent labeled COVID-19 virus amplicon; hybridizing the fluorescent labeled COVID-19 virus amplicons to a plurality of nucleic acid probes, each having a sequence corresponding to a sequence determinant that discriminates among the clade variants of the COVID-19 virus, where the nucleic acid probes are attached to a solid microarray support; washing the microarray at least once; and imaging the microarray to detect at least one fluorescent signal from the hybridized fluorescent labeled COVID-19 virus amplicons, thereby detecting the clade variants of the COVID-19 virus in the sample. In a related embodiment after the imaging step, a step is performed of generating an intensity distribution profile from the at least one fluorescent signal that is unique to one of the clade variants thereby detecting the clade variant of the COVID-19 virus in the sample.

The total RNA is isolated as described supra and any COVID-19 virus RNA in the total RNA isolate is used as a template in a combined reverse transcription/amplification reaction (RT-PCR). In this step, the nucleic acid sequences in the COVID-19 virus RNA are transcribed using a reverse transcriptase enzyme to generate COVID-19 complementary DNA (cDNA) that is amplified in the same reaction using COVID-19 virus selective fluorescent labeled primer pairs to generate fluorescent labeled COVID-19 virus amplicons. Each fluorescent labeled primer pair comprises an unlabeled primer and a fluorescently labeled primer in about 4-fold to about 8-fold excess of the unlabeled primer whereby, upon completion of the reaction, the fluorescently labelled amplicon is primarily single stranded (that is, the reaction is a type of “asymmetric PCR”).

Hybridization of the fluorescent labeled COVID-19 virus amplicons to the plurality of nucleic acid probes attached at specific positions on a microarray support is performed as described supra. The nucleic acid probes may have a sequence corresponding to a sequence determinant that discriminates among the Clade variants of the COVID-19 virus and are specific to the target region of the gene in the COVID-19 virus, as discussed supra. This enables hybridization of the one fluorescent labeled COVID-19 virus amplicon generated to the Spike gene-specific nucleic acid probe or nucleoprotein specific nucleic acid probe thereby discriminating the Clade variants of the COVID-19 virus in the sample. In a non-limiting example, the target region is in a Spike gene and/or a nucleoprotein gene in the COVID-19 virus and the nucleic acid probes have a sequence shown in SEQ ID NO: 31 to SEQ ID: 63 (Table 3). Controls are as described supra and shown in (Table 3).

Further to this embodiment, prior to the harvesting step, the method comprises mixing the sample with an RNA stabilizer. A representative RNA stabilizer is a chemical stabilizer, as described supra.

In both embodiments at least one of the fluorescent labeled second primer pair is selective for a panel of target nucleotide sequences within a target region of a gene in the COVID-19 virus; and the nucleic acid probes are specific to the target region of the gene, whereby the at least one fluorescent labeled COVID-19 virus amplicon generated is hybridized to the nucleic acid probe thereby discriminating the clade variants of the COVID-19 virus in the sample. Further to this embodiment the method comprises detecting the at least one fluorescent signal from the hybridized at least one fluorescent labeled COVID-19 virus amplicons associated with the panel of target nucleotide sequences within the target region of the gene; and generating an intensity distribution profile unique to each of the clade variants, whereby each of the clade variants is distinguishable from others. Particularly, the gene may be the Spike gene and/or a nucleoprotein gene.

In a non-limiting example, the target region may be in the Spike gene and/or a nucleoprotein gene in the COVID-19 virus and the fluorescent labeled second primer pairs may have forward (odd numbers) and reverse (even number) sequences shown in SEQ ID NO: 9 to SEQ ID NO: 29 (Tables 2 and 11) and the paired fluorescent labeled second primer sequences shown in Table 27. Controls including, but not limited to a RNAse P control having a primer pair with forward primer SEQ ID NO: 66 and reverse primer SEQ ID NO: 67 are also used herein (Table 2).

In all embodiments the COVID-19 gene, the clade variants of the COVID-19 virus, the at least one fluorescent labeled primer pair, the fluorescent label, the nucleic acid probes, and the samples are as described supra. Also in all embodiments the fluorescently labeled primer may be in an excess of about 4-fold to about 8-fold over the unlabeled primer in the fluorescent labeled primer pair. Exemplary nucleotide sequences for the fluorescent labeled primer pairs including, for example, RNAse P controls, are shown in Tables 2, 11 and 27. Exemplary nucleotide sequences for the nucleic acid probes, including, for example, RNAse P controls and negative controls, are shown in Tables 3, 28A and 28B.

In yet another embodiment of the present invention there is provided a method for detecting severe acute respiratory syndrome coronavirus 2 (SARS-CoV-2) in a subject, comprising obtaining a sample from the subject; isolating total RNA from the sample; performing in a single assay a combined reverse transcription and asymmetric PCR amplification reaction on the total RNA using a plurality of fluorescently labeled primer pairs comprising an unlabeled primer and a fluorescently labeled primer, selective for target sequences within a Spike gene in the SARS-CoV-2 virus to generate a plurality of fluorescent labeled SARS-CoV-2 amplicons; hybridizing the plurality of fluorescently labeled SARS-CoV-2 amplicons to a plurality of nucleic acid probes comprising a plurality of universal probes, wild type probes and mutant probes, each having a sequence that specifically base-pairs with one of the target sequences in the fluorescently labeled SARS-CoV-2 amplicons and at least one control probe to which the fluorescently labeled SARS-CoV-2 amplicons do not hybridize, each of the nucleic acid probes attached to specific positions on a solid microarray support; washing the microarray at least once; imaging the microarray to detect fluorescent signals above a threshold for all the nucleic acid probes upon hybridization to the fluorescently labeled SARS-CoV-2 amplicons.

Further to this embodiment the Spike gene is genotyped at each target sequence where the method further comprises measuring the fluorescent signal from hybridization of the fluorescently labeled SARS-CoV-2 amplicons to the wild type probes and from the hybridation of the fluorescently labeled SARS-CoV-2 amplicons to the mutant probes at each of the target sequences; analyzing directly a relative size of the fluorescent signal from hybridization to the mutant probes vs. the fluorescent signal from hybridization to the wild type probes to produce a hybridization pattern of wild type vs. mutant genotyping among all the target sites in SARS-CoV-2; and comparing the hybridization pattern to a known pattern of wild type vs. mutant genotype variation among known SARS-CoV-2 variants to identify the SARS-CoV-2 in the sample as a known variant of concern or a known variant of interest or a combination thereof or as an unknown variant.

In one aspect of both embodiments the plurality of fluorescently labeled primer pairs may be a set of nucleotide sequences comprising SEQ ID NO: 266 and SEQ ID NO: 138, SEQ ID NO: 11 and SEQ ID NO: 139, SEQ ID NO: 267 and SEQ ID NO: 141, SEQ ID NO: 268 and SEQ ID NO: 16, and SEQ ID NO: 141 and SEQ ID NO: 269. Further to this aspect the plurality of fluorescently labeled primer pairs comprises an internal control primer pair to amplify RNase P and the control probe comprises a sequence that specifically base-pairs with a fluorescently labeled RNase P amplicon. In this further aspect the internal control primer pair may comprise the nucleotide sequences of SEQ ID NO: 132 and SEQ ID NO: 270. Also the control probe may comprise the nucleotide sequence of SEQ ID NO: 134.

In another aspect of both embodiments the universal probes may comprise the nucleotide sequences of SEQ ID NOS: 33, 36, 42, 61, 153, 174, 235, 236, 251-252, 256, 259, 283, 287, 291, 293-294, 299, 301, 302 304, and 305. In yet another aspect the wild type probes may comprise the nucleotide sequences of SEQ ID NOS: 37, 40, 43, 47, 52, 59, 62, 126, 154, 164, 179, 182, 235, 282, 288, and 298. In yet another aspect the mutant probes may comprise the nucleotide sequences of SEQ ID NOS: 35, 38, 41, 44, 45, 53, 54, 129, 155, 161, 176, 180, 183, 189, 190, 236, 289, 290, 292, 295, 296, 297, 300, 303, 306, and 307.

In both embodiments and all aspects thereof during the imaging step, SARS-CoV-2 may be detected by measuring at least N fluorescent signals above the threshold from hybridizing of the fluorescently labeled SARS-CoV-2 amplicons to the universal probes. Also in both embodiments and all aspects thereof the fluorescently labeled primer may be in an excess of about 4-fold to about 8-fold over the unlabeled primer in the fluorescent labeled primer pair. In addition the sample may be, but is not limited to, a nasopharyngeal swab, a nasal swab, a mouth swab, or saliva.

In yet another embodiment of the present invention there is provided a method for detecting, genotyping and identifying a variant of severe acute respiratory syndrome coronavirus 2 (SARS-CoV-2) in a subject, comprising obtaining a sample from the subject; isolating total RNA from the sample; performing in a single assay a combined reverse transcription and asymmetric PCR amplification reaction on the total RNA using a set of fluorescently labeled primer pairs, each comprising an unlabeled primer and a fluorescently labeled primer, selective for sequences within a Spike gene in the SARS-CoV-2 virus to generate a plurality of fluorescent labeled SARS-CoV-2 amplicons; hybridizing the plurality of fluorescently labeled SARS-CoV-2 amplicons to a plurality of nucleic acid probes comprising a set of universal probes, wild type probes and mutant probes, each having a sequence that specifically base-pairs with one of the target sequences in the fluorescently labeled SARS-CoV-2 amplicons and at least one control probe to which the fluorescently labeled SARS-CoV-2 amplicons do not hybridize, each of the nucleic acid probes attached to specific positions on a solid microarray support; washing the microarray at least once; imaging the microarray to detect fluorescent signals above threshold for all the nucleic acid probes produced upon hybridization to the fluorescently labeled SARS-CoV-2 amplicons; measuring at least N fluorescent signals above the threshold from hybridization of the fluorescently labeled SARS-CoV-2 amplicons to the universal probes thereby detecting the SARS-CoV-2 in the sample; genotyping the Spike gene at each target sequence, the step comprising comparing the fluorescent signals from hybridization of the fluorescently labeled SARS-CoV-2 amplicons to the wild type probes and from the hybridation of the fluorescently labeled SARS-CoV-2 amplicons to the mutant probes at each position on the microarray; and analyzing directly a relative size of the fluorescent signal from hybridization to the mutant probes vs. the fluorescent signal from hybridization to the wild type probes to produce a hybridization pattern of wild type vs. mutant genotyping at each target sequence in SARS-CoV-2; and identifying a variant of SARS-CoV-2 as a known variant of concern or a known variant of interest or a combination thereof or as an unknown variant by comparing the hybridization pattern to a known pattern of wild type vs. mutant genotype variation among known SARS-CoV-2 variants.

In one aspect of this embodiment the set of fluorescently labeled primer pairs may comprise the nucleotide sequences of SEQ ID NO: 266 and SEQ ID NO: 138, SEQ ID NO: 11 and SEQ ID NO: 139, SEQ ID NO: 267 and SEQ ID NO: 141, SEQ ID NO: 268 and SEQ ID NO: 16, SEQ ID NO: 141 and SEQ ID NO: 269, and SEQ ID NO: 132 and SEQ ID NO: 270. The fluorescently labeled primer pairs are described in Table 29.

In another aspect of this embodiment the probes are described in Tables 30 and 31. Particularly, the probe hybridizing to the fluorescently labeled RNase P amplicon may comprise the nucleotide sequence of SEQ ID NO: 134. Also the universal probes may comprise the nucleotide sequences of SEQ ID NOS: 33, 36, 42, 61, 153, 174, 235, 236, 251-252, 256, 259, 283, 287, 291, 293-294, 299, 301, 302 304, and 305. In addition the wild type probes may comprise the nucleotide sequences of SEQ ID NOS: 37, 40, 43, 47, 52, 59, 62, 126, 154, 164, 179, 182, 235, 282, 288, and 298. Furthermore the mutant probes may comprise the nucleotide sequences of SEQ ID NOS: 35, 38, 41, 44, 45, 53, 54, 129, 155, 161, 176, 180, 183, 189, 190, 236, 289, 290, 292, 295, 296, 297, 300, 303, 306, and 307.

In this embodiment and aspects thereof N may be equal to or greater than 6 fluorescent signals. Also in this embodiment and aspects thereof the excess of the fluorescently labeled primer in the primer pair and the sample are as described supra.

In yet another embodiment of the present invention, there is a provided a method for detecting a pathogen in a subject comprising obtaining a sample from the subject; isolating total nucleic acids from the sample; performing in a single assay a combined reverse transcription and/or an asymmetric PCR amplification reaction on the total nucleic acids using a plurality of fluorescently labeled primer pairs comprising an unlabeled primer and a fluorescently labeled primer, selective for target sequences within a gene of interest in the pathogen to generate a plurality of fluorescently labeled pathogen amplicons; hybridizing the plurality of fluorescently labeled pathogen amplicons to a plurality of nucleic acid probes comprising a plurality of universal probes, wild type probes and mutant probes, each having a sequence that specifically base-pairs with one of the target sequences in the fluorescently labeled pathogen amplicons and at least one control probe to which the fluorescently labeled pathogen amplicons do not hybridize, each of the nucleic acid probes attached to specific positions on a solid microarray support; washing the microarray at least once; imaging the microarray to detect fluorescent signals above a threshold for all the nucleic acid probes upon hybridization to the fluorescently labeled pathogen amplicons.

Further to this embodiment the gene of interest is genotyped at each target sequence where the method comprises measuring the fluorescent signal from hybridization of the fluorescently labeled pathogen amplicons to the wild type probes and from the hybridation of the fluorescently labeled pathogen amplicons to the mutant probes at each of the target sequences; analyzing directly a relative size of the fluorescent signal from hybridization to the mutant probes vs. the fluorescent signal from hybridization to the wild type probes to produce a hybridization pattern of wild type vs. mutant genotyping among all the target sites in the pathogen; and comparing the hybridization pattern to a known pattern of wild type vs. mutant genotype variation among known pathogens to identify the variant.

In one aspect of both embodiments the pathogen may be an RNA virus or a DNA virus. For example, the RNA virus may be, but is not limited to, an influenza virus, such as influenza A or influenza B. A DNA virus may be, but is not limited to, an adenovirus, a herpesviruses, a papillomavirus, or a smallpox virus. In another aspect the pathogen may be a pathogenic bacteria, such as, but not limited to, a respiratory disease-causing bacterium, for example, a Mycobacterium species, a Streptococcus species, a Mycoplasma species, an Enterococcus species, a Haemophilus species, a Klebsiella species, a Moraxella species, a Corynebacterium species, or a combination thereof. In yet another aspect the pathogen may be a pathogenic fungus, such as, but not limited to, a disease-causing molds and yeasts, for example, a Histoplasma species, a Coccidioides species, a Blastomyces species, a Rhizopus species, an Aspergillus species, a Pneumocystis species, a Candida species or a Cryptococcus species, or a combination thereof.

In this embodiment and aspects thereof during the imaging step, the pathogen is detected by measuring at least N fluorescent signals above the threshold from hybridizing of the fluorescently labeled pathogen amplicons to the universal probes. Particularly, N is equal to or greater than 6 fluorescent signals. In this embodiment and aspects thereof the fluorescently labeled primer may be in an excess of about 4-fold to about 8-fold over the unlabeled primer in the fluorescent labeled primer pair. In addition in this embodiment and aspects thereof the sample may be an individual sample or a pooled sample from a nasopharyngeal swab, a nasal swab, a mouth swab, a mouthwash, an aerosol, or a swab from a hard surface or a combination thereof.

Provided herein are methods of nucleic acid analysis to detect stable genetic variation such as a clade variation in a viral pathogen which is based on simultaneous analysis of multiple sequence domains in a gene, such as for example the Spike (S) gene in the RNA genome of CoV-2, or the Nucleoprotein (N) gene in SARS-CoV-2 or a combination of the Spike gene and the Nucleoprotein gene in SARS-CoV-2 to measure clade variation in SARS-CoV-2.

In one method for detecting the stable genetic variation, total RNA from the harvested viruses is isolated and used in a combined reverse transcription and first amplification reaction (RT-PCR) to generate COVID-19 virus cDNA amplicons. These amplicons are used as template in a second amplification reaction that uses fluorescent labeled second primer pair selective for a panel of target nucleotide sequences within a target region of a gene in the COVID-19 virus such as for example, the gene for the Spike protein, to generate fluorescent labeled COVID-19 virus amplicons. In a second method, a combined reverse transcription and asymmetric PCR amplification reaction is performed using at least one fluorescent labeled primer pair selective for the panel of target nucleotide sequences within a target region of a gene in the COVID-19 virus to generate fluorescent labeled COVID-19 virus amplicons. In either method, the fluorescent labeled COVID-19 virus amplicons are hybridized to nucleic acid probes attached at specific positions on a microarray.

This method allows positive hybridization signals to be validated on each sample tested based on internal “mismatched” and “sequence specific” controls. Additionally, at least one fluorescent signal from the hybridized amplicons associated with the panel of target nucleotide sequences within the target region of the gene is detected and an intensity distribution profile unique to each of the Clade variants generated, whereby each of the Clade variants is distinguishable from others.

In the embodiments described supra, the solid microarray support is made of any suitable material known in the art including but not limited to borosilicate glass, a thermoplastic acrylic resin (e.g., poly(methyl methacrylate-VSUVT) a cycloolefin polymer (e.g. ZEONOR 1060R), a metal including, but not limited to gold and platinum, a plastic including, but not limited to polyethylene terephthalate, polycarbonate, nylon, a ceramic including, but not limited to TiO₂, and Indium tin oxide (ITO) and engineered carbon surfaces including, but not limited to graphene. A combination of these materials may also be used. The solid support has a front surface and a back surface and is activated on the front surface by chemically activatable groups for attachment of the nucleic acid probes. In this embodiment, the chemically activatable groups include but are not limited to epoxysilane, isocyanate, succinimide, carbodiimide, aldehyde and maleimide. These materials are well known in the art and one of ordinary skill in this art would be able to readily functionalize any of these supports as desired. In a preferred embodiment, the solid support is epoxysilane functionalized borosilicate glass support.

The nucleic acid probes are attached either directly to the solid microarray support, or indirectly attached to the support using bifunctional polymer linkers. In this embodiment, the bifunctional polymer linker has a top domain and a bottom end. On the bottom end is attached a first reactive moiety that allows covalent attachment to the chemically activatable groups in the solid support. Examples of first reactive moieties include but are not limited to an amine group, a thiol group and an aldehyde group. In one aspect the first reactive moiety is an amine group. On the top domain of the bifunctional polymer linker is provided a second reactive moiety that allows covalent attachment to the oligonucleotide probe. Examples of second reactive moieties include but are not limited to nucleotide bases like thymidine, adenine, guanine, cytidine, uracil and bromodeoxyuridine and amino acid like cysteine, phenylalanine, tyrosine glycine, serine, tryptophan, cystine, methionine, histidine, arginine and lysine. The bifunctional polymer linker may be an oligonucleotide such as OLIGOdT, an amino polysaccharide such as chitosan, a polyamine such as spermine, spermidine, cadaverine and putrescine, a polyamino acid, with a lysine or histidine, or any other polymeric compounds with dual functional groups which can be attached to the chemically activatable solid support on the bottom end, and the nucleic acid probes on the top domain. Preferably, the bifunctional polymer linker is OLIGOdT having an amine group at the 5′ end.

The bifunctional polymer linker may be unmodified with a fluorescent label. Alternatively, the bifunctional polymer linker has a fluorescent label attached covalently to the top domain, the bottom end, or internally. The second fluorescent label is different from the fluorescent label in the fluorescent labeled primers. Having a fluorescent label (fluorescent tag) attached to the bifunctional polymer linker is beneficial since it allows the user to image and detect the position of the individual nucleic acid probes (“spot”) printed on the microarray. By using two different fluorescent labels, one for the bifunctional polymer linker and the second for the amplicons generated from the viral RNA being queried, the user can obtain a superimposed image that allows parallel detection of those nucleic acid probes which have been hybridized with amplicons. This is advantageous since it helps in identifying the virus comprised in the sample using suitable computer and software, assisted by a database correlating nucleic acid probe sequence and microarray location of this sequence with a known RNA signature in viruses. Examples of fluorescent labels include, but are not limited to CY5, DYLIGHT™ DY647, ALEXA FLUOR 647, CY3, DYLIGHT™ DY547, or ALEXA FLUOR 550. The fluorescent labels may be attached to any reactive group including but not limited to, amine, thiol, aldehyde, sugar amido and carboxy on the bifunctional polymer linker. In one aspect, the bifunctional polymer linker is CY5-labeled OLIGOdT having an amino group attached at its 3′terminus for covalent attachment to an activated surface on the solid support.

Moreover, when the bifunctional polymer linker also is fluorescently labeled a second fluorescent signal image is detected in the imaging step. Superimposing the first fluorescent signal image and second fluorescent signal image allows identification of the virus by comparing the sequence of the nucleic acid probe at one or more superimposed signal positions on the microarray with a database of signature sequence determinants for a plurality of viral RNA. This embodiment is particularly beneficial since it allows identification of more than one type of virus in a single assay.

The following examples are given for the purpose of illustrating various embodiments of the invention and are not meant to limit the present invention in any fashion.

### Example 1

### Microarray Assay for Clade Variant Detection

Provided herein is a method of nucleic acid analysis to detect stable genetic variation in a pathogen which is based on simultaneous analysis of multiple sequence domains in a gene, such as the Spike gene in the RNA genome of CoV-2, to measure clade variation in SARS-CoV-2. For CoV-2, the sequence domains are processed for nucleic acid analysis by converting them into a set of amplicons via a multiplex RT-PCR reaction. In a present preferred implementation, the sequence of those multiplex RT-PCR products is identified relative to that of the underlying CoV-2 Spike gene, by the Horizontal Black Bars in the bottom of Tables 4-8.

The product of that multiplex RT-PCR reaction is analyzed by hybridization to a matrix of synthetic oligonucleotide probes positioned as a microarray test (see the boxes in Tables 4-8). As seen in Tables 4-8, in a preferred implementation of the present invention for CoV-2, there are (15) such Spike Gene Target Regions containing meaningful local sequence variation. (See top Row of Tables 4-8 for their identification).

In terms of detailed test design, the oligonucleotide probes resident at each Target Region of the Spike surface protein are each produced as 3 closely related probe variants, which may be referred to as “Wild Type”, “Mutant” and “Universal”.

### Wild Type Probes

In the present invention, a “Wild Type” probe refers to an oligonucleotide probe sequence, generally 14-25 bases long that is specific to the Wuhan progenitor Clade sequence. The pattern of Multiplex RT-PCR amplicon binding to such Wild Type Probes in the present invention are displayed as superscript “2” in Table 4 and as superscript “1” in Table 6.

### Mutant Probes

“Mutant” probes correspond to an oligonucleotide probe sequence, also 15-25 bases long specific to the Sequence Change relative to the Wuhan progenitor manifest at the Spike gene location of interest are displayed as superscript “1” in Table 4 and as superscript “1” in Table 5.

### Universal Probes

A “Universal” probe refers to an oligonucleotide probe sequence (15-30 bases long) which has been designed to bind to both “Wild Type” and “Mutant” sequences at each site with similar affinity. The patterns of Multiplex RT-PCR amplicon binding to such “Universal” Probes in the present invention are displayed are displayed as superscript “1” in Table 7.

TABLE 4
Combinatorial Analysis of CoV-2 Variants

| Spike Gene Target Region | | | | | | | | | | | | | | | | | | | |
|---|---|---|---|---|---|---|---|---|---|---|---|---|---|---|---|---|---|---|---|
| (Codon) Amino Acid Change | | | S13I | L18F | T20N | P26S | Δ69-70 | D80A | D138Y | Y144DEL | W152C | R190S | D215G | A243del | R246I | K417N/T | N440K | L452R | Y453F |
| Mutation specific Probe coverage | | | ✓¹ | | ✓¹ | | ✓¹ | ✓¹ | ✓¹ | | ✓¹ | | ✓¹ | | | ✓¹ | ✓¹ | ✓¹ | |
| Wuhan reference specific probe/s coverage | | | ✓² | | ✓² | | ✓² | ✓² | ✓² | | ✓² | | ✓² | | | ✓² | ✓² | ✓² | |
| Locus specific Probe coverage | | | ✓ | | ✓ | | N/A | ✓ | ✓ | | ✓ | | ✓ | | | ✓ | ✓ | ✓ | |
| | LINEAGE DESIGNATION | | | | | | | | | | | | | | | | | | |
| REGION | var | | | | | | | | | | | | | | | | | | |
| Denmark | Mink V | B.1.1.298 | S² | L³ | T² | P³ | Δ¹ | D² | D² | Y³ | W² | R³ | D² | A³ | R³ | K² | N² | L² | F³ |
| UK | GR/501Y.V1 | B.1.1.7 | S² | L³ | T² | P³ | Δ¹ | D² | D² | Δ³ | W² | R³ | D² | A³ | R³ | K² | N² | L² | Y³ |
| SA | GH/501Y.V2 | B.1.351 | S² | L³ | T² | P³ | HV² | A¹ | D² | Y³ | W² | R³ | G¹ | Δ³ | I³ | N¹ | N² | L² | Y³ |
| Brazil/Japan | P.1 | | S² | F³ | N¹ | S³ | HV² | D² | Y¹ | Y³ | W² | S³ | D² | A³ | R³ | T² | N² | L² | Y³ |
| Brazil | P.2 | | S² | L³ | T² | P³ | HV² | D² | D² | Y³ | W² | R³ | D² | A³ | R³ | K² | N² | L² | Y³ |
| California | CAL.20C-GH/ | B. 1.429 | I¹ | L³ | T² | P³ | HV² | D² | D² | Y³ | C¹ | R³ | D² | A³ | R³ | K² | N² | R¹ | Y³ |
| | 452R.V1 | | | | | | | | | | | | | | | | | | |
| India | (Andhra Pradesh) | N440K | S² | L³ | T² | P³ | HV² | D² | D² | Y³ | W² | R³ | D² | A³ | R³ | K² | K¹ | L² | Y³ |
| WUHAN | | WUHAN | S² | L³ | T² | P³ | HV² | D² | D² | Y³ | W² | R³ | D² | A³ | R³ | K² | N² | L² | Y³ |

| | | | | | |
|---|---|---|---|---|---|
| PCR Amplimer length (bases) | (1) 101 | (2) 104 | (3) 129 | (4) 160 | (5) 199 |

| Spike Gene Target Region | | | | | | | | | | | | | | | | |
|---|---|---|---|---|---|---|---|---|---|---|---|---|---|---|---|---|
| (Codon) Amino Acid Change | | | E484K | N501Y | A570D | D6143G | H655Y | P681H | I692V | A701V | T716I | S982A | T1027I | D1118H | V11176F | M1229I |
| Mutation specific Probe coverage | | | ✓¹ | ✓¹ | | ✓¹ | | ✓¹ | | ✓¹ | | | | | | |
| Wuhan reference specific probe/s coverage | | | ✓² | ✓² | | ✓² | | ✓² | | ✓² | | | | | | |
| Locus specific Probe coverage | | | ✓ | ✓ | | ✓ | | ✓ | | ✓ | | | | | | |
| | LINEAGE DESIGNATION | | | | | | | | | | | | | | | |
| REGION | var | | | | | | | | | | | | | | | |
| Denmark | Mink V | B.1.1.298 | E² | N² | A³ | G¹ | H³ | P² | V | A² | T³ | S³ | T³ | D³ | V³ | I³ |
| UK | GR/501Y.V1 | B.1.1.7 | E² | Y¹ | D³ | G¹ | H³ | H¹ | I | A² | I³ | A³ | T³ | H³ | V³ | M³ |
| SA | GH/501Y.V2 | B.1.351 | K¹ | Y¹ | A³ | G¹ | H³ | P² | I | V¹ | T³ | S³ | T³ | D³ | V³ | M³ |
| Brazil/Japan | P.1 | | K¹ | Y¹ | A³ | G¹ | Y³ | P² | I | A² | T³ | S³ | I³ | D³ | F³ | M³ |
| Brazil | P.2 | | K¹ | N² | A³ | G¹ | H³ | P² | I | A² | T³ | S³ | T³ | D³ | F³ | M³ |
| California | CAL.20C-GH/ | B. 1.429 | E² | N² | A³ | G¹ | H³ | P² | I | A² | T³ | S³ | T³ | D³ | V³ | M³ |
| | 452R.V1 | | | | | | | | | | | | | | | |
| India | (Andhra Pradesh) | N440K | E² | N² | A³ | G¹ | H³ | P² | I | A² | T³ | S³ | T³ | D³ | V³ | M³ |
| WUHAN | | WUHAN | E² | N² | A³ | D² | H³ | P² | I | A² | T³ | S³ | T³ | D³ | V³ | M³ |

| | | | |
|---|---|---|---|
| PCR Amplimer length (bases) | (6) 151 | (7) 88 | (8) 135 |

¹AA mutation - hybridizes to mutation specific probe
²AA identical to hCoV-19/Wuhan/WIV04/2019 (WIV04) - official reference sequence employed by GISAID (EPI_ISL_402124) Hybridizes to reference specific probe
³Potential probe target

TABLE 5
Combinatorial Analysis of CoV-2 Variants - Mutant Probes

| Spike Gene Target Region | | | | | | | | | | | | | | | | | | | |
|---|---|---|---|---|---|---|---|---|---|---|---|---|---|---|---|---|---|---|---|
| (Codon) Amino Acid Change | | | S13I | L18F | T20N | P26S | Δ69-70 | D80A | D138Y | Y144DE [symbol] | W152C | R190S | D215G | A243del | R246I | K417N [symbol] | N440K | L452R | Y453F |
| Mutation specific Probe coverage | | | ✓¹ | | ✓¹ | | ✓¹ | ✓¹ | ✓¹ | | ✓¹ | | ✓¹ | | | ✓¹ | ✓¹ | ✓¹ | |
| REGION | LINEAGE DESIGNATION var | | | | | | | | | | | | | | | | | | |
| Denmark | Mink V | B.1.1.298 | S | L | T | P | Δ¹ | D | D | Y | W | R | D | A | R | K | N | L | F |
| UK | GR/501Y.V1 | B.1.1.7 | S | L | T | P | Δ¹ | D | D | Δ | W | R | D | A | R | K | N | L | Y |
| SA | GH/501Y.V2 | B.1.351 | S | L | T | P | HV | A | D | Y | W | R | G¹ | Δ | I | N¹ | N | L | Y |
| Brazil/Japan | P.1 | | S | F | N¹ | S | HV | D | Y¹ | Y | W | S | D | A | R | T¹ | N | L | Y |
| Brazil | P.2 | | S | L | T | P | HV | D | D | Y | W | R | D | A | R | K | N | L | Y |
| California | CAL.20C-GH/452R.V1 | B.1.429 | I¹ | L | T | P | HV | D | D | Y | C¹ | R | D | A | R | K | N | R¹ | Y |
| India | (Andhra Pradesh) | N440K | S | L | T | P | HV | D | D | Y | W | R | D | A | R | K | K¹ | L | Y |
| WUHAN | | WUHAN | S | L | T | P | HV | D | D | Y | W | R | D | A | R | K | N | L | Y |

| | | | | | |
|---|---|---|---|---|---|
| PCR Amplimer length (bases) | (1) 101 | (2) 104 | (3) 129 | (4) 160 | (5) 199 |

| Spike Gene Target Region | | | | | | | | | | | | | | | | |
|---|---|---|---|---|---|---|---|---|---|---|---|---|---|---|---|---|
| (Codon) Amino Acid Change | | | E484K | N501Y | A570D | D614G | H655Y | P681H | I692V | A701V | T716I | S982A | T1027I | D1118H | V1176F | M1229I |
| Mutation specific Probe coverage | | | ✓¹ | ✓¹ | | ✓¹ | | ✓¹ | | ✓¹ | | | | | | |
| REGION | LINEAGE DESIGNATION var | | | | | | | | | | | | | | | |
| Denmark | Mink V | B.1.1.298 | E | N | A | G¹ | H | P | V | A | T | S | T | D | V | I |
| UK | GR/501Y.V1 | B.1.1.7 | E | Y¹ | D | G¹ | H | H¹ | I | A | I | A | T | H | V | M |
| SA | GH/501Y.V2 | B.1.351 | K¹ | Y¹ | A | G¹ | H | P | I | V¹ | T | S | T | D | V | M |
| Brazil/Japan | P.1 | | K¹ | Y¹ | A | G¹ | Y | P | I | A | T | S | I | D | F | M |
| Brazil | P.2 | | K¹ | N | A | G¹ | H | P | I | A | T | S | T | D | F | M |
| California | CAL.20C-GH/452R.V1 | B.1.429 | E | N | A | G¹ | H | P | I | A | T | S | T | D | V | M |
| India | (Andhra Pradesh) | N440K | E | N | A | G¹ | H | P | I | A | T | S | T | D | V | M |
| WUHAN | | WUHAN | E | N | A | D | H | P | I | A | T | S | T | D | V | M |

| | | | |
|---|---|---|---|
| PCR Amplimer length (bases) | (6) 151 | (7) 88 | (8) 135 |

¹AA mutation - hybridizes to mutation specific probe [symbol]
indicates data missing or illegible when filed

TABLE 6
Combinatorial Analysis of CoV-2 Variants - Wild Type Probes

| Spike Gene Target Region (Codon) | | | | | | | | | | | | | | | | | | | |
|---|---|---|---|---|---|---|---|---|---|---|---|---|---|---|---|---|---|---|---|
| Amino Acid Change | | | S13I | L18F | T20N | P26S | Δ69-70 | D80A | D138Y | Y144DEL | W152C | R190S | D215G | A243del | R246I | K417N/T | N440K | L452R | Y453F |
| Wuhan reference specific probe/s coverage | | | ✓¹ | | ✓¹ | | ✓¹ | ✓¹ | ✓¹ | | ✓¹ | | ✓¹ | | | ✓¹ | ✓¹ | ✓¹ | |
| REGION | LINEAGE DESIGNATION var | | | | | | | | | | | | | | | | | | |
| Denmark | Mink V | B.1.1.298 | S¹ | L | T¹ | P | Δ | D¹ | D¹ | Y | W¹ | R | D¹ | A | R | K¹ | N¹ | L¹ | F |
| UK | GR/501Y.V1 | B.1.1.7 | S¹ | L | T¹ | P | Δ | D¹ | D¹ | Δ | W¹ | R | D¹ | A | R | K¹ | N¹ | L¹ | Y |
| SA | GH/501Y.V2 | B.1.351 | S¹ | L | T¹ | P | HV | A | D¹ | Y | W¹ | R | G | Δ | I | N | N¹ | L¹ | Y |
| Brazil/Japan | P.1 | | S¹ | F | N | S | HV | D¹ | Y | Y | W¹ | S | D¹ | A | R | T | N¹ | L¹ | Y |
| Brazil | P.2 | | S¹ | L | T¹ | P | HV | D¹ | D¹ | Y | W¹ | R | D¹ | A | R | K¹ | N¹ | L¹ | Y |
| California | CAL.20C-GH/ | B.1.429 | I | L | T¹ | P | HV | D¹ | D¹ | Y | C | R | D¹ | A | R | K¹ | N¹ | R | Y |
| | 452R.V1 | | | | | | | | | | | | | | | | | | |
| India | (Andhra Pradesh) | N440K | S¹ | L | T¹ | P | HV | D¹ | D¹ | Y | W¹ | R | D¹ | A | R | K¹ | K | L¹ | Y |
| WUHAN | | WUHAN | S¹ | L | T¹ | P | HV | D¹ | D¹ | Y | W¹ | R | D¹ | A | R | K¹ | N¹ | L¹ | Y |

| | | | | | |
|---|---|---|---|---|---|
| PCR Amplimer length (bases) | (1) 101 | (2) 104 | (3) 129 | (4) 160 | (5) 199 |

| Spike Gene Target Region (Codon) | | | | | | | | | | | | | | | | |
|---|---|---|---|---|---|---|---|---|---|---|---|---|---|---|---|---|
| Amino Acid Change | | | E484K | N501Y | A570D | D614G | H655Y | P681H | I692V | A701V | T716I | S982A | T1027I | D1118H | V1176F | M1229I |
| Wuhan reference specific probe/s coverage | | | ✓¹ | ✓¹ | | ✓¹ | | ✓¹ | | ✓¹ | | | | | | |
| REGION | LINEAGE DESIGNATION var | | | | | | | | | | | | | | | |
| Denmark | Mink V | B.1.1.298 | E¹ | N¹ | A | G | H | P¹ | V | A¹ | T | S | T | D | V | I |
| UK | GR/501Y.V1 | B.1.1.7 | E¹ | Y | D | G | H | H | I | A¹ | I | A | T | H | V | M |
| SA | GH/501Y.V2 | B.1.351 | K | Y | A | G | H | P¹ | I | V | T | S | T | D | V | M |
| Brazil/Japan | P.1 | | K | Y | A | G | Y | P¹ | I | A¹ | T | S | I | D | F | M |
| Brazil | P.2 | | K | N¹ | A | G | H | P¹ | I | A¹ | T | S | T | D | F | M |
| California | CAL.20C-GH/ | B.1.429 | E¹ | N¹ | A | G | H | P¹ | I | A¹ | T | S | T | D | V | M |
| | 452R.V1 | | | | | | | | | | | | | | | |
| India | (Andhra Pradesh) | N440K | E¹ | N¹ | A | G | H | P¹ | I | A¹ | T | S | T | D | V | M |
| WUHAN | | WUHAN | E¹ | N¹ | A | D¹ | H | P¹ | I | A¹ | T | S | T | D | V | M |

| | | | |
|---|---|---|---|
| PCR Amplimer length (bases) | (6) 151 | (7) 88 | (8) 135 |

¹AA identical to hCoV-19/Wuhan/WIV04/2019 (WIV04) - official reference sequence employed by GISAID (EPI_ISL_402124) Hybridizes to reference specific probe

TABLE 7
Combinatorial Analysis of CoV-2 Variants - Universal Probes

| Spike Gene Target Region | | | | | | | | | | | | | | | | | | | |
|---|---|---|---|---|---|---|---|---|---|---|---|---|---|---|---|---|---|---|---|
| (Codon) Amino Acid Change | | | S13I | L18F | T20N | P26S | Δ69-70 | D80A | D138Y | Y144DEL | W152C | R190S | D215G | A243del | R246I | K417N/T | N440K | L452R | Y453F |
| Locus specific Probe coverage | | | ✓¹ | | ✓¹ | | N/A | ✓¹ | ✓¹ | | ✓¹ | | ✓¹ | | | ✓¹ | ✓¹ | ✓¹ | |
| REGION | LINEAGE DESIGNATION var | | | | | | | | | | | | | | | | | | |
| Denmark | Mink V | B.1.1.298 | S¹ | L | T¹ | P | Δ | D¹ | D¹ | Y | W¹ | R | D¹ | A | R | K¹ | N¹ | L¹ | F |
| UK | GR/501Y.V1 | B.1.1.7 | S¹ | L | T¹ | P | Δ | D¹ | D¹ | Δ | W¹ | R | D¹ | A | R | K¹ | N¹ | L¹ | Y |
| SA | GH/501Y.V2 | B.1.351 | S¹ | L | T¹ | P | HV | A¹ | D¹ | Y | W¹ | R | G¹ | Δ | I | N¹ | N¹ | L¹ | Y |
| Brazil/Japan | P.1 | | S¹ | F | N¹ | S | HV | D¹ | Y¹ | Y | W¹ | S | D¹ | A | R | T¹ | N¹ | L¹ | Y |
| Brazil | P.2 | | S¹ | L | T¹ | P | HV | D¹ | D¹ | Y | W¹ | R | D¹ | A | R | K¹ | N¹ | L¹ | Y |
| California | CAL.20C-GH/ | B.1.429 | I¹ | L | T¹ | P | HV | D¹ | D¹ | Y | C¹ | R | D¹ | A | R | K¹ | N¹ | R¹ | Y |
| | 452R.V1 | | | | | | | | | | | | | | | | | | |
| India | (Andhra Pradesh) | N440K | S¹ | L | T¹ | P | HV | D¹ | D¹ | Y | W¹ | R | D¹ | A | R | K¹ | K¹ | L¹ | Y |
| WUHAN | | WUHAN | S¹ | L | T¹ | P | HV | D¹ | D¹ | Y | W¹ | R | D¹ | A | R | K¹ | N¹ | L¹ | Y |

| | | | | | |
|---|---|---|---|---|---|
| PCR Amplimer length (bases) | (1) 101 | (2) 104 | (3) 129 | (4) 160 | (5) 199 |

| Spike Gene Target Region | | | | | | | | | | | | | | | | |
|---|---|---|---|---|---|---|---|---|---|---|---|---|---|---|---|---|
| (Codon) Amino Acid Change | | | E484K | N501Y | A570D | D614G | H655Y | P681H | I692V | A701V | T716I | S982A | T1027I | D1118H | V1176F | M1229I |
| Locus specific Probe coverage | | | ✓ | ✓ | | ✓ | | ✓ | | ✓ | | | | | | |
| REGION | LINEAGE DESIGNATION var | | | | | | | | | | | | | | | |
| Denmark | Mink V | B.1.1.298 | E¹ | N¹ | A | G¹ | H | P¹ | V | A¹ | T | S | T | D | V | I |
| UK | GR/501Y.V1 | B.1.1.7 | E¹ | Y¹ | D | G¹ | H | H¹ | I | A¹ | I | A | T | H | V | M |
| SA | GH/501Y.V2 | B.1.351 | K¹ | Y¹ | A | G¹ | H | P¹ | I | V¹ | T | S | T | D | V | M |
| Brazil/Japan | P.1 | | K¹ | Y¹ | A | G¹ | Y | P¹ | I | A¹ | T | S | I | D | F | M |
| Brazil | P.2 | | K¹ | N¹ | A | G¹ | H | P¹ | I | A¹ | T | S | T | D | F | M |
| California | CAL.20C-GH/ | B.1.429 | E¹ | N¹ | A | G¹ | H | P¹ | I | A¹ | T | S | T | D | V | M |
| | 452R.V1 | | | | | | | | | | | | | | | |
| India | (Andhra Pradesh) | N440K | E¹ | N¹ | A | G¹ | H | P¹ | I | A¹ | T | S | T | D | V | M |
| WUHAN | | WUHAN | E¹ | N¹ | A | D¹ | H | P¹ | I | A¹ | T | S | T | D | V | M |

| | | | |
|---|---|---|---|
| PCR Amplimer length (bases) | (6) 151 | (7) 88 | (8) 135 |

¹Both Mutant and Wuhan reference sequence virus hybridize to Locus specific probe

TABLE 8
Combinatorial Analysis of CoV-2 Variants

| | | | Spike_ | Spike_ | | Spike_ | Spike_ | Spike_ | Spike_ | Spike_ | Spike_ | Spike_ | Spike_ | Spike_ | Spike_ | Spike_ | Spike_ | Spike_ |
|---|---|---|---|---|---|---|---|---|---|---|---|---|---|---|---|---|---|---|
| | | | S13I | L18F | | T20N | P26S | Δ69-70 | D80A | D138Y | Y144DE | W152C | R190S | D215G | A243del | R246I | K417N | N440K |
| Mutation specific Probe coverage | | | ✓³ | | | ✓³ | | ✓³ | ✓³ | ✓³ | | ✓³ | | ✓³ | | | ✓³ | ✓³ |
| Wuhan reference specific probe/s coverage | | | ✓¹ | | | ✓¹ | | ✓¹ | ✓¹ | ✓¹ | | ✓¹ | | ✓¹ | | | ✓¹ | ✓¹ |
| Locus specific Probe coverage | | | ✓⁴ | | | ✓⁴ | | n/a | ✓⁴ | ✓⁴ | | ✓⁴ | | ✓⁴ | | | ✓⁴ | ✓⁴ |
| REGION | LINEAGE DESIGNATION var | | | | | | | | | | | | | | | | | |
| Denmark | Mink V | B.1.1.298 | S² | L⁴ | | T² | P⁴ | Δ³ | D² | D² | Y⁴ | W² | R⁴ | D² | A⁴ | R⁴ | K² | N² |
| UK | GR/501Y.V1 | B.1.1.7 | S² | L⁴ | | T² | P⁴ | Δ³ | D² | D² | Δ⁵ | W² | R⁴ | D² | A⁴ | R⁴ | K² | N² |
| SA | GH/501Y.V2 | B.1.351 | S² | L⁴ | | T² | P⁴ | HV | A³ | D² | Y⁴ | W² | R⁴ | G³ | Δ⁵ | I⁵ | N³ | N² |
| Brazil/Japan | P.1 | | S² | F⁵ | | N³ | S⁵ | HV | D² | Y³ | Y⁴ | W² | S⁵ | D² | A⁴ | R⁴ | T³ | N² |
| Brazil | P.2 | | S | L⁴ | | T² | P⁴ | HV | D² | D² | Y⁴ | W² | R⁴ | D² | A⁴ | R⁴ | K² | N² |
| California | CAL.20C-GH/452R.V1 | B.1.429 | I³ | L⁴ | | T² | P⁴ | HV | D² | D² | Y⁴ | C³ | R⁴ | D² | A⁴ | R⁴ | K² | N² |
| India | (Andhra Pradesh) | N440K | S² | L⁴ | | T² | P⁴ | HV | D² | D² | Y⁴ | W² | R⁴ | D² | A⁴ | R⁴ | K² | K³ |
| WUHAN | | WUHAN | S² | L⁴ | | T¹ | P⁴ | HV | D¹ | D¹ | Y⁴ | W¹ | R⁴ | D¹ | A⁴ | R⁴ | K¹ | N¹ |
| | | | AMP 1 | | | | | AMP 2 | | AMP 3 | | | | AMP 4 | | | AMP 5 | |
| | | | 101 BASE | | | | | 104 BASE | | 129 BASE | | | | 160 BASE | | | 199 BASE | |
| | | | Spike_ | Spike_ | Spike_ | Spike_ | Spike_ | Spike_ | Spike_ | Spike_ | Spike_ | Spike_ | Spike_ | Spike_ | Spike_ | Spike_ | Spike_ | Spike_ |
| | | | L452R | Y453F | E484K | N501Y | A570D | D614G | H655Y | P681H | I692V | A701V | T716I | S982A | T1027I | D1118H | V1176F | M1229I |
| Mutation specific Probe coverage | | | ✓³ | | ✓³ | ✓³ | | ✓³ | | ✓³ | | ✓³ | | | | | | |
| Wuhan reference specific probe/s coverage | | | ✓¹ | | ✓¹ | ✓¹ | | ✓¹ | | ✓¹ | | ✓¹ | | | | | | |
| Locus specific Probe coverage | | | ✓⁴ | | ✓⁴ | ✓⁴ | | ✓⁴ | | ✓⁴ | | ✓⁴ | | | | | | |
| REGION | LINEAGE DESIGNATION var | | | | | | | | | | | | | | | | | |
| Denmark | Mink V | B.1.1.298 | L² | F⁵ | E² | N² | A⁴ | G³ | H⁴ | P² | V⁵ | A² | T⁴ | S⁴ | T⁴ | D⁴ | V⁴ | I⁵ |
| UK | GR/501Y.V1 | B.1.1.7 | L² | Y⁴ | E² | Y³ | D⁵ | G³ | H⁴ | H³ | I⁴ | A² | I⁵ | A⁵ | T⁴ | H⁵ | V⁴ | M⁴ |
| SA | GH/501Y.V2 | B.1.351 | L² | Y⁴ | K³ | Y³ | A⁴ | G³ | H⁴ | P² | I⁴ | V³ | T⁴ | S⁴ | T⁴ | D⁴ | V⁴ | M⁴ |
| Brazil/Japan | P.1 | | L² | Y⁴ | K³ | Y³ | A⁴ | G³ | Y⁵ | P² | I⁴ | A² | T⁴ | S⁴ | I⁵ | D⁴ | F⁵ | M⁴ |
| Brazil | P.2 | | L² | Y⁴ | K³ | N² | A⁴ | G³ | H⁴ | P² | I⁴ | A² | T⁴ | S⁴ | T⁴ | D⁴ | F⁵ | M⁴ |
| California | CAL.20C-GH/452R.V1 | B.1.429 | R³ | Y⁴ | E² | N² | A⁴ | G³ | H⁴ | P² | I⁴ | A² | T⁴ | S⁴ | T⁴ | D⁴ | V⁴ | M⁴ |
| India | (Andhra Pradesh) | N440K | L² | Y⁴ | E² | N² | A⁴ | G³ | H⁴ | P² | I⁴ | A² | T⁴ | S⁴ | T⁴ | D⁴ | V⁴ | M⁴ |
| WUHAN | | WUHAN | L¹ | Y⁴ | E¹ | N¹ | A⁴ | D¹ | H⁴ | P¹ | I⁴ | A¹ | T⁴ | S⁴ | T⁴ | D⁴ | V⁴ | M⁴ |

| | | | |
|---|---|---|---|
| AMP 5 | AMP 6 | AMP 7 | AMP 8 |
| 199 BASE | 151 BASE | 88 BASE | 135 BASE |

¹AA of hCoV-19/Wuhan/WIV04/2019 (WIV04) - official reference sequence employed by GISAID (EPI_ISL_402124)
²AA Identical to (WIV04) - hybridizes to Wuhan reference probe
³AA mutation - hybridizes to mutation specific probe
⁴Potential probe target identical to (WIV04)
⁵Potential AA mutation probe target

### Example 2

### Biological Rationale for the Design of the Present Invention

The oligonucleotide probes of the microarray and the PCR primers to generate RT-PCR amplicons were developed to accommodate a specific CoV-2 Clade Variant set of international interest in 2021, as specified in the Left-most Column in Tables 4-8. But, as can already be seen among the Clade Variant strain these tables, the pattern of local sequence change manifest in each Clade Variant comprises a unique combination derived from a larger set of specific local sequence variation chosen at discrete sites in the Spike gene.

For the Spike protein of the CoV-2 virus and for pathogens more universally, spontaneous local mutation in a surface protein such as Spike is likely to inactivate the protein, thus disabling the pathogen. As such, most spontaneous mutations in surface proteins do not propagate and hence go undetected.

On occasion, however, such random mutation produces a surface protein change that confers a selective advantage to a pathogen, such as enhanced infectivity, better resistance to vaccination or drug therapy and thus the mutation propagates in an infection and ultimately be detected at population scale. Such positively selected local mutational changes are generally rare and thus localized to a relatively small number of discrete segments within a pathogen surface protein such as Spike, often localized to specific sites where the protein contacts host cells, or sites which present peptides for interaction with a protective host antibody or sites where a drug might bind. In many cases such altered surface protein features may function in an additive way (enhanced cell binding+diminished neutralizing antibody binding may be selected for, concurrently) to produce a Clade Variant presenting a combination derived from the set of available local sequence changes that confer functional superiority to the pathogen.

The present invention takes advantage of the fundamental matrix-like character of such selectable (discrete, local) surface protein changes and the ability of a matrix of hybridization probes (as in a microarray) to query many sites of local surface protein sequence change simultaneously (Tables 4-7). As such, this oligonucleotide probe set can interrogate (at the nucleic acid level) many possible combinations of such surface protein change as a single combinatorial test.

Based on the core design test design embodied in Tables 4-7, new, as-yet unknown, functionally relevant local sequence change can be added, once known, as new probes to the microarray (cells with superscript “3” in Table 4). It is expected that many other CoV-2 Clades could be detected and discriminated, in a similar combinatorial fashion, via such relatively minor expansion of the core invention depicted in Table 4.

### Example 3

### Test Manufacturing Considerations

The present implementation of such an oligonucleotide probe panel for analysis of the CoV-2 Spike gene is based upon detection of 15 positively selected local mutational changes in the Spike gene, i.e. Tables 4-7, each with 3 probe sequence variants at each site, “Mutant”, “Wild Type”, “Universal”, thus generating a set of 15×3=45 oligonucleotide probes to be used for the purpose of combinatorial Clade Variant Analysis.

In the present implementation, if that set of 45 probes is manufactured in triplicate, a 3×45=135 probe microarray is thus generated, which when printed along with positive and negative controls appropriate for CoV-2 testing (such as RNAse P) the present Clade Chip Assay consumes the full microarray content capacity presented by the standard 150 probe, 96-Well format described in applications U.S. Ser. No. 16/950,171 and U.S. Ser. No. 16/950,210, both hereby incorporated by reference in their entireties.

It is useful to note that the information content of such a 150-probe microarray becomes resident in a single well of the 96-well microarray format and thus generates information content similar to that of re-sequencing of the entire gene and content that is equivalent to that obtained from 150 q-RT-PCR assays performed in parallel. As seen below, a first preferred implementation of such a Clade Chip prototype has been fabricated via standard mass production methods described in the above-referenced patent applications.

### Performance

In a first preferred implementation, the sample preparation methods of the Clade Chip are optimized for both NP-Swab and Saliva collection and designed to detect CoV-2 at 5 virus/RT-PCR reaction sensitivity (500 cp/ml) and resolve multiple CoV-2 Clade variants of present international concern (Denmark, UK, S Africa, Brazil/Japan, India, CA L452R, Wuhan), as depicted in Tables 4-7.

So long as any new CoV-2 Clade may be detected and discriminated via its pattern of Spike gene gRNA sequence change, that additional Clade sequence content (cells with superscript “3” in Table 4) can be designed and added to the manufacture of the present invention in less than 2-weeks, as the need for new or broader-range CoV-2 Clade Variant detection emerges.

The Clade-Chip Assay in the present preferred implementation is based on a standard 96-well plate microarray processing workflow already described in applications having U.S. Ser. No. 16/950,171 and U.S. Ser. No. 16/950,210 both hereby incorporated by reference in their entireties and is deployed in that standard 96-well format as a manual or automated test. However, the matrix of oligonucleotide probes of the present invention to detect CoV-2 Clade Variants via Combinatorial Analysis could, in principle, be implemented by other methods of microarray manufacture or via alternative methods of bead-based solution phase nucleic acid hybridization. Additionally, the same principles of Combinatorial Analysis could be used to develop analogous tests for clade variation in other viruses, bacterial and fungi in the microarray or other hybridization formats.

### Example 4

### Clade Array Manufacturing Quality and Functional Characterization

The Clade Chip Test Design summary is shown in Table 9 and is suited for combinatorial analysis among multiple Spike Targets. The following are its features;
1. Core Content, Completed (11) Target Sites, ≥3 Probes Each (Universal, Mutant, Wild Type)=11×3×3=99 probe spots
2. Additional (Future Clade) Content Array Real-estate,
   a. Up to 8 additional Target sites can be added to current Clade Array
   b. 9×3×3=81 additional probe spots.

### Clade Variant Content as Printed

A Clade Chip Probe layout was set up in duplicate. The probe content included three (3) probes for each Spike target site (Universal, Mutant, Wild Type). Validation testing was used to pick the best” of the two closely related “redundant” lead designs for each of the three probes. In addition to the core set of 11 spike targets, new probe designs were included to expand the content of the assay. The full set of redundant probe content was printed in duplicate as a 12×16 probe array in a 96-well format (Table 10). The forward (odd numbers) and reverse (even numbers) primer sequences for each amplimer employed in this assay are shown in Table 11.

TABLE 10
Clade chip probe layout

| | | | Amplimer | | |
|---|---|---|---|---|---|
| Row | Col. | SEQ ID NOS. | # | Target | Probe Sequence (5′ to 3′) |
| 1 | 1 | SEQ ID NO: 30 | 1 | AA S13I | TTTTTCTAGTCTCTAKTCAGTGTGTTTTTT |
| 1 | 2 | SEQ ID NO: 64 | 1 | AA 513_(1) | TTTTTTTGTCTCTAGTCAGTGTTTTTTTTT |
| 1 | 3 | SEQ ID NO: 65 | 1 | AA 513_(2) | TTTTTTTAGTCTCTAGTCAGTGTTTTTTTT |
| 1 | 4 | SEQ ID NO: 66 | 1 | AA_13I (1) | TTTTTTAGTCTCTATTCAGTGTTTTTTTTT |
| 1 | 5 | SEQ ID NO: 67 | 1 | AA_13I (2) | TTTTTTTAGTCTCTATTCAGTGTTTTTTTT |
| 1 | 6 | SEQ ID NO: 68 | 1 | AA T20N | TTTTTTAATYTTACAAMCAGAACTCTTTTT |
| 1 | 7 | SEQ ID NO: 69 | 1 | AA T20_(1) | TTTTTTTATCTTACAACCAGAACCTTTTTT |
| 1 | 8 | SEQ ID NO: 70 | 1 | AA T20_(2) | TTTTTTTATCTTACAACCAGAACTTTTTTT |
| 1 | 9 | SEQ ID NO: 71 | 1 | AA_20N (1) | TTTTTTATTTTACAAACAGAACTTTTTTTT |
| 1 | 10 | SEQ ID NO: 72 | 1 | AA_20N (2) | TTTTTCAATTTTACAAACAGAACTTTTTTT |
| 1 | 11 | SEQ ID NO: 68 | | RNAse P control | TTTTTTTTCTGACCTGAAGGCTCTGCGCGTTTTT |
| 1 | 12 | SEQ ID NO: 73 | 2 | AA69-70 HV (1) | TTTTTTATGCTATACATGTCTCTGTTTTTT |
| 2 | 1 | SEQ ID NO: 31 | 2 | AA69-70 HV (2) | TTTTTCCCATGCTATACATGTCTCTGTTTTTT |
| 2 | 2 | SEQ ID NO: 74 | 2 | AA69-70 DEL(1) | TTTTTTACCATGCTATCTCTGGGATTTTTT |
| 2 | 3 | SEQ ID NO: 32 | 2 | AA69-70 DEL(2) | TTTTTTTTTCCATGCTATCTCTGGGATTTTTT |
| 2 | 4 | SEQ ID NO: 33 | 2 | AA D80A | TTTTTCAGAGGTTTGMTAACCCTGTCTTTTTT |
| 2 | 5 | SEQ ID NO: 34 | 2 | AA D80_(1) | TTTTTTTGGTTTGATAACCCTGCTTTTTTT |
| 2 | 6 | SEQ ID NO: 75 | 2 | AA D80_(2) | TTTTTCTAGGTTTGATAACCCTGCTTTTTT |
| 2 | 7 | SEQ ID NO: 76 | 2 | AA_80A (1) | TTTTTTTAGGTTTGCTAACCCTCTTTTTTT |
| 2 | 8 | SEQ ID NO: 35 | 2 | AA_80A (2) | TTTTTTTGGTTTGCTAACCCTGCTTTTTTT |
| 2 | 9 | SEQ ID NO: 36 | 3 | AA D138Y | TTTTATTTTGTAATKATCCATTTTTGTTTT |
| 2 | 10 | SEQ ID NO: 37 | 3 | AA D138_(1) | TTTTTCTTTGTAATGATCCATTTTCTTTTT |
| 2 | 11 | SEQ ID NO: 77 | 3 | AA D138_(2) | TTTTTTCTTTGTAATGATCCATTTCTTTTT |
| 2 | 12 | SEQ ID NO: 38 | 3 | AA_138Y (1) | TTTTTTTTTGTAATTATCCATTTTCTTTTT |
| 3 | 1 | SEQ ID NO: 78 | 3 | AA_138Y (2) | TTTTTCTTTGTAATTATCCATTTTCTTTTT |
| 3 | 2 | SEQ ID NO: 39 | 3 | AA W1520 | TTTTTAGTTGKATGGAAAGTGAGTTCTTTT |
| 3 | 3 | SEQ ID NO: 40 | 3 | AA W152_(1) | TTTCTCTAAAAGTTGGATGGAAACTCTTCT |
| 3 | 4 | SEQ ID NO: 79 | 3 | AA W152_(2) | TTTTCTTCAAAGTTGGATGGAAACTCTTTT |
| 3 | 5 | SEQ ID NO: 41 | 3 | AA_152C (1) | TTTCTTCAAAGTTGTATGGAAAGCCTTCTT |
| 3 | 6 | SEQ ID NO: 80 | 3 | AA_152C (2) | TTTCTCTAAAAGTTGTATGGAAACTCTTCT |
| 3 | 7 | SEQ ID NO: 81 | 4 | AA D215G | TTTTTTAGTGCGTGRTCTCCCTCATTTTTT |
| 3 | 8 | SEQ ID NO: 82 | 4 | AA D215_(1) | TTTTTTCTGCGTGATCTCCCTCATTTTTTT |
| 3 | 9 | SEQ ID NO: 83 | 4 | AA D215_(2) | TTTTTTTCTGCGTGATCTCCCTCTTTTTTT |
| 3 | 10 | SEQ ID NO: 84 | 4 | AA_215G (1) | TTTTTTTTGCGTGGTCTCCCTCTTTTTTTT |
| 3 | 11 | SEQ ID NO: 85 | 4 | AA_215G (2) | TTTTTTTTTGCGTGGTCTCCCTTTTTTTTT |
| 3 | 12 | SEQ ID NO: 86 | 5 | AA K417N | TTTTAACTGGAAAKATTGCTGATTATTTTT |
| 4 | 1 | SEQ ID NO: 87 | 5 | AA K417_(1) | TTTCTTCTCTGGAAAGATTGCTGCTTTTTT |
| 4 | 2 | SEQ ID NO: 88 | 5 | AA K417_(2) | TTCTTCTCTGGAAAGATTGCTGACTTTTTT |
| 4 | 3 | SEQ ID NO: 89 | 5 | AA_417N (1) | TTTTTCTCTGGAAATATTGCTGACTTTTTT |
| 4 | 4 | SEQ ID NO: 90 | 5 | AA_417N (2) | TTTTCTCTGGAAATATTGCTGATCTTTTTT |
| 4 | 5 | SEQ ID NO: 91 | 5 | AA_417T (1) | TTTTTTTACTGGAACGATTGCTTTTTTTTT |
| 4 | 6 | SEQ ID NO: 92 | 5 | AA_417T (2) | TTTTTTCCTGGAACGATTGCTGTTTTTTTT |
| 4 | 7 | SEQ ID NO: 42 | 5 | AA N439K+N440K | TTTTTAATTCTAAMAAKCTTGATTCTAATTTT |
| 4 | 8 | SEQ ID NO: 93 | 5 | AA N439_+N440_(1) | TTTTTTATTCTAACAATCTTGATTTCTTTT |
| 4 | 9 | SEQ ID NO: 43 | 5 | AA N439_+N440_(2) | TTTTTAATTCTAACAATCTTGATTTCTTTT |
| 4 | 10 | SEQ ID NO: 94 | 5 | AA N439_+_440K (1) | TTTTTTTTTCTAACAAGCTTGATTTTTTTT |
| 4 | 11 | SEQ ID NO: 44 | 5 | AA N439_+_440K (2) | TTTTTTATTCTAACAAGCTTGATTTTTTTT |
| 4 | 12 | SEQ ID NO: 45 | 5 | AA_439K+N440_(1) | TTTTCTATTCTAAAAATCTTGATTTCTTTT |
| 5 | 1 | SEQ ID NO: 95 | 5 | AA_439K+N440_(2) | TTCTTAATTCTAAAAATCTTGATTTCTTTT |
| 5 | 2 | SEQ ID NO: 46 | 5 | AA L452R | TTTCTATAATTACCTGTATAGATTGTCTTT |
| 5 | 3 | SEQ ID NO: 96 | 5 | AA L452_(1) | TTTTTCATAATTACCTGTATAGACTTTCTT |
| 5 | 4 | SEQ ID NO: 47 | 5 | AA L452_(2) | TTTTTTTAATTACCTGTATAGATTTCTTTT |
| 5 | 5 | SEQ ID NO: 48 | 5 | AA_452R (1) | TTTTTCATAATTACTGGTATAGATCTTTTT |
| 5 | 6 | SEQ ID NO: 97 | 5 | AA_452R (2) | TTTTTTCAATTACCGGTATAGATCTTTTTT |
| 5 | 7 | SEQ ID NO: 49 | 6 | AA S477_ | TTTTTTCGCCGGTAGCACACCTCTTTTTTT |
| 5 | 8 | SEQ ID NO: 98 | 6 | AA_478I (1) | TTTTTTTTGGTAGCATACCTTGTTTTTTTT |
| 5 | 9 | SEQ ID NO: 99 | 6 | AA_478I (2) | TTTTTTTCGGTAGCATACCTTGTTTTTTT |
| 5 | 10 | SEQ ID NO: 50 | 6 | AA_477N (1) | TTTTCTTCCGGTAACACACCTTTTTTTTTT |
| 5 | 11 | SEQ ID NO: 100 | 6 | AA_477N (2) | TTTTTTCGCCGGTAACACACCTCTTTTTTT |
| 5 | 12 | SEQ ID NO: 101 | 6 | AA_476S (1) | TTTTTTTTCAGGCCAGTAGCACTTTTTTTT |
| 6 | 1 | SEQ ID NO: 51 | 6 | AA V483A+E484K | TTTTTTAATGGTGTTRAAGGTTTTAATTTTTT |
| 6 | 2 | SEQ ID NO: 52 | 6 | AA V483_+E484_(1) | TTTTTTCTGGTGTTGAAGGTTTTACTTTTT |
| 6 | 3 | SEQ ID NO: 102 | 6 | AA V483_+E484_(2) | TTTTTCTGGTGTTGAAGGTTTTATCTTTTT |
| 6 | 4 | SEQ ID NO: 103 | 6 | AA V483_+_484K (1) | TTTTTTCTGGTGTTAAAGGTTTTACTTTTT |
| 6 | 5 | SEQ ID NO: 53 | 6 | AA V483_+_484K (2) | TTTTTTTATGGTGTTAAAGGTTTTCTTTTT |
| 6 | 6 | SEQ ID NO: 54 | 6 | AA_483A+E484_(1) | TTTTTTTATGGTGCTGAAGGTTCTTTTTTT |
| 6 | 7 | SEQ ID NO: 104 | 6 | AA_483A+E484_(2) | TTTTTTCAATGGTGCTGAAGGTTCTTTTTT |
| 6 | 8 | SEQ ID NO: 55 | 6 | AA N501Y | TTTTTTTTCCAACCCACTWATGGTGTTTTTTTT |
| 6 | 9 | SEQ ID NO: 56 | 6 | AA N501_(1) | TTTTTTTTACCCACTAATGGTGTCTTTTTT |
| 6 | 10 | SEQ ID NO: 105 | 6 | AA N501_(2) | TTTTTTTAACCCACTAATGGTGTCTTTTTT |
| 6 | 11 | SEQ ID NO: 57 | 6 | AA_501Y (1) | TTTTTTTTACCCACTTATGGTGTCTTTTTT |
| 6 | 12 | SEQ ID NO: 106 | 6 | AA_501Y (2) | TTTTTTTAACCCACTTATGGTGTCTTTTTT |
| 7 | 1 | SEQ ID NO: 107 | 7 | AA D614G | TTTTTCTCTTTATCARGRTGTTAACTGCTT |
| | | | | | TTTT |
| 7 | 2 | SEQ ID NO: 108 | 7 | AA D614_ | TTTTTCTTATCAGGATGTTAACTTTTTTTT |
| 7 | 3 | SEQ ID NO: 109 | 7 | AA_614G +613(CAG) | TTTTTTCCTATCAGGGTGTTAACTTTTTTT |
| 7 | 4 | SEQ ID NO: 110 | 7 | AA_614G +613(CAA) | TTTTTTCCTATCAAGGTGTTAACTTTTTTT |
| 7 | 5 | SEQ ID NO: 111 | 7 | AA_614G | TTTTTTCCTATCARGGTGTTAACTTTTTTT |
| 7 | 6 | SEQ ID NO: 58 | 8 | AA P681H | TTTTTTCAGACTAATTCTCMTCGGCTTTTT |
| 7 | 7 | SEQ ID NO: 112 | 8 | AA P681_(1) | TTTTTTTTAATTCTCCTCGGCGTTTTTTTT |
| 7 | 8 | SEQ ID NO: 59 | 8 | AA P681_(2) | TTTTTTTCTAATTCTCCTCGGCGTTTTTTT |
| 7 | 9 | SEQ ID NO: 60 | 8 | AA_681H (1) | TTTTTTTTTAATTCTCATCGGCGTTTTTTT |
| 7 | 10 | SEQ ID NO: 113 | 8 | AA_681H (2) | TTTTTTTCTAATTCTCATCGGCGTTTTTTT |
| 7 | 11 | SEQ ID NO: 61 | 8 | AA A701V | TTTTCACTTGGTGYAGAAAATTCAGTTTTT |
| 7 | 12 | SEQ ID NO: 62 | 8 | AA A701_(1) | TCTTCTTCTTGGTGCAGAAAATTATTCTTT |
| 8 | 1 | SEQ ID NO: 114 | 8 | AA A701_(2) | TTCTTCTACTTGGTGCAGAAAATTATTCTT |
| 8 | 2 | SEQ ID NO: 63 | 8 | AA_701V (1) | TCTTCTTCTTGGTGTAGAAAATTATTCTTT |
| 8 | 3 | SEQ ID NO: 115 | 8 | AA_701V (2) | TTTCTTTCTTGGTGTAGAAAATTCTTTTTT |
| 8 | 4 | SEQ ID NO: 116 | | N2 | TTTTTTACAATTTGCCCCCAGCGTCTTTTT |
| 8 | 5 | SEQ ID NO: 117 | | SARS-2003 N2 | TTTTTTTTTGCTCCRAGTGCCTCTTTTTTT |
| 8 | 6 | SEQ ID NO: 70 | | Negative Control | TTTTTTCTACTACCTATGCTGATTCACTCT |
| | | | | | TTTT |
| 8 | 7 | EMPTY | | | |
| 8 | 8 | EMPTY | | | |
| 8 | 9 | EMPTY | | | |
| 8 | 10 | EMPTY | | | |
| 8 | 11 | EMPTY | | | |
| 8 | 12 | EMPTY | | | |

TABLE 11
Amplimer primer sequences

| | Amplimer | | | |
|---|---|---|---|---|
| SEQ ID NOS. | # | Target | Gene | Primer Sequence (5′ to 3′) |
| SEQ ID NO: 25 | 2 | AA66-85 | Spike | TTCTTTTCCAATGTTACTTGGTT |
| | | | | CCATG |
| SEQ ID NO: 26 | 2 | AA66-85 | Spike | Cy3-TTTCAAAATAAACACCATC |
| | | | | ATTAAATGG |
| SEQ ID NO: 11 | 3 | AA126-157 | Spike | TTTCTTATTGTTAATAACGCTAC |
| | | | | TAATG |
| SEQ ID NO: 12 | 3 | AA126-157 | Spike | Cy3-TTTCATTCGCACTAGAATA |
| | | | | AACTCTGAA |
| SEQ ID NO: 27 | 5 | AA413-458 | Spike | TTTGATGAAGTCAGACAAATCG |
| | | | | CTCCAG |
| SEQ ID NO: 28 | 5 | AA413-458 | Spike | Cy3-TTTCTCTCAAAAGGTTTGA |
| | | | | GATTAGACT |
| SEQ ID NO: 15 | 6 | AA475-506 | Spike | TTTTATTTCAACTGAAATYTATCA |
| | | | | GGCC |
| SEQ ID NO: 16 | 6 | AA475-506 | Spike | Cy3-TTTAAAGTACTACTACTCT |
| | | | | GTATGGTTG |
| SEQ ID NO: 29 | 7 | AA610-618 | Spike | TTTCAAATACTTCTAACCAGGTT |
| | | | | GCTGT |
| SEQ ID NO: 24 | 7 | AA610-618 | Spike | Cy3-TTTTGCATGAATAGCAACA |
| | | | | GGGACTTCT |
| SEQ ID NO: 17 | 8 | AA677-707 | Spike | TTTTATATGCGCTAGTTATCAGA |
| | | | | CTCAG |
| SEQ ID NO: 18 | 8 | AA677-707 | Spike | Cy3-TTTTGGTATGGCAATAGAG |
| | | | | TTATTAGAG |

### Example 5

### Clade Array Functional Characterization

### Experiment 1:

Samples Used for Testing. Analysis was performed with a highly characterized, purified Wuhan gRNA standard (Quantitative Standard obtained from ATCC-BEI) or with synthetic “mutant” targets designed by PDx, obtained by SGI fabrication (IDT).

RT-PCR Conditions. RT-PCR was performed using the [UNG+One-Step RT-PCR] protocol. As is customary in optimization of multiplex RT-PCR, the data presented comprise the use of Single PCR primer pairs as a single reaction. Based on these data multiplex RT-PCR conditions are optimized.

Clade Array Hybridization & Imaging. Conditions of Hybridization, Washing and Imaging were exactly as described. Following the completion of the multiplex RT-PCR, the DNA microarray was prepared for hybridization with brief water washes, and an incubation in prehybridization buffer (0.6M NaCl, 0.06M sodium citrate solution, 0.1% Ficoll, 0.1% Polyvinylpyrrolidone 0.1% Bovine Serum Albumin). Following aspiration of the prehybridization buffer, a mixture of amplicon and hybridization buffer (0.6M NaCl, 0.06M sodium citrate solution, 0.1% Ficoll, 0.1% Polyvinylpyrrolidone 0.1% Bovine Serum Albumin) was added to the DNA microarray and allowed to incubate for 2 hours. The microarray was prepared for imaging with one quick wash of wash buffer (22.5 mM NaCl, 2.25 mM sodium citrate solution) and a 10-minute incubation (22.5 mM NaCl, 2.25 mM sodium citrate solution). The microarray plate was then spun dry for 5 minutes at 2200 rpm. The underside of the plate was wiped clean with 70% ethanol and lens tissue until all dust particles were removed. The plate was scanned on the Sensospot utilizing Sensovation software. Cy5 exposure time was set at 312 ms, and the Cy3 exposure times at 115 ms and 578 ms. Upon image scanning completion, the folder containing all of the scanned data was saved to a thumb drive and uploaded to Dropbox for Augury Analysis.

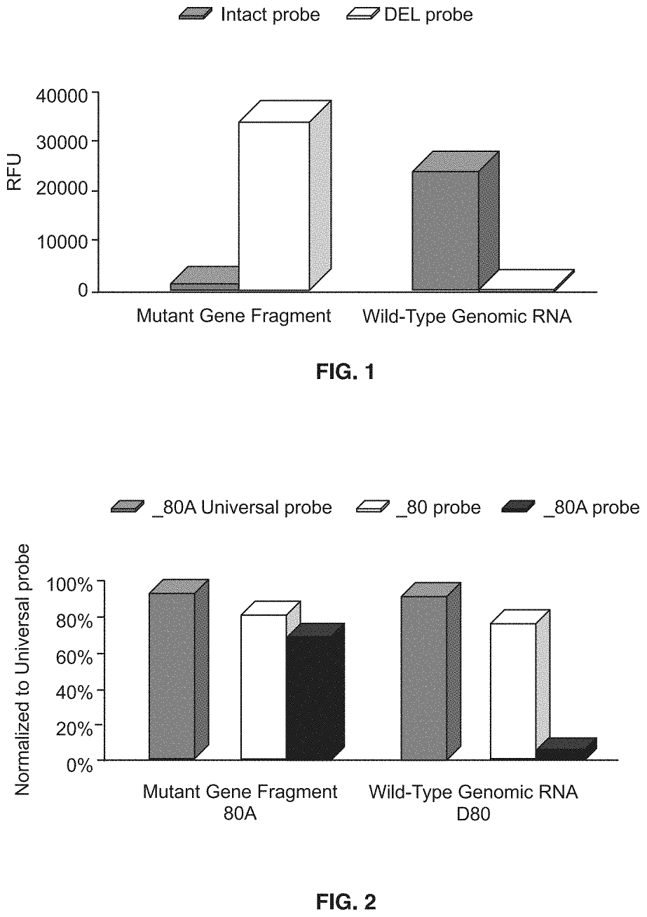

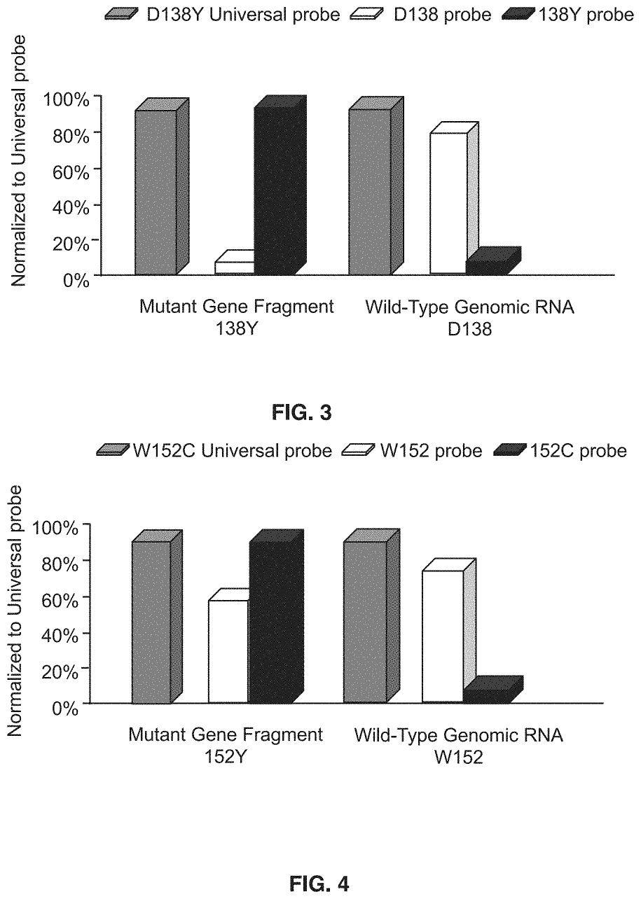

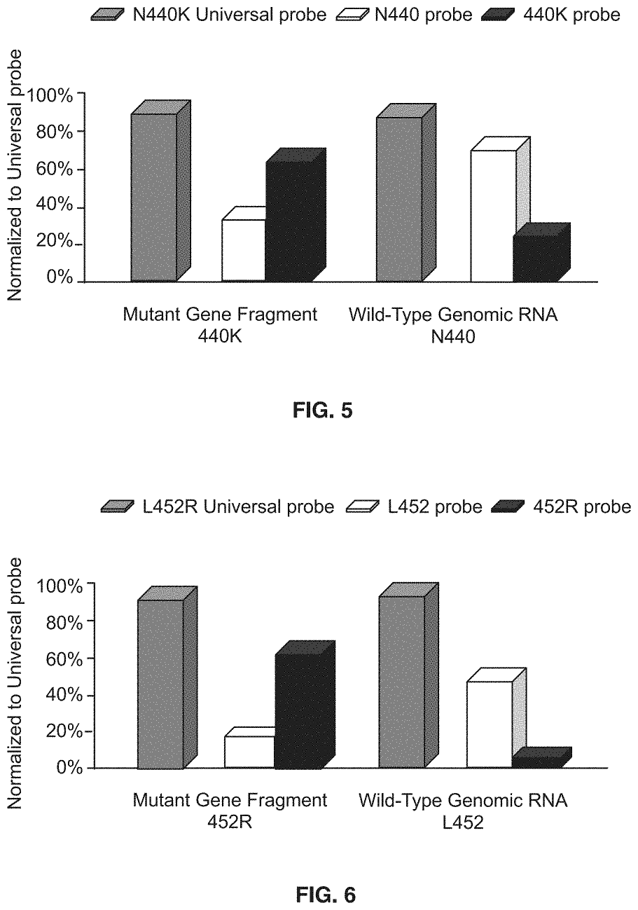

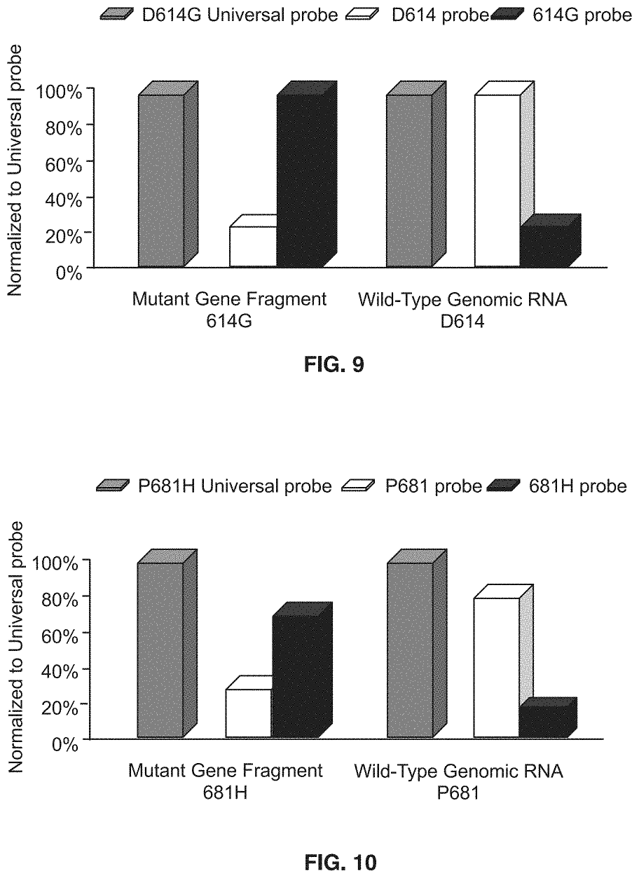

Data Analysis. Data for all (11) Core Spike Target Sites are presented in FIGS. 1-11. For all Spike Target sites, data is presented as a bar graph, where the ratio of hybridization signal strength for Wild Type vs Mutant Probes define the specificity of analysis.
a) Left Side of each bar graph shows hybridization data derived from analysis of the “Mutant” synthetic CoV-2 target sequence appropriate for that site.
b) Right Side of each bar graph displays the corresponding data obtained for the “Wild Type” Wuhan gRNA reference sequence.
c) In the analysis of each bar graph at each target site, the parameter of analytical importance is the hybridization signal strength (RFU) ratio obtained by comparison of RT-PCR amplimer hybridization to the Mutant-Specific vs Wild-Type specific probe which can be presented as [RFU_{wt}/RFUₘₜ].
i. Criterion #1. Primary Data. The [RFU_{wt}/RFUₘₜ] ratio should differ significantly (>5×) upon comparison of “Wild Type” CoV2 genome hybridization at that site, vs “Mutant” Template hybridization measured at that same site.
ii. Criterion #2. Optimal but not necessarily. The [RFU_{wt}/RFUₘₜ] ratio should change qualitatively upon CoV2 hybridization analysis of a “Wild Type” CoV2 genome at that site, vs a “Mutant” Template measured at that site. i.e.
   Wild-Type Template→[RFU_{wt}/RFUₘₜ]>1
   Mutant Template→[RFU_{wt}/RFUₘₜ]<1

### Clade Array Results

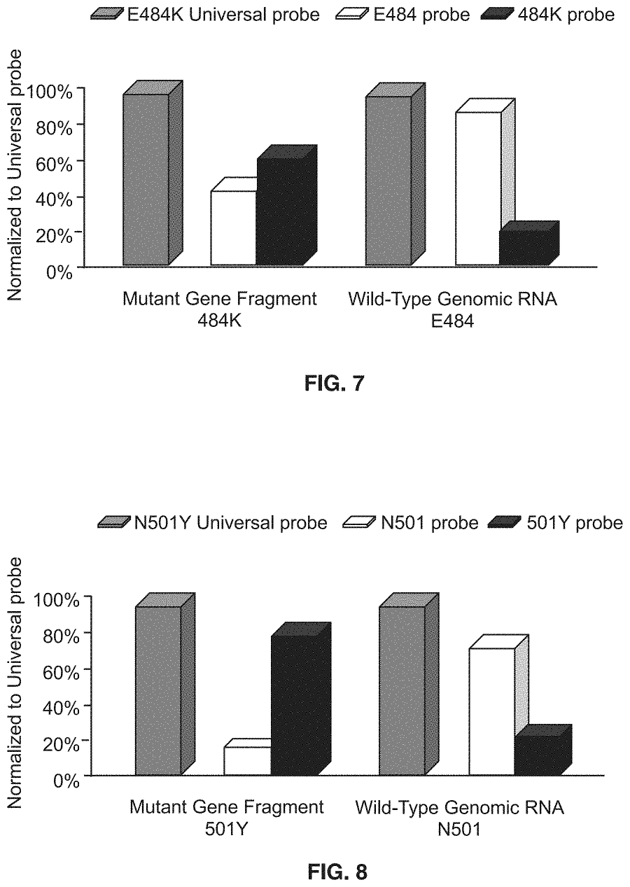

a) The Data, Presented in FIGS. 1-11 demonstrate that all (11) Spike Target sites show generally excellent hybridization-based discrimination between Wild Type vs Mutant Sequence Variants by the primary Criterion #1 listed above.
b) For (9) of the 11, Both Criterion #1 and Criterion #2 have been met. All such sites are thus marked among FIGS. 1-11 as “No Additional Probe Optimization Required” and are marked by a “*” below their location in the probe matrix of Table 9.
c) For two of the 11 Spike Target sites (D80A and E484K, FIGS. 2 and 7), Criterion #2 was not met adequately.
d) Only two Probes need Optimization. In two cases (Target Sites D80A and E484K) although the Wild Type (Wuhan gRNA) is easily distinguished from the Mutant based on significant differences in relative hybridization (Criterion #1, above), the Mutant/Wild Type distinction does not change sign (Criterion #2). Thus, although the data obtained at those (2) sites (D80A, E484K) is adequate at present to make accurate “Clade Calls”, modest Optimization (1 Probe at each site) can be deployed quickly to enhance the quality of the data obtained there.
e) Experience and general understanding of nucleic acid biophysics suggest that the needed optimization (Target Sites D80A and E484K) was obtainable by simply reducing the length of one probe at each site by 2 bases. As soon as the need for such.

### Summary

The Clade Array Probe Content was found to be fully functional. An optimized shorter probe was seen to improve Cov-2 mutant analysis at target sites D80A and E484K.

### Experiment 2:

A second 15 Plate Manufacturing Run (#2) of DETECTX-Cv, similar to the one described in Experiment 1 above was implemented to complete validation of the Multiplex assay. In this assessment, print quality passed the test for all 96 (160 probe) arrays among all 15 plates.

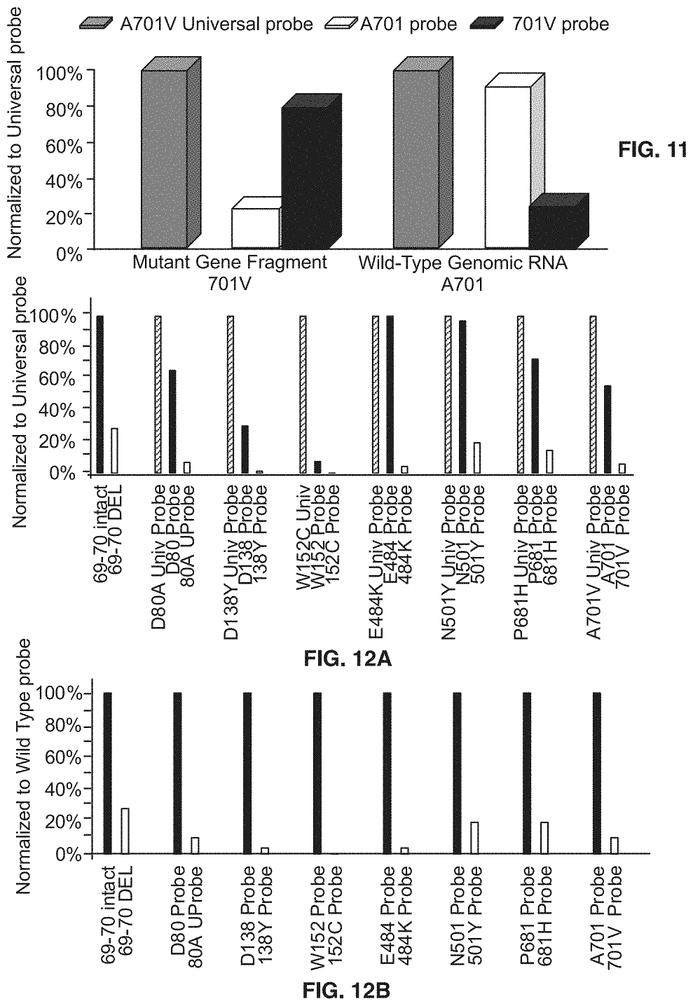

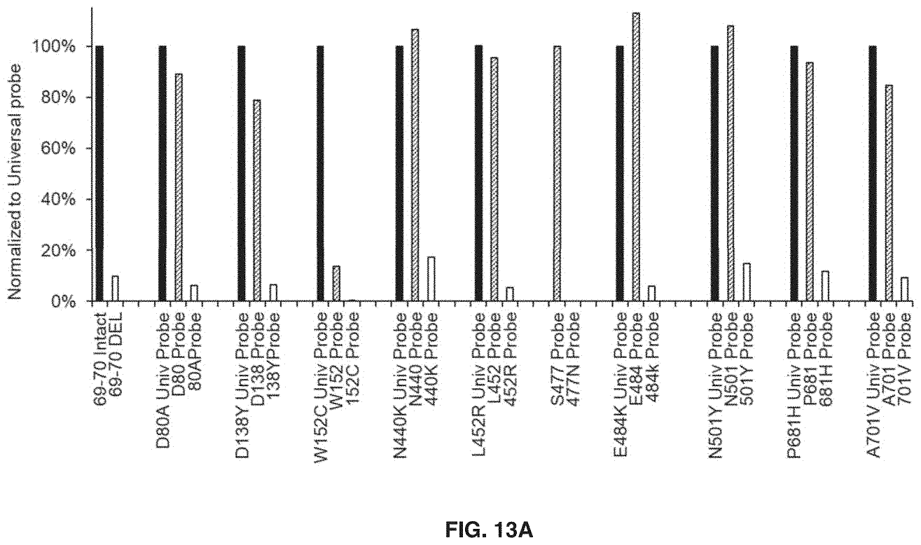

The second set of validation tests sought to evaluate the preferred method of multiplexing of the RT-PCR reaction, using the UNG combined with One Step RT-PCR condition. The primary goal was to deliver a first RT-PCR Multiplex capable of distinguishing the five prevalent Clade Variants—UK (B.1.1.7), S Africa (B.1.351) Brazil (P.1) Brazil (P.2) US California (B.1.429) shown in Table 12. The validation materials comprised a purified Wuhan gRNA reference (ATCC-BEI). The data obtained subsequent to RT-PCR, hybridization and washing revealed that an initial deployment of a specific 4-plex RT-PCR reaction, comprising amplimers [2, 3, 6, 8] was sufficient to distinguish, as a single multiplex assay, these five prevalent Clade variants (FIGS. 12A-12B).

FIG. 12A shows the raw microarray hybridization data for the eight (8) target sites covered by amplimer sets 2, 3, 6 and 8 with data normalized to the Universal Probe at “100%” to emphasize the sensitivity of Universal vs Wild-Type/Mutant Target Detection. FIG. 12B shows the same raw hybridization data for the eight (8) target sites covered by the amplimer sets 2, 3, 6 and 8, but normalized to the Wild-Type probe signal, to emphasize the specificity of discrimination between Wild-Type vs Mutant target sequence.

### Experiment 3:

### Fully Multiplexed DETECTX-Cv.

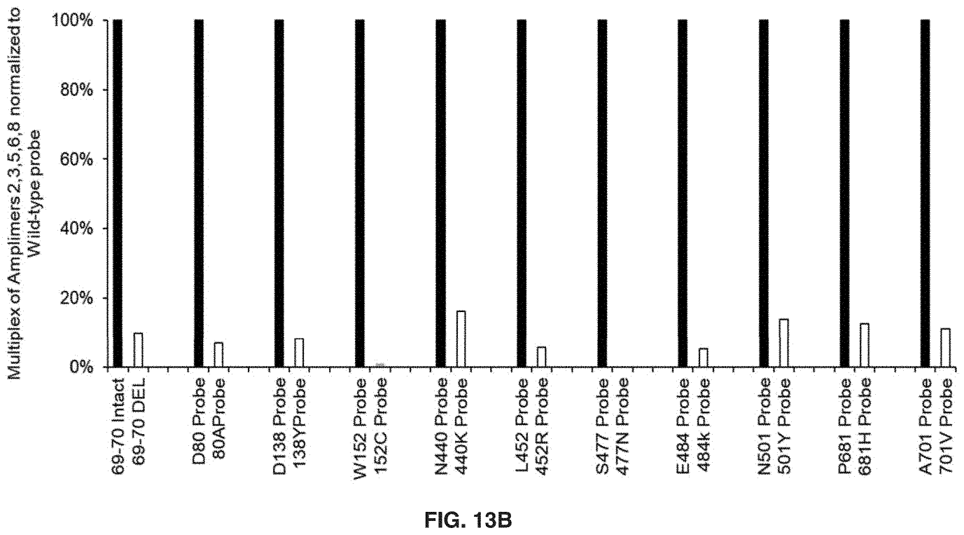

A third round of validation was performed to evaluate the preferred method of multiplexing of the RT-PCR reaction, using the UNG combined with One Step RT-PCR condition. The primary goal was to deliver a second (N=5) RT-PCR Multiplex capable of distinguishing the six (6) prevalent US Clade Variants—UK (B.1.1.7), S Africa (B.1.351) Brazil (P.1) Brazil (P.2) a second redundant target in US California (B.1.429) and India N440K. shown in Table 12. The validation materials comprised a purified Wuhan gRNA reference (ATCC-BEI). The data obtained subsequent to RT-PCR, hybridization and washing revealed that a second deployment of a specific 5-plex RT-PCR reaction, comprising a N=5 multiplex of amplimers [2, 3, 5, 6, 8] was sufficient to distinguish, as a single multiplex assay, these six prevalent Clade Variants (FIGS. 13A-13B).

FIG. 13A shows the raw microarray hybridization data for the eleven (11) target sites covered by amplimer sets 2, 3, 5, 6 and 8 with data normalized to the Universal Probe at “100%” to emphasize the sensitivity of Universal vs Wild-Type/Mutant Target Detection. FIG. 13B shows the same raw hybridization data for the eleven (11) target sites covered by the amplimer sets 2, 3, 5, 6 and 8, but normalized to the Wild-Type probe signal, to emphasize the specificity of discrimination between Wild-Type vs Mutant target sequence.

Table 13 shows the information content for the fully multiplexed (2, 3, 5, 6, 8) data obtained via the multiplex RT-PCR reaction in this DETECTX-Cv assay, which is sufficient to discriminate the five clade variants (superscript “1”). It was determined that including Amplimer 5 to the multiplex adds redundancy (superscript “2”) thereby allowing unambiguous discrimination of the India Mutant (B.1.36.29). Similarly, addition of amplimer 4 (for NY B.1.526) and Q677P/H probes (for Southern US B.1.596/13.1.2) to the multiplex enabled discrimination of Southern US and NY Clade variants (superscript “3”). Importantly, the emerging Southern US Clade variants (B.1.596/1.1.2) does not require modification of the present multiplex reaction since inclusion of probes at Q677P/H would be sufficient. Analytical specificity was established as described earlier via analysis of both wild type (Wuhan) gRNA and synthetic, Clade specific fragments.

TABLE 13
Information content obtained by addition of amplimers

| | | | | | Information |
|---|---|---|---|---|---|
| | | | | | obtained by |
| | | | | | adding |
| | | | | Information | Amplimer 4 |
| | | | Information | obtained | + New |
| | | | obtained with | by adding | Clade |
| | | | Amplimers 2, 3, | Amplimer | variant |
| Region | Lineage Designation Var | | 6, 8 | 5 | probes |
| Denmark | Mink V | B.1.1.298 | ✓¹ | | |
| UK | GR/501Y.V1 | B.1.1.7 | ✓¹ | | |
| SA | GH/501Y.V2 | B.1.351 | ✓¹ | ✓² | ✓³ |
| Brazil/Japan | P.1 | | ✓¹ | ✓² | |
| Brazil | P.2 | | ✓¹ | | |
| California | CAL.20C-GH/452R.V1 | B.1.429 | ✓¹ | ✓² | |
| India | (Andhra Pradesh) | N440K | | ✓² | |
| S. US | B.1.596/B.1.2 | Q677P /H | | | ✓³ |
| NY | Ho et al. | B.1.526 | | | ✓³ |
| | | B.1.526.2 | | | ✓³ |
| WUHAN | | | ✓¹ | ✓² | ✓³ |

### Augury Software Modification

Current deployment of Augury software was modified to include automated capacity for determining “Wild-Type” vs “Mutant” at each of the (11) Spike target sites of the present DETECTX-Cv assay described above (the columns in Table 12. As modified, Augury is capable of calling the identity of the clade variant, based on the pattern of mutant presentation among the sites (that is, a “look-up” table comprising the pattern of each row of Table 12). Coding to enable such autonomous calling is based on allelotyping methods previously developed for HLA allelotyping. In the present case, the clade variant test is also an exercise in spike gene allelotyping. Such spike gene allelotypes (the rows in Table 12) have already been determined as being the preferred marker for CoV-2 Clade Variation.

TABLE 14
New Clade variant probe″ sequences

| | Amplimer | | |
|---|---|---|---|
| SEQ ID NOS. | # | Target | Probe Sequence (5′ to 3′) |
| SEQ ID NO: 118 | 4 | AA A243_ | TTTTTTTTCAAACTTTACTTGCTTTACTC |
| | | | TTT |
| SEQ ID NO: 119 | 4 | AA_243DEL | TTTTTTTTCAAACTTTACATAGAAGCCTT |
| | | | TTT |
| SEQ ID NO: 120 | 4 | AA R246_ | TTTTCTACATAGAAGTTATTTGACTCCCT |
| | | | TTT |
| SEQ ID NO: 121 | 4 | AA_246I | TTTTCTGCTTTACATATGACTCCTGGTTT |
| | | | TTT |
| SEQ ID NO: 122 | 4 | AA D253G | TTTCTACTCCTGGTGRTTCTTCTTCATTT |
| | | | T |
| SEQ ID NO: 123 | 4 | AA D253_ | TTTTTTCCCTGGTGATTCTTCTTTCTTTT |
| | | | T |
| SEQ ID NO: 124 | 4 | AA_253G | TTTTTTCCCTGGTGGTTCTTCTTTTTTTT |
| | | | T |
| SEQ ID NO: 125 | 8 | AA Q677P/H | TTTTTTATCAGACTCMGACTAATTCTCTT |
| | | | TTT |
| SEQ ID NO: 126 | 8 | AA Q677_ | TTTTTTCCAGACTCAGACTAATTTCTTTT |
| | | | T |
| SEQ ID NO: 127 | 8 | AA_677P | TTTTTCTTCAGACTCCGACTAATCTTTTT |
| | | | T |
| SEQ ID NO: 128 | 8 | AA_677H1 | TTTTTTCCAGACTCATACTAATTTCTTTT |
| | | | T |
| SEQ ID NO: 129 | 8 | AA_677H2 | TTTTTTCCAGACTCACACTAATTTCTTTT |
| | | | T |

### Example 6

Augury Modification with Clade ID Module

The current deployment of the Augury software for wild type COV-2 was modified to include automated capacity for determining “Wild-Type” vs “Mutant” at each of the Spike target sites of the DETECTX-Cv assay (the columns in Table 15) and to identify the Clade variant based on the pattern of mutant presentation among the sites (the rows in Tables 15 and 16). Coding for the software is based on allelotyping formalism previously developed for HLA allelotyping.

### Augury Software for DETECTX-Cv

All DETECTX-Cv probe sequences and their information content were added to a database (“Dot Score” file) within Augury. This database defined the DETECTX-Cv probe content (Mutant, Wild Type, Universal) at each of the eleven (11) Spike target regions (the columns in Table 15).

### Establishment of DETECTX-Cv Version Control

The Augury Software is configured to read the bar code associated with each 96-well plate of microarrays for DETECTX-Cv and use the information in the bar code to create a “Dot Score” file for the probe content introduced into DETECTX-Cv. Further, Augury is configured to incorporate a new “Dot Score” file as appropriate for any new Clade Variant content with additional probes in the array (Table 15). Additionally, Augury is intrinsically cloud enabled and configured to deploy software modification downloaded from the cloud. When useful for analysis of DETECTX-Cv, data such as those from the RADx Rosalind initiative can also be introduced directly into Augury autonomously, to update the list of prevalent clade variants.

### Manual Deployment Version of Augury for DETECTX-Cv

The core functionality of Augury has been used as a manual product for deployment at TriCore. This version of Augury automatically is enabled to read DETECTX-Cv plate bar codes, perform microarray image analysis, create “Dot Score” files and present the resulting averaged, background subtracted DETECTX-Cv data as a spread sheet matrix, which can be compared to the Clade Variant Hybridization patterns such as described in Table 15. This manual deployment version has been tested on DETECTX-Cv synthetic Clade variant standards.

### Clade Variant “Look Up Table”

All prevalent Cov-2 Clades have been programmed into Augury to generate a “Look-up Table” (equivalent in content to the pattern of boxes having superscript 1 and 2 in Table 15). The Augury internal “Lookup Table” is formatted to function as part of a Boolean pattern search as developed previously for allelotype analysis of all genes.

TABLE 15
Validation of Clade variants

| Spike Gene Target Region | | | | | | | A67V/ | | | | | | | | | | | | | |
|---|---|---|---|---|---|---|---|---|---|---|---|---|---|---|---|---|---|---|---|---|
| (Codon) Amino Acid Change | | S13I | L18F | T20N | P26S | Q52R | Δ69-70 | D80A | T95I | D138Y | Y144DEL | W152C | F157L | R190S | D215G | A222V | A243del | R246I | D253G | V367F |
| Mutation specific Probe coverage | | | | | | | ✓¹ | ✓¹ | | ✓¹ | ✓¹ | ✓¹ | | | | | ✓¹ | ✓¹ | ✓¹ | |
| Wuhan reference specific probe/s | | | | | | | ✓² | ✓² | | ✓² | ✓² | ✓² | | | | | ✓² | ✓² | ✓² | |
| coverage | | | | | | | | | | | | | | | | | | | | |
| Locus specific Probe coverage | | | | | | | N/A | ✓ | | ✓ | ✓ | ✓ | | | | | ✓ | ✓ | ✓ | |
| | Pango lineage - | | | | | | | | | | | | | | | | | | | |
| Street name | (Clade Nexstrain) | | | | | | | | | | | | | | | | | | | |
| UK | B.1.1.7 - | S³ | L³ | T³ | P³ | Q³ | Δ¹ | D² | T³ | D² | Δ¹ | W² | F³ | R³ | D³ | A³ | A² | R² | D² | V³ |
| | (20I/501Y.V1) | | | | | | | | | | | | | | | | | | | |
| SA | B.1.351 - | S³ | L³ | T³ | P³ | Q³ | HV² | A¹ | T³ | D² | Y² | W² | F³ | R³ | G | A³ | Δ¹ | I¹ | D² | V³ |
| | (20H/501Y.V2) | | | | | | | | | | | | | | | | | | | |
| US | B.1.375 | S³ | L³ | T³ | P³ | Q³ | Δ¹ | D² | T³ | D² | Y² | W² | F³ | R³ | D³ | A³ | A² | R² | D² | V³ |
| Brazil | P.1 - (20J/501Y.V3) | S³ | F | N | S | Q³ | HV² | D² | T³ | Y¹ | Y² | W² | F³ | S | D³ | A³ | A² | R² | D² | V³ |
| Cal L452R | B.1.429/427 - | I | L³ | T³ | P³ | Q³ | HV² | D² | T³ | D² | Y² | C¹ | F³ | R³ | D³ | A³ | A² | R² | D² | V³ |
| | (20C/S:452R) | | | | | | | | | | | | | | | | | | | |
| Rio de Jan. | B.1.1.28 | S³ | L³ | T³ | P³ | Q³ | HV² | D² | T³ | D² | Y² | W² | F³ | R³ | D³ | A³ | A² | R² | D² | V³ |
| Andrah Pradesh | B.1.36.29 | S³ | L³ | T³ | P³ | Q³ | HV² | D² | T³ | D² | Y² | W² | F³ | R³ | D³ | A³ | A² | R² | D² | V³ |
| S. US/Q677P/H | (S:677P.B.1.596) | S³ | L³ | T³ | P³ | Q³ | HV² | D² | T³ | D² | Y² | W² | F³ | R³ | D³ | A³ | A² | R² | D² | V³ |
| | (S:677H.B.1.2) | S³ | L³ | T³ | P³ | Q³ | HV² | D² | T³ | D² | Y² | W² | F³ | R³ | D³ | A³ | A² | R² | D² | V³ |
| NYC (Ho etal) | B.1.526a - | S³ | L³ | T³ | P³ | Q³ | HV² | D² | I | D² | Y² | W² | F³ | R³ | D³ | A³ | A² | R² | G¹ | V³ |
| | (20C/S:484K) | | | | | | | | | | | | | | | | | | | |
| | B.1.526b | S³ | L³ | T³ | P³ | Q³ | HV² | D² | I | D² | Y² | W² | F³ | R³ | D³ | A³ | A² | R² | G¹ | V³ |
| NYC | B.1.525 - | S³ | L³ | T³ | P³ | R | V/Δ¹ | D² | T³ | D | Δ¹ | W | F³ | R³ | D³ | A³ | A² | R | D | V³ |
| | (20A/S:484K) | | | | | | | | | | | | | | | | | | | |
| | A.23.1 | S³ | L³ | T³ | P³ | Q³ | HV² | D² | T³ | D² | Y² | W² | L | R³ | D³ | A³ | A² | R² | D² | F |
| | B.1.258 - | S³ | L³ | T³ | P³ | Q³ | Δ¹ | D² | T³ | D² | Y² | W² | F³ | R³ | D³ | A³ | A² | R² | D² | V³ |
| | (20A/S:439K) | | | | | | | | | | | | | | | | | | | |
| | B.1.1.33 | S³ | L³ | T³ | P³ | Q³ | HV² | D² | T³ | D² | Y² | W² | F³ | R³ | D³ | A³ | A² | R² | D² | V³ |
| | B.1.177 - | S³ | L³ | T³ | P³ | Q³ | HV² | D² | T³ | D² | Y² | W² | F³ | R³ | D³ | V | A² | R² | D² | V³ |
| | (20E (EU1) | | | | | | | | | | | | | | | | | | | |
| | (S:A222V)) | | | | | | | | | | | | | | | | | | | |
| | B.1.1.207 | S³ | L³ | T³ | P³ | Q³ | HV² | D² | T³ | D² | Y² | W² | F³ | R³ | D³ | A³ | A² | R² | D² | V³ |
| Mink/Cluster V | B.1.1.298 | S³ | L³ | T³ | P³ | Q³ | Δ¹ | D² | T³ | D² | Y² | W² | F³ | R³ | D³ | A³ | A² | R² | D² | V³ |
| | (S:Y453F) | | | | | | | | | | | | | | | | | | | |

TABLE 16
Information content obtained by addition of amplimers

| | | | | Potential Probe | Amplimer 4 + |
|---|---|---|---|---|---|
| | Pango lineage | Amplimers | Amplimer | content as described | New Clade |
| Street Name | (Clade Nextstrain open-source toolkit) | 2, 3, 6, 8 | 5 | in Table 14 | variant probes |
| UK | B.1.1.7 - (201/501Y.V1) | ✓¹ | | ✓³ | |
| SA | B.1.351 - (20H/501Y.V2) | ✓¹ | ✓² | | ✓⁴ |
| US | B.1.375 | ✓¹ | | | |
| Brazil | P.1 - (20J/501Y.V3) | ✓¹ | ✓² | | |
| California L452R | B.1.429/427 - (20C/S:452R) | ✓¹ | ✓² | | |
| Rio de Janeiro | B.1.1.28 | ✓¹ | | | |
| Andhra Pradesh | | | ✓² | | |
| S.US/Q677P/H | (S:677P.Pelican) (S:677H.Robin1) | | | ✓³ | |
| NYC (Ho et al.) | B.1.526a - (20C/S:484K) | | | | ✓⁴ |
| | B.1.526a - (20C/S:484K) | | | | ✓⁴ |
| NYC | B.1.525 - (20A/5:484K) | | | ✓³ | |
| Mink / Cluster V | (S:Y453F) | ✓¹ | | ✓³ | |
| WUHAN | | ✓¹ | ✓² | ✓³ | ✓⁴ |

¹Information obtained by adding Amplimers 2, 3, 6 and 8
²Information obtained by adding Amplimer 5
³Information obtained by adding Potential probe content
⁴Information obtained by adding Amplimer 4 + New Clade variant probes

### Analytical Threshold Values

Multiplex RT-PCR [2, 3, 5, 6, 8] were performed in the absence of template (0 copies/reaction) to obtain the mean and STD from the mean for LoB signals. This “blank” data collection data is used by Augury to obtain the analytical threshold for each probe (3.2×STD+Mean) to yield Mutant threshold (Tm), Wild Type threshold (Tw) and Universal threshold (Tu) values for all thirty-three (33) probes comprising the content of DETECTX-Cv.

### Deployment of Automatic Mutant Vs Wild Type Detection (“Delta”).

Threshold values were introduced as constants into Augury for autonomous Mutant vs Wild Type determination at all eleven (11) sites. This was performed using the following relationship analytical approach;

Delta=([RFUₘ−Tₘ]/Tₘ)−([RFU_{w}−T_{w}]/T_{w}) (Equation 1)

where,
RFUₘ=mutant probe RFU signal in a sample
RFU_{w}=wild type probe RFU signal in a sample
Tₘ=mutant probe RFU Threshold—a constant obtained from CLSI (LoB) analysis
T_{w}=wild type probe RFU Threshold—a constant obtained from CLSI (LoB) analysis
[RFUₘ−Tₘ]=Mutant Probe Signal strength above Threshold. By definition, this is a non-zero value.
[RFU_{w}−T_{w}]=Wild Type Signal strength above Threshold. By definition, this is a non-zero value.
Delta=Difference in Signal Strength above Threshold normalized to Threshold

If Delta >0, within experimental accuracy, then “Mutant” (i.e. boxes having superscript 1 in Table 15). If Delta <0, within experimental accuracy, then “Wild Type” (i.e. boxes having superscript 2 in Table 15).

### Example 7

### Clade Variant Array Deployment-1

1. Analytical LoD Determination. A first determination of analytical LoD was performed for DETECTX-Cv, among all eleven (11) Spike target sites deployed using the [UNG+ One Step RT-PCR] conditions. For this analysis, validation materials comprised a purified Wuhan gRNA reference (ATCC-BEI) and a cocktail of five (5) synthetic fragments designed by PathogenDx and fabricated by Integrated DNA Technologies, Inc. (IDT, Coralville, Iowa), comprising each region targeted for amplification via the [2, 3, 5, 6, 8] multiplex RT-PCR reaction (deployed as N=5 multiplex).

To support the multiplex reaction, all 5 synthetic CoV-2 fragments were mixed [1:1:1:1:1] in strand equivalents. Copy number values listed in Table 15 refer to the copy number of each fragment (in the equimolar mix) applied to the RT-PCR reaction. The primary goal here is to deploy the (N=5) RT-PCR multiplex to obtain a preliminary analytical LoD in units of copies/reaction for each of the probes comprising the set associated with each of the (n) target sites—LoDₙ (Universal), LoDₙ (Wild Type), LoDₙ (Mutant). The analytical LoD associated with the Universal probes (LoDₙ) were lower than that of either LoDₙ or LoDₙ, due to the intentionally longer probe sequence for the universal probe, which is associated with a higher affinity for its complementary amplicon sequence.

### Results

Subsequent to RT-PCR the DNA microarray was prepared for hybridization with brief water washes, and an incubation in prehybridization buffer (0.6M NaCl, 0.06M sodium citrate solution, 0.1% Ficoll, 0.1% Polyvinylpyrrolidone 0.1% Bovine Serum Albumin). Following aspiration of the prehybridization buffer, a mixture of amplicon and hybridization buffer (0.6M NaCl, 0.06M sodium citrate, 0.1% Ficoll, 0.1% Polyvinylpyrrolidone 0.1% Bovine Serum Albumin) was added to the DNA microarray and allowed to incubate for 2 hours. The microarray is then washed with wash buffer (22.5 mM NaCl, 2.25 mM sodium citrate) and dried via centrifugation. The glass portion of the microarray was cleaned with lens tissue and 70% ethanol and images were acquired on the Sensospot. Images were then uploaded for Augury analysis. Following image acquisition and upload to Augury, it was found that the 5-plex RT-PCR reaction, comprising a N=5 multiplex of amplimers [2, 3, 5, 6, 8] was sufficient to obtain a first determination of analytical LODs [LoDₙ (Universal), LoDₙ (Wild Type), and LoDₙ (Mutant)].

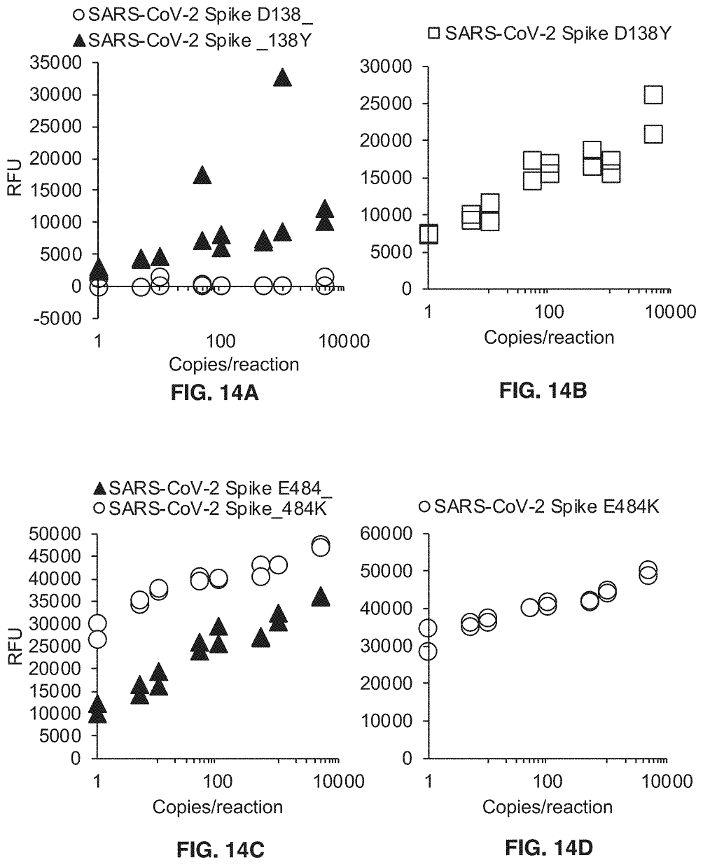

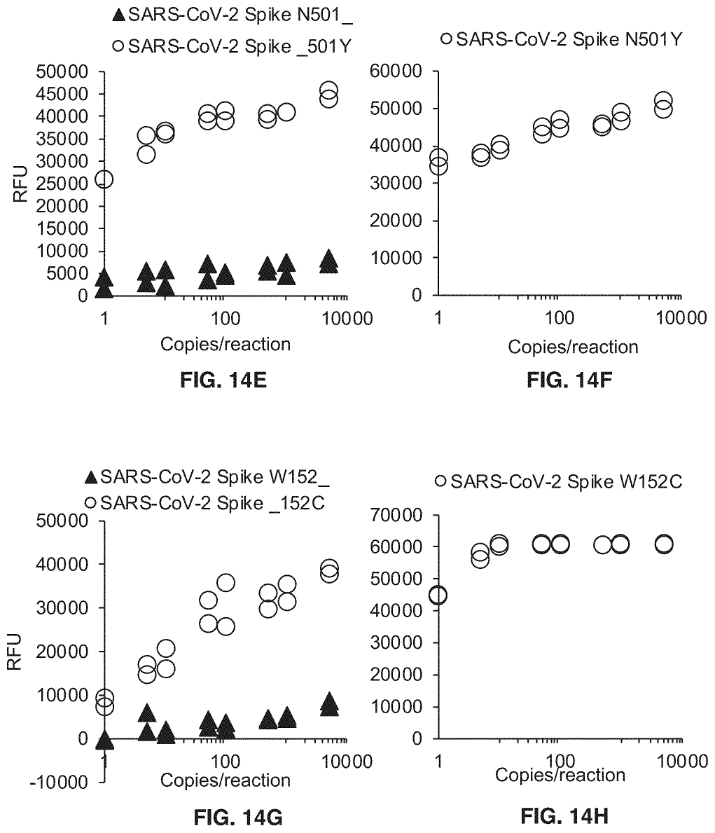

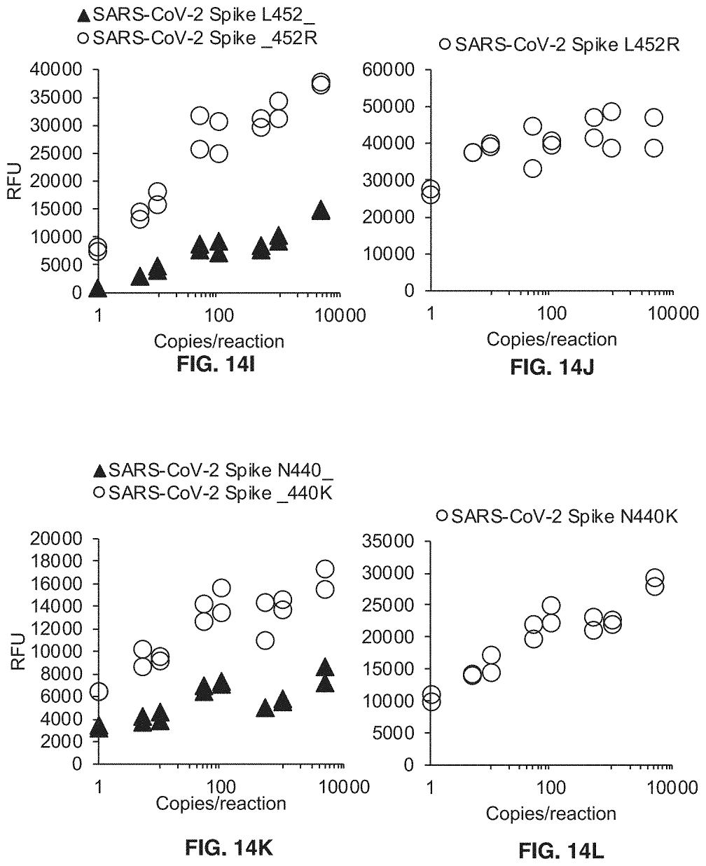

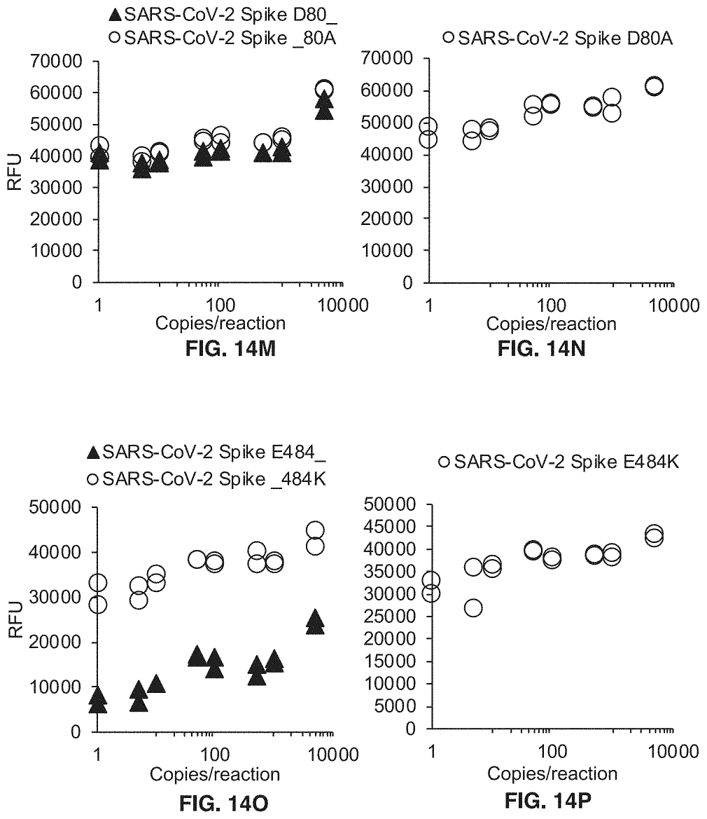

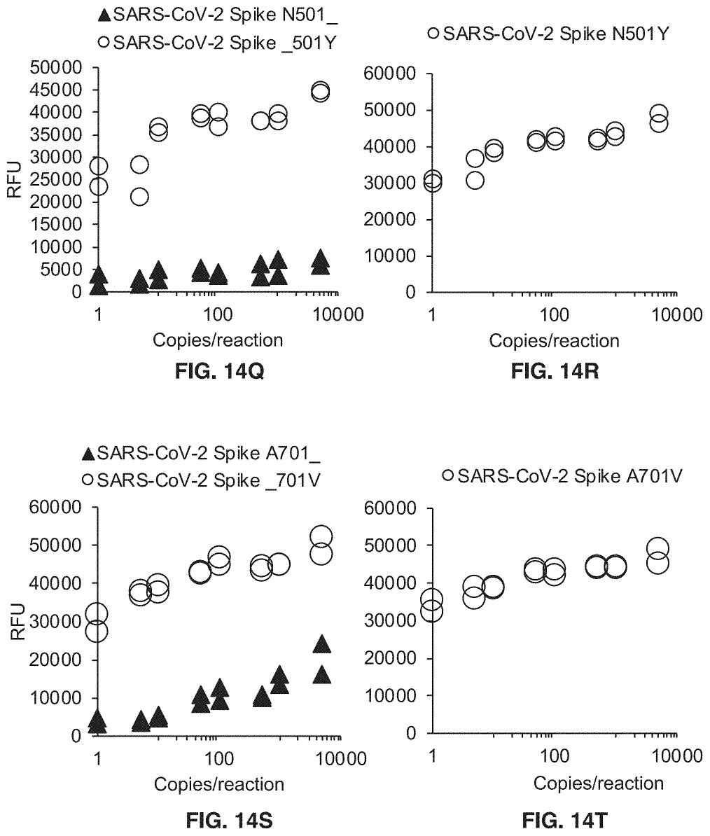

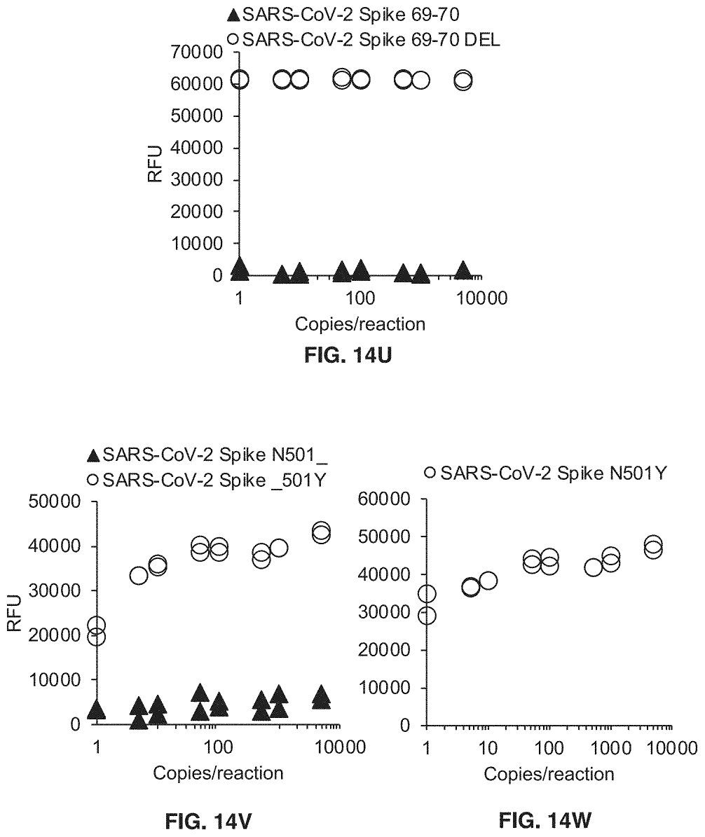

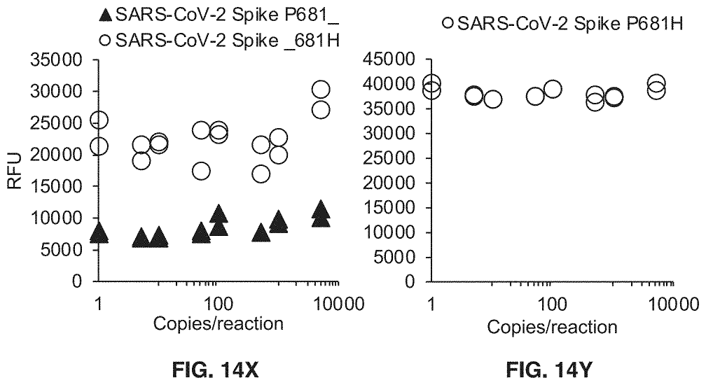

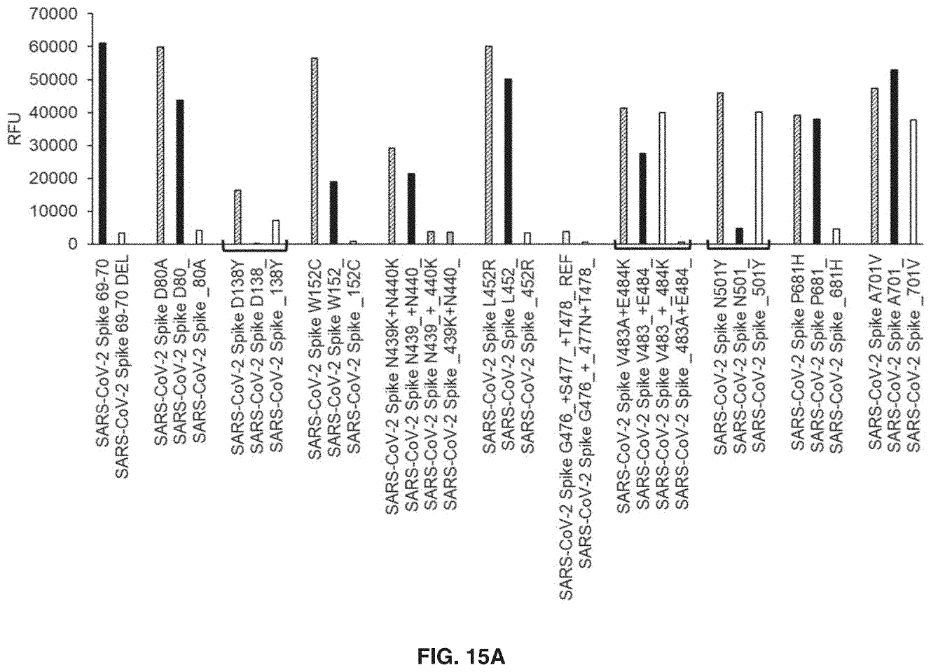

FIGS. 14A-14Y shows analytical LoD data for a series of synthetic G-block fragments corresponding to domains 2-8. The synthetic copy number was determined by IDT and used as such, in subsequent dilutions. Fragments fabricated to display “signature” mutations as defined in boxes showing superscript 1 in Table 15 were mixed into a series of “cocktails” to emulate different clade variant types.

FIGS. 14A, 14C, 14E, 14G, 14I, 14K, 14M, 14O, 14Q, 14S, 14U, 14V and 14X FIG. 4(a-q) show a comparison of signals for Mutant (Synthetic) Cov-2, followed by hybridization to DETECTX-Cv to yield Wild-Type Probe (open circle) vs Mutant Probe (closed triangle) to emphasize the specificity of discrimination between Wild-Type vs Mutant target sequence. The signals were derived from microarray hybridization data (N=2 Repeats) for the N=5 multiplex RT-PCR amplification of Mutant (Synthetic) Cov-2, followed by hybridization to DETECTX-Cv. Table 17 summarizes the analytical LoDₙ (Wild Type) and LoDₙ (Mutant) values. FIGS. 14B, 14D, 14F, 14H, 14J, 14L, 14N, 14P, 14R, 14T, 14W and 14Y FIG. 4(a-q) show microarray hybridization data (N=2 Repeats) for the N=5 multiplex RT-PCR amplification of Mutant (Synthetic) Cov-2, followed by hybridization to DETECTX-Cv, to yield Universal Probe Hybridization probe signals (open square). These data emphasize the high sensitivity of analysis obtained via hybridization to the (longer) Universal Probe, generally manifested as a lower LoDₙ (Universal).

TABLE 17
Summary of analytical LoD values measured for each of the eleven Spike gene target
sites, for Universal, Wild-Type and Mutant probes.

| Target Site | | LoDₙ* | LoDₙ* | LoDₙ^{§} | LoDₙ ^{¶} |
|---|---|---|---|---|---|
| (n) | Amplicon | (Universal) WT | (Universal) MT | (Wild Type) | (Mutant) |
| 69-70 | 2 | NA | NA | 50 | <10 ** |
| (del) | | | | | |
| D80A | 2 | 50 | <10 | 50 | <10 |
| D138Y | 3 | 100 | <10 | 100 | <10 |
| W152C | 3 | 100 | <10 | 500 | <10 |
| N440K | 5 | 50 | <10 | 50 | <10 |
| L452R | 5 | 50 | <10 | 50 | <10 |
| S477N | 6 | 10 | <10 | 50 | <10 |
| E484K | 6 | 10 | <10 | 10 | <10 |
| N501Y | 6 | 100 | <10 | 100 | <10 |
| P681H | 8 | 10 | <10 | 10 | <10 |
| A701V | 8 | 10 | <10 | 50 | <10 |

*LoDₙ (Universal). Analytical LoD Values for Universal Probes as defined from the input target density (in copies per RT-PCR reaction) at which the signal obtained from the Universal probe becomes indistinguishable from the present estimate of background. There are two related values obtained for LoDₙ (Universal).
One value is obtained upon titration with Wild Type (Wuhan) genomic gRNA (LoDₙ (Universal) and the other obtained upon titration with Mutant Synthetic Fragments (LoDₙ (Universal) MT)?
^{§}LoDₙ (Wild Type). Analytical LoD Values for Analysis of Wild Type (Wuhan)as defined from the input target density (measured in copies per RT-PCR reaction) at which the signal obtained from the Wild Type probe becomes indistinguishable from background.
^{¶}LoDₙ (Mutant). Analytical LoD Values for Analysis of Mutant (Synthetic Fragment)as defined from the input target density (measured in copies per RT-PCR reaction) at which the signal obtained from the Mutant probe becomes indistinguishable from background.

### Example 8

### Analysis of “Synthetic Clade Variant” Standards for Deployment to TriCore and Other Labs

### 1. Synthetic Clade Variant Analysis.

The (N=5) RT-PCR Multiplex (2, 3, 5, 6, 8) described in Example 7 was deployed to obtain a full eleven (11) site Clade variant profile using standard hybridization and wash procedures described above.

### 2. Synthetic Clade Variant Cocktails.

A set of five (5) different “Synthetic Clade Variant Standards” corresponding to UK (B.1.1.7), SA (B.1.351), CA452 (B.1.429), Brazil (P.1) and India N440K (B.1.36.29) were prepared each containing a synthetic gene fragment (IDT, Coralville, Iowa) identical to each of the Spike domains amplified by the present RT-PCR multiplex.

### 3. Synthetic Clade Variant Data Analysis.

Data were obtained at 100 copies/reaction for each of the five (5) synthetic cocktails. Hybridization analysis was performed, and the hybridization data thus obtained was plotted as described above.

### 4. Results.

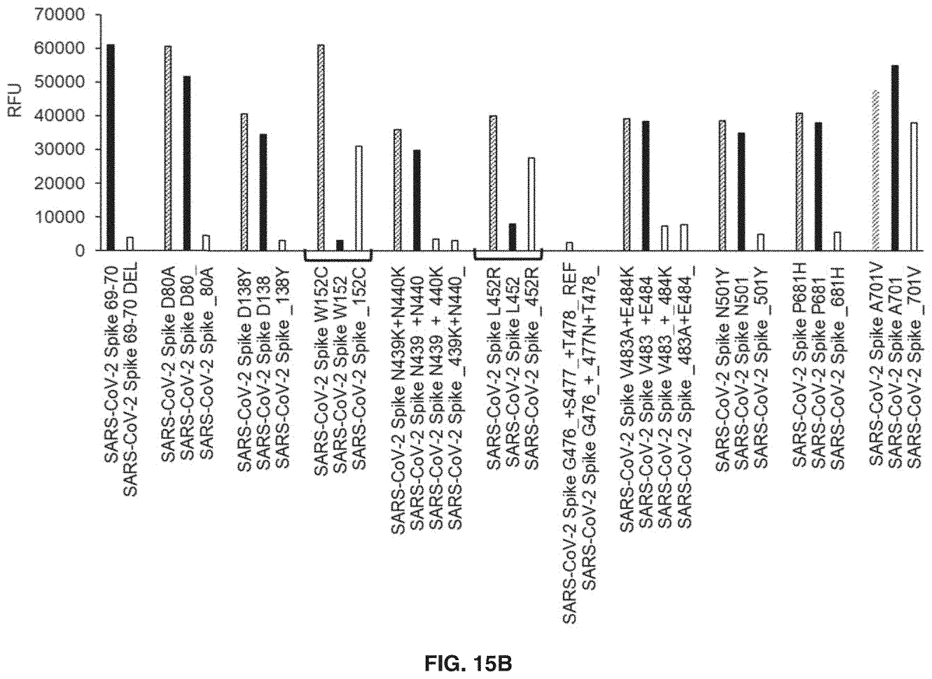

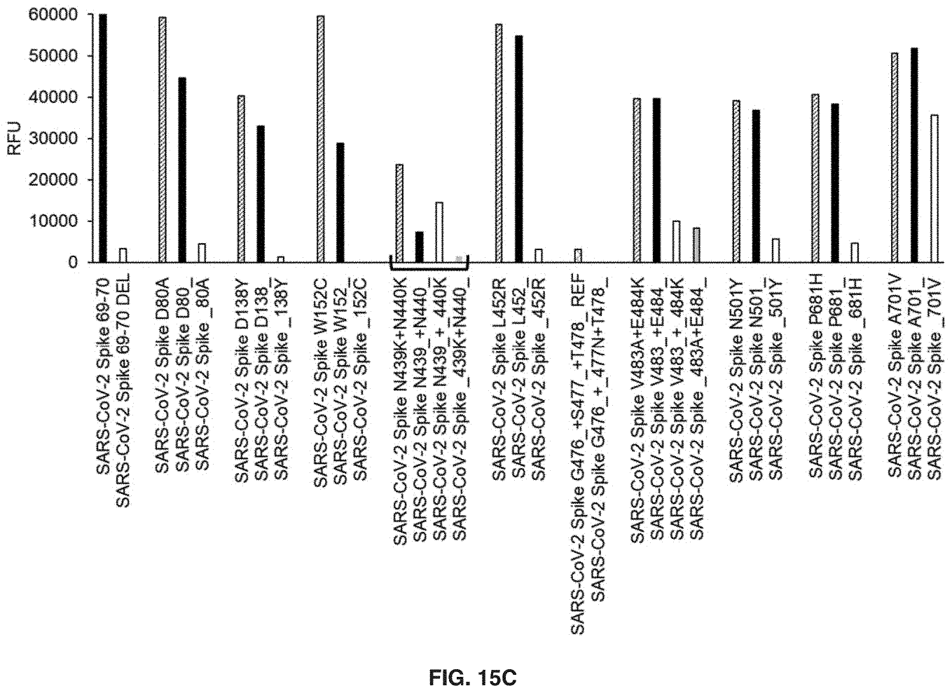

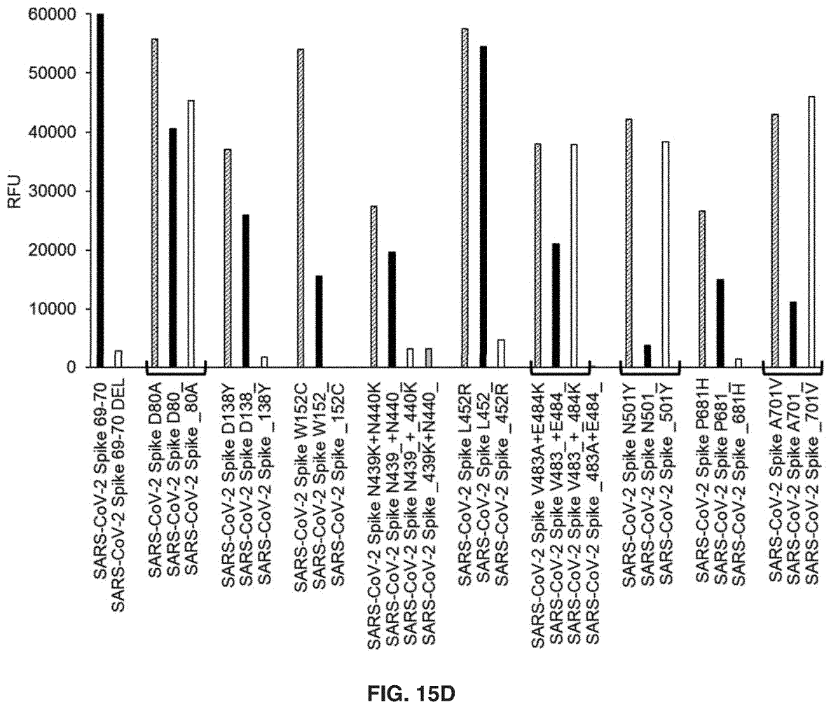

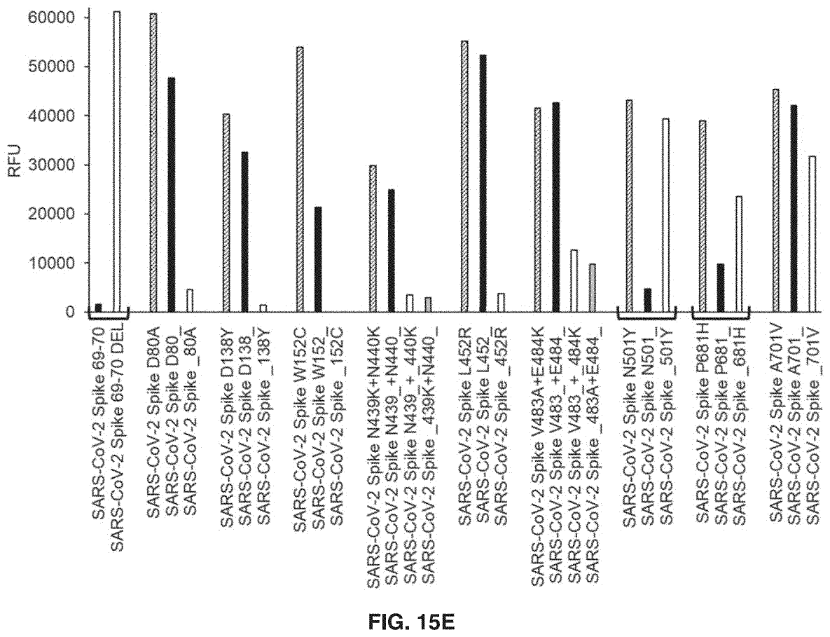

Raw data from this analysis presented in FIGS. 15A-15E shows that the ratio of Mutant (open bars) to Wild Type signal (black bars) readily identify the state of each of the eleven (11) target domains. Spike target sites expected to display a “Mutant” Signal (i.e. open bars >black bars) are marked with brackets.

### Example 9

### CoV-2 Detection and Pooling Via (Oasis) Pure-SAL Saliva Collection.

Clinical LoD Range Finding and Clinical LoD analysis were performed on contrived samples, comprising clinical negatives from healthy volunteers, collected in PURE-SAL™ collection device (OASIS DIAGNOSTICS® Corporation, WA). The samples were contrived with heat attenuated CoV-2 (Wuhan, BEI).

Contrived samples were subjected to viral gRNA capture and purification on Zymo silica magnetic beads or Ceres magnetic beads. Five microliters of purified RNA was added to the RT-PCR mix in a PCR plate. The plate was sealed and placed in a thermocycler to undergo 20 minutes of reverse transcription and 45 cycles of asymmetric PCR. Upon PCR completion, the DNA microarray was prepared for hybridization with brief water washes, and an incubation in prehybridization buffer (0.6M NaCl, 0.06M sodium citrate solution, 0.1% Ficoll, 0.1% Polyvinylpyrrolidone 0.1% Bovine Serum Albumin). Following aspiration of the prehybridization buffer, a mixture of amplicon and hybridization buffer (0.6M NaCl, 0.06M sodium citrate, 0.1% Ficoll, 0.1% Polyvinylpyrrolidone 0.1% Bovine Serum Albumin) was added to the DNA microarray and allowed to incubate for 2 hours. The microarray is then washed with wash buffer (22.5 mM NaCl, 2.25 mM sodium citrate) and dried via centrifugation. The glass portion of the microarray was cleaned with lens tissue and 70% ethanol and images were acquired on the Sensospot. Images were then uploaded for Augury analysis.

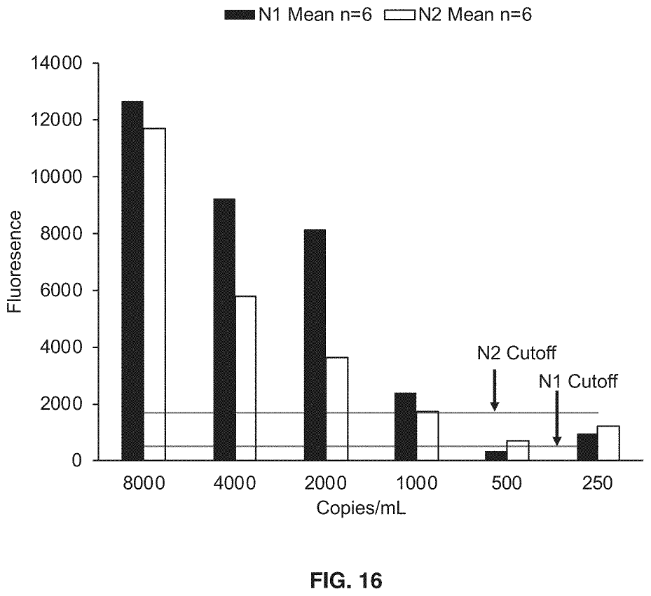

Clinical LoD Results: Clinical LoD range finding was performed as described above (N=6 repeats) using clinically negative saliva samples (PURE-SAL™) to which were added heat inactivated CoV-2 that were processed using Zymo bead capture. FIG. 16 shows that the clinical LoD is close to 1000 copies/ml. A follow-up experiment was performed at N=20, where the resulting clinical LoD is defined as the point at which nineteen of the twenty (19/20) repeated samples produced positive detection (Table 18), which corresponds to a clinical LoD of 1000 copies/ml, a value that is identical within experimental accuracy to that obtained via the same DETECTX-Cv assay of contrived NP-VTM samples with Ceres bead collection as follows. Twenty microliters of beads were added to 400 μL of clinical sample and 800 μL of viral DNA/RNA buffer and mixed on a shaker at 1200 rpm for 10 minutes. The samples were placed on the magnet and supernatant was removed before the addition and pipette-mixing of Zymo Wash Buffer 1. This was repeated for Zymo Wash Buffer 2 and two washes with 100% ethanol. All washes were performed at a volume of 500 μL. The beads were dried at 55° C. Once completely dried, 50 μL of water was added to the beads and mixed well. After placing the samples on the magnet, the supernatant was transfer to another plate for RNA storage. Five microliters of purified RNA were added to the RT-PCR mix in a PCR plate. The plate was sealed and placed in a thermocycler to undergo 20 minutes of reverse transcription and 45 cycles of asymmetric PCR. Upon PCR completion, the DNA microarray was prepared for hybridization with brief water washes, and an incubation in prehybridization buffer (0.6M NaCl, 0.06M sodium citrate solution, 0.1% Ficoll, 0.1% Polyvinylpyrrolidone 0.1% Bovine Serum Albumin). Following aspiration of the prehybridization buffer, a mixture of amplicon and hybridization buffer (0.6M NaCl, 0.06M sodium citrate solution, 0.1% Ficoll, 0.1% Polyvinylpyrrolidone 0.1% Bovine Serum Albumin) was added to the DNA microarray and allowed to incubate for 2 hours. The microarray is then washed with wash buffer (22.5 mM NaCl, 2.25 mM sodium citrate) and dried via centrifugation. The glass portion of the microarray was cleaned with lens tissue and 70% ethanol and images were acquired on the Sensospot. Images were then uploaded for Augury analysis.

TABLE 18
Summary of LoD Experiment Results

| Input | SARS-CoV-2 | SARS- | | |
|---|---|---|---|---|
| Concentration | N1 | CoV-2 N2 | Positive Final Call | % Positive Final Call |
| 1500 cp/mL | 20/20 | 19/20 | 20/20 | 100% |
| 1000 cp/mL | 19/20 | 13/20 | 19/20 | 95% |

Final LoD

| | | | | |
|---|---|---|---|---|
| 1000 cp/mL | 19/20 | 13/20 | 19/20 | 95% |

Pure-SAL Saliva. Pooling Range Finding.

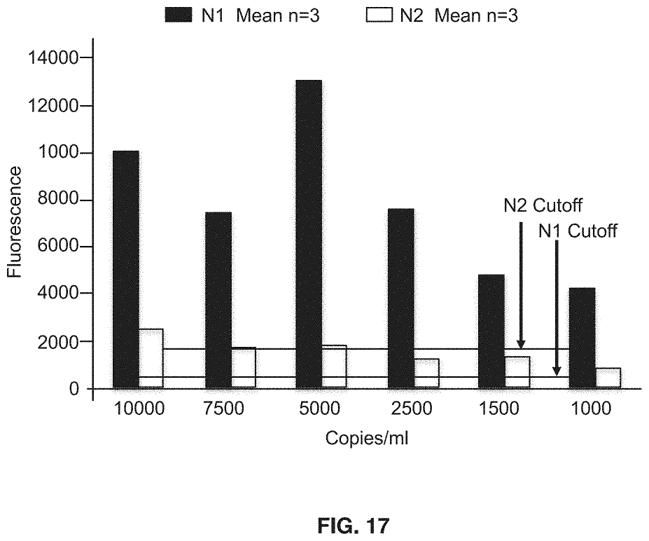

The ability to pool CoV-2 contrived clinical negative PURE-SAL™ saliva samples was tested. Contrived clinical negative samples were pooled at (1) Positive Clinical Sample (100 IL)+(4) Clinical samples (100 IL each), to yield a final pooled sample where the viral complement of the original contrived clinical positive is diluted 5×. The entire pooled specimen was then subjected to Zymo magnetic bead purification, RT-PCR and Hybridization to DETECTX-Cv as described above. The results shown in FIG. 17 suggest that subsequent to 5× pooling, the LoD is reduced less than the full 5× expected from a simple 5× dilution, thus demonstrating feasibility of the N=5 PURE-SAL™ pooling.

### Example 10

### Autonomous Analysis

DETECTX-Cv analysis was performed by hands-free, autonomous analysis of raw DETECTX-microarray data obtained from Sensovation Scans to generate “Mutant” vs “Wild Type” calls among the ten (10) Spike target sites Table 19. These calls were subsequently used for Clade identification. The autonomous analysis is presented here along with manual Augury analysis.

The following multiple functional modules were added to Augury to enable autonomous analysis of DETECTX-Cv data as follows;
(1) Look-Up Table. A database (a “Look-Up Table”) directly related to a Clade Variant vs Mutation data matrix (Table 19) was programmed into Augury. The database is flexible, resident within Augury and can be increased in size as needed to include a larger number of Spike Gene Targets (i.e. more columns as in Table 19) or Clade Variant Targets (i.e. more Rows as in Table 19). Augury is intrinsically linked to the cloud. Further, the Clade Variant Look-Up Table in Augury can be updated in real time via secure inputs such as those which could be provided by Rosalind (San Diego, Calif.).
(2) Comparison among probe data sets. Augury was modified to compare probe information to be used for data quality (QA/QC) and for interpretation of the RFU data (Clade ID):
   a) QA/QC based on signal strength (signal intensity). The universal probes described earlier were used to measure data quality. If universal probe signals were <10,000 (resulting from sample degradation or low concentration), the data associated with the corresponding Mutant and Wild type data at a Spike Target Site are not used by Augury for Clade variant identification.
   b) Data Interpretation: Primary. “Wild Type” and “Mutant” Probe data (RFU) were compared automatically, along with clinical threshold data stored in Augury to generate a “Delta” value (see Example 6). A Delta value greater than 0 returns a “Mutant” call, whereas a Delta value less than 0 returns a “Wild Type” call at each Spike Target Site.
   c) Data Interpretation: Secondary. The pattern of Wild Type vs Mutant calls (i.e. the rows in Table 19) obtained from the Primary Data Interpretation were automatically compared to patterns associated with known Clade variants. The most likely Clade variant pattern is automatically reported. A statistical probability is also assignable to the Clade Variant call and alternative calls based on DETECTX-Cv analysis of multiple Clade Variant samples.
   d) Data Reports. A Standard Report Format was chosen.

### DETECTX-Cv Analysis of Synthetic Clade Variant Standards at TriCore.

Five (5) synthetic Clade variant standards described earlier (UK, SA, CA452, Brazil P.1, India, Examples 8 and 9) were used for on-site validation. Each standard contained a synthetic gene fragment (IDT) identical to each of the Spike domains amplified by the RT-PCR multiplex. DETECTX-Cv data were obtained at TriCore at 100 copies/reaction for each of the five (5) synthetic cocktails. Analysis of the hybridization data were plotted as described previously. Table 20 shows a plate map, PCR recipe and cycling conditions for this analysis. DNA fragment cocktails were utilized as reference.

TABLE 19
Spike Gene Target Region (Codon) Amino Acid Change

| | | | | | L5F | S13I | L18F | T20N | P26S | Q52R | A67V | Δ69-70 | D80A/G | T95I | D138Y |
|---|---|---|---|---|---|---|---|---|---|---|---|---|---|---|---|
| | | | CDC % | | | | | | | | | | | | |
| | | | Mar 14- | Incidence % | | | | | | | | | | | |
| | | Pango | 27 2021 | Gisaid Mar | Signal | | S1 subunit (14-685) | | | | | | | | |
| | Street name | lineage | (US) | 2021 | (1-13) | | N-terminal domain (14-305) | | | | | | | | |
| VOC | UK | B.1.1.7 | 44.10% | 49.81% | L³ | S² | L³ | T² | P³ | Q³ | A³ | Δ¹ | D² | T³ | D² |
| VOC | California | B.1.427 | 6.90% | 2.08% | L³ | I¹ | L³ | T² | P³ | Q³ | A³ | HV² | D² | T³ | D² |
| | L452R | | | | | | | | | | | | | | |
| VOC | | B.1.427 | 2.90% | 0.90% | L³ | S/I¹ | L³ | T² | P³ | Q³ | A³ | HV² | D² | T³ | D² |
| VOC | Brazil | P.1 | 1.40% | 0.39% | L³ | S² | F | N¹ | S | Q³ | A³ | HV² | D² | T³ | Y¹ |
| VOC | SA | B.1.351 | 0.70% | 1.13% | L³ | S² | L³ | T² | P³ | Q³ | A³ | HV² | A¹ | T³ | D² |
| VOC | NYC | B.1.526 | 9.20% | 0.82% | L/F | S² | L³ | T² | P³ | Q³ | A³ | HV² | D² | I | D² |
| | (Ho et al.) | | | | | | | | | | | | | | |
| VOC | NYC | B.1.525 | 0.50% | 0.10% | L³ | S² | L³ | T² | P³ | R | V | Δ¹ | D² | T³ | D² |
| VOC | Rio de | P.2 | 0.30% | 0.36% | L³ | S² | L³ | T² | P³ | Q³ | A³ | HV² | D² | T³ | D² |
| | Janeiro | | | | | | | | | | | | | | |
| | | B.1.2 | 10.00% | 7.83% | L³ | S² | L³ | T² | P³ | Q³ | A³ | HV² | D² | T³ | D² |
| | | B.1, B.1.1, | 2.4%/0.9%/ | 2.6%/1.5%/ | L³ | S² | L³ | T² | P³ | Q³ | A³ | HV² | D² | T³ | D² |
| | | B.1.234 | 0.5% | 0.5% | | | | | | | | | | | |
| | | B.1.1.519 | 4.10% | 1.50% | L³ | S² | L³ | T² | P³ | Q³ | A³ | HV² | D² | T³ | D² |
| | | B.1.526.1 | 3.90% | 0.35% | F | S² | L³ | T² | P³ | Q³ | A³ | HV² | D² | T³ | D² |
| | | B.1.526.2 | 2.90% | 0.18% | F | S² | L³ | T² | P³ | Q³ | A³ | HV² | D² | T³ | D² |
| | | B.1.596 | 1.70% | 1.04% | L³ | S² | L³ | T² | P³ | Q³ | A³ | HV² | D² | T³ | D² |
| | | R.1 | 1.20% | 0.20% | L³ | S² | L³ | T² | P³ | Q³ | A³ | HV² | D² | T³ | D² |
| | | B.1.575 | 1.10% | 0.19% | L³ | S² | L³ | T² | P³ | Q³ | A³ | HV² | D² | T³ | D² |
| | | B.1.243, | 0.60% | 0.84% | L³ | S² | L³ | T² | P³ | Q³ | A³ | HV² | D² | T³ | D² |
| | | B.1.1.207 | | | | | | | | | | | | | |
| | US | B.1.375 | <1% | 0.03% | L³ | S² | L³ | T² | P³ | Q³ | A³ | Δ¹ | D² | T³ | D² |
| | | B.1.1.1, | <1% | 0.50% | L³ | S² | L³ | T² | P³ | Q³ | A³ | HV² | D² | T³ | D² |
| | | B.1.416, | | | | | | | | | | | | | |
| | | B.1.1.33, | | | | | | | | | | | | | |
| | | B.1.311, | | | | | | | | | | | | | |
| | | B.1.1.122 | | | | | | | | | | | | | |
| | Brazil | B.1.1.28 | <1% | 0.10% | L³ | S² | L³ | T² | P³ | Q³ | A³ | HV² | D² | T³ | D² |
| | (Original) | | | | | | | | | | | | | | |
| | Andhra | B.1.36.29 | <1% | 0.08% | L³ | S² | F | T² | P³ | Q³ | A³ | HV² | D² | T³ | D² |
| | Pradesh | | | | | | | | | | | | | | |
| | | A.23.1 | <1% | 0.05% | L³ | S² | L³ | T² | P³ | Q³ | A³ | HV² | D² | T³ | D² |
| | | A.27 | <1% | 0.05% | L³ | S² | F | T² | P³ | Q³ | A³ | HV² | D² | T³ | D² |
| | | A.28 | <1% | 0.02% | L³ | S² | L³ | T² | P³ | Q³ | A³ | Δ¹ | D² | T³ | D² |
| | Mink/ | B.1.1.298 | <1% | 0.00% | L³ | S² | L³ | T² | P³ | Q³ | A³ | Δ¹ | D² | T³ | D² |
| | Cluster V | | | | | | | | | | | | | | |
| | | B.1.1.318 | <1% | 0.01% | L³ | S² | L³ | T² | P³ | Q³ | A³ | HV² | D² | T³ | D² |
| | | B.1.160 | <1% | 1.76% | L³ | S² | L³ | T² | P³ | Q³ | A³ | HV² | D² | T³ | D² |
| | | B.1.177 | <1% | 3.19% | L³ | S² | F | T² | P³ | Q³ | A³ | HV² | D² | T³ | D² |
| | | B.1.177.80 | <1% | 0.04% | L³ | S² | F | T² | P³ | Q³ | A³ | HV² | D² | T³ | D² |
| | | B.1.258 | <1% | 1.15% | L³ | S² | L³ | T² | P³ | Q³ | A³ | HV/Δ¹ | D² | T³ | D² |
| | | B.1.258.14 | <1% | 0.06% | L³ | S² | L³ | T² | P³ | Q³ | A³ | HV² | D² | T³ | D² |
| | | B.1.258.17 | <1% | 1.02% | L³ | S² | L³ | T² | P³ | Q³ | A³ | Δ¹ | D² | T³ | D² |
| | | B.1.517 | <1% | 0.25% | L³ | S² | L³ | T² | P³ | Q³ | A³ | HV² | D² | T³ | D² |
| | WUHAN | WUHAN | — | — | L³ | S² | L³ | T² | P³ | Q³ | A³ | HV² | D² | T³ | D² |

| | | | |
|---|---|---|---|
| PCR Amplimer length (bases) | (1) 101 | (2_{B}) 150 | (3) 129 |

| | | | | | | | | | |
|---|---|---|---|---|---|---|---|---|---|
| Y144DEL | W152C | F157L/S | L189F | R190S | D215G | A222V | A243del | G252V | D253G |

| | | | CDC % | | | | | | | | | | | |
|---|---|---|---|---|---|---|---|---|---|---|---|---|---|---|
| | | | Mar 14- | Incidence % | | | | | | | | | | |
| | | Pango | 27 2021 | Gisaid Mar | S1 subunit (14-685) | | | | | | | | | |
| | Street name | lineage | (US) | 2021 | N-terminal domain (14-305) | | | | | | | | | |
| VOC | UK | B.1.1.7 | 44.10% | 49.81% | Δ¹ | W² | F³ | L³ | R³ | D³ | A³ | A² | G³ | D² |
| VOC | California | B.1.427 | 6.90% | 2.08% | Y² | C¹ | F³ | L³ | R³ | D³ | A³ | A² | G³ | D² |
| | L452R | | | | | | | | | | | | | |
| VOC | | B.1.427 | 2.90% | 0.90% | Y² | W/C² | F³ | L³ | R³ | D³ | A³ | A² | G³ | D² |
| VOC | Brazil | P.1 | 1.40% | 0.39% | Y² | W² | F³ | L³ | S | D³ | A³ | A² | G³ | D² |
| VOC | SA | B.1.351 | 0.70% | 1.13% | Y² | W² | F³ | L³ | R³ | G | A³ | A² | G³ | D² |
| VOC | NYC | B.1.526 | 9.20% | 0.82% | Y² | W² | F³ | L³ | R³ | D³ | A³ | A² | G³ | G¹ |
| | (Ho et al.) | | | | | | | | | | | | | |
| VOC | NYC | B.1.525 | 0.50% | 0.10% | Δ¹ | W² | F³ | L³ | R³ | D³ | A³ | A² | G³ | D² |
| VOC | Rio de | P.2 | 0.30% | 0.36% | Y² | W² | F³ | L³ | R³ | D³ | A³ | A² | G³ | D² |
| | Janeiro | | | | | | | | | | | | | |
| | | B.1.2 | 10.00% | 7.83% | Y² | W² | F³ | L³ | R³ | D³ | A³ | A² | G³ | D² |
| | | B.1, B.1.1, | 2.4%/0.9%/ | 2.6%/1.5%/ | Y² | W² | F³ | L³ | R³ | D³ | A³ | A² | G³ | D² |
| | | B.1.234 | 0.5% | 0.5% | | | | | | | | | | |
| | | B.1.1.519 | 4.10% | 1.50% | Y² | W² | F³ | L³ | R³ | D³ | A³ | A² | G³ | D² |
| | | B.1.526.1 | 3.90% | 0.35% | Y² | W² | S | L³ | R³ | D³ | A³ | A² | G³ | D/G¹ |
| | | B.1.526.2 | 2.90% | 0.18% | Y² | W² | F³ | L³ | R³ | D³ | A³ | A² | G³ | G¹ |
| | | B.1.596 | 1.70% | 1.04% | Y² | W² | F³ | L³ | R³ | D³ | A³ | A² | G³ | D² |
| | | R.1 | 1.20% | 0.20% | Y² | L | F³ | L³ | R³ | D³ | A³ | A² | G³ | D² |
| | | B.1.575 | 1.10% | 0.19% | Y² | W² | F³ | L³ | R³ | D³ | A³ | A² | G³ | D² |
| | | B.1.243, | 0.60% | 0.84% | Y² | W² | F³ | L³ | R³ | D³ | A³ | A² | G³ | D² |
| | | B.1.1.207 | | | | | | | | | | | | |
| | US | B.1.375 | <1% | 0.03% | Y² | W² | F³ | L³ | R³ | D³ | A³ | A² | G³ | D² |
| | | B.1.1.1, | <1% | 0.50% | Y² | W² | F³ | L³ | R³ | D³ | A³ | A² | G³ | D² |
| | | B.1.416, | | | | | | | | | | | | |
| | | B.1.1.33, | | | | | | | | | | | | |
| | | B.1.311, | | | | | | | | | | | | |
| | | B.1.1.122 | | | | | | | | | | | | |
| | Brazil | B.1.1.28 | <1% | 0.10% | Y² | W² | F³ | L³ | R³ | D³ | A³ | A² | G³ | D² |
| | (Original) | | | | | | | | | | | | | |
| | Andhra | B.1.36.29 | <1% | 0.08% | Y² | W² | F³ | L³ | R³ | D³ | A³ | A² | G³ | D² |
| | Pradesh | | | | | | | | | | | | | |
| | | A.23.1 | <1% | 0.05% | Y² | W² | L | L³ | R³ | D³ | A³ | A² | G³ | D² |
| | | A.27 | <1% | 0.05% | Y² | W² | F³ | L³ | R³ | D³ | A³ | A² | G³ | D² |
| | | A.28 | <1% | 0.02% | Y² | W² | F³ | L³ | R³ | D³ | A³ | A² | G³ | D² |
| | Mink/ | B.1.1.298 | <1% | 0.00% | Y² | W² | F³ | L³ | R³ | D³ | A³ | A² | G³ | D² |
| | Cluster V | | | | | | | | | | | | | |
| | | B.1.1.318 | <1% | 0.01% | Δ¹ | W² | F³ | L³ | R³ | D³ | A³ | A² | G³ | D² |
| | | B.1.160 | <1% | 1.76% | Y² | W² | F³ | L³ | R³ | D³ | A³ | A² | G³ | D² |
| | | B.1.177 | <1% | 3.19% | Y² | W² | F³ | L³ | R³ | D³ | V | A² | G³ | D² |
| | | B.1.177.80 | <1% | 0.04% | Δ/Y¹ | W² | F³ | L³ | R³ | D³ | V | A² | G³ | D² |
| | | B.1.258 | <1% | 1.15% | Y² | W² | F³ | L³ | R³ | D³ | A³ | A² | G³ | D² |
| | | B.1.258.14 | <1% | 0.06% | Y² | W² | F³ | L³ | R³ | D³ | A³ | A² | G³ | D² |
| | | B.1.258.17 | <1% | 1.02% | Y² | W² | F³ | F | R³ | D³ | A³ | A² | G³ | D² |
| | | B.1.517 | <1% | 0.25% | Y² | W² | F³ | L³ | R³ | D³ | A³ | A² | G/V | D² |
| | WUHAN | WUHAN | — | — | Y² | W² | F³ | L³ | R³ | D³ | A³ | A² | G³ | D² |

| | | |
|---|---|---|
| PCR Amplimer length (bases) | (3) 129 | (4_{B}) 160 |

| | | | | | V367F | K417N/T | N439K | N440K | L452R | Y453F | S477N | V483A | E484K | S494P | N501Y/T | A570D | Q613H | D614G |
|---|---|---|---|---|---|---|---|---|---|---|---|---|---|---|---|---|---|---|
| | | | CDC % | | | | | | | | | | | | | | | |
| | | | March | Incidence | | | | | | | | | | | | | | |
| | | | 14-27 | % Gisaid | | | | | | | | | | | | | | |
| | Street | Pango | 2021 | March | S1 subunit (14-685) | | | | | | | | | | | | | |
| | name | lineage | (US) | 2021 | RBD (319-541) | | | | | | | | | | | | | |
| VOC | UK | B.1.1.7 | 44.10% | 49.81% | V³ | K² | N² | N² | L² | Y² | S² | V² | E/K¹ | S/P | Y¹ | D | Q² | G¹ |
| VOC | California | B.1.427 | 6.90% | 2.08% | V³ | K² | N² | N² | R¹ | Y² | S² | V² | E² | S³ | N² | A³ | Q² | G¹ |
| | L452R | | | | | | | | | | | | | | | | | |
| VOC | | B.1.429 | 2.90% | 0.90% | V³ | K² | N² | N² | R¹ | Y² | S² | V² | E² | S³ | N² | A³ | Q² | G¹ |
| VOC | Brazil | P.1 | 1.40% | 0.39% | V³ | K² | N² | N² | L² | Y² | S² | V² | K¹ | S³ | Y¹ | A³ | Q² | G¹ |
| VOC | SA | B.1.351 | 0.70% | 1.13% | V³ | K² | N² | N² | L² | Y² | S² | V² | K¹ | S³ | Y¹ | A³ | Q² | G¹ |
| VOC | NYC | B.1.526 | 9.20% | 0.82% | V³ | K² | N² | N² | L² | Y² | S/N¹ | V² | E/K¹ | S³ | N² | A³ | Q² | G¹ |
| | (Ho et al.) | | | | | | | | | | | | | | | | | |
| VOC | NYC | B.1.525 | 0.50% | 0.10% | V³ | K² | N² | N² | L² | Y² | S² | V² | K¹ | S³ | N² | A³ | Q² | G¹ |
| VOC | Rio de | P.2 | 0.30% | 0.36% | V³ | K² | N² | N² | L² | Y² | S² | V² | K¹ | S³ | N² | A³ | Q² | G¹ |
| | Janeiro | | | | | | | | | | | | | | | | | |
| | | B.1.2 | 10.00% | 7.83% | V³ | K² | N² | N² | L² | Y² | S² | V² | E² | S³ | Y¹ | A³ | Q² | G¹ |
| | | B.1, | 2.4%/ | 2.6%/ | V³ | K² | N² | N² | L² | Y² | S² | V² | E² | S³ | N² | A³ | Q² | G¹ |
| | | B.1.1, | 0.9%/ | 1.5%/ | | | | | | | | | | | | | | |
| | | B.1.234 | 0.5% | 0.5% | | | | | | | | | | | | | | |
| | | B.1.1.519 | 4.10% | 1.50% | V³ | K² | N² | N² | L² | Y² | S² | V² | E² | S³ | N² | A³ | Q² | G¹ |
| | | B.1.526.1 | 3.90% | 0.35% | V³ | K² | N² | N² | R¹ | Y² | S² | V² | K¹ | S³ | N² | A³ | Q² | G¹ |
| | | B.1.526.2 | 2.90% | 0.18% | V³ | N/T¹ | N² | N² | L² | Y² | N¹ | V² | E² | S³ | N² | A³ | Q² | G¹ |
| | | B.1.596 | 1.70% | 1.04% | V³ | N¹ | N² | N² | L² | Y² | S² | V² | E² | S³ | N² | A³ | Q² | G¹ |
| | | R.1 | 1.20% | 0.20% | V³ | K² | N² | N² | L² | Y² | S² | V² | K¹ | S³ | N² | A³ | Q² | G¹ |
| | | B.1.575 | 1.10% | 0.19% | V³ | K² | N² | N² | L² | Y² | S² | V² | E² | P | N² | A³ | Q² | G¹ |
| | | B1.243, | 0.60% | 0.84% | V³ | K² | N² | N² | L² | Y² | S² | V² | E² | S³ | N² | A³ | Q² | G¹ |
| | | B1.1.207 | | | | | | | | | | | | | | | | |
| | US | B.1.375 | <1% | 0.03% | V³ | K² | N² | N² | L² | Y² | S² | V² | E² | S³ | N² | A³ | Q² | G¹ |
| | | B.1.1.1, | <1% | 0.50% | V³ | K² | N² | N² | L² | Y² | S² | V² | E² | S³ | N² | A³ | Q² | G¹ |
| | | B.1.416, | | | | | | | | | | | | | | | | |
| | | B.1.1.33, | | | | | | | | | | | | | | | | |
| | | B.1.311, | | | | | | | | | | | | | | | | |
| | | B.1.1.122 | | | | | | | | | | | | | | | | |
| | Brazil | B.1.1.28 | <1% | 0.10% | V³ | K² | N² | N² | L² | Y² | S² | V² | E² | S³ | N² | A³ | Q² | G¹ |
| | Original | | | | | | | | | | | | | | | | | |
| | Andhra | B.1.36.29 | <1% | 0.08% | V³ | K² | N² | K¹ | L² | Y² | S² | V² | E² | S³ | N² | A³ | Q² | G¹ |
| | Pradesh | | | | | | | | | | | | | | | | | |
| | | A.23.1 | <1% | 0.05% | F | K² | N² | N² | L² | Y² | S² | V² | E/K¹ | S³ | N² | A³ | H¹ | D² |
| | | A.27 | <1% | 0.05% | V³ | K² | N² | N² | R¹ | Y² | S² | V² | E² | S³ | Y¹ | A³ | Q² | D² |
| | | A.28 | <1% | 0.02% | V³ | K² | N² | N² | L² | Y² | S² | V² | E² | S³ | T | A³ | Q² | D² |
| | Mink/ | B.1.1.298 | <1% | 0.00% | V³ | K² | N² | N² | L² | F¹ | S² | V² | E² | S³ | N² | A³ | Q² | G¹ |
| | Cluster V | | | | | | | | | | | | | | | | | |
| | | B.1.1.318 | <1% | 0.01% | V³ | K² | N² | N² | L² | Y² | S² | V² | K¹ | S³ | N² | A³ | Q² | G¹ |
| | | B.1.1.160 | <1% | 1.76% | V³ | K² | N² | N² | L² | Y² | N¹ | V² | E² | S³ | N² | A³ | Q² | G¹ |
| | | B.1.177 | <1% | 3.19% | V³ | K² | N² | N² | L² | Y² | S² | V² | E² | S³ | N² | A³ | Q² | G¹ |
| | | B.1.177.80 | <1% | 0.04% | V³ | K² | N² | N² | L² | Y² | S² | V² | E² | S³ | N² | A³ | Q² | G¹ |
| | | B.1.258 | <1% | 1.15% | V³ | K² | K¹ | N² | L² | Y² | S² | V² | E² | S³ | N² | A³ | Q² | G¹ |
| | | B.1.258.14 | <1% | 0.06% | V³ | K² | K¹ | N² | L² | Y² | S² | V² | E² | S³ | N² | A³ | Q² | G¹ |
| | | B.1.258.17 | <1% | 1.02% | V³ | K² | K¹ | N² | L² | Y² | S² | V² | E² | S³ | N² | A³ | Q² | G¹ |
| | | B.1.517 | <1% | 0.25% | V³ | K² | N² | N² | L² | Y² | S² | V² | E² | S³ | T | A³ | Q/H¹ | G¹ |
| | WUHAN | WUHAN | — | — | V³ | K² | N² | N² | L² | Y² | S² | V² | E² | S³ | N² | A³ | Q² | D² |

| | | | |
|---|---|---|---|
| PCR Amplimer (bases) | (5) 199 | (6) 151 | (7) 88 |

| | | | | | H655Y | Q677P/H | P681H | I692V | A701V | T716I | G769V | D769V | F888L | S982A | T1027I | D1118H | V1176F |
|---|---|---|---|---|---|---|---|---|---|---|---|---|---|---|---|---|---|
| | | | CDC % | | | | | | | | | | | | | | |
| | | | March | Incidence | | | | | | | | | | | | | |
| | | | 14-27 | % Gisaid | S2 subunit (686-1273) | | | | | | | | | | | | |
| | Street | Pango | 2021 | March | Fusion peptide | | | | | | | | | | | | |
| | name | lineage | (US) | 2021 | (788-806) | | | | | | | | | | | | |
| VOC | UK | B.1.1.7 | 44.10% | 49.81% | H³ | Q² | H¹ | I² | A² | I | G³ | D³ | F³ | A | T³ | H | V³ |
| VOC | California | B.1.427 | 6.90% | 2.08% | H³ | Q² | P² | I² | A² | T³ | G³ | D³ | F³ | S³ | T³ | D³ | V³ |
| | L452R | | | | | | | | | | | | | | | | |
| VOC | | B.1.429 | 2.90% | 0.90% | H³ | Q² | P² | I² | A² | T³ | G³ | D³ | F³ | S³ | T³ | D³ | V³ |
| VOC | Brazil | P.1 | 1.40% | 0.39% | Y | Q² | P² | I² | A² | T³ | G³ | D³ | F³ | S³ | I | D³ | F |
| VOC | SA | B.1.351 | 0.70% | 1.13% | H³ | Q² | P² | I² | V¹ | T³ | G³ | D³ | F³ | S³ | T³ | D³ | V³ |
| VOC | NYC | B.1.526 | 9.20% | 0.82% | H³ | Q² | P² | I² | A/V¹ | T³ | G³ | D³ | F³ | S³ | T³ | D³ | V³ |
| | (Ho et al.) | | | | | | | | | | | | | | | | |
| VOC | NYC | B.1.525 | 0.50% | 0.10% | H³ | H¹ | P² | I² | A² | T³ | G³ | D³ | L | S³ | T³ | D³ | V³ |
| VOC | Rio de | P.2 | 0.30% | 0.36% | H³ | Q² | P² | I² | A² | T³ | G³ | D³ | F³ | S³ | T³ | D³ | F |
| | Janeiro | | | | | | | | | | | | | | | | |
| | | B.1.2 | 10.00% | 7.83% | H³ | Q² | P² | I² | A² | T³ | G³ | D³ | F³ | S³ | T³ | D³ | V³ |
| | | B.1, | 2.4%/ | 2.6%/ | H³ | Q² | P² | I² | A² | T³ | G³ | D³ | F³ | S³ | T³ | D³ | V³ |
| | | B.1.1, | 0.9%/ | 1.5%/ | | | | | | | | | | | | | |
| | | B.1.234 | 0.5% | 0.5% | | | | | | | | | | | | | |
| | | B.1.1.519 | 4.10% | 1.50% | H³ | Q² | H¹ | I² | A² | T³ | G³ | D³ | F³ | S³ | T³ | D³ | V³ |
| | | B.1.526.1 | 3.90% | 0.35% | H³ | Q² | P² | I² | A/V¹ | T³ | G³ | D³ | F³ | S³ | T³ | D³ | V³ |
| | | B.1.526.2 | 2.90% | 0.18% | H³ | Q² | P² | I² | A² | T³ | G³ | D³ | F³ | S³ | T³ | D³ | V³ |
| | | B.1.596 | 1.70% | 1.04% | H³ | Q/P¹ | P² | I² | A² | T³ | G³ | D³ | F³ | S³ | T³ | D³ | V³ |
| | | R.1 | 1.20% | 0.20% | H³ | Q² | P² | I² | A² | T³ | V | D³ | F³ | S³ | T³ | D³ | V³ |
| | | B.1.575 | 1.10% | 0.19% | H³ | Q² | H¹ | I² | A² | I | G³ | D³ | F³ | S³ | T³ | D³ | V³ |
| | | B1.243, | 0.60% | 0.84% | H³ | Q² | H¹ | I² | A² | T³ | G³ | D³ | F³ | S³ | T³ | D³ | V³ |
| | | B1.1.207 | | | | | | | | | | | | | | | |
| | US | B.1.375 | <1% | 0.03% | H³ | Q² | P² | I² | A² | T³ | G³ | D³ | F³ | S³ | T³ | D³ | V³ |
| | | B.1.1.1, | <1% | 0.50% | H³ | Q² | P² | I² | A² | T³ | G³ | D³ | F³ | S³ | T³ | D³ | V³ |
| | | B.1.416, | | | | | | | | | | | | | | | |
| | | B.1.1.33, | | | | | | | | | | | | | | | |
| | | B.1.311, | | | | | | | | | | | | | | | |
| | | B.1.1.122 | | | | | | | | | | | | | | | |
| | Brazil | B.1.1.28 | <1% | 0.10% | H³ | Q² | P² | I² | A² | T³ | G³ | D³ | F³ | S³ | T³ | D³ | F |
| | Original | | | | | | | | | | | | | | | | |
| | Andhra | B.1.36.29 | <1% | 0.08% | H³ | Q² | P² | I² | A² | T³ | G³ | D³ | F³ | S³ | T³ | D³ | V³ |
| | Pradesh | | | | | | | | | | | | | | | | |
| | | A.23.1 | <1% | 0.05% | Y | Q² | R¹ | I² | A² | T³ | G³ | D³ | F³ | S³ | T³ | D³ | V³ |
| | | A.27 | <1% | 0.05% | Y | Q² | P² | I² | A² | T³ | G³ | Y | F³ | S³ | T³ | D³ | V³ |
| | | A.28 | <1% | 0.02% | H³ | Q² | P² | I² | A² | T³ | G³ | D³ | F³ | S³ | T³ | D³ | V³ |
| | Mink/ | B.1.1.298 | <1% | 0.00% | H³ | Q² | P² | I² | A² | T³ | G³ | D³ | F³ | S³ | T³ | D³ | V³ |
| | Cluster V | | | | | | | | | | | | | | | | |
| | | B.1.1.318 | <1% | 0.01% | H³ | Q² | H¹ | I² | A² | T³ | G³ | D³ | F³ | S³ | T³ | D³ | V³ |
| | | B.1.1.160 | <1% | 1.76% | H³ | Q² | P² | I² | A² | T³ | G³ | D³ | F³ | S³ | T³ | D³ | V³ |
| | | B.1.177 | <1% | 3.19% | H³ | Q² | P² | I² | A² | T³ | G³ | D³ | F³ | S³ | T³ | D³ | V³ |
| | | B.1.177.80 | <1% | 0.04% | H³ | Q² | P² | I² | A² | T³ | G³ | D³ | F³ | S³ | T³ | D³ | V³ |
| | | B.1.258 | <1% | 1.15% | H³ | Q² | P² | I² | A² | T³ | G³ | D³ | F³ | S³ | T³ | D³ | V³ |
| | | B.1.258.14 | <1% | 0.06% | H³ | Q² | P² | I² | A² | T³ | G³ | D³ | F³ | S³ | T³ | D³ | V³ |
| | | B.1.258.17 | <1% | 1.02% | H³ | Q² | P² | I² | A² | T³ | G³ | D³ | F³ | S³ | T³ | D³ | V³ |
| | | B.1.517 | <1% | 0.25% | H³ | Q² | P² | I² | A² | T³ | G³ | D³ | F³ | S³ | T³ | D³ | V³ |
| | WUHAN | WUHAN | — | — | H³ | Q² | P² | I² | A² | T³ | G³ | D³ | F³ | S³ | T³ | D³ | V³ |

| | |
|---|---|
| PCR Amplimer (bases) | (8) 135 |

¹AA mutation - hybridizes to mutation specific probe
²AA identical to hCoV-19/Wuhan/WIV04/2019 (WIV04) - official reference sequence employed by GISAID (EPI_ISL_402124)
³Potential probe target

TABLE 20
Plate map, PCR recipe and Cycling conditions used in the analysis

| | | | RT-PCR Mix | | | | | |
|---|---|---|---|---|---|---|---|---|
| | | | | Per | RT-PCR conditions | | | |
| Plate Map | | | | reaction | | Temp | | |
| | 1 | 2 | Components | (μL) | Steps | (° C.) | Time | Cycles |

| | | | | | | | | | |
|---|---|---|---|---|---|---|---|---|---|
| A | 300 copies | 300 | ACCESSQUICK ™ | 25 | 1 | 55 | 20 | min | 1 |
| | S. Africa | copies | Mastermix | | | | | | |
| | | UK | | | | | | | |
| B | 100 copies | 100 | Primer | 2 | 2 | 94 | 2 | min | 1 |
| | S. Africa | copies | | | | | | | |
| | | UK | | | | | | | |
| C | 300 copies | 300 | Avian | 1 | 3 | 94 | 30 | s | 45 |
| | California | copies | Myeloblastosis | | | | | | |
| | | Wuhan | Virus (AMV) | | | | | | |
| | | gRNA | Enzyme mix | | | | | | |
| D | 100 copies | 100 | Water | 17 | 4 | 55 | 30 | s | |
| | California | copies | | | | | | | |
| | | Wuhan | | | | | | | |
| | | gRNA | | | | | | | |
| E | 300 copies | NTC | Total | 45 | 5 | 68 | 30 | s | |
| | India | | | | | | | | |
| F | 100 copies | NTC | | | 6 | 68 | 7 | min | 1 |
| | India | | | | | | | | |

| | | | | | |
|---|---|---|---|---|---|
| G | 300 copies | NTC | 7 | 4 | ∞ |
| | Brazil | | | | |
| H | 100 copies | NTC | | | |
| | Brazil | | | | |

### Results

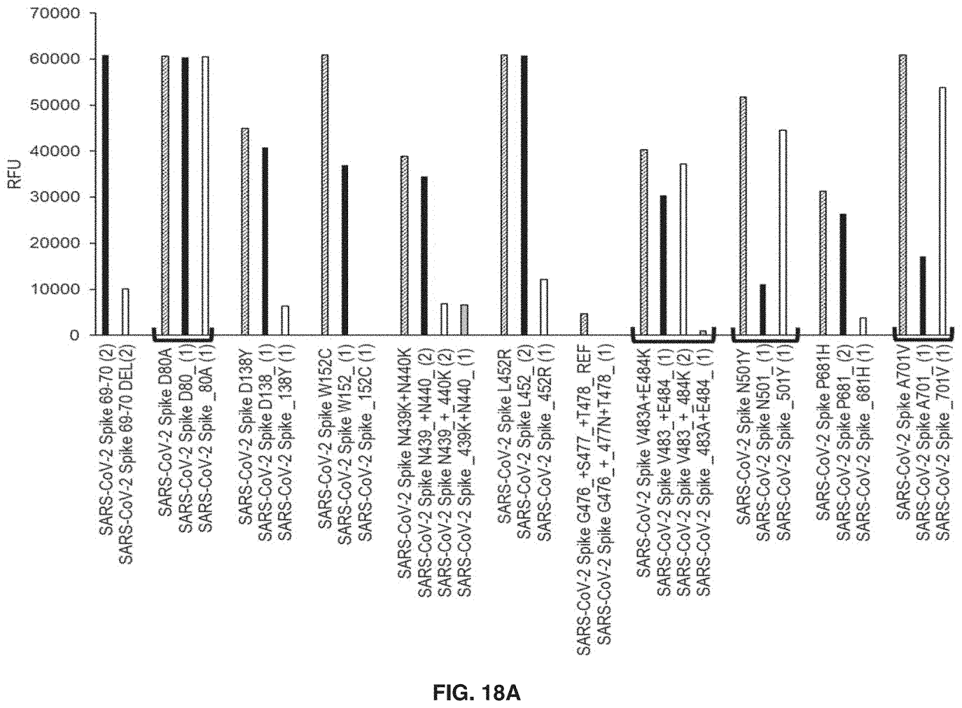

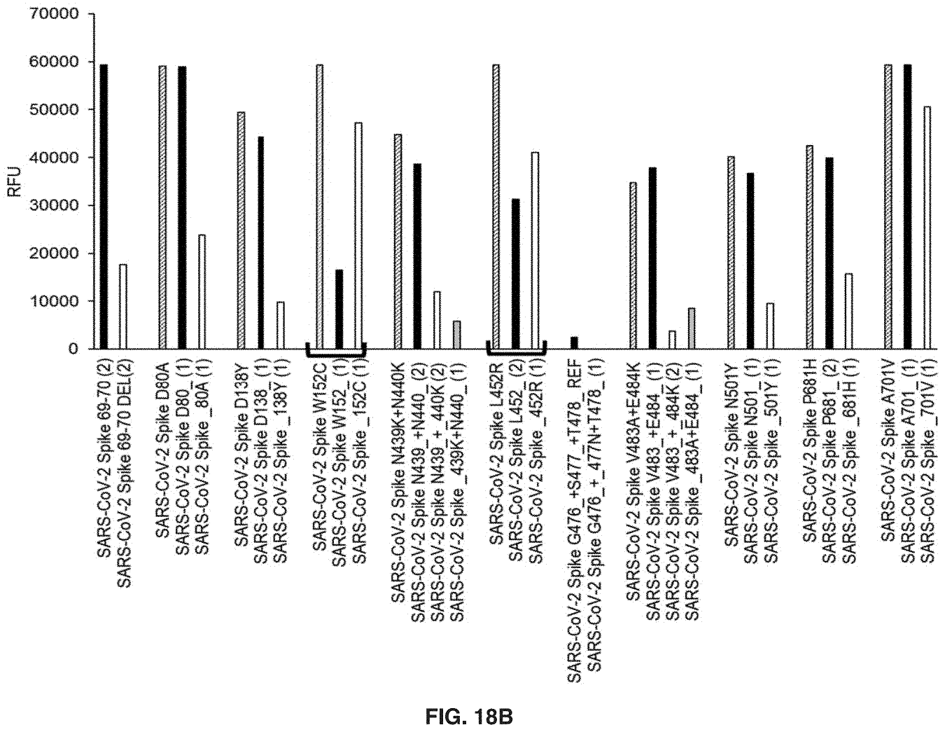

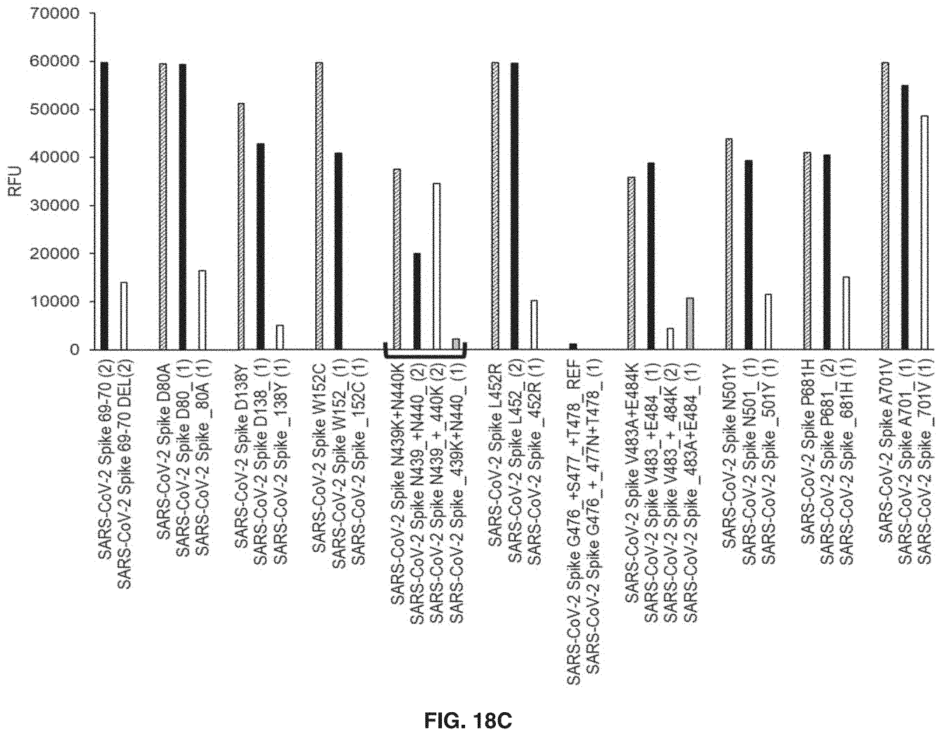

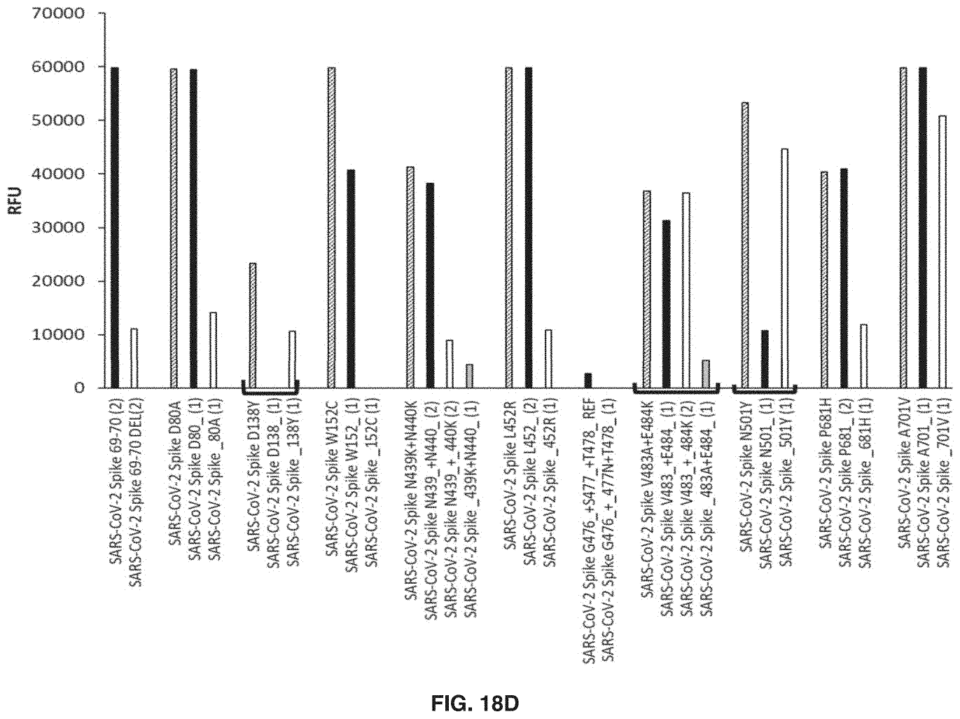

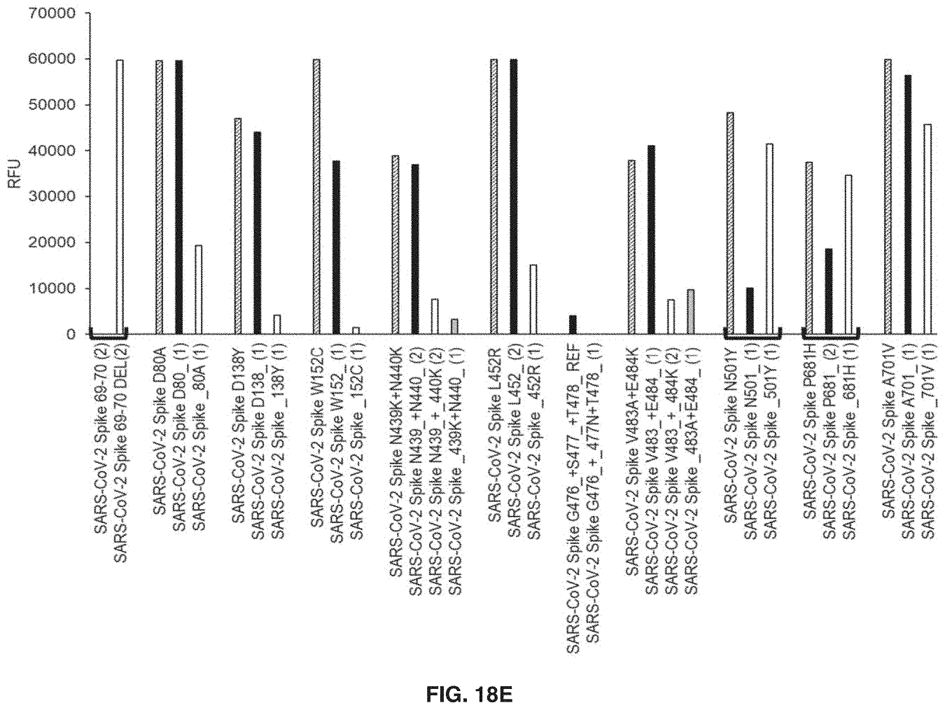

FIGS. 18A-18E and Table 21 show the results from analysis of synthetic clade variant standards at TriCore. The data shows that the raw data, i.e. the ratio of Mutant (open bars) to Wild Type signal (black bars) readily identifies the state of each of the ten (10) target domains. Spike target sites, which were expected to display a “Mutant” signal (i.e. open bars >black bars), are marked in square brackets. As shown, the Mutant vs Wild type signals obtained by TriCore on synthetic Clade variant standards were as expected. The data established that the DETECTX-Cv workflow is easily deployable in any high throughput COVID-19 clinical testing lab.

TABLE 21
DETECTX-Cv analysis of synthetic Clade variant standards at TriCore
Reference FIG. 18A
HYBRIDIZED PROBES

| Probe | Wild Type | Mutant Type | Confidence |
|---|---|---|---|
| H/V69_ | ON | | 100.00% |
| _80A | | ON | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | | ON | 100.00% |
| _501Y | | ON | 100.00% |
| P681_ | ON | | 100.00% |
| _701V | | ON | 100.00% |

UNHYBRIDIZED PROBES

| Probe | Wild Type | Mutant Type | Confidence |
|---|---|---|---|
| _152C | | OFF | 98.80% |
| _681H | | OFF | 100.00% |
| A701_ | OFF | | 97.40% |
| Pattern consistent with | S Africa | | |
| | (B.1.351) | | |
| Reference FIG. 18B | | | |

HYBRIDIZED PROBES

| Probe | Wild Type | Mutant Type | Confidence |
|---|---|---|---|
| H/V69_ | ON | | 100.00% |
| D80_ | ON | | 100.00% |
| D138_ | ON | | 100.00% |
| _152C | | ON | 100.00% |
| N439_ | ON | | 100.00% |
| N440_ | ON | | 100.00% |
| _452R | | ON | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _484K | | OFF | 100.00% |
| _501Y | | OFF | 99.80% |
| Pattern consistent with | California | | |
| | (B.1.429/427) | | |
| Reference FIG. 18C | | | |

HYBRIDIZED PROBES

| Probe | Wild Type | Mutant Type | Confidence |
|---|---|---|---|
| H/V69_ | ON | | 100.00% |
| D80_ | ON | | 100.00% |
| D138_ | ON | | 100.00% |
| W152_ | ON | | 100.00% |
| N439_ | ON | | 100.00% |
| _440K | | ON | 100.00% |
| L452_ | ON | | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _152C | | OFF | 98.80% |
| _484K | | OFF | 100.00% |
| _501Y | | OFF | 98.20% |
| Pattern consistent with | India (N440K) | | |
| Reference FIG. 18D | | | |

HYBRIDIZED PROBES

| Probe | Wild Type | Mutant Type | Confidence |
|---|---|---|---|
| H/V69_ | ON | | 100.00% |
| D80_ | ON | | 100.00% |
| _138Y | | ON | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| _501Y | | ON | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| D138_ | OFF | | 100.00% |
| _152C | | OFF | 98.80% |
| Pattern consistent with | Brazil (P1) | | |
| Reference FIG. 18E | | | |

HYBRIDIZED PROBES

| Probe | Wild Type | Mutant Type | Confidence |
|---|---|---|---|
| _69(del) | | ON | 100.00% |
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| _501Y | | ON | 100.00% |
| _681H | | ON | 100.00% |
| A701_ | ON | | 100.00% |

UNHYBRIDIZED PROBES

| Probe | Wild Type | Mutant Type | Confidence |
|---|---|---|---|
| H/V69_ | OFF | | 100.00% |
| _484K | | OFF | 100.00% |
| Pattern consistent with | UK (B.1.1.7) | | |

TABLE 22
Hybridization plate map for 28 SARS-CoV-2 positive clinical samples

| | 3 | 4 | 5 | 6 |
|---|---|---|---|---|
| A | Sample 1 | Sample 9 | Sample 17 | Sample 25 |
| B | Sample 2 | Sample 10 | Sample 18 | Sample 26 |
| C | Sample 3 | Sample 11 | Sample 19 | Sample 27 |
| D | Sample 4 | Sample 12 | Sample 20 | Sample 28 |
| E | Sample 5 | Sample 13 | Sample 21 | |
| F | Sample 6 | Sample 14 | Sample 22 | |
| G | Sample 7 | Sample 15 | Sample 23 | |
| H | Sample 8 | Sample 16 | Sample 24 | |

### DETECTX-Cv Analysis of Clinical Positive Samples Performed at Tricore

The Biomerieux EASYMAG® Magnetic Bead platform (bioMérieux, St. Louis, Mo.) was used to extract Covid-19 RNA from 28 clinical positive (NP-VTM) samples (positivity previously determined by Cobas 6800 analysis). The extracted RNA (5 IL) was processed using the DETECTX-Cv method. Table 22 shows a plate map for 28 SARS-CoV-2 positive clinical samples. The PCR recipe and cycling conditions were as described in Table 20.

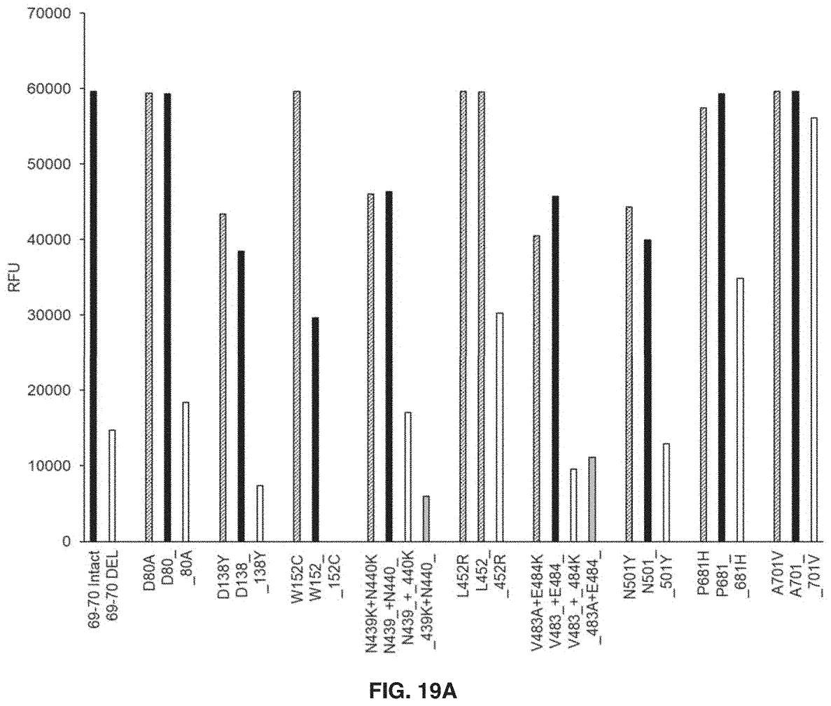

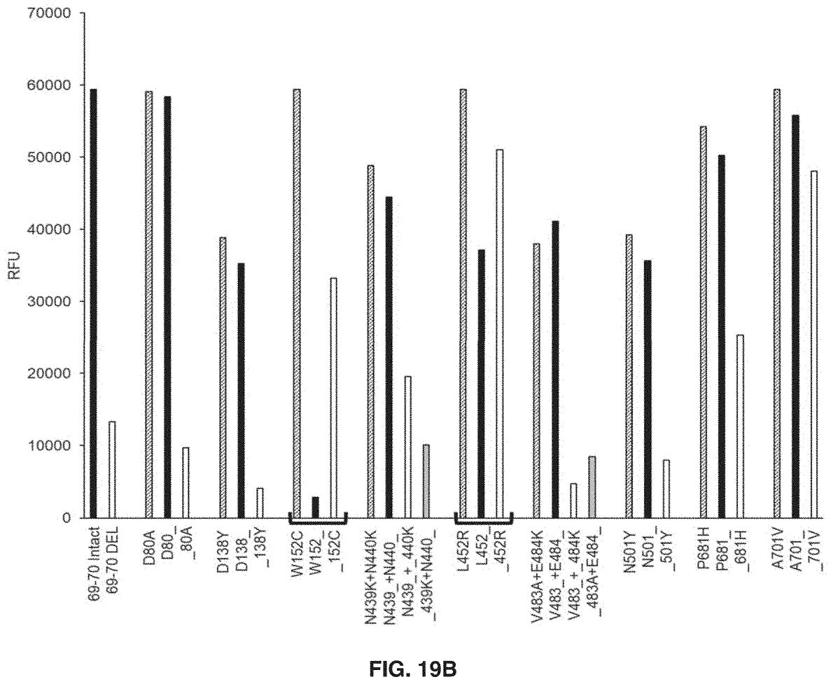

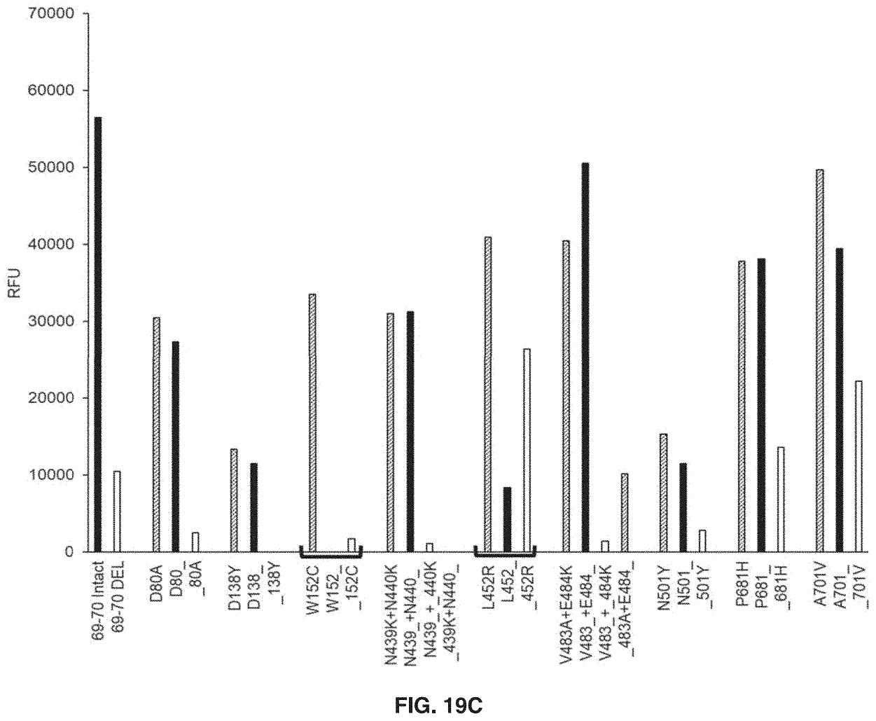

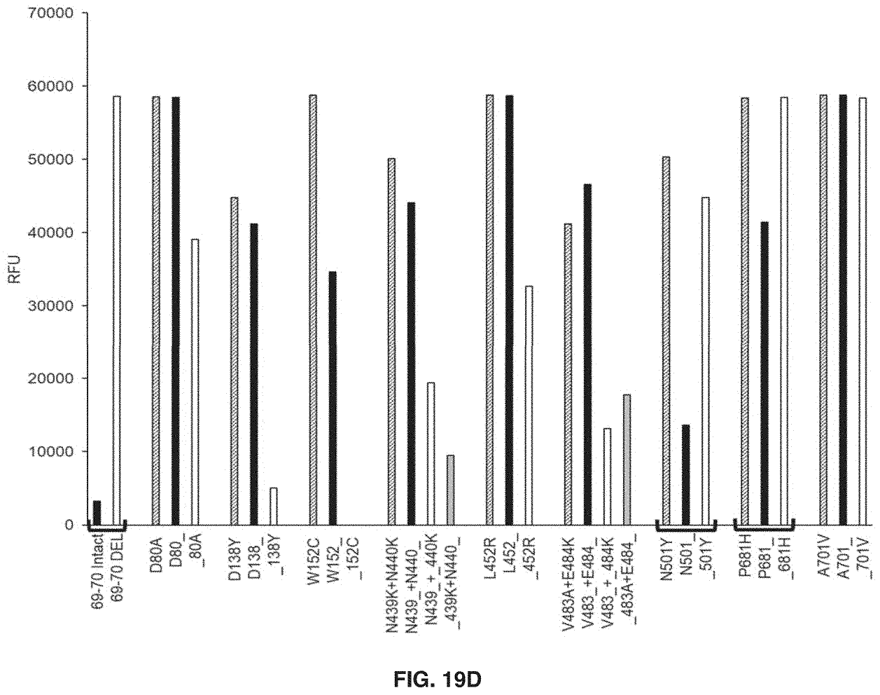

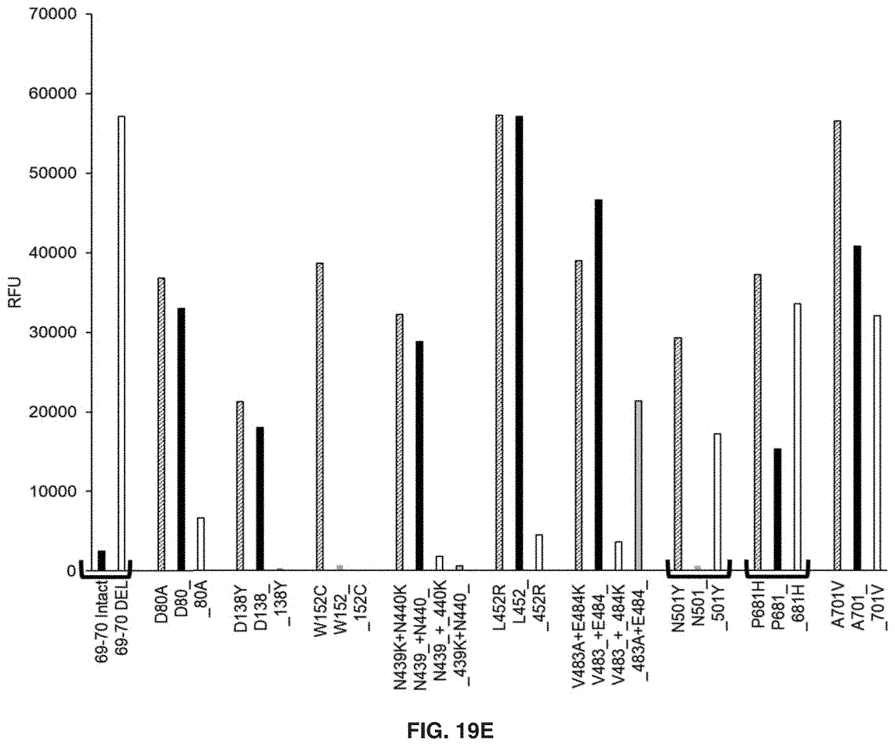

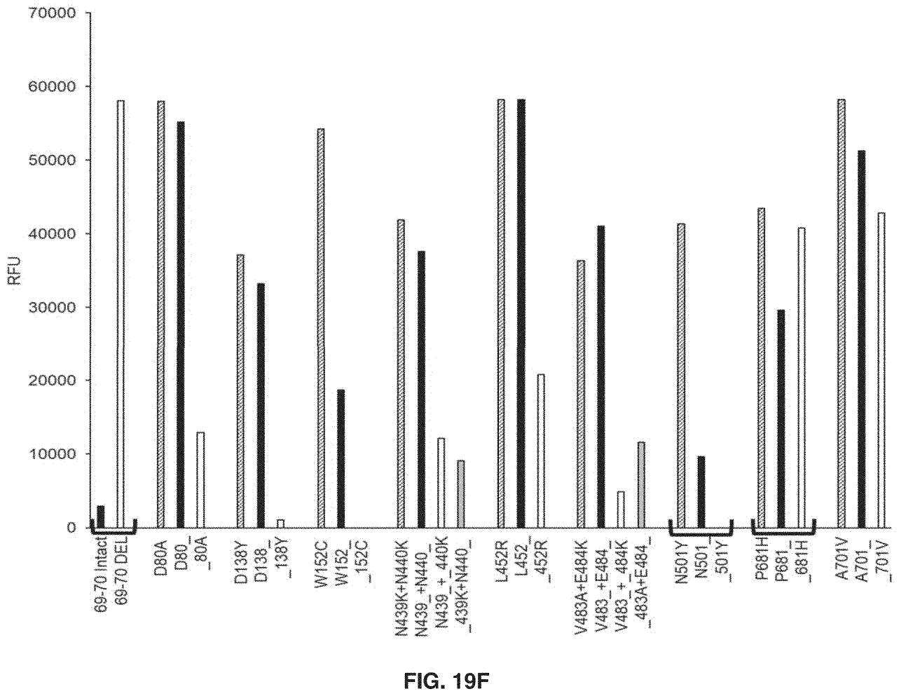

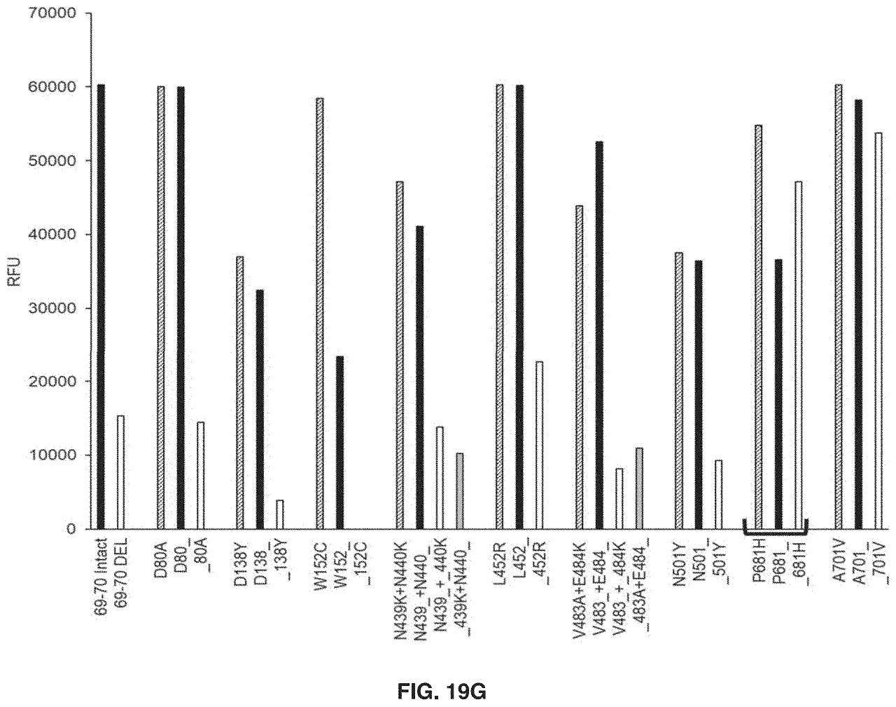

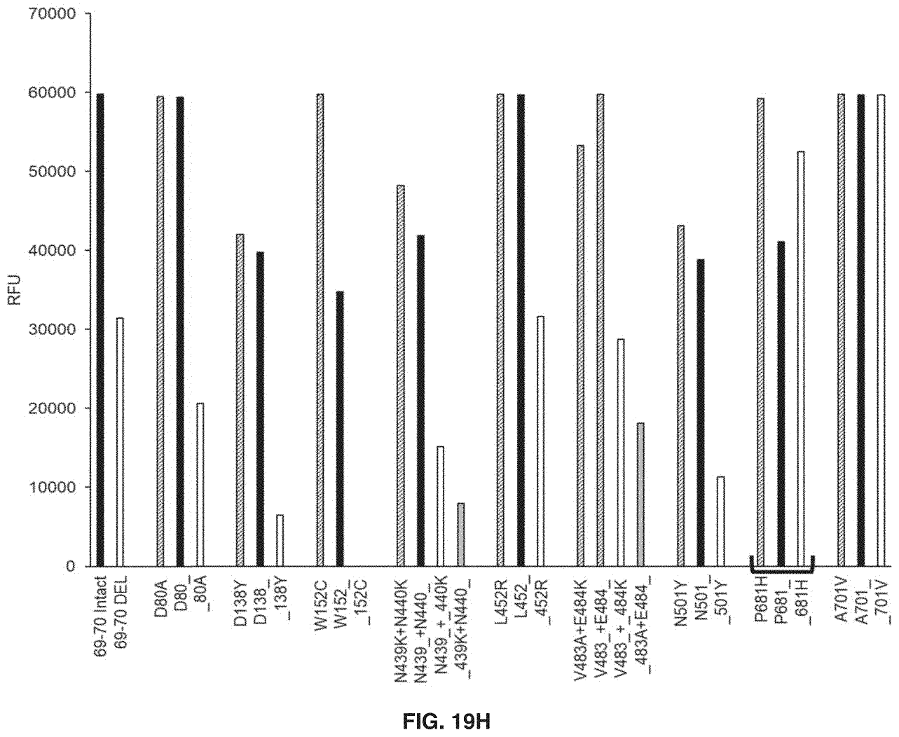

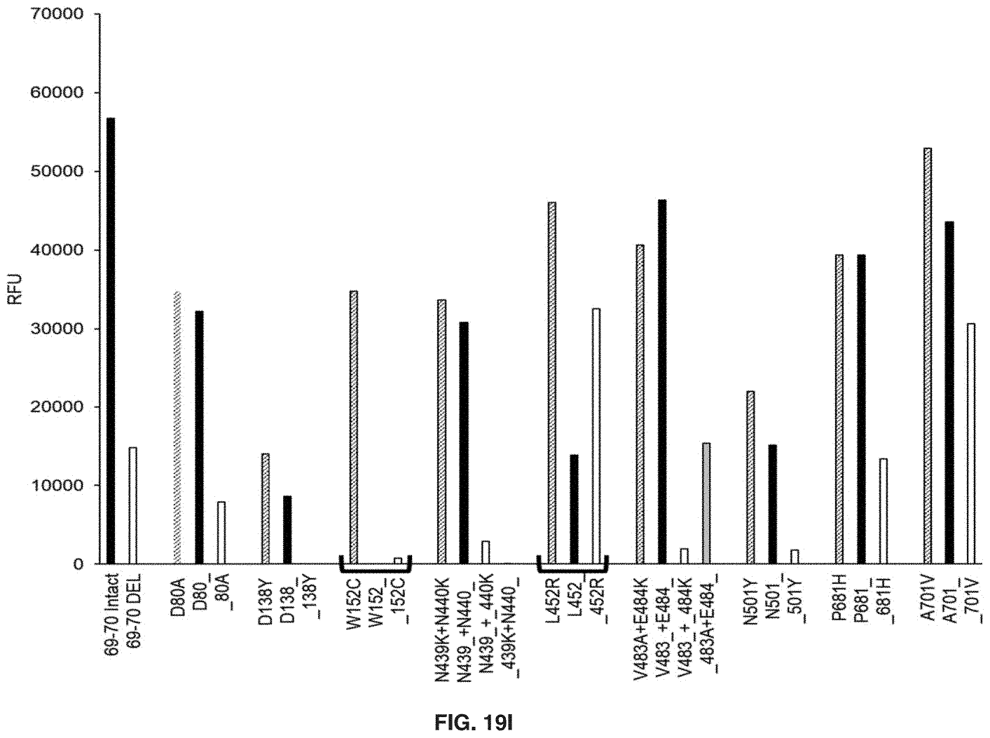

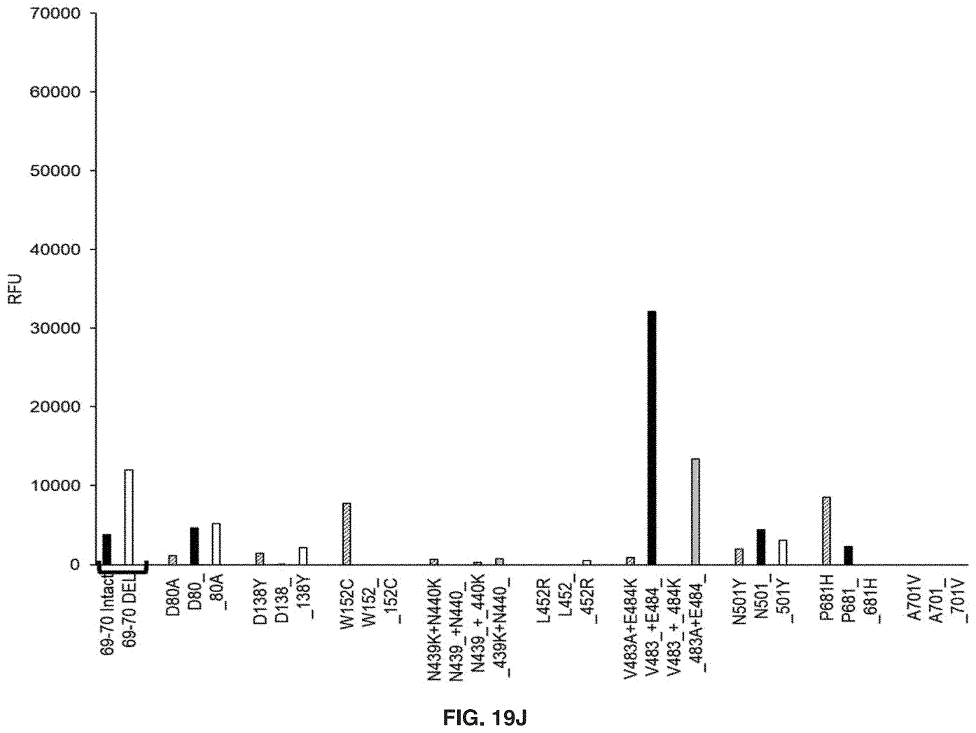

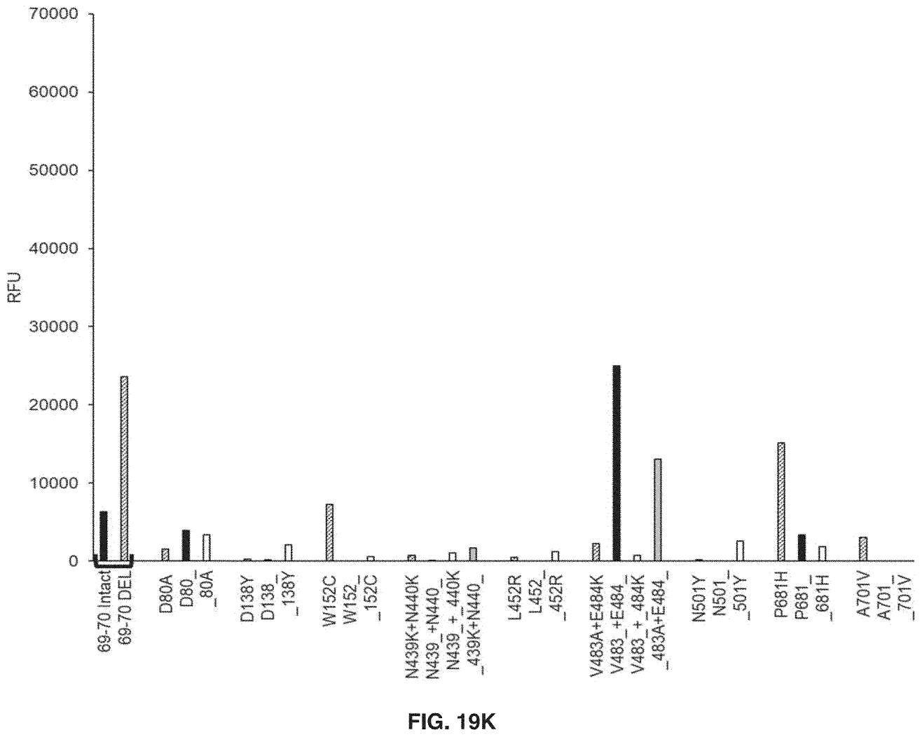

Results FIGS. 19A-19K and Table 23 show the results of the DETECTX-Cv analysis for the clinical samples. It was determined that 68% (19/28) of samples generated data that passed QA/QC in terms of Universal Probe signal strength and were therefore fit for manual or autonomous Augury Clade calls. These data thus demonstrate that high quality DETECTX-Cv data is obtainable with minimal training on clinical positive samples.

TABLE 23
DETECTX-Cv analysis of clinical positive samples performed at TriCore

| | |
|---|---|
| Reference FIG. 19A | TriCore Sample 2-B |

HYBRIDIZED PROBES

| Probe | Wild Type | Mutant Type | Confidence |
|---|---|---|---|
| H/V69_ | ON | | 100.00% |
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _152C | | OFF | 98.80% |
| _484K | | OFF | 100.00% |
| _501Y | | OFF | 93.30% |
| Pattern consistent with: | Wuhan Progenitor | | |

| | |
|---|---|
| Reference FIG. 19B | TriCore Sample 7-B |

HYBRIDIZED PROBES

| Probe | Wild Type | Mutant Type | Confidence |
|---|---|---|---|
| H/V69_ | ON | | 100.00% |
| D80_ | ON | | 100.00% |
| D138_ | ON | | 100.00% |
| _152C | | ON | 100.00% |
| N439_ | ON | | 100.00% |
| N440_ | ON | | 100.00% |
| _452R | | ON | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _80A | | OFF | 100.00% |
| _484K | | OFF | 100.00% |
| _501Y | | OFF | 100.00% |
| Pattern consistent with: | California (B.1.429/427) | | |

| | |
|---|---|
| Reference FIG. 19C | TriCore Sample 9-B |

HYBRIDIZED PROBES

| Probe | Wild Type | Mutant Type | Confidence |
|---|---|---|---|
| H/V69_ | ON | | 100.00% |
| D80_ | ON | | 100.00% |
| D138_ | ON | | 100.00% |
| N439_ | ON | | 100.00% |
| N440_ | ON | | 100.00% |
| _452R | | ON | 100.00% |
| E484_ | ON | | 100.00% |
| N501_ | ON | | 69.70% |
| P681_ | ON | | 100.00% |
| A701_ | ON | | 100.00% |

UNHYBRIDIZED PROBES

| Probe | Wild Type | Mutant Type | Confidence |
|---|---|---|---|
| _80A | | OFF | 100.00% |
| _138Y | | OFF | 100.00% |
| W152_ | OFF | | 100.00% |
| N439_/_440K | | OFF | 100.00% |
| L452_ | OFF | | 97.40% |
| _484K | | OFF | 100.00% |
| _501Y | | OFF | 100.00% |
| _701V | | OFF | 95.40% |
| Pattern consistent with: | California (B.1.429/427) | | |

| | |
|---|---|
| Reference FIG. 19D | TriCore Sample 17-B |

HYBRIDIZED PROBES

| Probe | Wild Type | Mutant Type | Confidence |
|---|---|---|---|
| _69(del) | | ON | 100.00% |
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| _501Y | | ON | 100.00% |
| _681H | | ON | 100.00% |
| A701_ | ON | | 100.00% |

UNHYBRIDIZED PROBES

| Probe | Wild Type | Mutant Type | Confidence |
|---|---|---|---|
| H/V69_ | OFF | | 100.00% |
| _152C | | OFF | 98.80% |
| _484K | | OFF | 100.00% |
| Pattern consistent with: | UK (B.1.1.7) | | |

| | |
|---|---|
| Reference FIG. 19E | TriCore Sample 18-B |

HYBRIDIZED PROBES

| Probe | Wild Type | Mutant Type | Confidence |
|---|---|---|---|
| _69(del) | | ON | 100.00% |
| D80_ | ON | | 100.00% |
| D138_ | ON | | 100.00% |
| N439_ | ON | | 100.00% |
| N440_ | ON | | 100.00% |
| L452_ | ON | | 100.00% |
| E484_ | ON | | 100.00% |
| 501Y | | ON | 100.00% |
| 681H | | ON | 100.00% |
| A701_ | ON | 100.00% | |

UNHYBRIDIZED PROBES

| Probe | Wild Type | Mutant Type | Confidence |
|---|---|---|---|
| H/V69_ | OFF | | 100.00% |
| _80A | | OFF | 100.00% |
| _138Y | | OFF | 100.00% |
| _152C | | OFF | 98.80% |
| W152_ | OFF | | 98.30% |
| N439_/_440K | | OFF | 100.00% |
| _484K | | OFF | 100.00% |
| N501_ | OFF | | 100.00% |
| Pattern consistent with: | UK ( B.1.1.7) | | |

| | |
|---|---|
| Reference FIG. 19F | TriCore Sample 21-B |

HYBRIDIZED PROBES

| Probe | Wild Type | Mutant Type | Confidence |
|---|---|---|---|
| _69(del) | | ON | 100.00% |
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| _501Y | | ON | 100.00% |
| _681H | | ON | 100.00% |
| A701_ | ON | | 100.00% |

UNHYBRIDIZED PROBES

| Probe | Wild Type | Mutant Type | Confidence |
|---|---|---|---|
| H/V69_ | OFF | | 100.00% |
| _138Y | | OFF | 100.00% |
| _152C | | OFF | 98.80% |
| _484K | | OFF | 100.00% |
| Pattern consistent with: | UK (B.1.1.7) | | |

| | |
|---|---|
| Reference FIG. 19G | TriCore Sample 22-B |

HYBRIDIZED PROBES

| Probe | Wild Type | Mutant Type | Confidence |
|---|---|---|---|
| H/V69_ | ON | | 100.00% |
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| N501_ | ON | | 100.00% |
| _681H | | ON | 100.00% |
| A701_ | ON | | 100.00% |

UNHYBRIDIZED PROBES

| Probe | Wild Type | Mutant Type | Confidence |
|---|---|---|---|
| _152C | | OFF | 98.80% |
| _484K | | OFF | 100.00% |
| _501Y | | OFF | 99.80% |
| Pattern consistent with: | B.1.1.207 | | |

| | |
|---|---|
| Reference FIG. 19H | TriCore Sample 24-B |

HYBRIDIZED PROBES

| Probe | Wild Type | Mutant Type | Confidence |
|---|---|---|---|
| H/V69_ | ON | | 100.00% |
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| N501_ | ON | | 100.00% |
| _681H | | ON | 100.00% |
| A701_ | ON | | 100.00% |

UNHYBRIDIZED PROBES

| Probe | Wild Type | Mutant Type | Confidence |
|---|---|---|---|
| _152C | | OFF | 97.60% |
| _501Y | | OFF | 98.60% |
| Pattern consistent with: | B.1.1.207 | | |

| | |
|---|---|
| Reference FIG. 19I | TriCore Sample 27-B |

HYBRIDIZED PROBES

| Probe | Wild Type | Mutant Type | Confidence |
|---|---|---|---|
| H/V69_ | ON | | 100.00% |
| D80_ | ON | | 100.00% |
| D138_ | ON | | 100.00% |
| 152C | | ON | 85.40% |
| N439_ | ON | | 100.00% |
| N440_ | ON | | 100.00% |
| _452R | | ON | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _80A | | OFF | 100.00% |
| _138Y | | OFF | 100.00% |
| _152C | | OFF | 69.40% |
| W152_ | OFF | | 100.00% |
| N439_/_440K | | OFF | 98.30% |
| _484K | | OFF | 100.00% |
| _501Y | | OFF | 100.00% |
| Pattern consistent with: | California (B.1.429/427) | | |

| | |
|---|---|
| Reference FIG. 19J | TriCore Sample 1-B |

HYBRIDIZED PROBES

| Probe | Wild Type | Mutant Type | Confidence |
|---|---|---|---|
| _69(del) | | ON | 87.50% |

UNHYBRIDIZED PROBES

| Probe | Wild Type | Mutant Type | Confidence |
|---|---|---|---|
| H/V69_ | OFF | | 99.70% |
| Pattern consistent with: | | | |
| 1 | UK (B.1.1.7) | | |
| 2 | B.1.525 | | |
| 3 | B.1.375 | | |
| 4 | Denmark | | |
| 5 | B.1.258 | | |
| Reference FIG. 19K | TriCore Sample 4-B | | |
| START_WELL_20 | | | |
| SPECIMEN_ID | Sample #20 | | |

HYBRIDIZED PROBES

| Probe | Wild Type | Mutant Type | Confidence |
|---|---|---|---|
| _69(del) | | ON | 100.00% |

UNHYBRIDIZED PROBES

| Probe | Wild Type | Mutant Type | Confidence |
|---|---|---|---|
| Pattern consistent with : | NA | | |
| END_WELL_20 | | | |

### DETECTX-Cv Analysis of TriCore Clinical Positive Samples at PathogenDx.

Sixty (60) clinical positive NP-VTM samples collected by TriCore were sent to PathogenDx for DETECTX-Cv analysis. RNA was extracted from these samples using the Zymo Magnetic Bead platform. The extracted RNA (5 IL) was processed using the DETECTX-Cv method. The PCR recipe and cycling conditions were as described in Table 20.

### Results

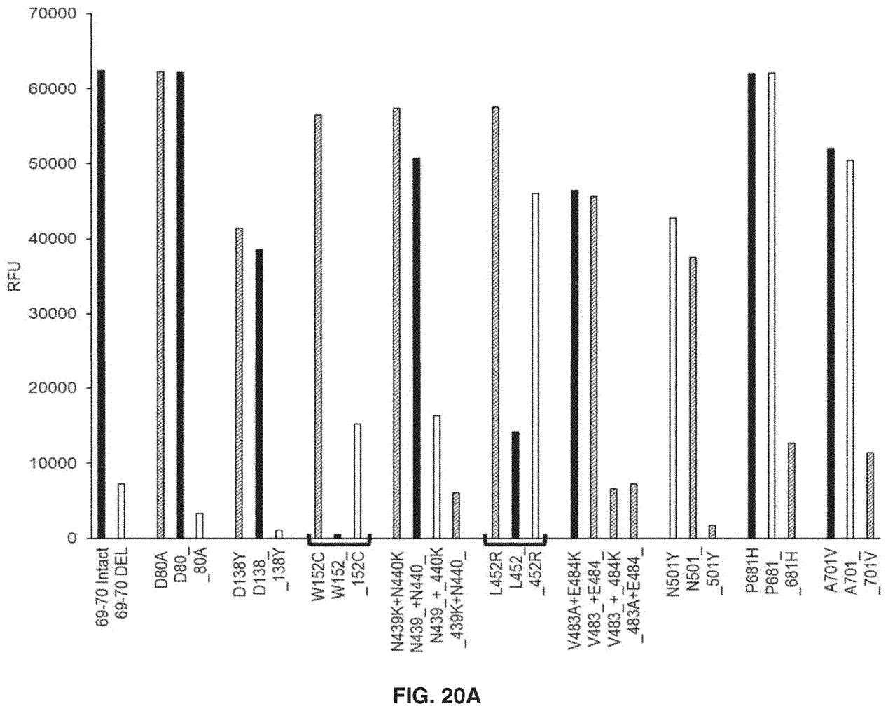

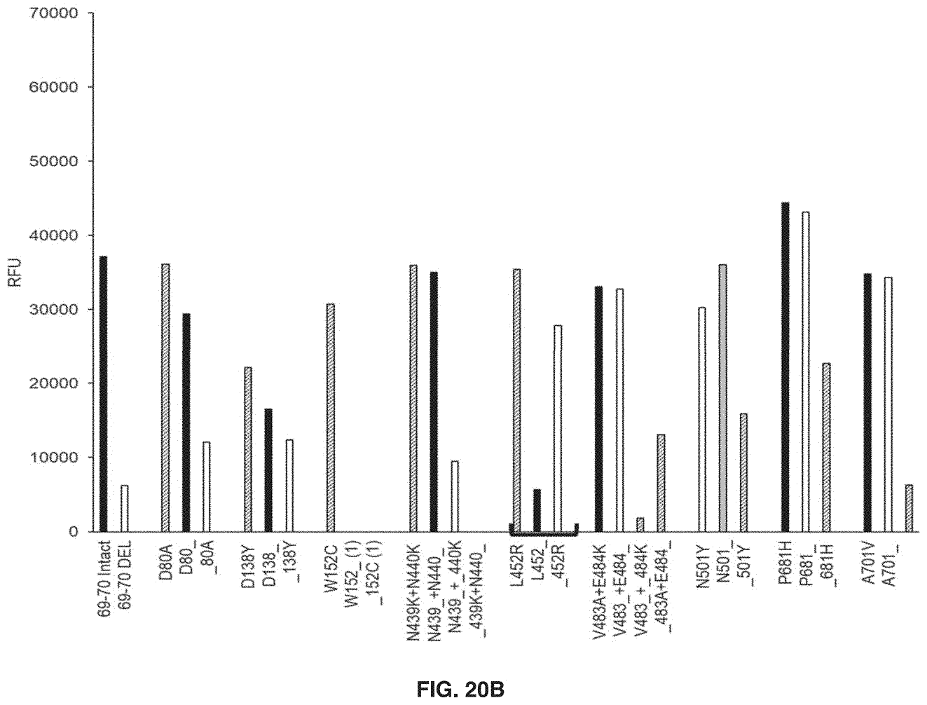

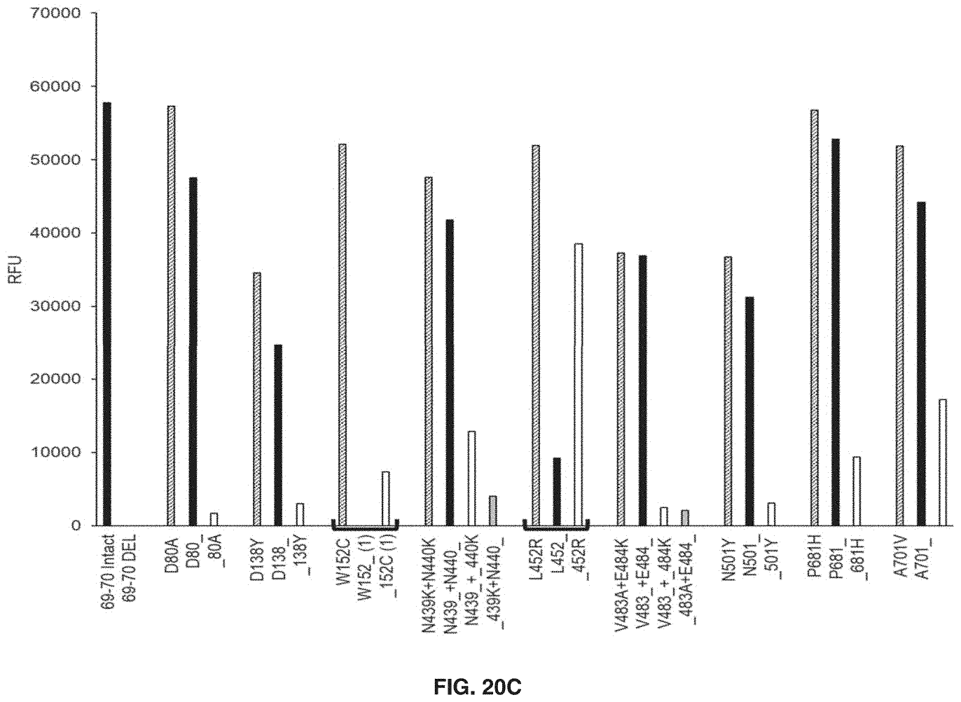

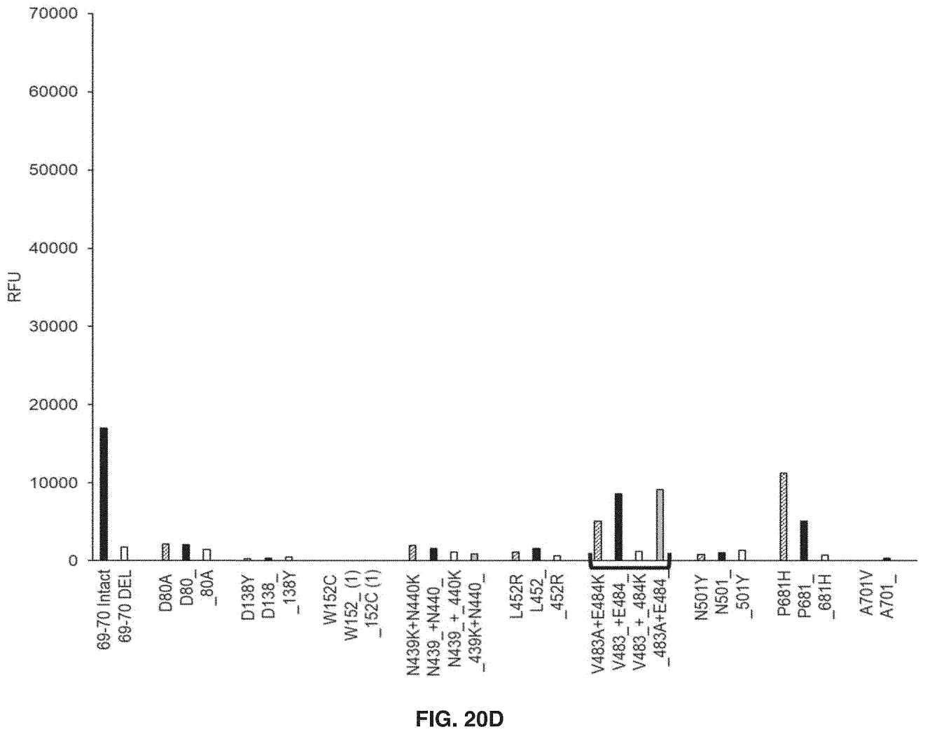

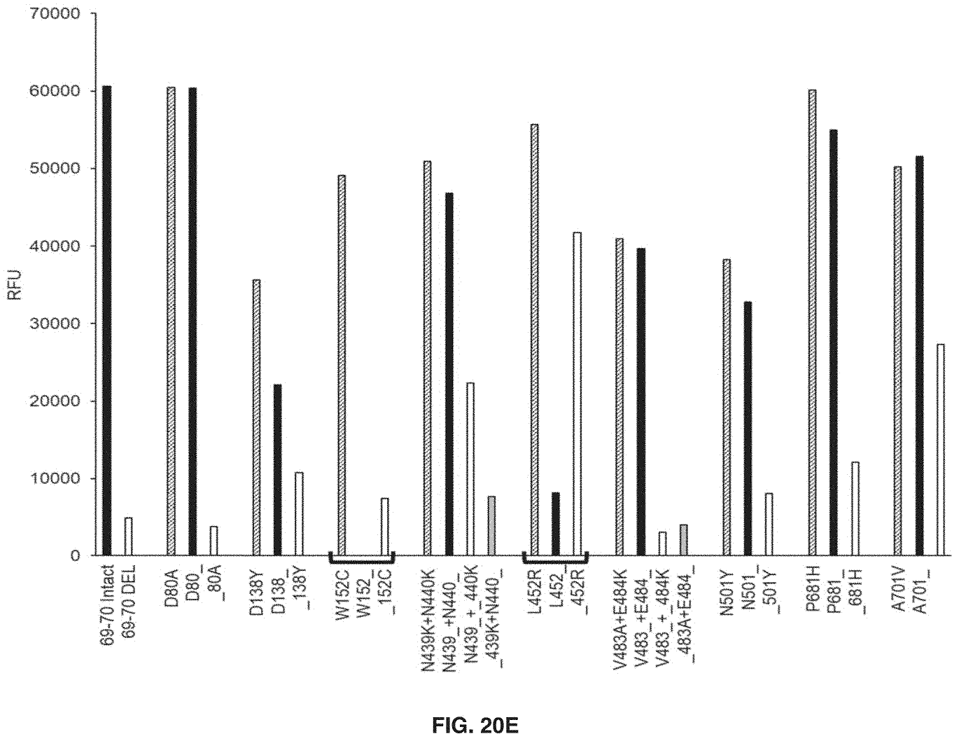

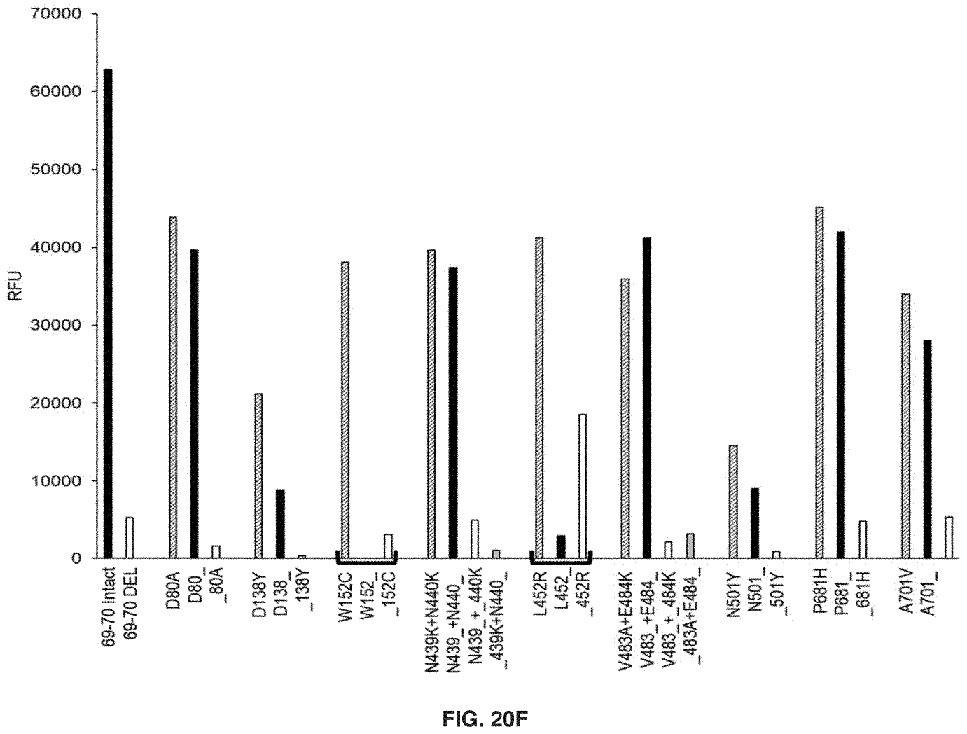

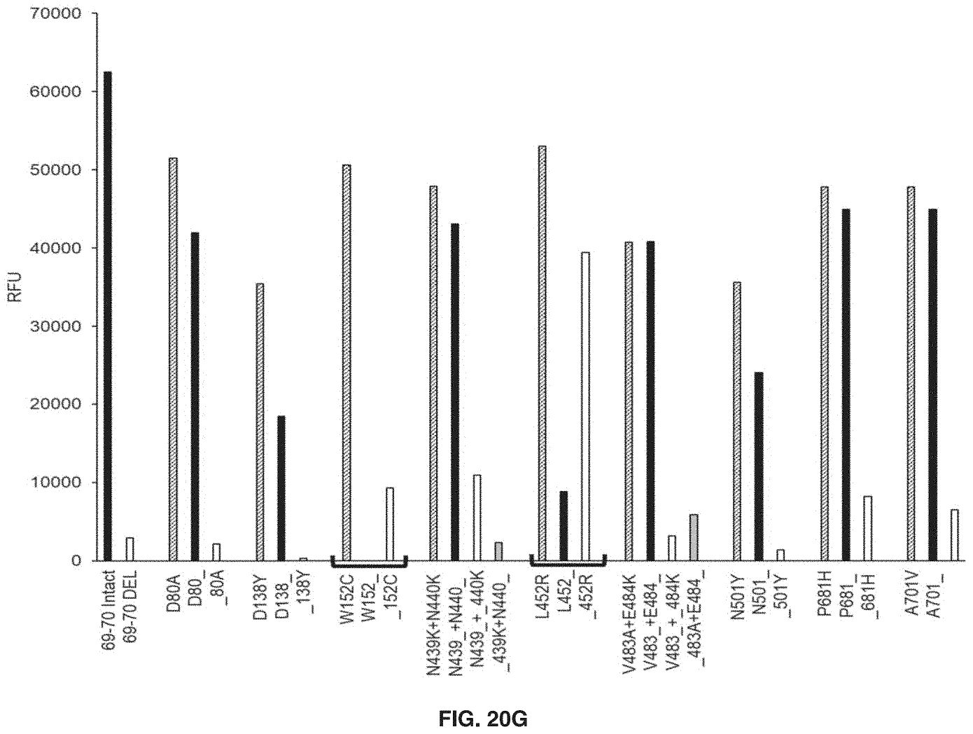

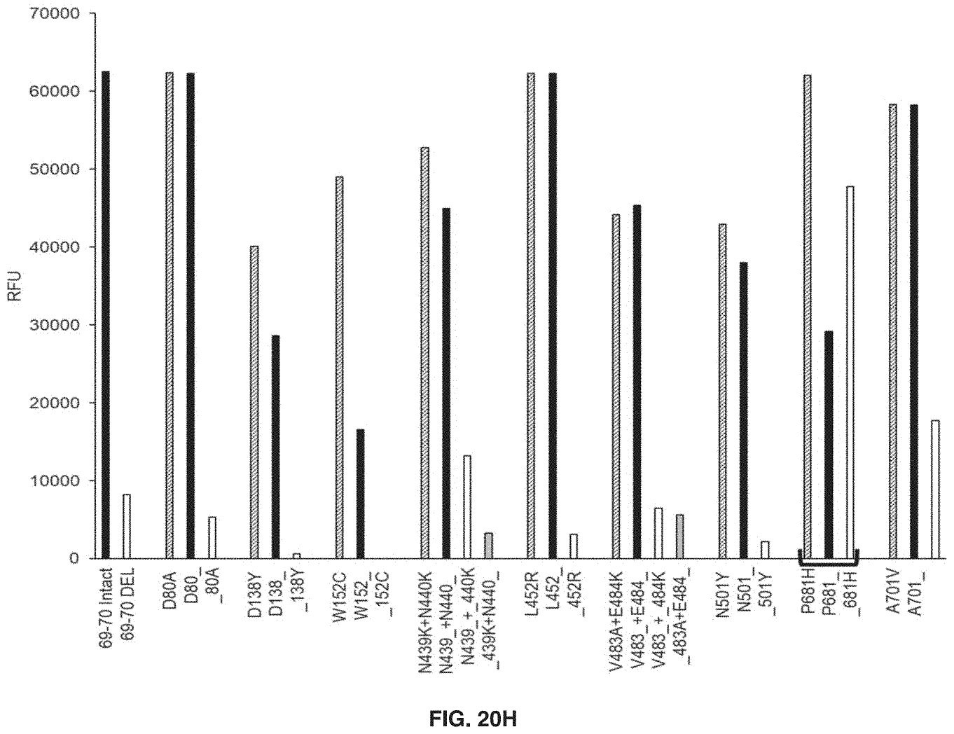

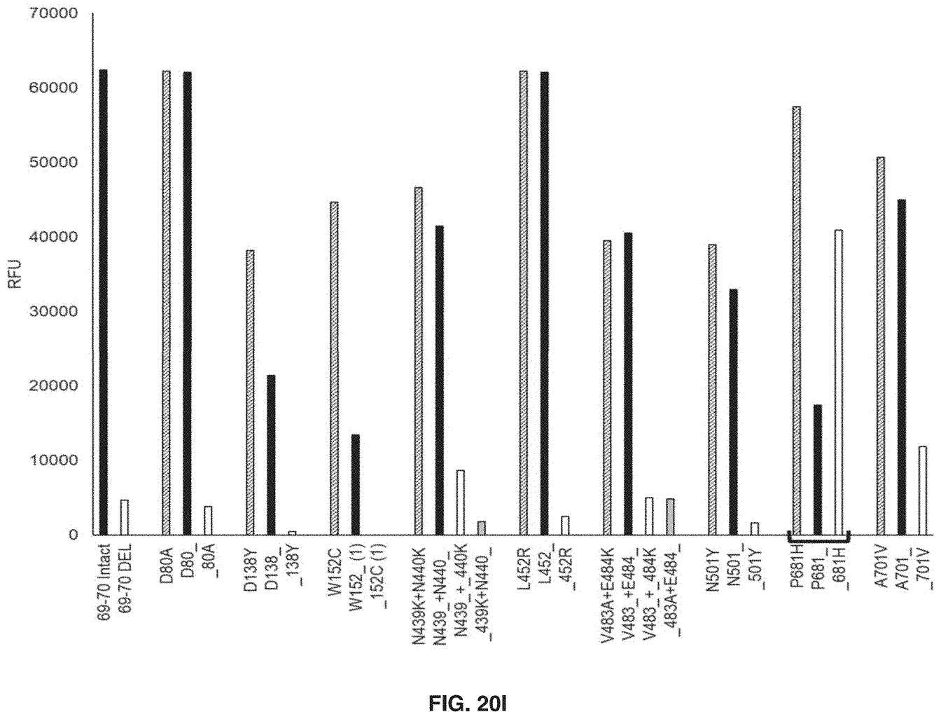

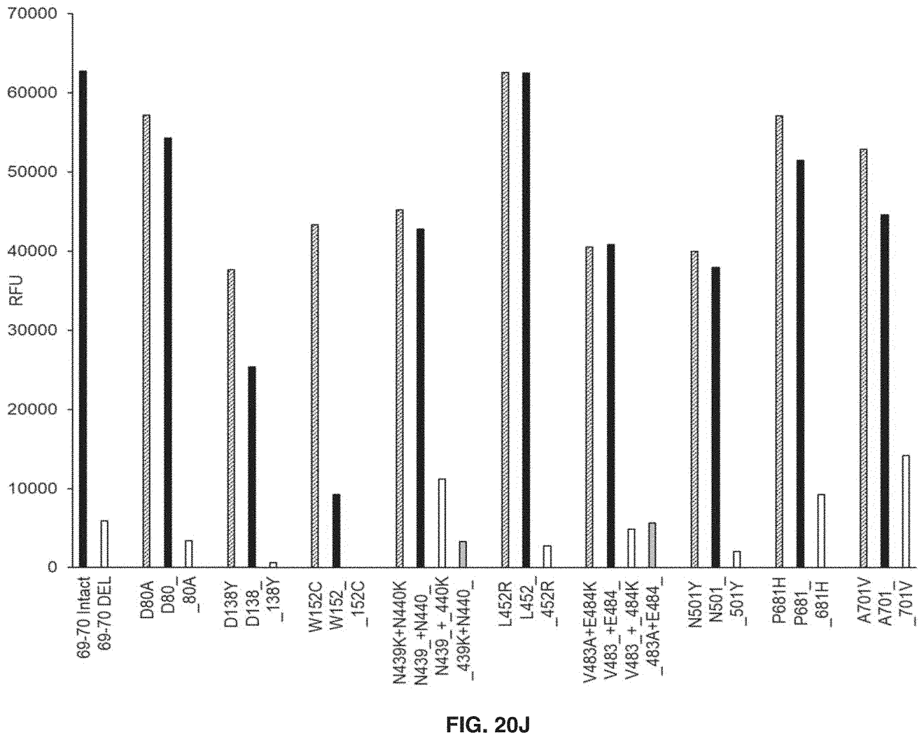

FIGS. 20A-20J and Table 24 show the results of this analysis. It was determined that 65% (39/60) of these samples generated data which passed QC/QA in terms of signal strength and were thus fit for manual or autonomous Augury Clade calls. The data shows all NP-VTM specimens which passed QA/QC and which displayed nonstandard clade variants (other than Wuhan) and representative data (2) for which QA/QC were inadequate either due to low RNA concentration or degraded RNA in the sample.

TABLE 24
DETECTX-Cv analysis of clinical positive samples performed at PathogenDx

| | |
|---|---|
| Reference FIG. 20A | TriCore 238480-d Sample 1 |

HYBRIDIZED PROBES

| Probe | Wild Type | Mutant Type | Confidence |
|---|---|---|---|
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _69(del) | | OFF | 100.00% |
| _80A | | OFF | 100.00% |
| _138Y | | OFF | 100.00% |
| _152C | | OFF | 98.80% |
| _452R | | OFF | 98.00% |
| _484K | | OFF | 100.00% |
| _501Y | | OFF | 100.00% |
| _681H | | OFF | 87.00% |
| _701V | | OFF | 100.00% |
| Pattern consistent with: | Wuhan Progenitor | | |

| | |
|---|---|
| Reference FIG. 20B | TriCore 238484-d Sample 4 |

HYBRIDIZED PROBES

| Probe | Wild Type | Mutant Type | Confidence |
|---|---|---|---|
| D80_ | ON | | 100.00% |
| D138_ | ON | | 100.00% |
| N439_ | ON | | 100.00% |
| N440_ | ON | | 100.00% |
| _452R | | ON | 100.00% |
| E484_ | ON | | 100.00% |
| N501_ | ON | | 100.00% |
| P681_ | ON | | 100.00% |
| A701_ | ON | | 99.90% |

UNHYBRIDIZED PROBES

| Probe | Wild Type | Mutant Type | Confidence |
|---|---|---|---|
| _69(del) | | OFF | 100.00% |
| _80A | | OFF | 89.00% |
| _152C | | OFF | 98.80% |
| W152_ | OFF | | 100.00% |
| L452_ | OFF | | 100.00% |
| _484K | | OFF | 100.00% |
| _701V | | OFF | 100.00% |
| Pattern consistent with: | No Clade Call, likely California | | |

| | |
|---|---|
| Reference FIG. 20C | TriCore 238485-d Sample 5 |

HYBRIDIZED PROBES

| Probe | Wild Type | Mutant Type | Confidence |
|---|---|---|---|
| D80_ | ON | | 100.00% |
| D138_ | ON | | 100.00% |
| _152C | | ON | 100.00% |
| N439_ | ON | | 100.00% |
| N440_ | ON | | 100.00% |
| _452R | | ON | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _69(del) | | OFF | 100.00% |
| _80A | | OFF | 100.00% |
| W152_ | OFF | | 100.00% |
| L452_ | OFF | | 90.50% |
| _484K | | OFF | 100.00% |
| _501Y | | OFF | 100.00% |
| _681H | | OFF | 83.60% |
| _701V | | OFF | 99.90% |
| Pattern consistent with: | California (B.1.429/427) | | |

| | |
|---|---|
| Reference FIG. 20D | TriCore 238487-d Sample 6 |

HYBRIDIZED PROBES

| | | | |
|---|---|---|---|
| Probe | Wild Type | Mutant Type | Confidence |

UNHYBRIDIZED PROBES

| Probe | Wild Type | Mutant Type | Confidence |
|---|---|---|---|
| _69(del) | | OFF | 100.00% |
| Pattern consistent with: | NA | | |

| | |
|---|---|
| Reference FIG. 20E | TriCore 238488-d Sample 7 |

HYBRIDIZED PROBES

| Probe | Wild Type | Mutant Type | Confidence |
|---|---|---|---|
| D80_ | ON | | 100.00% |
| D138_ | ON | | 100.00% |
| _152C | | ON | 100.00% |
| N439_ | ON | | 100.00% |
| N440_ | ON | | 100.00% |
| _452R | | ON | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _69(del) | | OFF | 100.00% |
| _80A | | OFF | 100.00% |
| W152_ | OFF | | 100.00% |
| L452_ | OFF | | 98.50% |
| _484K | | OFF | 100.00% |
| _501Y | | OFF | 100.00% |
| Pattern consistent with: | California (B.1.429/427) | | |

| | |
|---|---|
| Reference FIG. 20F | TriCore 238498-d Sample 12 |

HYBRIDIZED PROBES

| Probe | Wild Type | Mutant Type | Confidence |
|---|---|---|---|
| D80_ | ON | | 100.00% |
| D138_ | ON | | 100.00% |
| _152C | | ON | 97.60% |
| N439_ | ON | | 100.00% |
| N440_ | ON | | 100.00% |
| _452R | | ON | 100.00% |
| E484_ | ON | | 100.00% |
| P681_ | ON | | 100.00% |
| A701_ | ON | | 78.60% |

UNHYBRIDIZED PROBES

| Probe | Wild Type | Mutant Type | Confidence |
|---|---|---|---|
| _69(del) | | OFF | 100.00% |
| _80A | | OFF | 100.00% |
| _138Y | | OFF | 100.00% |
| W152_ | OFF | | 100.00% |
| L452_ | OFF | | 100.00% |
| _484K | | OFF | 100.00% |
| _501Y | | OFF | 100.00% |
| _681H | | OFF | 100.00% |
| _701V | | OFF | 100.00% |
| Pattern consistent with: | California (B.1.429/427) | | |

| | |
|---|---|
| Reference FIG. 20G | TriCore 238499-d Sample 13 |

HYBRIDIZED PROBES

| Probe | Wild Type | Mutant Type | Confidence |
|---|---|---|---|
| D80_ | ON | | 100.00% |
| D138_ | ON | | 100.00% |
| _152C | | ON | 100.00% |
| N439_ | ON | | 100.00% |
| N440_ | ON | | 100.00% |
| _452R | | ON | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _69(del) | | OFF | 100.00% |
| _80A | | OFF | 100.00% |
| _138Y | | OFF | 100.00% |
| W152_ | OFF | | 100.00% |
| L452_ | OFF | | 94.40% |
| _484K | | OFF | 100.00% |
| _501Y | | OFF | 100.00% |
| _681H | | OFF | 98.40% |
| _701V | | OFF | 100.00% |
| Pattern consistent with: | California (B.1.429/427) | | |

| | |
|---|---|
| Reference FIG. 20H | TriCore 238504-d Sample 16 |

HYBRIDIZED PROBES

| Probe | Wild Type | Mutant Type | Confidence |
|---|---|---|---|
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| N501_ | ON | | 100.00% |
| _681H | | ON | 100.00% |
| A701_ | ON | | 100.00% |

UNHYBRIDIZED PROBES

| Probe | Wild Type | Mutant Type | Confidence |
|---|---|---|---|
| _69(del) | | OFF | 99.70% |
| _80A | | OFF | 100.00% |
| _138Y | | OFF | 100.00% |
| _152C | | OFF | 97.40% |
| _452R | | OFF | 94.60% |
| _484K | | OFF | 100.00% |
| _501Y | | OFF | 100.00% |
| _701V | | OFF | 99.80% |
| Pattern consistent with: | B.1.1.207 | | |

| | |
|---|---|
| Reference FIG. 201 | TriCore 236310-P Sample 39 |

HYBRIDIZED PROBES

| Probe | Wild Type | Mutant Type | Confidence |
|---|---|---|---|
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| N501_ | ON | | 100.00% |
| _681H | | ON | 100.00% |
| A701_ | ON | | 100.00% |

UNHYBRIDIZED PROBES

| Probe | Wild Type | Mutant Type | Confidence |
|---|---|---|---|
| _69(del) | | OFF | 100.00% |
| _80A | | OFF | 100.00% |
| _138Y | | OFF | 100.00% |
| _152C | | OFF | 98.80% |
| _452R | | OFF | 98.90% |
| _484K | | OFF | 100.00% |
| _501Y | | OFF | 100.00% |
| _701V | | OFF | 100.00% |
| Pattern consistent with: | B.1.1.207 | | |

| | |
|---|---|
| Reference FIG. 20J | TriCore 236315-P Sample 42 |

HYBRIDIZED PROBES

| Probe | Wild Type | Mutant Type | Confidence |
|---|---|---|---|
| D80_ | ON | | 100.00% |
| D138_ | ON | | 100.00% |
| W152_ | ON | | 100.00% |
| N439_ | ON | | 100.00% |
| N440_ | ON | | 100.00% |
| L452_ | ON | | 100.00% |
| E484_ | ON | | 100.00% |
| N501_ | ON | | 100.00% |
| P681_ | ON | | 100.00% |
| A701_ | ON | | 100.00% |

UNHYBRIDIZED PROBES

| Probe | Wild Type | Mutant Type | Confidence |
|---|---|---|---|
| _69(del) | | OFF | 100.00% |
| _80A | | OFF | 100.00% |
| _138Y | | OFF | 100.00% |
| _152C | | OFF | 98.80% |
| _452R | | OFF | 98.00% |
| _484K | | OFF | 100.00% |
| _501Y | | OFF | 100.00% |
| _681H | | OFF | 87.00% |
| _701V | | OFF | 100.00% |
| Pattern consistent with: | Wuhan Progenitor | | |

### Example 11

### Detection of Stable Genetic Variation

The method of nucleic acid analysis to detect stable genetic variations in a pathogen is based on simultaneous analysis of multiple sequence domains in a gene or group of genes, such as the Spike gene or the LINK domain of the Nucleoprotein (N) gene in SARS-CoV-2 or a combination of the Spike gene and the N gene in the RNA genome to measure clade variations in SARS-CoV-2. For SARS-CoV-2 (CoV-2), the sequence domains are processed for nucleic acid analysis by converting them into a set of amplicons via a multiplex RT-PCR reaction. In one preferred implementation, the sequence of the multiplex RT-PCR products is identified relative to that of the underlying CoV-2 Spike gene, by the Horizontal Black Bars in the bottom of Table 25 or is identified relative to that of the underlying CoV-2 N gene LINK region, by the Horizontal Black Bar at the bottom of Table 26.

The product of the multiplex RT-PCR reaction is analyzed by hybridization to a matrix of synthetic oligonucleotide probes positioned as a microarray. In one preferred implementation of the present invention for CoV-2, there are (32) such Spike Gene Target Regions (Table 25 & 28) and (11) N gene Target Regions (Table 26, 28) containing meaningful local sequence variation which may be used to measure a pattern of mutation for SARS-CoV2, which in combination, can be used for SARS-CoV-2 Variant Identification. See the top Row of Table 25 for localization of those Target sites in the Spike gene and the top row of Table 26 for their location in the LINK domain of the N protein.

In terms of detailed test design, the forward and reverse Primers deployed for multiplex amplification of the Spike gene (Amplicons S:1-S:8) and those for PCR amplification of the N gene LINK region (Amplicon N:9) are listed in Table 27.

In terms of detailed test design, the Hybridization Probes resident at each target region of the Spike surface protein and each target region of the N gene LINK domain are each produced as 3 closely related types of probe variants, which may be referred to as “Wild Type”, “Mutant” and “Universal”. Those Spike gene and N gene Hybridization probe sequences are listed in Table 28.

TABLE 25
Spike Gene Variant Lookup Table

| VOC/VOI/VUM LIST | | | | | | | | | | | | | | |
|---|---|---|---|---|---|---|---|---|---|---|---|---|---|---|
| COMBINED WHO & | | | | | | | | | | | | | | |
| CDC 10-21-2021 | | S: L5F | S: S13I | S: L18F | S: T19R | S: T20N | S: A67V | S: HV69-70del | S: G75V.T76I | S: D80A | S: D80G | S: T95I | S: D138Y | S: Y144del |
| Alpha | B.1.1.7(Q) | | | | | | | 97 | | | | | | 95 |
| Beta | B.1.351 | | | | | | | | | 97 | | | | |
| Gamma | P.1 | | | 98 | | 97 | | | | | | | 97 | |
| Delta | B.1.617.2(AY) | | | | 98 | | | | | | | 35 | | |
| Lambda | C.37 | | | | | | | | 96 | | | | | |
| Mu | B.1.621 | | | | | | | | | | | 94 | | |
| | R.1 | | | | | | | | | | | | | |
| | B.1.466.2 | | | | | | | | | | | | | |
| | B.1.1.318 | | | | | | | | | | | 98 | | 91 |
| | B.1.1.519 | | | | | | | | | | | | | |
| | C.36.3 | | | | | | | 78 | | | | | | |
| | B.1.214.2 | | | | | | | | | | | | | |
| Epsilon | B.1.429/427 | | 94 | | | | | | | | | | | |
| | B.1.523 | | | | | | | | | | | | | |
| | B.1.619 | | | | | | | | | | | | | |
| | B.1.620 | | | | | | | 98 | | | | | | 99 |
| | C.1.2 | | | | | | | | | | | | | 77 |
| Kappa | B.1.617.1 | | | | | | | | | | | 48 | | |
| Iota | B.1.526 | 96 | | | | | | | | | | 99 | | |
| Eta | B.1.525 | | | | | | 95 | 94 | | | | | | 93 |
| | B.1.630 | | | | | | | | | | | | | 92 |
| Zeta | P.2 | | | | | | | | | | | | | |
| Theta | P.3 | | | | | | | | | | | | | |
| | B.1.617.3 | | | | 95 | | | | | | | | | |
| VOC/VOI/VUM LIST | | | | | | | | | | | | | | |
| COMBINED WHO & | | | | S: L141Y.142- | | | | | | S: E156G.157- | | | | S: R246N.247- |
| CDC 10-21-2021 | | S: ins143T | S: G142D | 144del | S: Y145H | S: W152C | S: W152L | S: W152R | S: E154K | 158del | S: D215G | S: A222V | S: 242-244del | 253del |
| Alpha | B.1.1.7(Q) | | | | | | | | | | | | | |
| Beta | B.1.351 | | | | | | | | | | 94 | | 68 | |
| Gamma | P.1 | | | | | | | | | | | | | |
| Delta | B.1.617.2(AY) | | 59 | | | | | | | 91 | | | | |
| Lambda | C.37 | | | | | | | | | | | | | 85 |
| Mu | B.1.621 | 80 | | | | | | | | | | | | |
| | R.1 | | | | | | 99 | | | | | | | |
| | B.1.466.2 | | | | | | | | | | | | | |
| | B.1.1.318 | | | | | | | | | | | | | |
| | B.1.1.519 | | | | | | | | | | | | | |
| | C.36.3 | | | | | | | 97 | | | | | | |
| | B.1.214.2 | | | | | | | | | | | | | |
| Epsilon | B.1.429/427 | | | | | 92 | | | | | | | | |
| | B.1.523 | | | | | | | | | | | | | |
| | B.1.619 | | | | | | | | | | | | | |
| | B.1.620 | | | | | | | | | | | | 77 | |
| | C.1.2 | | | | | | | | | | 96 | | | |
| Kappa | B.1.617.1 | | 57 | | | | | | 63 | | | | | |
| Iota | B.1.526 | | | | | | | | | | | | | |
| Eta | B.1.525 | | | | | | | | | | | | | |
| | B.1.630 | | | | | | | | | | | 94 | | |
| Zeta | P.2 | | | | | | | | | | | | | |
| Theta | P.3 | | | | | | | | | | | | | |
| | B.1.617.3 | | 16 | | | | | | | 87 | | | | |
| VOC/VOI/VUM LIST | | | | | | | | | | | | | | |
| COMBINED WHO & | | | | | | | | | | | | | | |
| CDC 10-21-2021 | | S: D253G | S: K417N | S: K417T | S: N439K | S: N440K | S: L452R | S: L452Q | S: S477N | S: T478K | S: V483A | S: E484K | S: E484Q | S: S494P |
| Alpha | B.1.1.7(Q) | | | | | | | | | | | | | |
| Beta | B.1.351 | | 93 | | | | | | | | | 86 | | |
| Gamma | P.1 | | | 96 | | | | | | | | 95 | | |
| Delta | B.1.617.2(AY) | | | | | | 98 | | | 98 | | | | |
| Lambda | C.37 | | | | | | | 98 | | | | | | |
| Mu | B.1.621 | | | | | | | | | | | 94 | | |
| | R.1 | | | | | | | | | | | 99 | | |
| | B.1.466.2 | | | | 92 | | | | | | | | | |
| | B.1.1.318 | | | | | | | | | | | 97 | | |
| | B.1.1.519 | | | | | | | | | 93 | | | | |
| | C.36.3 | | | | | | 98 | | | | | | | |
| | B.1.214.2 | | | | | | | | | | | | | |
| Epsilon | B.1.429/427 | | | | | | 96 | | | | | | | |
| | B.1.523 | | | | | | | | | | | | | |
| | B.1.619 | | | | | 87 | | | | | | 97 | | |
| | B.1.620 | | | | | | | | 99 | | | 99 | | |
| | C.1.2 | | | | | | | | | 19 | | 86 | | |
| Kappa | B.1.617.1 | | | | | | 93 | | | | | | 93 | |
| Iota | B.1.526 | 97 | | | | | | | 38 | | | 55 | | |
| Eta | B.1.525 | | | | | | | | | | | 97 | | |
| | B.1.630 | | | | | | 96 | | | | | | 95 | |
| Zeta | P.2 | | | | | | | | | | | 94 | | |
| Theta | P.3 | | | | | | | | | | | 86 | | |
| | B.1.617.3 | | | | | | 89 | | | | | | 92 | |

| VOC/VOI/VUM LIST | | | | | | | | | | | | | |
|---|---|---|---|---|---|---|---|---|---|---|---|---|---|
| COMBINED WHO & | | | | | | | | | | | | | |
| CDC 10-21-2021 | | S: F490S | S: N501Y | S: N501T | S: Q613H | S: D614G | S: Q677P | S: Q677H2 | S: Q677H1 | S: P681R | S: P681H | S: A688V | S: A701V |
| Alpha | B.1.1.7(Q) | | 98 | | | 99 | | | | | 99 | | |
| Beta | B.1.351 | | 87 | | | 98 | | | | | | | 96 |
| Gamma | P.1 | | 95 | | | 99 | | | | | | | |
| Delta | B.1.617.2(AY) | | | | | 99 | | | | 99 | | | |
| Lambda | C.37 | 97 | | | | 99 | | | | | | | |
| Mu | B.1.621 | | 94 | | | 97 | | | | | 97 | | |
| | R.1 | | | | | 99 | | | | | | | |
| | B.1.466.2 | | | | | 99 | | | | 87 | | | |
| | B.1.1.318 | | | | | 99 | | | | | 98 | | |
| | B.1.1.519 | | | | | 99 | | | | | 98 | | |
| | C.36.3 | | | | | 99 | | 98 | | | | | |
| | B.1.214.2 | | | | | 99 | | | | | | | |
| Epsilon | B.1.429/427 | | | | | 99 | | | | | | | |
| | B.1.523 | | | | | 99 | | | | | | | |
| | B.1.619 | | | | | 99 | | | | | | | |
| | B.1.620 | | | | | 99 | | | | | 99 | | |
| | C.1.2 | | 84 | | | 99 | | | | | | | |
| Kappa | B.1.617.1 | | | | | 98 | | | | 97 | | | |
| Iota | B.1.526 | | | | | 99 | | | | | | | 65 |
| Eta | B.1.525 | | | | | 99 | | 98 | | | | | |
| | B.1.630 | | | | | 99 | | | | | | | |
| Zeta | P.2 | | | | | 99 | | | | | | | |
| Theta | P.3 | | 86 | | | 98 | | | | | 96 | | |
| | B.1.617.3 | | | 92 | | 99 | | | | 98 | | | |

N Gene (LINK domain) Variant Lookup Table

| | | NUCLEOCAPSID (N) GENE MUTATIONS | | | | | | | | | | | | | | | | |
|---|---|---|---|---|---|---|---|---|---|---|---|---|---|---|---|---|---|---|
| VOC/VOI/VUM LIST | | DETECTED IN THE PRESENT INVENTION | | | | | | | | | | | | | | | | |
| COMBINED WHO & | | N: | N: | N: | N: | N: | N: | N: | | | | | | | | | | |
| CDC 10-21-2021 | | S194L | S197L | P199L | S201I | S202N | R203M | R203K | N: K G204R | N: T205I | N: A208G | N: R209del | N: G212V | N: N213Y | N: G214C | N: G215C | N: M234I | N: S235F |
| Alpha | B.1.1.7(Q) | | | | | | | 91 | 91 | | | | | | | | | 99 |
| Beta | B.1.351 | | | | | | | | | 99 | | | | | | | | |
| Gamma | P.1 | | | | | | | 95 | 95 | | | | | | | | | |
| Delta | B.1.617.2(AY) | | | | | | 99 | | | | | | | | | 85 | | |
| Lambda | C.37 | | | | | | | 97 | 97 | | | | | | 98 | | | |
| Mu | B.1.621 | | | | | | | | | 95 | | | | | | | | |
| | R.1 | | | | | | | 99 | 99 | | | | | | | | | |
| | B.1.466.2 | | | | | | | | | 97 | | | | | | | | |
| | B.1.1.318 | | | | | | | 96 | 96 | | 94 | 94 | | | | | | |
| | B.1.1.519 | | | | | | | 97 | 97 | | | | | | | | | |
| | C.36.3 | | | | | | | 99 | 99 | | | | 98 | | | | | |
| | B.1.214.2 | | | | | | | | | 94 | | | | | | | | |
| Epsilon | B.1.429/427 | | | | | | | | | 98 | | | | | | | 29 | |
| | B.1.523 | | | | | | | | | | | | | | | | 99 | |
| | B.1.619 | | | | 97 | | | | | 97 | | | | | | | | |
| | B.1.620 | | | | | | | | | | | | | | | | | |
| | C.1.2 | | | | | | | 97 | 97 | | | | | | | | | |
| Kappa | B.1.617.1 | | | | | | 95 | | | | | | | | | | | |
| Iota | B.1.526 | | | 68 | | | | | | | | | | | | | 67 | |
| Eta | B.1.525 | | | | | | | | | 92 | | | | | | | | |
| | B.1.630 | | | | | | | | | 97 | | | | | | | | |
| Zeta | P.2 | | | | | | | 97 | 97 | | | | | | | | 97 | |
| Theta | P.3 | | | | | | | 93 | 93 | | | | | | | | | |
| | B.1.617.3 | | | | | | 93 | | | | | | | | | | | |

% Mutation prevalence across lineages

The number associated with each element of the matrix in Tables 25 and 26 is the prevalence of that mutation at each location in the N Gene being analyzed (columns) across each of the lineages comprising the Combined WHO and CDC VOC/VOI/VUM lists (rows). Those percentages are calculated by use of the informatics tools provided by Latif et al. based on the aggregated GISAID database (Oct. 20, 2021 update). Where there is no number presented, that location remains as Wild Type in that lineage, Wild Type being defined as the original Wuhan reference sequence.

Table 27 lists S-gene primers to generate amplimers S:1-S:8 and N-gene amplimer N:9 in the DetectX-Cv assay.

TABLE 27
RT-PCR Primers for S-Gene (Amplimers S:1-S:8)
and N-Gene (Amplimer N:9)

| | Amplimer | | | |
|---|---|---|---|---|
| SEQ ID NOS. | # | Target | Gene | Primer Sequence (5′ to 3′) |
| SEQ ID NO: 137 | S:1 | (AA ⁽⁻⁾15-⁽⁻⁾11) | S | TTTAGAGTTGTTATTTCTAGTG |
| | | | | ATGTTC |
| SEQ ID NO: 20 | | (AA 33-41) | | Cy3-TTTTTGTCAGGGTAATAAA |
| | | | | CACCACGTG |
| SEQ ID NO: 9 | S:2 | (AA 57-65) | S | ACCTTTCTTTTCCAATGTTACT |
| | | | | TGGTTC |
| SEQ ID NO: 138 | | (AA 98-105) | | Cy3-TTTAATCCAGCCTCTTATT |
| | | | | ATGTTAGAC |
| SEQ ID NO: 11 | S:3 | (AA 118-126) | S | TTTCTTATTGTTAATAACGCTA |
| | | | | CTAATG |
| SEQ ID NO: 139 | | (AA 161-169) | | Cy3-TTTCAAAAGTGCAATTATT |
| | | | | CGCACTAGA |
| SEQ ID NO: 21 | S:4 | (AA 205-213) | S | TTTTAAGCACACGCCTATTAAT |
| | | | | TTAGTG |
| SEQ ID NO: 22 | | (AA 260-268) | | Cy3-TTTCCACATAATAAGCTG |
| | | | | CAGCACCAGC |
| SEQ ID NO: 13 | S:5 | (AA 400-408) | S | TTTTGTAATTAGAGGTGATGAA |
| | | | | GTCAGA |
| SEQ ID NO: 14 | | (AA 456-464) | | Cy3-TTTAAAGGTTTGAGATTAG |
| | | | | ACTTCCTAA |
| SEQ ID NO: 140 | S:6 | (AA 471-463) | S | TTTCTTTTGAGAGAGATATTTC |
| | | | | AACTGA |
| SEQ ID NO: 16 | | (AA 506-514) | | Cy3-TTTAAAGTACTACTACTCT |
| | | | | GTATGGTTG |
| SEQ ID NO: 23 | S:7 | (AA 596-604) | S | TTTAGTGTTATAACACCAGGAA |
| | | | | CAAATA |
| SEQ ID NO: 24 | | (AA 618-626) | | Cy3-TTTTGCATGAAT |
| | | | | AGCAACAGGGACTTCT |
| SEQ ID NO: 141 | S:8 | (AA 666-673) | S | TTTATTGGTGCAGGTATATGC |
| | | | | GCTAG |
| SEQ ID NO: 18 | | (AA 707-715) | | Cy3-TTTTGGTATGGCAATA |
| | | | | GAGTTATTAGAG |
| SEQ ID NO: 142 | N:9 | (AA167-175) | N | TTTGCCAAAAGGCTTCTACGC |
| | | | | AGAAG |
| SEQ ID NO: 143 | | (AA 253-261) | | Cy3-TTTTTGCCGAGGCTTCTT |
| | | | | AGAAGCC |

Table 28A lists S-gene robes used with the N-gene specific probes to detect hybridization to the S gene regions in amplimers S:1-S:8, that are generated via RT-PCR from the representative primer pairs described in Table 27. Table 28B lists N-gene probes used with the S-gene specific probes to detect hybridization to the N gene region (aa183-aa252), amplimer N:9 that are generated via RT-PCR from the representative primer pairs described in Table 27.

TABLE 28A
Hybrdization Probes (Spike gene region)

| | Amplimer | | | Probe Sequence |
|---|---|---|---|---|
| SEQ ID NOS. | # | Target | U/W/M | (5′ to 3′) |
| SEQ ID NO: 144 | S:1 | S: L5F | U | TTTTCGTTTGTTTTTY |
| | | | | TTGTTTTATTGCTTTT |
| SEQ ID NO: 145 | | S: L5_ | W | TTTTCGTTTGTTTTTC |
| | | | | TTGTTTTATTTTTT |
| SEQ ID NO: 146 | | S:_5F | M | TTTTCGTTTGTTTTTTT |
| | | | | TGTTTTATTTTTT |
| SEQ ID NO: 30 | S:1 | S: S13I | U | TTTTTCTAGTCTCTAK |
| | | | | TCAGTGTGTTTTTT |
| SEQ ID NO: 64 | | S: S13_ | W | TTTTTTTGTCTCTAGT |
| | | | | CAGTGTTTTTTTTT |
| SEQ ID NO: 67 | | S:_13I | M | TTTTTTTAGTCTCTAT |
| | | | | TCAGTGTTTTTTTT |
| SEQ ID NO: 233 | S:1 | S: L18F.T19R.T20N | U | TTTTTTAATYTTASAA |
| | | | | MCAGAACTCTTTTT |
| SEQ ID NO: 69 | | S: L18_.T19_.T20_ | W | TTTTTTTATCTTACAA |
| | | | | CCAGAACCTTTTTT |
| SEQ ID NO: 234 | | S: L18F.T19_.T2ON | M | TTTTTCTTGTTAATTTT |
| | | | | ACAAMCATTTTTT |
| SEQ ID NO: 148 | | S: L18_.T9R.T20_ | M | TTTTTCTTTAATCTTA |
| | | | | GAACCAGACTTTTT |
| SEQ ID NO: 71 | | S: L18_.T19_._20N | M | TTTTTTATTTTACAAA |
| | | | | CAGAACTTTTTTTT |
| SEQ ID NO: 235 | S:2 | S: A67V.HV69-70del | U | TTTTTCCCATGYTATA |
| | | (MIX) | | CATGTCTCTGTTTTTT |
| SEQ ID NO: 236 | | | | TTTTTTTTTCCATGYT |
| | | | | ATCTCTGGGATTTTTT |
| SEQ ID NO: 149 | | S: A67_.HV69-70_ | W | TTTTTCTCCATGCTAT |
| | | | | ACATGTCCTTTTTT |
| SEQ ID NO: 150 | | S: A67_._69-70del | M | TTTTTTTTTCCATGCT |
| | | | | ATCTCTGTTTTTTT |
| SEQ ID NO: 151 | | S:_67V._69-70del | M | TTTTTTTTTCCATGTT |
| | | | | ATCTCTGTTTTTTT |
| SEQ ID NO: 31 | S:2 | S: HV69-70del (Mix) | U | TTTTTCCCATGCTATA |
| | | | | CATGTCTCTGTTTTTT |
| SEQ ID NO: 32 | | | | TTTTTTTTTCCATGCT |
| | | | | ATCTCTGGGATTTTTT |
| SEQ ID NO: 31 | | S: HV69-70_ | W | TTTTTCCCATGCTATA |
| | | | | CATGTCTCTGTTTTTT |
| SEQ ID NO: 32 | | S:_69-70del | M | TTTTTTTTTCCATGCT |
| | | | | ATCTCTGGGATTTTTT |
| SEQ ID NO: 237 | S:2 | S:G75V.T76I | U | TTTTTTGACCAATGGT |
| | | | | ACTAAGAGTTTTTT |
| SEQ ID NO: 238 | | S:G75_.T76_ | W | TTTTTTACCAATGGTA |
| | | | | CTAAGAGTCTTTTT |
| SEQ ID NO: 239 | | S:_75V._76I | M | TTTTTTACCAATGTTA |
| | | | | TTAAGAGTCTTTTT |
| SEQ ID NO: 240 | S:2 | S: D80A/G | U | TTTTTCAGAGGTTTGV |
| | | | | TAACCCTGTCTTTTTT |
| SEQ ID NO: 34 | | S: D80_ | W | TTTTTTTGGTTTGATA |
| | | | | ACCCTGCTTTTTTT |
| SEQ ID NO: 35 | | S:_80A | M | TTTTTTTGGTTTGCTA |
| | | | | ACCCTGCTTTTTTT |
| SEQ ID NO: 152 | | S:_80G | M | TTTTTTTGGTTTGGTA |
| | | | | ACCCTGCTTTTTTT |
| SEQ ID NO: 153 | S:2 | S: T95I | U | TTTCTTTTTGCTTCCA |
| | | | | YTGAGAAGTCTTTTTT |
| SEQ ID NO: 154 | | S: T95_ | W | TTTTTTCCGCTTCCAC |
| | | | | TGAGAAGCATTTTT |
| SEQ ID NO: 155 | | S:_95I | M | TTTTTTCCGCTTCCAT |
| | | | | TGAGAAGCATTTTT |
| SEQ ID NO: 36 | | S: D138Y | U | TTTTATTTTGTAATKAT |
| | | | | CCATTTTTGTTTT |
| SEQ ID NO: 37 | S:3 | S: D138_ | W | TTTTTCTTTGTAATGA |
| | | | | TCCATTTTCTTTTT |
| SEQ ID NO: 38 | | S:_138Y | M | TTTTTTTTTGTAATTAT |
| | | | | CCATTTTCTTTTT |
| SEQ ID NO: 241 | S:3 | L141Y.G142D.V143insT. | U | TTTCTTTGGRTGTTTA |
| | | Y144S/del.Y145N (Mix) | | TTACCACAAAAATTTT |
| SEQ ID NO: 157 | | | | TTTTTTTTTGGGTGTT |
| | | | | TACCACAAAAACTTTT |
| SEQ ID NO: 158 | | | | TTTATTTTTGGGTGTT |
| | | | | ACTTATTACCACATTT |
| SEQ ID NO: 160 | | | | TTCGTAATGATCCATT |
| | | | | TTATTACCACAAATTT |
| SEQ ID NO: 156 | | S: L141_.G142_. | W | TTTCTTTGGGTGTTTA |
| | | V143_.Y144_.Y145_ | | TTACCACAAAAATTTT |
| SEQ ID NO: 157 | | S: L141_.G142_. | M | TTTTTTTTTGGGTGTT |
| | | V143._144del.Y145_ | | TACCACAAAAACTTTT |
| SEQ ID NO: 158 | | S:L 141_.G142_. | M | TTTATTTTTGGGTGTT |
| | | ins143T._144S._145N | | ACTTATTACCACATTT |
| SEQ ID NO: 159 | | S: L141_._142D. | M | TTCTTTTGGATGTTTA |
| | | V143_.Y144_.Y145 | | TTACCACAAAAACTTT |
| SEQ ID NO: 160 | | S:_141Y._142del. | M | TTCGTAATGATCCATT |
| | | V143del._144del. | | TTATTACCACAAATTT |
| | | Y145_ | | |
| SEQ ID NO: 242 | S:3 | S: Y145H | U | TTTCTGTGTTTATYAC |
| | | | | CACAAAAACTTCTT |
| SEQ ID NO: 243 | | S: Y145_ | W | TTTTTCTGTGTTTATT |
| | | | | ACCACAAATCTTTTT |
| SEQ ID NO: 244 | | S:_145H | M | TTTTTTATGTTTATCA |
| | | | | CCACAAATCTTTTT |
| SEQ ID NO: 39 | S:3 | S: W152C/L/R.E154K | U | TTTTTAGTWKKATGG |
| | | | | AAAGTGAGTTCTTTT |
| SEQ ID NO: 40 | | S: W152_.E154_ | W | TTTCTCTAAAAGTTGG |
| | | | | ATGGAAACTCTTCT |
| SEQ ID NO: 41 | | S:_1520.E154_ | M | TTTCTTCAAAGTTGTA |
| | | | | TGGAAAGCCTTCTT |
| SEQ ID NO: 161 | | 5:_152L.E154_ | M | TCTCTTCAAAAGTTTG |
| | | | | ATGGAAATCTCTTT |
| SEQ ID NO: 162 | | 5:_152R.E154_ | M | TTTCTTTACAAAAGTA |
| | | | | GGATGGATTTCTTT |
| SEQ ID NO: 163 | | S:_1520._154K | M | TTTCTTTGTTGGATGA |
| | | | | AAAGTGATCTTCTT |
| SEQ ID NO: 164 | S:3 | S: E156G/del.F157del. | U | TTTTGAAAGTGAGTTC |
| | | R158del (Mix) | | AGAGTTTACCTTTT |
| SEQ ID NO: 165 | | | | UTTCTTTGGAAAGTGG |
| | | | | AGTTTATTCTCTTTT |
| SEQ ID NO: 164 | | S:_E156_.F157_.R158_ | W | TTTTGAAAGTGAGTTC |
| | | | | AGAGTTTACCTTTT |
| SEQ ID NO: 165 | | S: 156G._157del._ | M | TTCTTTGGAAAGTGG |
| | | 158del | | AGTTTATTCTCTTTT |
| SEQ ID NO: 81 | | S: D215G | U | TTTTTTAGTGCGTGRT |
| | | | | CTCCCTCATTTTTT |
| SEQ ID NO: 245 | S:4 | S: D215_ | M | TTTTTTTTTGCGTGAT |
| | | | | CTCCCTTTTTTTTT |
| SEQ ID NO: 246 | | S:_215G | W | TTTTTTTTTGCGTGGT |
| | | | | CTCCCCTTTTTTTT |
| SEQ ID NO: 247 | S:4 | S: A222V | U | TTTTGTTTTTCGGYTT |
| | | | | TAGAACCATCTTTT |
| SEQ ID NO: 248 | | S: A222_ | M | TTTTTTTTTTTCGGCT |
| | | | | TTAGAACTTTTTTT |
| SEQ ID NO: 249 | | S:_222V | W | TTTTTTGTTTTTCGGT |
| | | | | TTTAGAATTTTTTT |
| SEQ ID NO: 166 | S:4 | S: L242del.A243del. | U | TTTTTTTTCAAACTTTA |
| | | | | CTTGCTTTACTCTTT |
| SEQ ID NO: 167 | | L244deL (Mix) | | TTTTTTTTCAAACTTTA |
| | | | | CATAGAAGCCTTTTT |
| SEQ ID NO: 166 | | S: L242_.A243_.L244 | W | TTTTTTTTCAAACTTTA |
| | | | | CTTGCTTTACTCTTT |
| SEQ ID NO: 167 | | S: 242del._243del._ | M | TTTTTTTTCAAACTTTA |
| | | 244deL | | CATAGAAGCCTTTTT |
| SEQ ID NO: 168 | S:4 | S: R246N.5247del. | U | TTTTCTACATAGAAGT |
| | | Y248del.L249del. | | TATTTGACTCCCTTTT |
| SEQ ID NO: 169 | | T250del.P251del. | | TTTTCTGCTTTACATA |
| | | D253del (Mix) | | TGACTCCTGGTTTTTT |
| SEQ ID NO: 168 | | S: R246_.S247_.Y248_. | W | TTTTCTACATAGAAGT |
| | | L249_.T250_.P251_. | | TATTTGACTCCCTTTT |
| | | D253_ | | |
| SEQ ID NO: 169 | | S:_246N._247del._ | M | TTTTCTGCTTTACATA |
| | | 248del._ 249del._ | | TGACTCCTGGTTTTTT |
| | | 250del._251del._ | | |
| | | 253del (Mix) | | |
| SEQ ID NO: 170 | S:4 | S: D253G | U | TTTCTACTCCTGGTG |
| | | | | RTTCTTCTTCATTTT |
| SEQ ID NO: 171 | | S: D253_ | W | TTTTTTCCCTGGTGAT |
| | | | | TCTTCTTTCTTTTT |
| SEQ ID NO: 172 | | S:_253G | M | TTTTTTCCCTGGTGGT |
| | | | | TCTTCTTTTTTTTT |
| SEQ ID NO: 250 | S:5 | S: K417N/T | U | TTTTAACTGGAAMKAT |
| | | | | TGCTGATTATTTTT |
| SEQ ID NO: 88 | | S: K417 | W | TTCTTCTCTGGAAAGA |
| | | | | TTGCTGACTTTTTT |
| SEQ ID NO: 89 | | S:_417N | M | TTTTTCTCTGGAAATA |
| | | | | TTGCTGACTTTTTT |
| SEQ ID NO: 92 | | S:_417T | M | TTTTTTCCTGGAACGA |
| | | | | TTGCTGTTTTTTTT |
| SEQ ID NO: 42 | S:5 | S: N439K.N440K | U | TTTTTAATTCTAAMAA |
| | | | | KCTTGATTCTAATTTT |
| SEQ ID NO: 43 | | S: N439_.N440_ | W | TTTTTAATTCTAACAA |
| | | | | TCTTGATTTCTTTT |
| SEQ ID NO: 44 | | S: N439_._440K | M | TTTTTTATTCTAACAA |
| | | | | GCTTGATTTTTTTT |
| SEQ ID NO: 45 | | S:_439K.N440_ | M | TTTTCTATTCTAAAAA |
| | | | | TCTTGATTTCTTTT |
| SEQ ID NO: 251 | S:5 | S: L452R/Q | U | TTTCTATAATTACCDG |
| | | | | TATAGATTGTCTTT |
| SEQ ID NO: 47 | | S: L452_ | W | TTTTTTTAATTACCTG |
| | | | | TATAGATTTCTTTT |
| SEQ ID NO: 48 | | S:_452R | M | TTTTTCATAATTACCG |
| | | | | GTATAGATCTTTTT |
| SEQ ID NO: 173 | | S:_452Q | M | TTTTTTTAATTACCAG |
| | | | | TATAGATCCTTTTT |
| SEQ ID NO: 252 | S:6 | S: 5477N.T478K | U | TTTTTCGCCGGTARC |
| | | | | AMACCTTGTATTTTT |
| SEQ ID NO: 253 | | S: 5477_.T478_ | W | TTTTTTTCCGGTAGCA |
| | | | | CACCTTTTTTTTTT |
| SEQ ID NO: 50 | | S:_477N.T478_ | M | TTTTCTTCCGGTAACA |
| | | | | CACCTTTTTTTTTT |
| SEQ ID NO: 254 | | S: 5477_._478K | M | TTTTTCTGGTAGCAAA |
| | | | | CCTTGTTTTTTTTT |
| SEQ ID NO: 174 | S:6 | S: T478K | U | TTTTTCGGTAGCAMA |
| | | | | CCTTGTAATGTTTTT |
| SEQ ID NO: 175 | | S: 478K | W | TTTTTTTGTAGCACAC |
| | | | | CTTGTATTTTTTTT |
| SEQ ID NO: 176 | | S: T478_ | M | TTTTTTTGTAGCAAAC |
| | | | | CTTGTATTTTTTTT |
| SEQ ID NO: 255 | S:6 | S: V483A. E484K/Q | U | TTTTTTAATGGTGYTR |
| | | | | AAGGTTTTAATTTTTT |
| SEQ ID NO: 52 | | S: V483_.E484_ | W | TTTTTTCTGGTGTTGA |
| | | | | AGGTTTTACTTTTT |
| SEQ ID NO: 53 | | S: V483_._484K | M | TTTTTTTATGGTGTTA |
| | | | | AAGGTTTTCTTTTT |
| SEQ ID NO: 177 | | S: V483_._484Q | M | TTTTTTATGGTGTTCA |
| | | | | AGGTTTTCTTTTTT |
| SEQ ID NO: 54 | | S:_483A. E484_ | M | TTTTTTTATGGTGCTG |
| | | | | AAGGTTCTTTTTTT |
| SEQ ID NO: 256 | S:6 | S: F490S | U | TTTTCTAATTGTTACT |
| | | | | YTCCTTTACAATTTTT |
| SEQ ID NO: 179 | | S: F490_ | W | TTTTTTTTTGTTACTTT |
| | | | | CCTTTACTTTTTT |
| SEQ ID NO: 180 | | S:_4905 | M | TTTTTTTTTGTTACTCT |
| | | | | CCTTTACTTTTTT |
| SEQ ID NO: 181 | S:6 | S: S494P | U | TTTTTCTCCTTTACAA |
| | | | | YTATATGGTTTTTTTT |
| SEQ ID NO: 182 | | S: 5494_ | W | TTTTTCTCTTTACAAT |
| | | | | CATATGGTCTTTTT |
| SEQ ID NO: 183 | | S:_494P | M | TTTTTCTCTTTACAAC |
| | | | | CATATGGTCTTTTT |
| SEQ ID NO: 257 | S:6 | S: N501Y/T | U | TTTTTTTTCCAACCCA |
| | | | | CTWMTGGTGTTTTTT |
| | | | | TT |
| SEQ ID NO: 56 | | S: N501_ | W | TTTTTTTTACCCACTA |
| | | | | ATGGTGTCTTTTTT |
| SEQ ID NO: 57 | | S:_501Y | M | TTTTTTTTACCCACTT |
| | | | | ATGGTGTCTTTTTT |
| SEQ ID NO: 184 | | S:_501T | M | TTTTTTTACCCACTAC |
| | | | | TGGTGTCTTTTTTT |
| SEQ ID NO: 107 | S:7 | S: Q613H.D614G | U | TTTTTCTCTTTATCAR |
| | | | | GRTGTTAACTGCTTTT |
| | | | | TT |
| SEQ ID NO: 108 | | S: Q613_.D614_ | W | TTTTTCTTATCAGGAT |
| | | | | GTTAACTTTTTTTT |
| SEQ ID NO: 109 | | S: Q613_._614G | M | TTTTTTCCTATCAGGG |
| | | | | TGTTAACTTTTTTT |
| SEQ ID NO: 110 | | S: Q2613_._614G | M | TTTTTTCCTATCAAGG |
| | | | | TGTTAACTTTTTTT |
| SEQ ID NO: 258 | | 5:_613H.D614_ | M | TTTTTCCCTTTATCAT |
| | | | | GATGTTAATCTTTT |
| SEQ ID NO: 259 | S:8 | S: Q677P/H | U | TTTTTTATCAGACTCM |
| | | | | BACTAATTCTCTTTTT |
| SEQ ID NO: 186 | | S: Q677_ | W | TTTTTTCCAGACTCAG |
| | | | | ACTAATTTCTTTTT |
| SEQ ID NO: 187 | | S:_677P | M | TTTTTCTTCAGACTCC |
| | | | | GACTAATCTTTTTT |
| SEQ ID NO: 188 | | S:_677H2 | M | TTTTTTCCAGACTCAC |
| | | | | ACTAATTTCTTTTT |
| SEQ ID NO: 189 | | S:_677H1 | M | TTTTTTCCAGACTCAT |
| | | | | ACTAATTTCTTTTT |
| SEQ ID NO: 260 | S:8 | S: P681H/R | U | TTTTTTCAGACTAATT |
| | | | | CTCVTCGGCTTTTT |
| SEQ ID NO: 59 | | S: P68_ | W | TTTTTTTCTAATTCTC |
| | | | | CTCGGCGTTTTTTT |
| SEQ ID NO: 60 | | S:_681H | M | TTTTTTTTTAATTCTCA |
| | | | | TCGGCGTTTTTTT |
| SEQ ID NO: 190 | | S:_681R | M | TTTTTTTTTAATTCTC |
| | | | | GTCGGCGTTTTTTT |
| SEQ ID NO: 261 | S:8 | S: A688V-RE1.1 | U | TTTTTTAGTGTAGYTA |
| | | | | GTCAATCCACTTTT |
| SEQ ID NO: 262 | | S: A688_-RE1.1 | W | TTTTTTCAGTGTAGCT |
| | | | | AGTCAATTTTTTTT |
| SEQ ID NO: 263 | | S:_688V-RE1.1 | M | TTTTTTCAGTGTAGTT |
| | | | | AGTCAATTTTTTTT |
| SEQ ID NO: 61 | S:8 | S: A701V | U | TTTTCACTTGGTGYAG |
| | | | | AAAATTCAGTTTTT |
| SEQ ID NO: 62 | | S: A701_ | W | TCTTCTTCTTGGTGCA |
| | | | | GAAAATTATTCTTT |
| SEQ ID NO: 63 | | S:_701V | M | TCTTCTTCTTGGTGTA |
| | | | | GAAAATTATTCTTT |
| SEQ ID NO: 134 | | RNAseP control | | TTTTTTTTCTGACCTG |
| | | | | AAGGCTCTGCGCGTT |
| | | | | TTT |
| SEQ ID NO: 136 | | NEG CONTROL | | TTTTTTCTACTACCTA |
| | | | | TGCTGATTCACTCTTT |
| | | | | TT |

TABLE 28B
Hybrdization Probes (Nucleocapsid gene region)

| | | | | Probe Sequence |
|---|---|---|---|---|
| SEQ ID NOS. | Amplimer # | Target | U/W/M | (5′ to 3′) |
| SEQ ID NO: 191 | N:9 | N: 5194L | U | TTTTCCGCAACAGTTY |
| | (AA183-252) | | | AAGAAATTCATTTT |
| SEQ ID NO: 192 | | N: S194 | W | TTTTTTTAACAGTTCA |
| | | | | AGAAATTTTTTTTT |
| SEQ ID NO: 193 | | N:_194L | M | TTTTTTTAACAGTTTA |
| | | | | AGAAATTTTTTTTT |
| SEQ ID NO: 194 | N:9 | N: S197L | U | TTTTCTCAAGAAATTY |
| | (AA183-252) | | | AACTCCAGGCTTTT |
| SEQ ID NO: 195 | | N: S197 | W | TTTTTCTAAGAAATTC |
| | | | | AACTCCATTTTTTT |
| SEQ ID NO: 196 | | N:_197L | M | TTTTTCTAAGAAATTT |
| | | | | AACTCCATTTTTTT |
| SEQ ID NO: 197 | N:9 | N: P199L | U | TTTTTAAATTCAACTC |
| | (AA183-252) | | | YAGGCAGCATCTTT |
| SEQ ID NO: 198 | | N: P199 | W | TTTTTTTTTCAACTCC |
| | | | | AGGCAGCTTTTTTT |
| SEQ ID NO: 199 | | N:_199L | M | TTTTTTTTTCAACTCT |
| | | | | AGGCAGCTTTTTTT |
| SEQ ID NO: 200 | N:9 | N: S201I | U | TTTTTACTCCAGGCAK |
| | (AA183-252) | | | CWSTADRSGATTTT |
| SEQ ID NO: 201 | | N: S201_ | W | TTTTTTTTCCAGGCAG |
| | | | | CAGTADRTTTTTTT |
| SEQ ID NO: 202 | | N:_201I | M | TTTTTTTTCCAGGCAT |
| | | | | CAGTAGGTTTTTTT |
| SEQ ID NO: 203 | N:9 | N: S202N | U | TTTTCCCAGGCAGCA |
| | (AA183-252) | | | RTADRSGAACCTTTT |
| SEQ ID NO: 204 | | N: S202_ | W | TTTTTTTAGGCAGCA |
| | | | | GTADRSGATTTTTTT |
| SEQ ID NO: 205 | | N:_202N | M | TTTTTTTAGGCAGCAA |
| | | | | TAGGGGATTTTTTT |
| SEQ ID NO: 206 | N:9 | N: R203M/K G204R | U | TTTTGCAGCWSTADR |
| | (AA183-252) | | | SGAACTTCTCTTTTT |
| SEQ ID NO: 207 | | N: R203_ G204_ | W | TTTTTTTCAGCAGTAG |
| | | | | GGGAACTCTTTTTT |
| SEQ ID NO: 208 | | N:_203M G204_ | M | TTTTTTTCAGCAGTAT |
| | | | | GGGAACTCTTTTTT |
| SEQ ID NO: 219 | | N:_203K G204R | M | TTTTTTTCAGCAGTAA |
| | | | | ACGAACTCTTTTTT |
| SEQ ID NO: 210 | | N:_203K G204R (2) | M | TTTTTTTCAGCTCTAA |
| | | | | ACGAACTCTTTTTT |
| SEQ ID NO: 211 | N:9 | N: T205I | U | TTTTCSTADRSGAAYT |
| | (AA183-252) | | | TCTCCTGCTATTTT |
| SEQ ID NO: 212 | | N: T205 | W | TTTTTTTGGGGAACTT |
| | | | | CTCCTGCCTTTTTT |
| SEQ ID NO: 213 | | N:_2051 | M | TTTTTTTGGGGAATTT |
| | | | | CTCCTGCCTTTTTT |
| SEQ ID NO: 214 | N:9 | N: A208G R209del | U | TTTTAAYTTCTCCTGC |
| | (AA183-252) | (MIX) | | TAGAATGGCTGTTT |
| SEQ ID NO: 215 | | | | TTTTACGAACTTCTCC |
| | | | | TGGAATGGCTGTTT |
| SEQ ID NO: 216 | | N: A208_ R209_ | W | TTTTCTCTCCTGCTAG |
| | | | | AATGGCTGTTTTTT |
| SEQ ID NO: 217 | | N:_208G _209del | M | TTTTTACTTCTCCTGG |
| | | | | AATGGCTGTTTTTT |
| SEQ ID NO: 218 | N:9 | N: G212V N213Y | U | TTTTTTGGCTGKCWA |
| | (AA183-252) | | | TKGCKGTGATTTTTT |
| SEQ ID NO: 219 | | N: G212 N213Y | W | TTTTTTTAATGGCTGG |
| | | | | CWATKGCTTTTTTT |
| SEQ ID NO: 220 | | N:_212V N213_ | M | TTTTTTTAATGGCTGT |
| | | | | CAATGGCTTTTTTT |
| SEQ ID NO: 221 | | N: G212V N213_ | W | TTTTTTTTGGCTGKCA |
| | | | | ATKGCKGCTTTTTT |
| SEQ ID NO: 222 | | N: G212_ _213Y | M | TTTTTTTTGGCTGGCT |
| | | | | ATGGCGGCTTTTTT |
| SEQ ID NO: 223 | N:9 | N: G2140 G2150 | U | TTTTTTGGCTGKCWA |
| | (AA183-252) | | | TKGCKGTGATTTTTT |
| SEQ ID NO: 224 | | N: G214_ G2150 | W | TTTTTTTTGKCWATG |
| | | G | | CKGTGATTTTTTTT |
| SEQ ID NO: 225 | | N: 2140 G215 | M | TTTTTTTTGGCAATTG |
| | | | | CGGTGATTTTTTTT |
| SEQ ID NO: 226 | | N: G2140 G215_ | W | TTTTTTTCWATKGCG |
| | | | | GTGATGCTTTTTTTT |
| SEQ ID NO: 227 | | N: G214_ _2150 | M | TTTTTTTCAATGGCTG |
| | | | | TGATGCTTTTTTTT |
| SEQ ID NO: 228 | N:9 | N: M234I 5235F | U | TTTTTGAGCAAAATDT |
| | (AA183-252) | | | YTGGTAAAGTTTTT |
| SEQ ID NO: 229 | | N: M234_ 5235_ | W | TTTTTTTCAAAATGTC |
| | | | | TGGTAAATTTTTTT |
| SEQ ID NO: 230 | | N:_234I S235_ | M | TTTTTCTAGCAAAATT |
| | | | | TCTGGTATCTTTTT |
| SEQ ID NO: 231 | | N:_234I S235_ (2) | M | TTTTTCTAGCAAAATA |
| | | | | TCTGGTATCTTTTT |
| SEQ ID NO: 232 | | N: M234_ _235F | M | TTTTTCTCAAAATGTT |
| | | | | TGGTAAATCTTTTT |

“DETECTX-Cv” technology is designed to combine the practicality of field deployable Q-RT-PCR testing with the high-level information content of targeted NGS. Population scale deployment of DETECTX-Cv is enabled in a way that is simple enough that it can be “drop-shipped” with minimal set up cost and training into any laboratory performing conventional Q-RT-PCR based COVID-19 screening. Initial field deployment demonstrated the ability of DETECTX-Cv to identify clinical positives per shift per Q-RT-PCR screening and analysis without additional sample prep for a large panel of CoV-2 clade variants (UK, Denmark, South Africa, Brazil, US (California, New York, Southern US) and Wuhan and the remainder of the present list of Variants of Concern, Variants of Interest and other Variants as known and classified by the World Health Organization; who.int/en/activities/tracking-SARS-CoV-2-variants/) incorporated into the content of the assay.

The technology encompassed in this invention enables DETECTX-Cv to perform very low-cost microarray analyses in a field-deployable format. DETECTX-Cv is based on proprietary technology of PathogenDx for designing DNA microarray probes and so, the resulting microarrays can be mass produced to deliver >24,000 tests/day. DETECTX-Cv also enables sequence-based testing on these microarrays via open-format room temperature hybridization and washing, much like the processing of ELISA assays. Like an ELISA plate, DETECTX-Cv is mass produced in a 96-well format, ready for manual or automated fluid handling and has the capability to handle up to 576 probe spots per well, at full production scale.

DETECTX-Cv is a combinatorial assay with several targets in the CoV-2 Spike gene and the N gene comprising an exceptionally large set of gain-of-function Spike and/or N gene mutants, which are believed to be selected for enhanced infectivity or resistance to natural or vaccine induced host immunity. Based on analysis of the rapidly growing CoV-2 resequencing effort (600,000 genomes in GISAID, April 2021 and over 5,000,000 genomes in GISAID, November 2021; gisaid.org) “terminal differentiation” of the Spike gene marker “basis set” into a set of informative Spike gene and N gene target sites is expected, which can be built into and mass produced into the same 96-well format described above. DETECTX-Cv is therefore expected to be beneficial as a true discovery tool that is capable of unbiased identification of new CoV-2 clade variants based on detection of novel combinations of the underlying Spike Variant “basis set”. Thus, DETECTX-Cv is expected to become the basis for field deployed seasonal COVID testing with military and civilian applications

The DETECTX-Cv test content comprises Spike Gene Target sequence analysis among 32 discrete information-rich domains, and N gene Target sequence analysis among 11 discrete information-rich domains produced as triplicate tests per array, with positive and negative controls, to produce core content that is deployed as about 160 independent hybridization tests (per well) on each sample. The DETECTX-Cv technology described here is based on multiplex asymmetric, endpoint RT-PCR amplification of viral RNA purified as for Q-RT-PCR screening. The RT-PCR product resulting from amplification is fluorescently tagged and used as-is without cleanup for the subsequent steps of hybridization and washing, which are performed at room temperature (RT). Subsequent to hybridization and washing, the DETECTX-Cv plate is subjected to fluorescent plate reading, data processing and analysis that occurs automatically with no intervention by a human user to result in an output of detected CoV-2 clades which can be used locally for diagnosis and/or simultaneously uploaded to a secure, cloud-based portal for use by medical officials for military or public health tracking and epidemiology analysis.

### Example 12

Microarray Assay for Qualitative Detection and Genotyping of SARS-CoV-2 Detect^{X}-Cv⁺ Method and Materials

Detect^{X}-Cv⁺ microarray assay uses the same technology as Detect^{X}-Rv described herein and is designed to simultaneously detect SARS-CoV-2 and identify single nucleotide polymorphisms and small deletions associated with SARS-CoV-2 variants. The assay utilizes the Zymo Research Quick-DNA/RNA™ Viral MagBead (R2140) magnetic silica bead extraction kit, the Applied Biosystems MiniAmp™ Thermal Cycler and the Sensospot® (Miltenyi Imaging) fluorescence microarray imager. The fluorescence of each signal is measured by the Sensospot imager and analyzed by proprietary Augury™ software using a local computer, and the results are stored in a dedicated folder on a user's computer. The system may be used with upper respiratory specimens such as nasopharyngeal (NP) swabs, oropharyngeal (OP) swabs, mid-turbinate swabs, anterior nasal swabs, nasal aspirates, nasopharyngeal wash/aspirates and bronchoalveolar lavage (BAL) specimens obtained from patients suspected of COVID-19 disease.

SARS-CoV-2 detection is accomplished by hybridization analysis of 18 Spike gene nucleotide sequences, based upon the use of a set of 18 synthetic oligonucleotide probes (universal probes) that are insensitive to mutations that may occur at those 18 sites. Analysis of deletion or single nucleotide polymorphisms (SNPs) in the SARS-CoV-2 Spike gene is performed by hybridization analysis at 18 unique Spike gene sites by using a different set of synthetic oligonucleotide probes, mutant and wild type. During hybridization, the mutant and wild type probes are designed to base pair to labeled amplicons presenting with the wild type or mutant genetic sequence. Post-hybridization, the now immobilized fluorescently labeled amplicon withstands repeated washing away of non-hybridized amplicon. Remaining probe-bound labeled amplicon achieves single base pair specificity. Fluorescence quantification via Augury™ software at each probe site enables determination of presence or absence of mutations or deletions at 16 Spike gene sites.

The universal probe signals are used to detect the simple presence of SARS-CoV-2 independent of variant type. If N, such as, but not limited to, where N=6, universal probes are above their respective thresholds, then the sample is identified as SARS-CoV-2. By using universal probes the detection of SARS-CoV-2 is not materially affected by variant changes. Moreover, by having access to a large number of the universal probes in the same test, for example, 18, it is possible to require that several of the universal probes must be above threshold concurrently, i.e., ≥6, thus greatly diminishing the likelihood of a false positive determination as compared to a test where only the signal from 1 probe would be sufficient to detect the presence of the virus.

Probe Thresholds are assumed to be constant for the Detect^{X}-Cv⁺ assay and are determined as for the Detect^{X}-Cv. When the measured hybridization signal for each probe is above the probe threshold, the probe is said to be “ON”, i.e. hybridization detected above threshold. When the signal is less than the probe threshold, the probe is said to be “OFF” i.e. hybridization not detected above threshold.

### Universal Probes Used to Detect Spike Gene Mutations

The 18 unique Spike gene sites are located within the 5 segments of the Spike gene that are amplified as a single multiplex assay in the RT-PCR reaction which precedes microarray hybridization analysis. Table 29 identifies the primer target, the primer name, the primer sequence and the amino acid translation of the codons in the primer sequence. The sequence identifier includes the 5′-tag identified separately from the sequence.

TABLE 29
Detect^{X}-Cv⁺Primers

| Primer | | | | |
|---|---|---|---|---|
| Target | | | | |
| Primer | | | | |
| nucleotide | | | | |
| ref. | | | | Amino Acid |
| (WIV04)* | Name | 5′Tag | Sequences5′-3′ | Translation |
| 21728- | S:65-FP- | — | TTA.CCT.TTC.TTT.TCC. | LPFFSNVTWF |
| 21755 | 99324 | | AAT.GTT.ACT.TGG.T | SEQ ID NO: 271 |
| | | | SEQ ID NO: 266 | |
| 21853- | S:97-RPC3- | 5CY3/TTT | AAT.CCA.GCC.TCT.TAT. | K(F/S)NIIRGWI |
| 21877 | 99287 | | TAT.GTT.ARA.C | SEQ ID NO: 272 |
| | | | SEQ ID NO: 138 | |
| 21914- | S:126-FP- | TTT | CTT.ATT.GTT.AAT.AAC. | LIVNNATNV |
| 21938 | 99248 | | GCT.ACT.AAT.G | SEQ ID NO: 273 |
| | | | SEQ ID NO: 11 | |
| 22043- | S:161- | 5CY3/TTT | C.AAA.AGT.GCA.ATT. | SSANNCTFE |
| 22067 | RPC3- | | ATT.CGC.ACT.AGA | SEQ ID NO: 274 |
| | 99237 | | SEQ ID NO: 139 | |
| 22760- | S:408-FP- | T | TTT.GTA.ATT.AGA.GGT. | FVIRGDEVR |
| 22785 | 99230 | | GAT.GAA.GTC.AG | SEQ ID NO: 275 |
| | | | SEQ ID NO: 267 | |
| 22929- | S:456.RPC3- | 5CY3/TTT | ATT.GGT.GCA.GGT.ATA. | FRKSNLKPF |
| 22953 | 99266 | | TGC.GCT.AG | SEQ ID NO: 276 |
| | | | SEQ ID NO: 141 | |
| 22951- | S:473-FP- | TT | T.TTT.GAG.AGA.GAT. | PFFRDISTE |
| 22974 | 99278 | | ATT.TCA.ACT.GA | SEQ ID NO: 277 |
| | | | SEQ ID NO: 268 | |
| 23078- | S:501.RPC3- | 5CY3/TTT | A.AAG.TAC.TAC.TAC. | QPYRVVVLS |
| 23102 | 99243 | | TCT.GTA.TGG.TTG | SEQ ID NO: 278 |
| | | | SEQ ID NO: 16 | |
| 23558- | S:672-FP- | TTT | ATT.GGT.GCA.GGT. | IGAGICAS |
| 23580 | 99259 | | ATA.TGC.GCT.AG | SEQ ID NO: 279 |
| | | | SEQ ID NO: 141 | |
| 23684- | S:701- | 5CY3/TTTT | GG.TAT.GGC.AAT.AGA. | SNNSIAIP |
| 23706 | RPC3- | | GTT.ATT.AGA | SEQ ID NO: 280 |
| | 99214 | | SEQ ID NO: 269 | |
| RNase P | RNaseP- | TTT | GTTTGCAGATTTGGACCT | — |
| Internal | FP-99033 | | GCGAGCG | |
| Control | RNaseP- | | SEQ ID NO: 132 | |
| N/A | RPC3- | 5CY3/TTT | AAGGTGAGCGGCTGTCT | — |
| | 99004 | | CCACAAGT | |
| | | | SEQ ID NO: 270 | |

*hCoV-19/Wuhan/WIV04/2019 (WIV04) is the official reference sequence employed by GISAID (EPI_ISL_402124). WIV04 was chosen because of itshigh-quality genome sequence and because it represented the consensus of a handful of early submissions for the betacoronavirus responsible for COVID-19 (Pilailuk et al. 2020).

The presence of SARS-CoV-2 is determined by concurrent hybridization of the 5 Spike gene amplicons to the 18 universal probes. Table 30 lists and names 61 probes and the sequences thereof for which the 5′-tag sequences and the 3′-tag sequences are identified separately. Thus 18 independent hybridization tests are performed in parallel, within the microarray, on every sample. Table 31 identifies 18 of the probes from Table 30 where the 18 sites of interest in the Spike gene and the universal probes designed to bind to them can be identified by their location in the amino acid sequence in the Spike protein which they will ultimately encode.

TABLE 30
Table 2. Detect^{x}-Cv⁺Probes

| | | | | | Probe Function |
|---|---|---|---|---|---|
| | | | | | Relative to |
| | | | | | VOC, |
| | Probe | Probe | | Amino Acid | VOI and >0.1% |
| Index | Type | Name | Sequence 5′-3′ | Translations | targets |
| 1 | C | RNAse.P. | 5′Tag-TTTTTT | N/A | N/A |
| | | Probe- | TTCTGACCTGAAGGCTCT | | |
| | | pub1.1 | GCGCG | | |
| | | | TTTTT-3′Tag | | |
| | | | SEQ ID NO: 134 | | |
| 2 | U | 69-70 | 5′Tag-TTTTTC | 65•.66H.67A/V. | ALL |
| | | universal- | C.CAT.GYT.ATA.CAT.GTC. | 68I.69H/del.70V/ | VARIANTS |
| | | 1:1 mix | TCT.G | del.71S.72• | |
| | | of the wild | TTTTTT-3′Tag | | |
| | | type and | SEQ ID NO: 235 | | |
| | | deleted | 5′Tag-TTTTTTTTT | | |
| | | mutant | C.CAT.GYT.ATC.---.--- | | |
| | | sequence | .TCT.GGG.A | | |
| | | | TTTTTT-3′Tag | | |
| | | | SEQ ID NO: 236 | | |
| 3 | WT | 69-70.WT- | 5′Tag-TTTTTC | 65•.66H.67A/V.6 | WT |
| | | SE-RE1.2 | C.CAT.GYT.ATA.CAT.GTC. | 8I.69H.70V.715. | |
| | | | TCT.G | 72• | |
| | | | TTTTTT-3′Tag | | |
| | | | SEQ ID NO: 235 | | |
| 4 | M1 | 69- | 5′Tag-TTTTTTTTT | 65•.66H.67A/V. | OMICRON, |
| | | 70.DEL- | C.CAT.GYT.ATC.---. | 68I.69del.70del. | ALPHA, ETA, |
| | | SE-RE1.1 | ---.TCT.GGG.A | 71S.72G.73. | C.36.3 |
| | | | TTTTTT-3′Tag | | |
| | | | SEQ ID NO: 236 | | |
| 5 | U | 80U-SE- | 5′Tag-TTTTTC | 77•.78R.79F.80 | ALL |
| | | RE1.1 | AG.AGG.TTT.GMT.AAC. | D/A.81N.82P. | VARIANTS |
| | | | CCT.GTC | 83V | |
| | | | TTTTTT-3′Tag | | |
| | | | SEQ ID NO: 33 | | |
| 6 | WT | 80D-SE- | 5′Tag-TTTTTTTT | 78R.79F.80D.81N. | WT |
| | | RE1.10 | AGG.TTT.GAT.AAC.CC | 82• | |
| | | | CTTTTTTT-3′Tag | | |
| | | | SEQ ID NO: 281 | | |
| 7 | M1 | 80A-SE- | 5′Tag-TTTTTTT | 78•.79F.80A.81N. | BETA |
| | | RE1.1 | GG.TTT.GCT.AAC.CCT.G | 82P.83• | |
| | | | CTTTTTTT-3′Tag | | |
| | | | SEQ ID NO: 35 | | |
| 8 | U | T951-SE- | 5′Tag-TTT | 91L.92F.93A.94S. | ALL |
| | | RE1.1 | CTT.TTT.GCT.TCC.AYT.G | 95T/I.96E.97K. | VARIANTS |
| | | | AG.AAG.TCT | 98S | |
| | | | TTTTT-3′Tag | | |
| | | | SEQ ID NO: 153 | | |
| 9 | WT | T95-SE- | 5′Tag-TTTTTTCC | 93A.945 .95T.96E. | WT |
| | | RE1.3 | GCT.TCC.ACT.GAG.AAG | 97K.985 | |
| | | | CATTTTT-3′Tag | | |
| | | | SEQ ID NO: 154 | | |
| 10 | M1 | 95I-SE- | 5′Tag-TTTTTTCC | | OMICRON, |
| | | RE1.2 | GCT.TCC.ATT.GAG.AAG | 93A,94S,95I,96 | DELTA, MU, |
| | | | CATTTTT-3′Tag | E,97K,985 | AZ.5, KAPPA, |
| | | | SEQ ID NO: 155 | | IOTA, AV.1 |
| 11 | U | 138U-SE- | 5′Tag-TTTT | 134•.135F.136C. | ALL |
| | | RE1.2 | A.TTT.TGT.AAT.KAT. | 137N.138D/Y. | VARIANTS |
| | | | CCA.TTT.TTG | 139P.140F.141L | |
| | | | TTTT-3′Tag | | |
| | | | SEQ ID NO: 36 | | |
| 12 | WT | 138D-SE- | 5′Tag-TTTTTC | 135•.136C.137N. | WT |
| | | RE1.1 | TT.TGT.AAT.GAT.CCA. | 138D.139P.140 | |
| | | | TTT.T | F.141• | |
| | | | CTTTTT-3′Tag | | |
| | | | SEQ ID NO: 37 | | |
| 13 | M1 | 138Y-SE- | 5′Tag-TTTTT | 135F.136C.137N. | GAMMA |
| | | RE1.1 | TTT.TGT.AAT.TAT.CCA.T | 138Y.139P.140F. | |
| | | | TT.T | 141• | |
| | | | CTTTTT-3′Tag | | |
| | | | SEQ ID NO: 38 | | |
| 14 | U | 144U- | 5′Tag-TTC | 140•.141L.142D/G. | ALL |
| | | 1.1M | TT.TTG.GRT.GTT.♦♦♦. | 143V.144Y. | VARIANTS |
| | | | TAT.TAC.CAC.AAA.AAC | 145Y.146 H.147K. | |
| | | | TTT-3′Tag | 148N | |
| | | | SEQ ID NO: 282 | | |
| | | | 5′Tag-TTTCT | 142G.143V(insT). | |
| | | | GGT.GTT.ACT.TCT.AAC. | 1445.145N. | |
| | | | CAC.AA | 146H.147• | |
| | | | TTTTT-3′Tag | | |
| | | | SEQ ID NO: 283 | | |
| | | | 5′Tag-TTC | 138D.139P.140F. | |
| | | | GAT.CCA.TTT.TTG.GAC- | 141L.142 D.14 | |
| | | | --.♦♦♦.---.---. | 3del.144del.145 | |
| | | | CAC.AAA.AAC.AA | del.146H.147K. | |
| | | | TTT-3′Tag | 148N, 149• | |
| | | | SEQ ID NO: 284 | | |
| | | | 5′Tag-TTT | | |
| | | | A.TTT.TTG.GGT.GTT. | 139•.140F.141L. | |
| | | | ♦♦♦.---.TAC.CAC.AAA. | 142G.143V.144 | |
| | | | AAC.A | del.145Y.146H. | |
| | | | TTT-3′Tag | 147K. 48N.149• | |
| | | | SEQ ID NO: 285 | | |
| | | | 5′Tag-TTTTT. | 136.137N.138D. | |
| | | | | 139P.140F.141del. | |
| | | | AAT.GAT.CCA.TTT.---. | 142del.143del. | |
| | | | ---.---.♦♦♦.TAT.TAC. | 144Y.145Y.146H. | |
| | | | CAC.AA | 147• | |
| | | | 3′Tag-TTTT | | |
| | | | SEQ ID NO: 286 | | |
| 15 | WT | Y2144- | 5′Tag-TTC | 140•.141L.142D/ | WT |
| | | SE-RE1.2 | TT.TTG.GRT.GTT.♦♦♦.TA | G.143V.144Y. | |
| | | | T.TAC.CAC.AAA.AAC | 145Y.146H.147K. | |
| | | | TTT-3′Tag | 148N | |
| | | | SEQ ID NO: 287 | | |
| 16 | M1 | 144T-SE- | 5′Tag-TTTCT | 142G.143V(insT) | MU |
| | | RE1.1 | GGT.GTT.ACT.TCT.AAC. | 1445.145N. | |
| | | | CAC.AA | 146H. 147• | |
| | | | TTTTT-3′Tag | | |
| | | | SEQ ID NO: 288 | | |
| 17 | M2 | 144OMI- | 5′Tag-TTC | 138D.139P.140F. | OMICRON |
| | | RE1.2 | GAT.CCA.TTT.TTG.GAC.- | 141L.142D.143del. | |
| | | | --.♦♦♦.---.--- | 144del.145 | |
| | | | .CAC.AAA.AAC.AA | del. 146H.147K. | |
| | | | TTT-3′Tag | 148N. 149• | |
| | | | SEQ ID NO: 289 | | |
| 18 | U | 152U-SE- | 5′Tag-TTTTT | 1515.152W/C/L/R. | ALL |
| | | RE1.1 | AGT.WKK.ATG.GAA.AGT. | 153M.154E. | |
| | | | GAG.TTC | 155S. 156E.157F | VARIANTS |
| | | | TTTT-3′Tag | | |
| | | | SEQ ID NO: 290 | | |
| 19 | WT | 152W-SE- | 5′Tag-TTTCTCT | | WT |
| | | RE1.16 | AAA.AGT.TGG.ATG. | 150K.1515.152W. | |
| | | | GAA.A | 153M.154E.155• | |
| | | | CTCTTCT-3′Tag | | |
| | | | SEQ ID NO: 40 | | |
| 20 | M1 | 152C-SE- | 5′Tag-TTTCTTC | 150•.1515.152C. | EPSILON |
| | | RE1.10 | AA.AGT.TGT.ATG | 153M.154E.155• | |
| | | | GAA.AG | | |
| | | | CCTTCTT-3′Tag | | |
| | | | SEQ ID NO: 41 | | |
| 21 | M2 | 152L-SE- | 5′Tag-TTTCTCT | 150K.1515.152L. | R.1 |
| | | RE1.2 | AAA.AGT.TTG.ATG. | 153M.154E. | |
| | | | GAA.A | 155• | |
| | | | TCTTCTT-3′Tag | | |
| | | | SEQ ID NO: 161 | | |
| 22 | M3 | 152R-SE- | 5′Tag-TTTCTTT | 149•.150K.151S. | C.36.3 |
| | | RE1.4 | C.AAA.AGT.AGG. | 152R.153M.154E | |
| | | | ATG.GAA | | |
| | | | TCTTCTT-3′Tag | | |
| | | | SEQ ID NO: 291 | | |
| 23 | U | 157 | 5′Tag-TTTTC | 154•.155S.156E. | ALL |
| | | UNIVERSAL | AA.AGT.GAG.TTC.AGA. | 157F.158R.159V. | VARIANTS |
| | | | GTT.TA | 160• | |
| | | | CCTTTT-3′Tag | | |
| | | | SEQ ID NO: 292 | | |
| | | | 5′Tag-TTCTTTG. | 153•.154E.155S. | |
| | | | GAA.AGT.GGA.---.---. | 156G.157del. | |
| | | | GTT.TAT.TC | 158del.159V. | |
| | | | CTTTTT-3′Tag | 160Y.170• | |
| | | | SEQ ID NO: 293 | | |
| 24 | WT | F157-SE- | 5′Tag-TTTTC | 154•.155S.156E. | WT |
| | | RE1.3 | AA.AGT.GAG.TTC.AGA. | 157F.158R.159 | |
| | | | GTT.TA | V. 160• | |
| | | | CCTTTT-3′Tag | | |
| | | | SEQ ID NO: 164 | | |
| 25 | M1 | SE-RE1.7 | 5′Tag-TTCTTTG. | 153•.154E.155S. | DELTA |
| | | | GAA.AGT.GGA.---.---. | 156G.157del. | |
| | | | GTT.TAT.TC | 158del.159V. | |
| | | | CTTTTT-3′Tag | 160Y.170• | |
| | | | SEQ ID NO: 293 | | |
| 26 | U | 439U440 | 5′Tag-TTTTT | 437N.438S.439N/ | ALL |
| | | U-SE- | AAT.TCT.AAM.AAK.CTT. | K.440N/K.441L. | VARIANTS |
| | | RE1.1 | GAT.TCT.AA | 442D.443S. | |
| | | | TTTT-3′Tag | 444• | |
| | | | SEQ ID NO: 42 | | |
| 27 | WT | 439N440 | 5′Tag-TTTTT | 437N.438S.439N. | WT |
| | | N-SE- | AAT.TCT.AAC.AAT.CTT. | 440N.441L.44 | |
| | | RE1.1 | GAT.T | 2D. 443• | |
| | | | TCTTTT-3′Tag | | |
| | | | SEQ ID NO: 43 | | |
| 28 | M1 | 439N440k- | 5′Tag-TTTTTT | 437•.438S.439N. | OMICRON, |
| | | SE- | AT.TCT.AAC.AAG.CTT. | 440K.441L.442 | B.1.619 |
| | | RE1.1 | GAT.T | D. 443• | |
| | | | TTTTTT-3′Tag | | |
| | | | SEQ ID NO: 44 | | |
| 29 | M2 | 439K440N- | 5′Tag-TTTTCT | | |
| | | SE- | AT.TCT.AAA.AAT.CTT. | 437•.438S.439K. | |
| | | RE1.5 | GAT.T | 440N.441L.442D. | B.1.466.2, |
| | | | TCTTTT-3′Tag | 443• | AV.1 |
| | | | SEQ ID NO: 45 | | |
| 30 | U | 452U-SE- | 5′Tag-TTTC | 449Y.450N.451Y. | ALL |
| | | RE1.1 | TAT.AAT.TAC.CDG.TAT. | 452L/R/Q.453Y. | VARIANTS |
| | | | AGA.TTG.T | 454R.455L. | |
| | | | CTTT-3′Tag | 456• | |
| | | | SEQ ID NO: 251 | | |
| 31 | WT | 452L-SE- | 5′Tag-TTTTTT | 449•.450N.451Y. | WT |
| | | RE1.2 | T.AAT.TAC.CTG.TAT. | 452L.453Y.454R. | |
| | | | AGA.TT | 455• | |
| | | | TCTTTT-3′Tag | | |
| | | | SEQ ID NO: 47 | | |
| 32 | M1 | 452R-SE- | 5′Tag-TTTTTC | 449•.450N.451Y. | DELTA, |
| | | RE1.5 | AT.AAT.TAC.CGG.TAT.A | 452R.453Y.454R. | KAPPA, |
| | | | GA.T | 455• | B.1.630, |
| | | | CTTTTT-3′Tag | | C.36.3, |
| | | | SEQ ID NO: 294 | | EPSILON, |
| | | | | | A.27 |
| 33 | M2 | 452Q-SE- | 5′Tag-TTTTTTT | 449•.450N.451Y. | LAMBDA |
| | | RE1.2 | T.AAT.TAC.CAG.TAT. | 452Q.453Y. | |
| | | | AGA | 454R | |
| | | | CTTTTTT-3′Tag | | |
| | | | SEQ ID NO: 295 | | |
| 34 | U | S477N- | 5′Tag-TTTTTC | 475A.476G.477S/ | ALL |
| | | SE-RE1.2 | GCC.GGT.ARC.AMA. | N.478T/K.479P. | VARIANTS |
| | | | CCT.TGT.A | 480• | |
| | | | TTTTT-3′Tag | | |
| | | | SEQ ID NO: 252 | | |
| 35 | U | T478K- | 5′Tag-TTTTT | 475•.476G.477S. | ALL |
| | | SE-RE1.1 | C.GGT.AGC.AMA.CCT. | 478T/K.479P. | VARIANTS |
| | | | TGT.AAT.G | 480C.481N.482• | |
| | | | TTTTT-3′Tag | | |
| | | | SEQ ID NO: 174 | | |
| 36 | WT | T478-SE- | 5′Tag-TTTTTTT | 476G.477S.478T. | WT |
| | | RE1.2 | GGT.AGC.ACA.CCT.TGT | 479P.480C | |
| | | | TTTTTTTT-3′Tag | | |
| | | | SEQ ID NO: 296 | | |
| 37 | M1 | 478K-SE- | 5′Tag-TTTTTTT | 476•.4775.478K. | DELTA, |
| | | RE1.5 | GT.AGC.AAA.CCT.TGT.A | 479P.480C. | C.1.2, |
| | | | TTTTTTTT-3′Tag | 481• | B.1.1.519 |
| | | | SEQ ID NO: 176 | | |
| 38 | U | 484U-SE- | 5′Tag-TTTTT | 480•.481N.482G. | ALL |
| | | RE1.1 | T AAT GGT.GYT.RMA. | 483V/A.484E/K/A. | VARIANTS |
| | | | GG | 485G.486F. | |
| | | | T.TTT.AAT.T | 487N. 488• | |
| | | | TTTT-3′Tag | | |
| | | | SEQ ID NO: 297 | | |
| 39 | WT | 483V484E-SE- | 5′Tag-TTTTTTCT. | 481•.482G.483V. | WT |
| | | RE1.7 | GGT.GTT.GAA.GGT. | 484E.485G. | |
| | | | TTT.A | 486F. 487• | |
| | | | CTTTTT-3′Tag | | |
| | | | SEQ ID NO: 52 | | |
| 40 | M1 | 484K-SE- | 5′Tag-TTTTTTTA | 481•.482G.483V. | BETA, |
| | | RE1.5 | T′GGT.GTT.AAA.GGT. | 484K.485G. | GAMMA, |
| | | | TTT | 486F | MU,AZ.5, |
| | | | CTTTTT-3′Tag | | C.1.2. |
| | | | SEQ ID NO: 53 | | IOTA(a), |
| | | | | | ETA, R.1, |
| | | | | | AV.1, AT.1, |
| | | | | | ZETA, THETA, |
| | | | | | B.1.523, |
| | | | | | B.1.619, |
| | | | | | B.1.620 |
| 41 | M2 | 483A-SE- | 5′Tag-TTTTTTT | 481•.482G.483A. | B.1.616 |
| | | RE1.3 | AT.GGT.GCT.GAA. | 484E.485G.486. | |
| | | | GGT.T | | |
| | | | CTTTTTTT-3′Tag | | |
| | | | SEQ ID NO: 54 | | |
| 42 | M3 | V483_._4 | 5′Tag-TTTTTTC | 482G.483V.484A. | OMICRON |
| | | 84A- | GGT.GTT.GCA.GGT. | 485G.486F. | |
| | | RE1.2 | TTT.A | 487• | |
| | | | TCTTTTT-3′Tag | | |
| | | | SEQ ID NO: 298 | | |
| 43 | U | F490S- | 5′Tag-TTTTC | 486•.487N.488C. | ALL |
| | | SE-RE1.1 | T.AAT.TGT.TAC.TYT.CCT | 489Y.490F/S. | VARIANTS |
| | | | TTA.CAA.T | 491P.492L.493Q. | |
| | | | TTTT-3′Tag | 494• | |
| | | | SEQ ID NO: 256 | | |
| 44 | WT | F490-SE- | 5′Tag-TTTTTTT | 487•.488C.489Y. | WT |
| | | RE1.1 | T.TGT.TAC.TTT.CCT. | 490F.491P.492L. | |
| | | | TTA.C | 493• | |
| | | | TTTTTT-3′Tag | | |
| | | | SEQ ID NO: 179 | | |
| 45 | M1 | 490S-SE- | 5′Tag-TTTTTTT | 487•.488C.489Y. | LAMBDA |
| | | RE1.1 | T.TGT.TAC.TCT.CCT. | 490S.491P.492L. | |
| | | | TTA.C | 493. | |
| | | | TTTTTT-3′Tag | | |
| | | | SEQ ID NO: 180 | | |
| 46 | U | 493.494- | 5′Tag-TTTTTCT. | 490•.491P.492L. | ALL |
| | | SE-RE1.3 | CCT.TTA.CAA.YCA.TAT. | P.493Q/R.4945/ | VARIANTS |
| | | | GGT.TT | 495Y.496G/ | |
| | | | TTTTT-3′Tag | S.497. | |
| | | | SEQ ID NO: 299 | | |
| | | | 5′Tag-TTTTTCT | | |
| | | | TTA.CGA.TCA.TAT.AGT. | | |
| | | | TTC | | |
| | | | CTTTTT-3′Tag | | |
| | | | SEQ ID NO: 300 | | |
| 47 | WT | S494-SE- | 5′Tag-TTTTTCT | 491•.492L.493Q. | WT |
| | | RE1.1 | CT.TTA.CAA.TCA.TAT | 4945.495Y. | |
| | | | GGT | 496G | |
| | | | CTTTTT-3′Tag | | |
| | | | SEQ ID NO: 182 | | |
| 48 | M1 | 494P-SE- | 5′Tag-TTTTTCT | 491•.492L.493Q. | B.1.575, |
| | | RE1.1 | CT.TTA.CAA.CCA.TAT. | 494P.495Y. | B.1.1.523 |
| | | | GGT | 496G | |
| | | | CTTTTT-3′Tag | | |
| | | | SEQ ID NO: 183 | | |
| 49 | M2 | _493R._496S- | 5′Tag-TTTTTC | 492L.493R.494S. | OMICRON |
| | | RE1.1 | TTA.CGA.TCA.TAT.AGT. | 495Y.496S. | |
| | | | TTC | 497F | |
| | | | TTTTTT-3′Tag | | |
| | | | SEQ ID NO: 301 | | |
| 50 | U | 501UNI- | 5′Tag-TTTTT | 496•.497F.498Q. | ALL |
| | | SE-RE1.1 | T.TTC.CAA.CCC.ACT | 499P.500T.501N/ | VARIANTS |
| | | | WAT.GGT.GTT | Y.502G.503V | |
| | | | TTTTTT-3′Tag | | |
| | | | SEQ ID NO: 302 | | |
| 51 | U | 677U-SE- | 5′Tag-TTTT | 673•.674Y.675Q. | ALL |
| | | RE1.1 | T.TAT.CAG.ACT.CMB.AC | 676T.677Q/P/H. | VARIANTS |
| | | | T.AAT.TCT.0 | 678T.679N. | |
| | | | TTTTT-3′Tag | 680S. 681• | |
| | | | SEQ ID NO: 259 | | |
| 52 | WT | Q677-SE- | 5′Tag-TTTTTTC | 675Q.676T.677Q. | WT |
| | | RE1.1 | CAG.ACT.CAG.ACT. | 678T.679N. | |
| | | | AAT.T | 680• | |
| | | | TCTTTTT-3′Tag | | |
| | | | SEQ ID NO: 126 | | |
| 53 | M1 | 677H2- | 5′Tag-TTTTTTC | 675Q.676T.677H. | ETA, R.2 |
| | | SE-RE1.1 | CAG.ACT.CAC.ACT. | 678T.679N. | |
| | | | AAT.T | 680• | |
| | | | TCTTTTT-3′Tag | | |
| | | | SEQ ID NO: 129 | | |
| 54 | M2 | 677H1- | 5′Tag-TTTTTTC | 675Q.676T.677H. | C.36.3 |
| | | SE-RE1.1 | CAG.ACT.CAT.ACT. | 678T.679N. | |
| | | | AAT.T | 680• | |
| | | | TCTTTTT-3′Tag | | |
| | | | SEQ ID NO: 189 | | |
| 55 | U | 681U-SE- | 5′Tag-TTTTT | 676•.677Q.678T. | ALL |
| | | RE1.1 | T.CAG.ACT.AAK.TCT. | 679N/K.680S. | VARIANTS |
| | | | CVT.CGG.C | 681P/H/R.682R. | |
| | | | TTTTT-3′Tag | 683• | |
| | | | SEQ ID NO: 303 | | |
| 56 | WT | 681P-SE- | 5′Tag-TTTTTTT | 678•.679N.680S. | WT |
| | | RE1.2 | CT.AAT.TCT.CCT. | 681P.682R. | |
| | | | CGG.CG | 683• | |
| | | | TTTTTTT-3′Tag | | |
| | | | SEQ ID NO: 59 | | |
| 57 | M1 | 681H- | 5′Tag-TTTTTTTT | 678•.679N/K.68 | Omicron, |
| | | OMIWOB- | T.AAK.TCT.CAT.CGG.CG | 0S.681H.682R. | ALPHA, MU, |
| | | RE1.1 | TTTTTTT-3′Tag | 683• | AZ.5, B.1.640, |
| | | | SEQ ID NO: 304 | | B.1.575, AV.1, |
| | | | | | THETA, |
| | | | | | B.1.1.519, |
| | | | | | B.1.620 |
| 58 | M2 | 681R-SE- | 5′Tag-TTTTTTTT | 678•.679N.680S. | DELTA, |
| | | RE1.1 | T.AAT.TCT.CGT.CGG.CG | 681R.682R. | KAPPA, |
| | | | TTTTTTT-3′Tag | 683• | B.1.466.2 |
| | | | SEQ ID NO: 190 | | |
| 59 | U | 701U- | 5′Tag-TTT | 698S.699L.700G. | ALL |
| | | 560E- | TCA.CTT.GGT.GYA.GAA | 701A/V.702E. | VARIANTS |
| | | RE1.1 | AAT.TCA.GTT | 703N.7045. | |
| | | | TTT-3′Tag | 705V | |
| | | | SEQ ID NO: 61 | | |
| 60 | WT | 701A-SE- | 5′Tag-TCTTCTT | 699L.700G.701A. | WT |
| | | RE1.13 | CTT.GGT.GCA.GAA. | 702E.703N. | |
| | | | AAT.T | 704• | |
| | | | ATTCTTT-3′Tag | | |
| | | | SEQ ID NO: 62 | | |
| 61 | M1 | 701V-SE- | 5′Tag-TCTTCTT | 699L.700G.701V. | BETA, IOTA |
| | | RE1.16 | CTT.GGT.GTA.GAA.AAT | 702E.703N | |
| | | | CTTCTTTT-3′Tag | | |
| | | | SEQ ID NO: 305 | | |

Probe Type:
C-CONTROL,
U-UNIVERSAL,
WT-WILD TYPE,
M1-MUTANT1,
M2-MUTANT2,
M3-MUTANT3
(_)-Underlined Target Mutation;
(•)-Incomplete codon;
(♦♦♦)-Gap due to alignment with a 3 nt base insertion;
(---)-Deleted codon

TABLE 31
Universal Probe Location in the Spike Gene, Sequence and Design Rational

| | | | | Method Used to |
|---|---|---|---|---|
| | | | | Generate the |
| | | Universal | | Universal Probe |
| | Location | ProbeSeq | Universal Probe | Composition |
| Index | (AA) | ID | Sequence | Affinity |
| 2 | 69-70 | 69-70 | TTTTTCCCATGYTATACATGT | Introduce Single Base |
| | universal | | CTCTGTTTTTT | Degeneracy |
| | | | SEQ ID NO: 235 | (Y) = CIT |
| | | | TTTTTTTTTCCATGYTATCTC | Mix WT + Deletion |
| | | | TGGGATTTTTT | Sequences |
| | | | SEQ ID NO: 236 | C32/T NEW |
| 5 | 80 | 80U-SE- | TTTCTTTTTGCTTCCAYTGAG | Expand Length of Probe |
| | | RE1.1 | AAGTCTTTTTT | to Mitigate Effect of Single |
| | | | SEQ ID NO: 33 | Base Change on Affinity |
| | | T951-SE- | TTTCTTTTTGCTTCCAYTGAG | Introduce Single Base |
| 8 | 95 | RE1.1 | AAGTCTTTTTT | Degeneracy (Y) = C/T |
| | | | SEQ ID NO: 153 | |
| 11 | 138 | 138U-SE- | TTTTATTTTGTAATKATCCAT | Expand Length of Probe |
| | | RE1.2 | TTTTGTTTT | to Mitigate Effect of Single |
| | | | SEQ ID NO: 36 | Base Change on Affinity |
| 14 | 14+ | 144U-1.1M | TTCTTTTGGRTGTTTATTACC | Mix WT + Deletion |
| | | | ACAAAAACTTT | Sequences |
| | | | SEQ ID NO: 283 | |
| | | | TTTCTGGTGTTACTTCTAAC | |
| | | | CACAATTTTT | |
| | | | SEQ ID NO: 284 | |
| | | | TTCGATCCATTTTTGGACCA | |
| | | | CAAAAACAATTT | |
| | | | SEQ ID NO: 285 | |
| | | | TTTATTTTTGGGTGTTTACCA | |
| | | | CAAAAACATTT | |
| | | | SEQ ID NO: 286 | |
| | | | TTTTTAATGATCCATTTTATT | |
| | | | ACCACAATTTT | |
| | | | SEQ ID NO: 287 | |
| 18 | 152 | 152U-SE- | TTTTTAGTWKKATGGAAAGT | Introduce Triple Single |
| | | RE1.1 | GAGTTCTTTT | Base Degeneracy |
| | | | SEQ ID NO: 291 | (W) = A/T (K) = G/T |
| | | | | Expand Length of Probe |
| | | | | to Mitigate Effect of Single |
| | | | | Base Change on Affinity |
| 23 | 157 | 157 | TTTTCAAAGTGAGTTCAGAG | Mix WT + Deletion |
| | | universal | TTTACCTTTT | Sequences |
| | | | SEQ ID NO: 293 | |
| | | | TTCTTTGGAAAGTGGAGTTT | |
| | | | ATTCCTTTTT | |
| | | | SEQ ID NO: 294 | |
| 26 | 439-440 | 439U440U- | TTTTTAATTCTAAMAAKCTTG | Introduce Triple Single |
| | | SE-RE1.1 | ATTCTAATTTT | Base Degeneracy |
| | | | SEQ ID NO: 42 | (W) = A/T (K) = G/T |
| | | | | Expand Length of Probe |
| | | | | to Mitigate Effect of Single |
| | | | | Base Change on Affinity |
| 30 | 452 | 452U-SE- | TTTCTATAATTACCDGTATAG | Expand Length of Probe |
| | | RE1.1 | ATTGTCTTT | to Mitigate Effect of Single |
| | | | SEQ ID NO: 251 | Base Change on Affinity |
| | | | | Introduce Triple Base |
| | | | | Degeneracy (D) = A/G/T |
| 34 | 477 | 5477N-SE- | TTTTTCGCCGGTARCAMACC | Introduce Double, Single |
| | | RE1.2 | TTGTATTTTT | Base Degeneracy |
| | | | SEQ ID NO: 252 | (R) = G/A (M) = C/A |
| | | | | Expand Length of Probe |
| | | | | to Mitigate Effect of Single |
| | | | | Base Change on Affinity |
| 35 | 478 | T478K-SE- | TTTTTCGGTAGCAMACCTTG | Introduce Single Base |
| | | RE1.1 | TAATGTTTTT | Degeneracy (M) = C/A |
| | | | SEQ ID NO: 174 | Expand Length of Probe |
| | | | | to Mitigate Effect of Single |
| | | | | Base Change on Affinity |
| 38 | 484 | 484U-SE- | TTTTTTAATGGTGYTRMAGG | Introduce Triple, Single |
| | | RE1.1 | TTTTAATTTTTT | Base Degeneracy |
| | | | SEQ ID NO: 299 | (Y) = C/T (R) = G/A |
| | | | | (M) = C/A |
| | | | | Expand Length of Probe |
| | | | | to Mitigate Effect of Single |
| | | | | Base Change on Affinity |
| 43 | 490 | F490S-SE- | TTTTCTAATTGTTACTYTCCT | Introduce Single Base |
| | | RE1.1 | TTACAATTTTT | Degeneracy (Y) = C/T |
| | | | SEQ ID NO: 256 | Expand Length of Probe |
| | | | | to Mitigate Effect of Single |
| | | | | Base Change on Affinity |
| 46 | 493/494/ | 493.494- | TTTTTCTCCTTTACAAYCATA | Introduce Single Base |
| | 496 | SE-RE1.3 | TGGTTTTTTTT | Degeneracy (Y) = C/T |
| | | | SEQ ID NO: 301 | |
| | | | TTTTTCTTTACGATCATATAG | Mix WT + Deletion |
| | | | TTTCCTTTTT | Sequences |
| | | | SEQ ID NO: 302 | |
| 50 | 501 | 501UNI- | TTTTTTTTCCAACCCACTWA | Introduce Single Base |
| | | SE-RE1.1 | TGGTGTTTTTTTT | Degeneracy |
| | | | SEQ ID NO: 55 | (W) = NT |
| | | | | Expand Length of Probe |
| | | | | to Mitigate Effect of Single |
| | | | | Base Change on Affinity |
| 51 | 677 | 677U-SE- | TTTTTTATCAGACTCMBACTA | Introduce Base |
| | | RE1.1 | ATTCTCTTTTT | Degeneracy |
| | | | SEQ ID NO 259 | B = C/G/T |
| | | | | Expand Length of Probe |
| | | | | to Mitigate Effect of Single |
| | | | | Base Change on Affinity |
| 55 | 681 | 681U-SE- | TTTTTTCAGACTAAKTCTCVT | Introduce Base |
| | | RE1.1 | CGGCTTTTT SEQ ID NO: 305 | Degeneracy |
| | | | | (K) = G/T (V) = C/A/G |
| | | | | Expand Length of Probe |
| | | | | to Mitigate Effect of Single |
| | | | | Base Change on Affinity |
| 59 | 701 | 701U-SE- | TTTTCACTTGGTGYAGAAAA | Introduce Single Base |
| | | RE1.1 | TTCAGTTTTT SEQ ID NO. 61 | Degeneracy (Y) = CIT |
| | | | | Expand Length of Probe |
| | | | | to Mitigate Effect of Single |
| | | | | Base Change on Affinity |

“Index”, Column 1, identifies the probe number associated with each Universal Probe among the full list of 61 probes of the Detect^{x}-Cv⁺assay, as described in Table 2.
“Location”, Column 2, refers to the amino acid position within the SARS-CoV-2 protein sequence where the Universal Probe binds to the corresponding gRNA sequence.
“Universal ProbeSeq ID”, Column 3, indicates the name of each Universal Probe.
“Universal Probe Sequence”, Column 4, describes the nucleotide sequence comprising each Universal Probe.
“Method Used to Generate the Universal Probe Composition”, Column 5, describes the molecular genetic design approach deployed to allow each Universal Probe to bind to both the Wild Type and Mutant sequence at its cognate site in the Spike Gene.

### Wild Type and Mutant Probes Used to Detect Spike Gene Mutations

Probes include universal probes, wild type probes and mutant probes. Probe design for Detect^{X}-Cv⁺ is identical to those for Detect^{X}-Rv. Briefly, all probes are designed to include several thymidine (T) bases at each end, i.e. 5′Tag and 3′Tag, which are used to facilitate probe assembly, by UV crosslinking, subsequent to oligonucleotide printing onto the microarray surface. 61 probes (Table 30) are printed in triplicate to form a single microarray, comprising 183 discrete 100 μm wide spots in a 183-element microarray, with each oligonucleotide probe immobilized on the bottom of each well of a SBS standard 96-well format, i.e., 96 identical microarrays per 96-well plate. Each well is thus capable of providing a complete set (61×3) of microarray hybridization tests for each sample.

As confirmed in Tables 32A-32D experimental specificity analysis of synthetic Spike gene templates with known mutational changes, all mutant probes (Table 30) are designed to bind 5- to 10-fold more tightly to their cognate, perfectly matched mutant spike gene target sequence, as compared to the same spike sequence from the wild type (Wuhan) progenitor. Thus, the wild type and mutant probes themselves have a sequence that differs by only one or a few bases (Table 30) and may be used to discriminate a mutational sequence change at the 16 sites in the SARS-CoV-2 sequence for which they were designed. The probes are designed to interrogate wild type vs mutant sequence change by hybridizing to a template sequence that is 15-20 bases long and, thus, additional mutations that might arise in the surrounding amplicon can only affect probe function if the mutation had occurred in that 15-20 base long probe binding domain.

TABLE 32A
Variant Mutation Site Analytical Detection Limits

| | Mutations | Copies/ | | | | |
|---|---|---|---|---|---|---|
| Variant | Validated | μL | 69-70 | 80 | 95 | 138 |
| B.1.351 | 80A, 484K, | 6 | | 20/20 | | |
| Beta | 701V | | | | | |
| Delta | 157 deletion, | 600 | | | | |
| | 452R, 478K and | | | | | |
| | 681R | | | | | |
| B.1.427/ | 152C and 452R | 200 | | | | |
| 429 | | | | | | |
| Epsilon | | | | | | |
| B.1.526a | 95I, 484K, | 200 | | | 20/20 | |
| Iota | 701V | | | | | |
| C.37 | 452Q and 490S | 60 | | | | |
| Lambda | | | | | | |
| B.1.621 | 95I, 144T, | 600 | | | 20/20 | |
| Mu | 681H | | | | | |
| Mut1 | 69-70 deletion, | 200 | 20/20 | | | |
| | 152R, 439K, | | | | | |
| | 452Q, | | | | | |
| | 483A, 494P, | | | | | |
| | 677H1, 701V | | | | | |
| Mut2 | 69/70 del, 95I, | 200 | 20/20 | | 20/20 | |
| | 152R, 483A and | | | | | |
| | 677H2. | | | | | |
| Omicron | 69-70 del, 95I, | 200 | 20/20 | | 20/20 | |
| | 144 del, 440K, | 60 | 20/20 | | 20/20 | |
| | 484A, 493R, | | | | | |
| | 496S, 681H | | | | | |
| P.1 | 138Y, 484K | 60 | | | | 20/20 |
| Gamma | | | | | | |
| R.1 | 152L, 484K | 20000 | | | | |

TABLE 32B
Variant Mutation Site Analytical Detection Limits

| | Mutations | Copies/ | | | 157/ | 439- |
|---|---|---|---|---|---|---|
| Variant | Validated | μL | 144 | 152 | 158 | 440 |
| B.1.351 | 80A, 484K, | 6 | | | | |
| Beta | 701V | | | | | |
| Delta | 157 deletion, | 600 | | | 20/20 | |
| | 452R, 478K and | | | | | |
| | 681R | | | | | |
| B.1.427/ | 152C and 452R | 200 | | 20/20 | | |
| 429 | | | | | | |
| Epsilon | | | | | | |
| B.1.526a | 95I, 484K, | 200 | | | | |
| Iota | 701V | | | | | |
| C.37 | 452Q and 490S | 60 | | | | |
| Lambda | | | | | | |
| B.1.621 | 95I, 144T, | 600 | 20/20 | | | |
| Mu | 681H | | | | | |
| Mut1 | 69-70 deletion, | 200 | | 20/20 | | 20/20 |
| | 152R, 439K, | | | | | |
| | 452Q, 483A, | | | | | |
| | 494P, | | | | | |
| | 677H1, 701V | | | | | |
| Mut2 | 69/70 del, 95I, | 200 | | 20/20 | | |
| | 152R, 483A and | | | | | |
| | 677H2. | | | | | |
| Omicron | 69-70 del, 95I, | 200 | 20/20 | | | 20/20 |
| | 144 del, 440K, | 60 | 20/20 | | | 20/20 |
| | 484A, 493R, | | | | | |
| | 496S, 681H | | | | | |
| P.1 | 138Y, 484K | 60 | | | | |
| Gamma | | | | | | |
| R.1 | 152L, 484K | 20000 | | 20/20 | | |

TABLE 32C
Variant Mutation Site Analytical Detection Limits

| | Mutations | Copies/ | | | 483- | |
|---|---|---|---|---|---|---|
| Variant | Validated | μL | 452 | 478 | 484 | 490 |
| B.1.351 | 80A, 484K, | 6 | | | 20/20 | |
| Beta | 701V | | | | | |
| Delta | 157 deletion, | 600 | 20/20 | 19/20 | | |
| | 452R, 478K and | | | | | |
| | 681R | | | | | |
| B.1.427/ | 152C and 452R | 200 | 20/20 | | | |
| 429 | | | | | | |
| Epsilon | | | | | | |
| B.1.526a | 95I, 484K, | 200 | | | 20/20 | |
| Iota | 701V | | | | | |
| C.37 | 452Q and 490S | 60 | 20/20 | | | 20/20 |
| Lambda | | | | | | |
| B.1.621 | 95I, 144T, | 600 | | | | |
| Mu | 681H | | | | | |
| Mut1 | 69-70 deletion, | 200 | 20/20 | | 19/20 | |
| | 152R, 439K, | | | | | |
| | 452Q, | | | | | |
| | 483A, 494P, | | | | | |
| | 677H1, 701V | | | | | |
| Mut2 | 69/70 del, 95I, | 200 | | | 19/20 | |
| | 152R, 483A and | | | | | |
| | 677H2. | | | | | |
| Omicron | 69-70 del, 95I, | 200 | | | 20/20 | |
| | 144 del, 440K, | 60 | | | 20/20 | |
| | 484A, 493R, | | | | | |
| | 496S, 681H | | | | | |
| P.1 | 138Y, 484K | 60 | | | 19/20 | |
| Gamma | | | | | | |
| R.1 | 152L, 484K | 20000 | | | 20/20 | |

TABLE 32D
Variant Mutation Site Analytical Detection Limits

| | Mutations | Copies/ | 493- | | | |
|---|---|---|---|---|---|---|
| Variant | Validated | μL | 494 | 677 | 681 | 701 |
| B.1.351 | 80A, 484K, | 6 | | | | |
| Beta | 701V | | | | | |
| Delta | 157 deletion, | 600 | | | | 19/20 |
| | 452R, 478K and | | | | | |
| | 681R | | | | | |
| B.1.427/ | 152C and 452R | 200 | | | | |
| 429 | | | | | | |
| Epsilon | | | | | | |
| B.1.526a | 95I, 484K, | 200 | | | | |
| Iota | 701V | | | | | |
| C.37 | 452Q and 490S | 60 | 20/20 | | | |
| Lambda | | | | | | |
| B.1.621 | 95I, 144T, | 600 | | | | 20/20 |
| Mu | 681H | | | | | |
| Mut1 | 69-70 deletion, | 200 | | 20/20 | 20/20 | |
| | 152R, 439K, | | | | | |
| | 452Q, | | | | | |
| | 483A, 494P, | | | | | |
| | 677H1, 701V | | | | | |
| Mut2 | 69/70 del, 95I, | 200 | | | 20/20 | |
| | 152R, 483A and | | | | | |
| | 677H2. | | | | | |
| Omicron | 69-70 del, 95I, | 200 | | 20/20 | | 20/20 |
| | 144 del, 440K, | 60 | | 5/20 | | 20/20 |
| | 484A, 493R, | | | | | |
| | 496S, 681H | | | | | |
| P.1 | 138Y, 484K | 60 | | | | |
| Gamma | | | | | | |
| R.1 | 152L, 484K | 20000 | | | | |

It was determined that the incidence of such new mutation in those adjacent probe binding domains is seen to be low, among the entirety of the approximately 7 million current entries in the GISAID database. However, if such a mutation were to occur, it would manifest as a significant loss of binding affinity for both the wild type and mutant probes at each site, given that both of these probes are designed to bind much more weakly upon induction of a single base change anywhere in the 15-20 base binding domain. Thus, in the presence of such unforeseen additional mutations, the hybridization signal associated with both wild type and mutant probe binding would be diminished, in parallel, since both would be subject to the same additional base pairing mismatch.

Two outcomes would result. In most cases, the hybridization signal for both the wild type and mutant probes would be reduced to below their respective threshold value. Augury software would report that mutational analysis as un-measurable or not detected at that site. If the addition of a flanking sequence mutation produced a more modest effect (i.e. wild type and mutant probe signals remained above threshold) as would occur if the viral gRNA target the patient sample was highly concentrated, then the Augury software would report the outcome. In that instance, the result would remain correct, as the addition of the extra mutant would affect the two probes equivalently and the resulting Mutation vs Wild Type distinction based on binding affinity difference due to the target mutation would remain accurate. Ultimately, a mutation would be detected and reported at that site. The universal probe at each site would be affected by flanking sequence mutation much as is the case for wild type and mutant probes. Should the universal probe hybridization signal be reduced to below its threshold, it would be reported as “Not Present”

### Limit of Detection (LoD) for SARS-CoV-2

The limit of detection for SARS-CoV-2 is 300 copies/mL as summarized in Table 33.

TABLE 33
Results of Final LoD Study

| | | Positive | Negative | |
|---|---|---|---|---|
| | Concentration | Detection of | Detection of | |
| Virus Type | (copies/mL) | SARS-CoV-2 | SARS-CoV-2 | Total |
| Heat Inactivated SARS-CoV-2 | 3000 | 20/20 | 0 | 20 |
| Gamma Irradiated SARS-CoV-2 | 300 | 19/20 | 1 | 20 |

### Variant Detection and Analytical Validation

The ability of Detect^{X}-Cv⁺ to detect Variants of Interest (VOI) and Variants of Concern (Variants of Concern) was verified using gBlocks and four next-generation sequencing (NGS) sequenced clinical samples. The four sequenced clinical samples were purchased from Fulgent and tested in Detect^{X}-Cv⁺ according to the Assay Protocol above. The results are summarized in Table 34A.

TABLE 34A
Clinical Variants by NGS tested with Detect^{X}-Cv⁺

| | | | Mutations detected by |
|---|---|---|---|
| Sample | Fulgent Sample ID | NGS Variant Call | Detect^{X}-Cv⁺ |
| 1 | NF203314622 | Delta + 417N | 157del, 452R, 478K, 681R |
| 2 | NF206722076 | Delta | 157del, 452R, 478K, 681R |
| 3 | NF202436378 | Delta | 157del, 452R, 478K, 681R |
| 4 | NF2000458256 | P.1 | 138Y, 501Y |

Reactivity with the variants shown in Tables 32A-32D was verified using synthetic gBlocks. Briefly, stock concentrations at 10⁸ copies/mL were thawed, and centrifuged for 1 minute at 14,000×g. Each stock was serially diluted in nuclease-free water to the working concentrations shown in Table 33 and the assay was performed according to the Assay Protocol above. Zymo MagBead extraction was not included when preparing gBlock samples. RNAse P (1,000 copies/μL) was added to the test wells. A no template control and a positive control composed of B.1.1.7 synthetic DNA (10,000 cp/μL) and RNase P synthetic DNA (100 cp/μL) were included. The samples were tested in replicates of 20 and detection of each individual mutation point was considered valid where at least 19/20 replicates at the mutation sites known for each variant were positive at the concentrations listed. Delta, Lambda and Omicron variant RNA was also tested at 3× and 10× of their cutoffs, along with gamma irradiated SARS-CoV-2 (BEI NR52287), in triplicate (Table 34B).

TABLE 34B
Clinical Variants by NGS tested with Detect^{X}-Cv⁺

| Sample | LOD (Copies/μL) | Detected at 3 X LOD | Detected at 10 X LOD |
|---|---|---|---|
| 1 | 300 | 3/3 | 3/3 |
| 2 | 300 | 3/3 | 3/3 |
| 3 | 60 | 3/3 | 3/3 |
| 4 | 200 | 3/3 | 3/3 |

What is claimed is:

1. A method for detecting severe acute respiratory syndrome coronavirus 2 (SARS-CoV-2) in a subject, comprising:
obtaining a sample from the subject;
isolating total RNA from the sample;
performing in a single assay a combined reverse transcription and asymmetric PCR amplification reaction on the total RNA using a plurality of fluorescently labeled primer pairs comprising an unlabeled primer and a fluorescently labeled primer, selective for target sequences within a Spike gene in the SARS-CoV-2 virus to generate a plurality of fluorescently labeled SARS-CoV-2 amplicons;
hybridizing the plurality of fluorescently labeled SARS-CoV-2 amplicons to a plurality of nucleic acid probes comprising a plurality of universal probes, wild type probes and mutant probes, each having a sequence that specifically base-pairs with one of the target sequences in the fluorescently labeled SARS-CoV-2 amplicons and at least one control probe to which the fluorescently labeled SARS-CoV-2 amplicons do not hybridize, each of said nucleic acid probes attached to specific positions on a solid microarray support;
washing the microarray at least once;
imaging the microarray to detect fluorescent signals above a threshold for all the nucleic acid probes upon hybridization to the fluorescently labeled SARS-CoV-2 amplicons.

2. The method of claim 1, wherein, during the imaging step, SARS-CoV-2 is detected by measuring at least N fluorescent signals above the threshold from hybridizing of the fluorescently labeled SARS-CoV-2 amplicons to the universal probes.

3. The method of claim 2, wherein N is equal to or greater than 6 fluorescent signals.

4. The method of claim 1, wherein the Spike gene is genotyped at each target sequence, the method further comprising:
measuring the fluorescent signal from hybridization of the fluorescently labeled SARS-CoV-2 amplicons to the wild type probes and from the hybridation of the fluorescently labeled SARS-CoV-2 amplicons to the mutant probes at each of the target sequences;
analyzing directly a relative size of the fluorescent signal from hybridization to the mutant probes vs. the fluorescent signal from hybridization to the wild type probes to produce a hybridization pattern of wild type vs. mutant genotyping among all the target sites in SARS-CoV-2; and
comparing the hybridization pattern to a known pattern of wild type vs. mutant genotype variation among known SARS-CoV-2 variants to identify the SARS-CoV-2 in the sample as a known variant of concern or a known variant of interest or a combination thereof or as an unknown variant.

5. The method of claim 1, wherein the plurality of fluorescently labeled primer pairs is a set of nucleotide sequences comprising SEQ ID NO: 266 and SEQ ID NO: 138, SEQ ID NO: 11 and SEQ ID NO: 139, SEQ ID NO: 267 and SEQ ID NO: 141, SEQ ID NO: 268 and SEQ ID NO: 16, and SEQ ID NO: 141 and SEQ ID NO: 269.

6. The method of claim 1, wherein the plurality of fluorescently labeled primer pairs further comprises an internal control primer pair to amplify RNase P and the control probe comprises a sequence that specifically base-pairs with a fluorescently labeled RNase P amplicon.

7. The method of claim 6, wherein the internal control primer pair comprises the nucleotide sequences of SEQ ID NO: 132 and SEQ ID NO: 270.

8. The method of claim 6, wherein the control probe comprises the nucleotide sequence of SEQ ID NO: 134.

9. The method of claim 1, wherein the universal probes comprise the nucleotide sequences of SEQ ID NOS: 33, 36, 42, 61, 153, 174, 235, 236, 251-252, 256, 259, 283, 287, 291, 293-294, 299, 301, 302 304, and 305.

10. The method of claim 1, wherein the wild type probes comprise the nucleotide sequences of SEQ ID NOS: 37, 40, 43, 47, 52, 59, 62, 126, 154, 164, 179, 182, 235, 282, 288, and 298.

11. The method of claim 1, wherein the mutant probes comprise the nucleotide sequences of SEQ ID NOS: 35, 38, 41, 44, 45, 53, 54, 129, 155, 161, 176, 180, 183, 189, 190, 236, 289, 290, 292, 295, 296, 297, 300, 303, 306, and 307.

12. The method of claim 1, wherein the fluorescently labeled primer is in an excess of about 4-fold to about 8-fold over the unlabeled primer in the fluorescent labeled primer pair.

13. The method of claim 1, wherein the sample is a nasopharyngeal swab, a nasal swab, a mouth swab, or saliva.

14. A method for detecting, genotyping and identifying a variant of severe acute respiratory syndrome coronavirus 2 (SARS-CoV-2) in a subject, comprising:
obtaining a sample from the subject;
isolating total RNA from the sample;
performing in a single assay a combined reverse transcription and asymmetric PCR amplification reaction on the total RNA using a set of fluorescently labeled primer pairs, each comprising an unlabeled primer and a fluorescently labeled primer, selective for sequences within a Spike gene in the SARS-CoV-2 virus to generate a plurality of fluorescent labeled SARS-CoV-2 amplicons;
hybridizing the plurality of fluorescently labeled SARS-CoV-2 amplicons to a plurality of nucleic acid probes comprising a set of universal probes, wild type probes and mutant probes, each having a sequence that specifically base-pairs with one of the target sequences in the fluorescently labeled SARS-CoV-2 amplicons and at least one control probe to which the fluorescently labeled SARS-CoV-2 amplicons do not hybridize, each of said nucleic acid probes attached to specific positions on a solid microarray support;
washing the microarray at least once;
imaging the microarray to detect fluorescent signals above threshold for all the nucleic acid probes produced upon hybridization to the fluorescently labeled SARS-CoV-2 amplicons;
measuring at least N fluorescent signals above the threshold from hybridization of the fluorescently labeled SARS-CoV-2 amplicons to the universal probes thereby detecting the SARS-CoV-2 in the sample;
genotyping the Spike gene at each target sequence, the step comprising:
comparing the fluorescent signals from hybridization of the fluorescently labeled SARS-CoV-2 amplicons to the wild type probes and from the hybridation of the fluorescently labeled SARS-CoV-2 amplicons to the mutant probes at each position on the microarray; and
analyzing directly a relative size of the fluorescent signal from hybridization to the mutant probes vs. the fluorescent signal from hybridization to the wild type probes to produce a hybridization pattern of wild type vs. mutant genotyping at each target sequence in SARS-CoV-2; and
identifying a variant of SARS-CoV-2 as a known variant of concern or a known variant of interest or a combination thereof or as an unknown variant by comparing the hybridization pattern to a known pattern of wild type vs. mutant genotype variation among known SARS-CoV-2 variants.

15. The method of claim 14, wherein N is equal to or greater than 6 fluorescent signals.

16. The method of claim 14, wherein the set of fluorescently labeled primer pairs comprises the nucleotide sequences of SEQ ID NO: 266 and SEQ ID NO: 138, SEQ ID NO: 11 and SEQ ID NO: 139, SEQ ID NO: 267 and SEQ ID NO: 141, SEQ ID NO: 268 and SEQ ID NO: 16, SEQ ID NO: 141 and SEQ ID NO: 269, and SEQ ID NO: 132 and SEQ ID NO: 270.

17. The method of claim 14, wherein and the probe hybridizing to the fluorescently labeled RNase P amplicon comprises the nucleotide sequence of SEQ ID NO: 134.

18. The method of claim 14, wherein the universal probes comprise the nucleotide sequences of SEQ ID NOS: 33, 36, 42, 61, 153, 174, 235, 236, 251-252, 256, 259, 283, 287, 291, 293-294, 299, 301, 302 304, and 305.

19. The method of claim 14, wherein the wild type probes comprise the nucleotide sequences of SEQ ID NOS: 37, 40, 43, 47, 52, 59, 62, 126, 154, 164, 179, 182, 235, 282, 288, and 298.

20. The method of claim 14, wherein the mutant probes comprise the nucleotide sequences of SEQ ID NOS: 35, 38, 41, 44, 45, 53, 54, 129, 155, 161, 176, 180, 183, 189, 190, 236, 289, 290, 292, 295, 296, 297, 300, 303, 306, and 307.

21. The method of claim 14, wherein the fluorescently labeled primer is in an excess of about 4-fold to about 8-fold over the unlabeled primer in the fluorescent labeled primer pair.

22. The method of claim 14, wherein the sample is a nasopharyngeal swab, a nasal swab, a mouth swab, or saliva.


SEQUENCE LISTING (305 sequences)

SEQ ID NO: 1 - DNA, 28 bp, Artificial Sequence
  Forward primer sequence for SARS CoV-2 Spike gene
tttaacagagttgttatttctagtgatg

SEQ ID NO: 2 - DNA, 28 bp, Artificial Sequence
  Reverse primer sequence for SARS CoV-2 Spike gene
ttttctaaagtagtaccaaaaatccagc

SEQ ID NO: 3 - DNA, 28 bp, Artificial Sequence
  Forward primer sequence for SARS CoV-2 Spike gene
tttccctacttattgttaataacgctac

SEQ ID NO: 4 - DNA, 28 bp, Artificial Sequence
  Reverse primer sequence for SARS CoV-2 Spike gene
tttagataacccacataataagctgcag

SEQ ID NO: 5 - DNA, 28 bp, Artificial Sequence
  Forward primer sequence for SARS CoV-2 Spike gene
tttatctctgctttactaatgtctatgc

SEQ ID NO: 6 - DNA, 28 bp, Artificial Sequence
  Reverse primer sequence for SARS CoV-2 Spike gene
tttacaaacagttgctggtgcatgtaga

SEQ ID NO: 7 - DNA, 28 bp, Artificial Sequence
  Forward primer sequence for SARS CoV-2 Spike gene
ttttggtgtcagtgttataacaccagga

SEQ ID NO: 8 - DNA, 28 bp, Artificial Sequence
  Reverse primer sequence for SARS CoV-2 Spike gene
ttttgtcttggtcatagacactggtaga

SEQ ID NO: 9 - DNA, 28 bp, Artificial Sequence
  Forward primer sequence for SARS CoV-2 Spike gene
acctttcttttccaatgttacttggttc

SEQ ID NO: 10 - DNA, 28 bp, Artificial Sequence
  Reverse primer sequence for SARS CoV-2 Spike gene
  modified_base 1: Thymidine at position 1 is modified with a fluorescent label
ttttatgttagacttctcagtggaagca

SEQ ID NO: 11 - DNA, 28 bp, Artificial Sequence
  Forward primer sequence for SARS CoV-2 Spike gene
tttcttattgttaataacgctactaatg

SEQ ID NO: 12 - DNA, 28 bp, Artificial Sequence
  Reverse primer sequence for SARS CoV-2 Spike gene
  modified_base 1: Thymidine at position 1 is modified with a fluorescent label
tttcattcgcactagaataaactctgaa

SEQ ID NO: 13 - DNA, 25 bp, Artificial Sequence
  Forward primer sequence for SARS CoV-2 Spike gene
tgtaattagaggtgatgaagtcaga

SEQ ID NO: 14 - DNA, 28 bp, Artificial Sequence
  Reverse primer sequence for SARS CoV-2 Spike gene
  modified_base 1: Thymidine at position 1 is modified with a fluorescent label
tttaaaggtttgagattagacttcctaa

SEQ ID NO: 15 - DNA, 28 bp, Artificial Sequence
  Forward primer sequence for SARS CoV-2 Spike gene
ttttatttcaactgaaatytatcaggcc

SEQ ID NO: 16 - DNA, 28 bp, Artificial Sequence
  Reverse primer sequence for SARS CoV-2 Spike gene
  modified_base 1: Thymidine at position 1 is modified with a fluorescent label
tttaaagtactactactctgtatggttg

SEQ ID NO: 17 - DNA, 28 bp, Artificial Sequence
  Forward primer sequence for SARS CoV-2 Spike gene
ttttatatgcgctagttatcagactcag

SEQ ID NO: 18 - DNA, 28 bp, Artificial Sequence
  Reverse primer sequence for SARS CoV-2 Spike gene
  modified_base 1: Thymidine at position 1 is modified with a fluorescent label
ttttggtatggcaatagagttattagag

SEQ ID NO: 19 - DNA, 28 bp, Artificial Sequence
  Forward primer sequence for SARS CoV-2 Spike gene
tttttttcttgttttattgccactagtc

SEQ ID NO: 20 - DNA, 28 bp, Artificial Sequence
  Reverse primer sequence for SARS CoV-2 Spike gene
  modified_base 1: Thymidine at position 1 is modified with a fluorescent label
tttttgtcagggtaataaacaccacgtg

SEQ ID NO: 21 - DNA, 28 bp, Artificial Sequence
  Forward primer sequence for SARS CoV-2 Clade variant Spike gene
ttttaagcacacgcctattaatttagtg

SEQ ID NO: 22 - DNA, 28 bp, Artificial Sequence
  Reverse primer sequence for SARS CoV-2 Clade variant Spike gene
  modified_base 1: Thymidine at position 1 is modified with a fluorescent label
tttccacataataagctgcagcaccagc

SEQ ID NO: 23 - DNA, 28 bp, Artificial Sequence
  Forward primer sequence for SARS CoV-2 Clade variant Spike gene
tttagtgttataacaccaggaacaaata

SEQ ID NO: 24 - DNA, 28 bp, Artificial Sequence
  Reverse primer sequence for SARS CoV-2 Clade variant Spike gene
  modified_base 1: Thymidine at position 1 is modified with a fluorescent label
ttttgcatgaatagcaacagggacttct

SEQ ID NO: 25 - DNA, 28 bp, Artificial Sequence
  Forward primer sequence for SARS CoV-2 Clade variant Spike gene
ttcttttccaatgttacttggttccatg

SEQ ID NO: 26 - DNA, 28 bp, Artificial Sequence
  Reverse primer sequence for SARS CoV-2 Clade variant Spike gene
  modified_base 1: Thymidine at position 1 is modified with a fluorescent label
tttcaaaataaacaccatcattaaatgg

SEQ ID NO: 27 - DNA, 28 bp, Artificial Sequence
  Forward primer sequence for SARS CoV-2 Clade variant Spike gene
tttgatgaagtcagacaaatcgctccag

SEQ ID NO: 28 - DNA, 28 bp, Artificial Sequence
  Reverse primer sequence for SARS CoV-2 Clade variant Spike gene
  modified_base 1: Thymidine at position 1 is modified with a fluorescent label
tttctctcaaaaggtttgagattagact

SEQ ID NO: 29 - DNA, 28 bp, Artificial Sequence
  Forward primer sequence for SARS CoV-2 Clade variant Spike gene
tttcaaatacttctaaccaggttgctgt

SEQ ID NO: 30 - DNA, 30 bp, Artificial Sequence
  Probe sequence for SARS CoV-2 Clade variant
tttttctagtctctaktcagtgtgtttttt

SEQ ID NO: 31 - DNA, 32 bp, Artificial Sequence
  Probe sequence for SARS CoV-2 Clade variant
tttttcccatgctatacatgtctctgtttttt

SEQ ID NO: 32 - DNA, 32 bp, Artificial Sequence
  Probe sequence for SARS CoV-2 Clade variant
tttttttttccatgctatctctgggatttttt

SEQ ID NO: 33 - DNA, 32 bp, Artificial Sequence
  Probe sequence for SARS CoV-2 Clade variant
tttttcagaggtttgmtaaccctgtctttttt

SEQ ID NO: 34 - DNA, 30 bp, Artificial Sequence
  Probe sequence for SARS CoV-2 Clade variant
tttttttggtttgataaccctgcttttttt

SEQ ID NO: 35 - DNA, 30 bp, Artificial Sequence
  Probe sequence for SARS CoV-2 Clade variant
tttttttggtttgctaaccctgcttttttt

SEQ ID NO: 36 - DNA, 30 bp, Artificial Sequence
  Probe sequence for SARS CoV-2 Clade variant
ttttattttgtaatkatccatttttgtttt

SEQ ID NO: 37 - DNA, 30 bp, Artificial Sequence
  Probe sequence for SARS CoV-2 Clade variant
tttttctttgtaatgatccattttcttttt

SEQ ID NO: 38 - DNA, 30 bp, Artificial Sequence
  Probe sequence for SARS CoV-2 Clade variant
tttttttttgtaattatccattttcttttt

SEQ ID NO: 39 - DNA, 30 bp, Artificial Sequence
  Probe sequence for SARS CoV-2 Clade variant
tttttagttgkatggaaagtgagttctttt

SEQ ID NO: 40 - DNA, 30 bp, Artificial Sequence
  Probe sequence for SARS CoV-2 Clade variant
tttctctaaaagttggatggaaactcttct

SEQ ID NO: 41 - DNA, 30 bp, Artificial Sequence
  Probe sequence for SARS CoV-2 Clade variant
tttcttcaaagttgtatggaaagccttctt

SEQ ID NO: 42 - DNA, 32 bp, Artificial Sequence
  Probe sequence for SARS CoV-2 Clade variant
tttttaattctaamaakcttgattctaatttt

SEQ ID NO: 43 - DNA, 30 bp, Artificial Sequence
  Probe sequence for SARS CoV-2 Clade variant
tttttaattctaacaatcttgatttctttt

SEQ ID NO: 44 - DNA, 30 bp, Artificial Sequence
  Probe sequence for SARS CoV-2 Clade variant
ttttttattctaacaagcttgatttttttt

SEQ ID NO: 45 - DNA, 30 bp, Artificial Sequence
  Probe sequence for SARS CoV-2 Clade variant
ttttctattctaaaaatcttgatttctttt

SEQ ID NO: 46 - DNA, 30 bp, Artificial Sequence
  Probe sequence for SARS CoV-2 Clade variant
tttctataattacctgtatagattgtcttt

SEQ ID NO: 47 - DNA, 30 bp, Artificial Sequence
  Probe sequence for SARS CoV-2 Clade variant
tttttttaattacctgtatagatttctttt

SEQ ID NO: 48 - DNA, 30 bp, Artificial Sequence
  Probe sequence for SARS CoV-2 Clade variant
tttttcataattactggtatagatcttttt

SEQ ID NO: 49 - DNA, 30 bp, Artificial Sequence
  Probe sequence for SARS CoV-2 Clade variant
ttttttcgccggtagcacacctcttttttt

SEQ ID NO: 50 - DNA, 30 bp, Artificial Sequence
  Probe sequence for SARS CoV-2 Clade variant
ttttcttccggtaacacacctttttttttt

SEQ ID NO: 51 - DNA, 32 bp, Artificial Sequence
  Probe sequence for SARS CoV-2 Clade variant
ttttttaatggtgttraaggttttaatttttt

SEQ ID NO: 52 - DNA, 30 bp, Artificial Sequence
  Probe sequence for SARS CoV-2 Clade variant
ttttttctggtgttgaaggttttacttttt

SEQ ID NO: 53 - DNA, 30 bp, Artificial Sequence
  Probe sequence for SARS CoV-2 Clade variant
tttttttatggtgttaaaggttttcttttt

SEQ ID NO: 54 - DNA, 30 bp, Artificial Sequence
  Probe sequence for SARS CoV-2 Clade variant
tttttttatggtgctgaaggttcttttttt

SEQ ID NO: 55 - DNA, 33 bp, Artificial Sequence
  Probe sequence for SARS CoV-2 Clade variant
ttttttttccaacccactwatggtgtttttttt

SEQ ID NO: 56 - DNA, 30 bp, Artificial Sequence
  Probe sequence for SARS CoV-2 Clade variant
ttttttttacccactaatggtgtctttttt

SEQ ID NO: 57 - DNA, 30 bp, Artificial Sequence
  Probe sequence for SARS CoV-2 Clade variant
ttttttttacccacttatggtgtctttttt

SEQ ID NO: 58 - DNA, 30 bp, Artificial Sequence
  Probe sequence for SARS CoV-2 Clade variant
ttttttcagactaattctcmtcggcttttt

SEQ ID NO: 59 - DNA, 30 bp, Artificial Sequence
  Probe sequence for SARS CoV-2 Clade variant
tttttttctaattctcctcggcgttttttt

SEQ ID NO: 60 - DNA, 30 bp, Artificial Sequence
  Probe sequence for SARS CoV-2 Clade variant
tttttttttaattctcatcggcgttttttt

SEQ ID NO: 61 - DNA, 30 bp, Artificial Sequence
  Probe sequence for SARS CoV-2 Clade variant
ttttcacttggtgyagaaaattcagttttt

SEQ ID NO: 62 - DNA, 30 bp, Artificial Sequence
  Probe sequence for SARS CoV-2 Clade variant
tcttcttcttggtgcagaaaattattcttt

SEQ ID NO: 63 - DNA, 30 bp, Artificial Sequence
  Probe sequence for SARS CoV-2 Clade variant
tcttcttcttggtgtagaaaattattcttt

SEQ ID NO: 64 - DNA, 30 bp, Artificial Sequence
  Probe sequence for SARS CoV-2 Clade variant
tttttttgtctctagtcagtgttttttttt

SEQ ID NO: 65 - DNA, 30 bp, Artificial Sequence
  Probe sequence for SARS CoV-2 Clade variant
tttttttagtctctagtcagtgtttttttt

SEQ ID NO: 66 - DNA, 30 bp, Artificial Sequence
  Probe sequence for SARS CoV-2 Clade variant
ttttttagtctctattcagtgttttttttt

SEQ ID NO: 67 - DNA, 30 bp, Artificial Sequence
  Probe sequence for SARS CoV-2 Clade variant
tttttttagtctctattcagtgtttttttt

SEQ ID NO: 68 - DNA, 30 bp, Artificial Sequence
  Probe sequence for SARS CoV-2 Clade variant
ttttttaatyttacaamcagaactcttttt

SEQ ID NO: 69 - DNA, 30 bp, Artificial Sequence
  Probe sequence for SARS CoV-2 Clade variant
tttttttatcttacaaccagaacctttttt

SEQ ID NO: 70 - DNA, 30 bp, Artificial Sequence
  Probe sequence for SARS CoV-2 Clade variant
tttttttatcttacaaccagaacttttttt

SEQ ID NO: 71 - DNA, 30 bp, Artificial Sequence
  Probe sequence for SARS CoV-2 Clade variant
ttttttattttacaaacagaactttttttt

SEQ ID NO: 72 - DNA, 30 bp, Artificial Sequence
  Probe sequence for SARS CoV-2 Clade variant
tttttcaattttacaaacagaacttttttt

SEQ ID NO: 73 - DNA, 30 bp, Artificial Sequence
  Probe sequence for SARS CoV-2 Clade variant
ttttttatgctatacatgtctctgtttttt

SEQ ID NO: 74 - DNA, 30 bp, Artificial Sequence
  Probe sequence for SARS CoV-2 Clade variant
ttttttaccatgctatctctgggatttttt

SEQ ID NO: 75 - DNA, 30 bp, Artificial Sequence
  Probe sequence for SARS CoV-2 Clade variant
tttttctaggtttgataaccctgctttttt

SEQ ID NO: 76 - DNA, 30 bp, Artificial Sequence
  Probe sequence for SARS CoV-2 Clade variant
tttttttaggtttgctaaccctcttttttt

SEQ ID NO: 77 - DNA, 30 bp, Artificial Sequence
  Probe sequence for SARS CoV-2 Clade variant
ttttttctttgtaatgatccatttcttttt

SEQ ID NO: 78 - DNA, 30 bp, Artificial Sequence
  Probe sequence for SARS CoV-2 Clade variant
tttttctttgtaattatccattttcttttt

SEQ ID NO: 79 - DNA, 30 bp, Artificial Sequence
  Probe sequence for SARS CoV-2 Clade variant
ttttcttcaaagttggatggaaactctttt

SEQ ID NO: 80 - DNA, 30 bp, Artificial Sequence
  Probe sequence for SARS CoV-2 Clade variant
tttctctaaaagttgtatggaaactcttct

SEQ ID NO: 81 - DNA, 30 bp, Artificial Sequence
  Probe sequence for SARS CoV-2 Clade variant
ttttttagtgcgtgrtctccctcatttttt

SEQ ID NO: 82 - DNA, 30 bp, Artificial Sequence
  Probe sequence for SARS CoV-2 Clade variant
ttttttctgcgtgatctccctcattttttt

SEQ ID NO: 83 - DNA, 30 bp, Artificial Sequence
  Probe sequence for SARS CoV-2 Clade variant
tttttttctgcgtgatctccctcttttttt

SEQ ID NO: 84 - DNA, 30 bp, Artificial Sequence
  Probe sequence for SARS CoV-2 Clade variant
ttttttttgcgtggtctccctctttttttt

SEQ ID NO: 85 - DNA, 30 bp, Artificial Sequence
  Probe sequence for SARS CoV-2 Clade variant
tttttttttgcgtggtctcccttttttttt

SEQ ID NO: 86 - DNA, 30 bp, Artificial Sequence
  Probe sequence for SARS CoV-2 Clade variant
ttttaactggaaakattgctgattattttt

SEQ ID NO: 87 - DNA, 30 bp, Artificial Sequence
  Probe sequence for SARS CoV-2 Clade variant
tttcttctctggaaagattgctgctttttt

SEQ ID NO: 88 - DNA, 30 bp, Artificial Sequence
  Probe sequence for SARS CoV-2 Clade variant
ttcttctctggaaagattgctgactttttt

SEQ ID NO: 89 - DNA, 30 bp, Artificial Sequence
  Probe sequence for SARS CoV-2 Clade variant
tttttctctggaaatattgctgactttttt

SEQ ID NO: 90 - DNA, 30 bp, Artificial Sequence
  Probe sequence for SARS CoV-2 Clade variant
ttttctctggaaatattgctgatctttttt

SEQ ID NO: 91 - DNA, 30 bp, Artificial Sequence
  Probe sequence for SARS CoV-2 Clade variant
tttttttactggaacgattgcttttttttt

SEQ ID NO: 92 - DNA, 30 bp, Artificial Sequence
  Probe sequence for SARS CoV-2 Clade variant
ttttttcctggaacgattgctgtttttttt

SEQ ID NO: 93 - DNA, 30 bp, Artificial Sequence
  Probe sequence for SARS CoV-2 Clade variant
ttttttattctaacaatcttgatttctttt

SEQ ID NO: 94 - DNA, 30 bp, Artificial Sequence
  Probe sequence for SARS CoV-2 Clade variant
tttttttttctaacaagcttgatttttttt

SEQ ID NO: 95 - DNA, 30 bp, Artificial Sequence
  Probe sequence for SARS CoV-2 Clade variant
ttcttaattctaaaaatcttgatttctttt

SEQ ID NO: 96 - DNA, 30 bp, Artificial Sequence
  Probe sequence for SARS CoV-2 Clade variant
tttttcataattacctgtatagactttctt

SEQ ID NO: 97 - DNA, 30 bp, Artificial Sequence
  Probe sequence for SARS CoV-2 Clade variant
ttttttcaattaccggtatagatctttttt

SEQ ID NO: 98 - DNA, 30 bp, Artificial Sequence
  Probe sequence for SARS CoV-2 Clade variant
ttttttttggtagcataccttgtttttttt

SEQ ID NO: 99 - DNA, 29 bp, Artificial Sequence
  Probe sequence for SARS CoV-2 Clade variant
tttttttcggtagcataccttgttttttt

SEQ ID NO: 100 - DNA, 30 bp, Artificial Sequence
  Probe sequence for SARS CoV-2 Clade variant
ttttttcgccggtaacacacctcttttttt

SEQ ID NO: 101 - DNA, 30 bp, Artificial Sequence
  Probe sequence for SARS CoV-2 Clade variant
ttttttttcaggccagtagcactttttttt

SEQ ID NO: 102 - DNA, 30 bp, Artificial Sequence
  Probe sequence for SARS CoV-2 Clade variant
tttttctggtgttgaaggttttatcttttt

SEQ ID NO: 103 - DNA, 30 bp, Artificial Sequence
  Probe sequence for SARS CoV-2 Clade variant
ttttttctggtgttaaaggttttacttttt

SEQ ID NO: 104 - DNA, 30 bp, Artificial Sequence
  Probe sequence for SARS CoV-2 Clade variant
ttttttcaatggtgctgaaggttctttttt

SEQ ID NO: 105 - DNA, 30 bp, Artificial Sequence
  Probe sequence for SARS CoV-2 Clade variant
tttttttaacccactaatggtgtctttttt

SEQ ID NO: 106 - DNA, 30 bp, Artificial Sequence
  Probe sequence for SARS CoV-2 Clade variant
tttttttaacccacttatggtgtctttttt

SEQ ID NO: 107 - DNA, 34 bp, Artificial Sequence
  Probe sequence for SARS CoV-2 Clade variant
tttttctctttatcargrtgttaactgctttttt

SEQ ID NO: 108 - DNA, 30 bp, Artificial Sequence
  Probe sequence for SARS CoV-2 Clade variant
tttttcttatcaggatgttaactttttttt

SEQ ID NO: 109 - DNA, 30 bp, Artificial Sequence
  Probe sequence for SARS CoV-2 Clade variant
ttttttcctatcagggtgttaacttttttt

SEQ ID NO: 110 - DNA, 30 bp, Artificial Sequence
  Probe sequence for SARS CoV-2 Clade variant
ttttttcctatcaaggtgttaacttttttt

SEQ ID NO: 111 - DNA, 30 bp, Artificial Sequence
  Probe sequence for SARS CoV-2 Clade variant
ttttttcctatcarggtgttaacttttttt

SEQ ID NO: 112 - DNA, 30 bp, Artificial Sequence
  Probe sequence for SARS CoV-2 Clade variant
ttttttttaattctcctcggcgtttttttt

SEQ ID NO: 113 - DNA, 30 bp, Artificial Sequence
  Probe sequence for SARS CoV-2 Clade variant
tttttttctaattctcatcggcgttttttt

SEQ ID NO: 114 - DNA, 30 bp, Artificial Sequence
  Probe sequence for SARS CoV-2 Clade variant
ttcttctacttggtgcagaaaattattctt

SEQ ID NO: 115 - DNA, 30 bp, Artificial Sequence
  Probe sequence for SARS CoV-2 Clade variant
tttctttcttggtgtagaaaattctttttt

SEQ ID NO: 116 - DNA, 30 bp, Artificial Sequence
  Probe sequence for SARS CoV-2 Clade variant
ttttttacaatttgcccccagcgtcttttt

SEQ ID NO: 117 - DNA, 30 bp, Artificial Sequence
  Probe sequence for SARS CoV-2 Clade variant
tttttttttgctccragtgcctcttttttt

SEQ ID NO: 118 - DNA, 32 bp, Artificial Sequence
  Probe sequence for SARS CoV-2 Clade variant
ttttttttcaaactttacttgctttactcttt

SEQ ID NO: 119 - DNA, 32 bp, Artificial Sequence
  Probe sequence for SARS CoV-2 Clade variant
ttttttttcaaactttacatagaagccttttt

SEQ ID NO: 120 - DNA, 32 bp, Artificial Sequence
  Probe sequence for SARS CoV-2 Clade variant
ttttctacatagaagttatttgactccctttt

SEQ ID NO: 121 - DNA, 32 bp, Artificial Sequence
  Probe sequence for SARS CoV-2 Clade variant
ttttctgctttacatatgactcctggtttttt

SEQ ID NO: 122 - DNA, 30 bp, Artificial Sequence
  Probe sequence for SARS CoV-2 Clade variant
tttctactcctggtgrttcttcttcatttt

SEQ ID NO: 123 - DNA, 30 bp, Artificial Sequence
  Probe sequence for SARS CoV-2 Clade variant
ttttttccctggtgattcttctttcttttt

SEQ ID NO: 124 - DNA, 30 bp, Artificial Sequence
  Probe sequence for SARS CoV-2 Clade variant
ttttttccctggtggttcttcttttttttt

SEQ ID NO: 125 - DNA, 32 bp, Artificial Sequence
  Probe sequence for SARS CoV-2 Clade variant
ttttttatcagactcmgactaattctcttttt

SEQ ID NO: 126 - DNA, 30 bp, Artificial Sequence
  Probe sequence for SARS CoV-2 Clade variant
ttttttccagactcagactaatttcttttt

SEQ ID NO: 127 - DNA, 30 bp, Artificial Sequence
  Probe sequence for SARS CoV-2 Clade variant
tttttcttcagactccgactaatctttttt

SEQ ID NO: 128 - DNA, 30 bp, Artificial Sequence
  Probe sequence for SARS CoV-2 Clade variant
ttttttccagactcatactaatttcttttt

SEQ ID NO: 129 - DNA, 30 bp, Artificial Sequence
  Probe sequence for SARS CoV-2 Clade variant
ttttttccagactcacactaatttcttttt

SEQ ID NO: 130 - DNA, 28 bp, Artificial Sequence
  Primer sequence for human RNAse P control
tttacttcagcatggcggtgtttgcaga

SEQ ID NO: 131 - DNA, 28 bp, Artificial Sequence
  Primer sequence for human RNAse P control
ttttgatagcaacaactgaatagccaag

SEQ ID NO: 132 - DNA, 28 bp, Artificial Sequence
  Primer sequence for human RNAse P control
tttgtttgcagatttggacctgcgagcg

SEQ ID NO: 133 - DNA, 28 bp, Artificial Sequence
  Primer sequence for human RNAse P control
tttaaggtgagcggctgtctccacaagt

SEQ ID NO: 134 - DNA, 34 bp, Artificial Sequence
  Probe sequence for human RNAse P control
ttttttttctgacctgaaggctctgcgcgttttt

SEQ ID NO: 135 - DNA, 30 bp, Artificial Sequence
  Probe sequence for human RNAse P control
tttttcttgacctgaaggctctgctttttt

SEQ ID NO: 136 - DNA, 34 bp, Artificial Sequence
  Probe sequence for Negative Control
ttttttctactacctatgctgattcactcttttt

SEQ ID NO: 137 - DNA, 28 bp, Artificial Sequence
  Forward primer sequence for SARS CoV-2 Spike gene
tttagagttgttatttctagtgatgttc

SEQ ID NO: 138 - DNA, 28 bp, Artificial Sequence
  Reverse primer sequence for SARS CoV-2 Spike gene
  modified_base 1: Thymidine at position 1 is modified with a fluorescent label
tttaatccagcctcttattatgttagac

SEQ ID NO: 139 - DNA, 28 bp, Artificial Sequence
  Reverse primer sequence for SARS CoV-2 Spike gene
  modified_base 1: Thymidine at position 1 is modified with a fluorescent label
tttcaaaagtgcaattattcgcactaga

SEQ ID NO: 140 - DNA, 28 bp, Artificial Sequence
  Forward primer sequence for SARS CoV-2 Spike gene
tttcttttgagagagatatttcaactga

SEQ ID NO: 141 - DNA, 26 bp, Artificial Sequence
  Forward primer sequence for SARS CoV-2 Spike gene
tttattggtgcaggtatatgcgctag

SEQ ID NO: 142 - DNA, 26 bp, Artificial Sequence
  Forward primer sequence for SARS CoV-2 Nucleoprotein gene
tttgccaaaaggcttctacgcagaag

SEQ ID NO: 143 - DNA, 26 bp, Artificial Sequence
  Reverse primer sequence for SARS CoV-2 Nucleoprotein gene
ttttttgccgaggcttcttagaagcc

SEQ ID NO: 144 - DNA, 32 bp, Artificial Sequence
  Reverse primer sequence for SARS CoV-2 Nucleocapsid gene
  modified_base 1: Thymidine at position 1 is modified with a fluorescent label
ttttcgtttgtttttyttgttttattgctttt

SEQ ID NO: 145 - DNA, 30 bp, Artificial Sequence
  Probe sequence for SARS CoV-2 Clade variant
ttttcgtttgtttttcttgttttatttttt

SEQ ID NO: 146 - DNA, 30 bp, Artificial Sequence; identical to SEQ ID NO: 145
  Probe sequence for SARS CoV-2 Clade variant

SEQ ID NO: 147 - DNA, 30 bp, Artificial Sequence
  Probe sequence for SARS CoV-2 Clade variant
tttttcttgttaattttacaaccatttttt

SEQ ID NO: 148 - DNA, 30 bp, Artificial Sequence
  Probe sequence for SARS CoV-2 Clade variant
tttttctttaatcttagaaccagacttttt

SEQ ID NO: 149 - DNA, 30 bp, Artificial Sequence
  Probe sequence for SARS CoV-2 Clade variant
tttttctccatgctatacatgtcctttttt

SEQ ID NO: 150 - DNA, 30 bp, Artificial Sequence
  Probe sequence for SARS CoV-2 Clade variant
tttttttttccatgctatctctgttttttt

SEQ ID NO: 151 - DNA, 30 bp, Artificial Sequence
  Probe sequence for SARS CoV-2 Clade variant
tttttttttccatgttatctctgttttttt

SEQ ID NO: 152 - DNA, 30 bp, Artificial Sequence
  Probe sequence for SARS CoV-2 Clade variant
tttttttggtttggtaaccctgcttttttt

SEQ ID NO: 153 - DNA, 32 bp, Artificial Sequence
  Probe sequence for SARS CoV-2 Clade variant
tttctttttgcttccaytgagaagtctttttt

SEQ ID NO: 154 - DNA, 30 bp, Artificial Sequence
  Probe sequence for SARS CoV-2 Clade variant
ttttttccgcttccactgagaagcattttt

SEQ ID NO: 155 - DNA, 30 bp, Artificial Sequence
  Probe sequence for SARS CoV-2 Clade variant
ttttttccgcttccattgagaagcattttt

SEQ ID NO: 156 - DNA, 32 bp, Artificial Sequence
  Probe sequence for SARS CoV-2 Clade variant
tttctttgggtgtttattaccacaaaaatttt

SEQ ID NO: 157 - DNA, 32 bp, Artificial Sequence
  Probe sequence for SARS CoV-2 Clade variant
tttttttttgggtgtttaccacaaaaactttt

SEQ ID NO: 158 - DNA, 32 bp, Artificial Sequence
  Probe sequence for SARS CoV-2 Clade variant
tttatttttgggtgttacttattaccacattt

SEQ ID NO: 159 - DNA, 32 bp, Artificial Sequence
  Probe sequence for SARS CoV-2 Clade variant
ttcttttggatgtttattaccacaaaaacttt

SEQ ID NO: 160 - DNA, 32 bp, Artificial Sequence
  Probe sequence for SARS CoV-2 Clade variant
ttcgtaatgatccattttattaccacaaattt

SEQ ID NO: 161 - DNA, 30 bp, Artificial Sequence
  Probe sequence for SARS CoV-2 Clade variant
tctcttcaaaagtttgatggaaatctcttt

SEQ ID NO: 162 - DNA, 30 bp, Artificial Sequence
  Probe sequence for SARS CoV-2 Clade variant
tttctttacaaaagtaggatggatttcttt

SEQ ID NO: 163 - DNA, 30 bp, Artificial Sequence
  Probe sequence for SARS CoV-2 Clade variant
tttctttgttggatgaaaagtgatcttctt

SEQ ID NO: 164 - DNA, 30 bp, Artificial Sequence
  Probe sequence for SARS CoV-2 Clade variant
ttttgaaagtgagttcagagtttacctttt

SEQ ID NO: 165 - DNA, 30 bp, Artificial Sequence
  Probe sequence for SARS CoV-2 Clade variant
ttctttggaaagtggagtttattctctttt

SEQ ID NO: 166 - DNA, 32 bp, Artificial Sequence; identical to SEQ ID NO: 118
  Primer sequence for SARS CoV-2 Clade variant

SEQ ID NO: 167 - DNA, 32 bp, Artificial Sequence; identical to SEQ ID NO: 119
  Primer sequence for SARS CoV-2 Clade variant

SEQ ID NO: 168 - DNA, 32 bp, Artificial Sequence; identical to SEQ ID NO: 120
  Probe sequence for SARS CoV-2 Clade variant

SEQ ID NO: 169 - DNA, 32 bp, Artificial Sequence; identical to SEQ ID NO: 121
  Probe sequence for SARS CoV-2 Clade variant

SEQ ID NO: 170 - DNA, 30 bp, Artificial Sequence; identical to SEQ ID NO: 122
  Probe sequence for SARS CoV-2 Clade variant

SEQ ID NO: 171 - DNA, 30 bp, Artificial Sequence; identical to SEQ ID NO: 123
  Probe sequence for SARS CoV-2 Clade variant

SEQ ID NO: 172 - DNA, 30 bp, Artificial Sequence; identical to SEQ ID NO: 124
  Probe sequence for SARS CoV-2 Clade variant

SEQ ID NO: 173 - DNA, 30 bp, Artificial Sequence
  Probe sequence for SARS CoV-2 Clade variant
tttttttaattaccagtatagatccttttt

SEQ ID NO: 174 - DNA, 30 bp, Artificial Sequence
  Probe sequence for SARS CoV-2 Clade variant
tttttcggtagcamaccttgtaatgttttt

SEQ ID NO: 175 - DNA, 30 bp, Artificial Sequence
  Probe sequence for SARS CoV-2 Clade variant
tttttttgtagcacaccttgtatttttttt

SEQ ID NO: 176 - DNA, 30 bp, Artificial Sequence
  Probe sequence for SARS CoV-2 Clade variant
tttttttgtagcaaaccttgtatttttttt

SEQ ID NO: 177 - DNA, 30 bp, Artificial Sequence
  Probe sequence for SARS CoV-2 Clade variant
ttttttatggtgttcaaggttttctttttt

SEQ ID NO: 178 - DNA, 32 bp, Artificial Sequence
  Probe sequence for SARS CoV-2 Clade variant
ttttctaattgttactttcctttacaattttt

SEQ ID NO: 179 - DNA, 30 bp, Artificial Sequence
  Probe sequence for SARS CoV-2 Clade variant
tttttttttgttactttcctttactttttt

SEQ ID NO: 180 - DNA, 30 bp, Artificial Sequence
  Probe sequence for SARS CoV-2 Clade variant
tttttttttgttactctcctttactttttt

SEQ ID NO: 181 - DNA, 32 bp, Artificial Sequence
  Probe sequence for SARS CoV-2 Clade variant
tttttctcctttacaaytatatggtttttttt

SEQ ID NO: 182 - DNA, 30 bp, Artificial Sequence
  Probe sequence for SARS CoV-2 Clade variant
tttttctctttacaatcatatggtcttttt

SEQ ID NO: 183 - DNA, 30 bp, Artificial Sequence
  Probe sequence for SARS CoV-2 Clade variant
tttttctctttacaaccatatggtcttttt

SEQ ID NO: 184 - DNA, 30 bp, Artificial Sequence
  Probe sequence for SARS CoV-2 Clade variant
tttttttacccactactggtgttttttttt

SEQ ID NO: 185 - DNA, 32 bp, Artificial Sequence; identical to SEQ ID NO: 125
  Probe sequence for SARS CoV-2 Clade variant

SEQ ID NO: 186 - DNA, 30 bp, Artificial Sequence; identical to SEQ ID NO: 126
  Probe sequence for SARS CoV-2 Clade variant

SEQ ID NO: 187 - DNA, 30 bp, Artificial Sequence; identical to SEQ ID NO: 127
  Probe sequence for SARS CoV-2 Clade variant

SEQ ID NO: 188 - DNA, 30 bp, Artificial Sequence; identical to SEQ ID NO: 129
  Probe sequence for SARS CoV-2 Clade variant

SEQ ID NO: 189 - DNA, 30 bp, Artificial Sequence; identical to SEQ ID NO: 128
  Probe sequence for SARS CoV-2 Clade variant

SEQ ID NO: 190 - DNA, 30 bp, Artificial Sequence
  Probe sequence for SARS CoV-2 Clade variant
tttttttttaattctcgtcggcgttttttt

SEQ ID NO: 191 - DNA, 30 bp, Artificial Sequence
  Probe sequence for SARS CoV-2 Clade variant
ttttccgcaacagttyaagaaattcatttt

SEQ ID NO: 192 - DNA, 30 bp, Artificial Sequence
  Probe sequence for SARS CoV-2 Clade variant
tttttttaacagttcaagaaattttttttt

SEQ ID NO: 193 - DNA, 30 bp, Artificial Sequence
  Probe sequence for SARS CoV-2 Clade variant
tttttttaacagtttaagaaattttttttt

SEQ ID NO: 194 - DNA, 30 bp, Artificial Sequence
  Probe sequence for SARS CoV-2 Clade variant
ttttctcaagaaattyaactccaggctttt

SEQ ID NO: 195 - DNA, 30 bp, Artificial Sequence
  Probe sequence for SARS CoV-2 Clade variant
tttttctaagaaattcaactccattttttt

SEQ ID NO: 196 - DNA, 30 bp, Artificial Sequence
  Probe sequence for SARS CoV-2 Clade variant
tttttctaagaaatttaactccattttttt

SEQ ID NO: 197 - DNA, 30 bp, Artificial Sequence
  Probe sequence for SARS CoV-2 Clade variant
tttttaaattcaactcyaggcagcatcttt

SEQ ID NO: 198 - DNA, 30 bp, Artificial Sequence
  Probe sequence for SARS CoV-2 Clade variant
tttttttttcaactccaggcagcttttttt

SEQ ID NO: 199 - DNA, 30 bp, Artificial Sequence
  Probe sequence for SARS CoV-2 Clade variant
tttttttttcaactctaggcagcttttttt

SEQ ID NO: 200 - DNA, 30 bp, Artificial Sequence
  Probe sequence for SARS CoV-2 Clade variant
tttttactccaggcakcwstadrsgatttt

SEQ ID NO: 201 - DNA, 30 bp, Artificial Sequence
  Probe sequence for SARS CoV-2 Clade variant
ttttttttccaggcagcagtadrttttttt

SEQ ID NO: 202 - DNA, 30 bp, Artificial Sequence
  Probe sequence for SARS CoV-2 Clade variant
ttttttttccaggcatcagtaggttttttt

SEQ ID NO: 203 - DNA, 30 bp, Artificial Sequence
  Probe sequence for SARS CoV-2 Clade variant
ttttcccaggcagcartadrsgaacctttt

SEQ ID NO: 204 - DNA, 30 bp, Artificial Sequence
  Probe sequence for SARS CoV-2 Clade variant
tttttttaggcagcagtadrsgattttttt

SEQ ID NO: 205 - DNA, 30 bp, Artificial Sequence
  Probe sequence for SARS CoV-2 Clade variant
tttttttaggcagcaataggggattttttt

SEQ ID NO: 206 - DNA, 30 bp, Artificial Sequence
  Probe sequence for SARS CoV-2 Clade variant
ttttgcagcwstadrsgaacttctcttttt

SEQ ID NO: 207 - DNA, 30 bp, Artificial Sequence
  Probe sequence for SARS CoV-2 Clade variant
tttttttcagcagtaggggaactctttttt

SEQ ID NO: 208 - DNA, 30 bp, Artificial Sequence
  Probe sequence for SARS CoV-2 Clade variant
tttttttcagcagtatgggaactctttttt

SEQ ID NO: 209 - DNA, 30 bp, Artificial Sequence
  Probe sequence for SARS CoV-2 Clade variant
tttttttcagcagtaaacgaactctttttt

SEQ ID NO: 210 - DNA, 30 bp, Artificial Sequence
  Probe sequence for SARS CoV-2 Clade variant
tttttttcagctctaaacgaactctttttt

SEQ ID NO: 211 - DNA, 30 bp, Artificial Sequence
  Probe sequence for SARS CoV-2 Clade variant
ttttcstadrsgaayttctcctgctatttt

SEQ ID NO: 212 - DNA, 30 bp, Artificial Sequence
  Probe sequence for SARS CoV-2 Clade variant
tttttttggggaacttctcctgcctttttt

SEQ ID NO: 213 - DNA, 30 bp, Artificial Sequence
  Probe sequence for SARS CoV-2 Clade variant
tttttttggggaatttctcctgcctttttt

SEQ ID NO: 214 - DNA, 30 bp, Artificial Sequence
  Probe sequence for SARS CoV-2 Clade variant
ttttaayttctcctgctagaatggctgttt

SEQ ID NO: 215 - DNA, 30 bp, Artificial Sequence
  Probe sequence for SARS CoV-2 Clade variant
ttttacgaacttctcctggaatggctgttt

SEQ ID NO: 216 - DNA, 30 bp, Artificial Sequence
  Probe sequence for SARS CoV-2 Clade variant
ttttctctcctgctagaatggctgtttttt

SEQ ID NO: 217 - DNA, 30 bp, Artificial Sequence
  Probe sequence for SARS CoV-2 Clade variant
tttttacttctcctggaatggctgtttttt

SEQ ID NO: 218 - DNA, 30 bp, Artificial Sequence
  Probe sequence for SARS CoV-2 Clade variant
ttttttggctgkcwatkgckgtgatttttt

SEQ ID NO: 219 - DNA, 30 bp, Artificial Sequence
  Probe sequence for SARS CoV-2 Clade variant
tttttttaatggctggcwatkgcttttttt

SEQ ID NO: 220 - DNA, 30 bp, Artificial Sequence
  Probe sequence for SARS CoV-2 Clade variant
tttttttaatggctgtcaatggcttttttt

SEQ ID NO: 221 - DNA, 30 bp, Artificial Sequence
  Probe sequence for SARS CoV-2 Clade variant
ttttttttggctgkcaatkgckgctttttt

SEQ ID NO: 222 - DNA, 30 bp, Artificial Sequence
  Probe sequence for SARS CoV-2 Clade variant
ttttttttggctggctatggcggctttttt

SEQ ID NO: 223 - DNA, 30 bp, Artificial Sequence; identical to SEQ ID NO: 218
  Probe sequence for SARS CoV-2 Clade variant

SEQ ID NO: 224 - DNA, 30 bp, Artificial Sequence
  Probe sequence for SARS CoV-2 Clade variant
ttttttttgkcwatggckgtgatttttttt

SEQ ID NO: 225 - DNA, 30 bp, Artificial Sequence
  Probe sequence for SARS CoV-2 Clade variant
ttttttttggcaattgcggtgatttttttt

SEQ ID NO: 226 - DNA, 30 bp, Artificial Sequence
  Probe sequence for SARS CoV-2 Clade variant
tttttttcwatkgcggtgatgctttttttt

SEQ ID NO: 227 - DNA, 30 bp, Artificial Sequence
  Probe sequence for SARS CoV-2 Clade variant
tttttttcaatggctgtgatgctttttttt

SEQ ID NO: 228 - DNA, 30 bp, Artificial Sequence
  Probe sequence for SARS CoV-2 Clade variant
tttttgagcaaaatdtytggtaaagttttt

SEQ ID NO: 229 - DNA, 30 bp, Artificial Sequence
  Probe sequence for SARS CoV-2 Clade variant
tttttttcaaaatgtctggtaaattttttt

SEQ ID NO: 230 - DNA, 30 bp, Artificial Sequence
  Probe sequence for SARS CoV-2 Clade variant
tttttctagcaaaatttctggtatcttttt

SEQ ID NO: 231 - DNA, 30 bp, Artificial Sequence
  Probe sequence for SARS CoV-2 Clade variant
tttttctagcaaaatatctggtatcttttt

SEQ ID NO: 232 - DNA, 30 bp, Artificial Sequence
  Probe sequence for SARS CoV-2 Clade variant
tttttctcaaaatgtttggtaaatcttttt

SEQ ID NO: 233 - DNA, 30 bp, Artificial Sequence
  Probe sequence for SARS CoV-2 Clade variant
ttttttaatyttasaamcagaactcttttt

SEQ ID NO: 234 - DNA, 30 bp, Artificial Sequence
  Probe sequence for SARS CoV-2 Clade variant
tttttcttgttaattttacaamcatttttt

SEQ ID NO: 235 - DNA, 32 bp, Artificial Sequence
  Probe sequence for SARS CoV-2 Clade variant
tttttcccatgytatacatgtctctgtttttt

SEQ ID NO: 236 - DNA, 32 bp, Artificial Sequence
  Probe sequence for SARS CoV-2 Clade variant
tttttttttccatgytatctctgggatttttt

SEQ ID NO: 237 - DNA, 30 bp, Artificial Sequence
  Probe sequence for SARS CoV-2 Clade variant
ttttttgaccaatggtactaagagtttttt

SEQ ID NO: 238 - DNA, 30 bp, Artificial Sequence
  Probe sequence for SARS CoV-2 Clade variant
ttttttaccaatggtactaagagtcttttt

SEQ ID NO: 239 - DNA, 30 bp, Artificial Sequence
  Probe sequence for SARS CoV-2 Clade variant
ttttttaccaatgttattaagagtcttttt

SEQ ID NO: 240 - DNA, 32 bp, Artificial Sequence
  Probe sequence for SARS CoV-2 Clade variant
tttttcagaggtttgvtaaccctgtctttttt

SEQ ID NO: 241 - DNA, 32 bp, Artificial Sequence
  Probe sequence for SARS CoV-2 Clade variant
tttctttggrtgtttattaccacaaaaatttt

SEQ ID NO: 242 - DNA, 30 bp, Artificial Sequence
  Probe sequence for SARS CoV-2 Clade variant
tttctgtgtttatyaccacaaaaacttctt

SEQ ID NO: 243 - DNA, 31 bp, Artificial Sequence
  Probe sequence for SARS CoV-2 Clade variant
tttttctgtgtttattaccacaaatcttttt

SEQ ID NO: 244 - DNA, 30 bp, Artificial Sequence
  Probe sequence for SARS CoV-2 Clade variant
ttttttatgtttatcaccacaaatcttttt

SEQ ID NO: 245 - DNA, 30 bp, Artificial Sequence
  Probe sequence for SARS CoV-2 Clade variant
tttttttttgcgtgatctcccttttttttt

SEQ ID NO: 246 - DNA, 30 bp, Artificial Sequence
  Probe sequence for SARS CoV-2 Clade variant
tttttttttgcgtggtctcccctttttttt

SEQ ID NO: 247 - DNA, 30 bp, Artificial Sequence
  Probe sequence for SARS CoV-2 Clade variant
ttttgtttttcggytttagaaccatctttt

SEQ ID NO: 248 - DNA, 30 bp, Artificial Sequence
  Probe sequence for SARS CoV-2 Clade variant
tttttttttttcggctttagaacttttttt

SEQ ID NO: 249 - DNA, 30 bp, Artificial Sequence
  Probe sequence for SARS CoV-2 Clade variant
ttttttgtttttcggttttagaattttttt

SEQ ID NO: 250 - DNA, 30 bp, Artificial Sequence
  Probe sequence for SARS CoV-2 Clade variant
ttttaactggaamkattgctgattattttt

SEQ ID NO: 251 - DNA, 30 bp, Artificial Sequence
  Probe sequence for SARS CoV-2 Clade variant
tttctataattaccdgtatagattgtcttt

SEQ ID NO: 252 - DNA, 30 bp, Artificial Sequence
  Probe sequence for SARS CoV-2 Clade variant
tttttcgccggtarcamaccttgtattttt

SEQ ID NO: 253 - DNA, 30 bp, Artificial Sequence
  Probe sequence for SARS CoV-2 Clade variant
tttttttccggtagcacacctttttttttt

SEQ ID NO: 254 - DNA, 30 bp, Artificial Sequence
  Probe sequence for SARS CoV-2 Clade variant
tttttctggtagcaaaccttgttttttttt

SEQ ID NO: 255 - DNA, 32 bp, Artificial Sequence
  Probe sequence for SARS CoV-2 Clade variant
ttttttaatggtgytraaggttttaatttttt

SEQ ID NO: 256 - DNA, 32 bp, Artificial Sequence
  Probe sequence for SARS CoV-2 Clade variant
ttttctaattgttactytcctttacaattttt

SEQ ID NO: 257 - DNA, 33 bp, Artificial Sequence
  Probe sequence for SARS CoV-2 Clade variant
ttttttttccaacccactwmtggtgtttttttt

SEQ ID NO: 258 - DNA, 30 bp, Artificial Sequence
  Probe sequence for SARS CoV-2 Clade variant
tttttccctttatcatgatgttaatctttt

SEQ ID NO: 259 - DNA, 32 bp, Artificial Sequence
  Probe sequence for SARS CoV-2 Clade variant
ttttttatcagactcmbactaattctcttttt

SEQ ID NO: 260 - DNA, 30 bp, Artificial Sequence
  Probe sequence for SARS CoV-2 Clade variant
ttttttcagactaattctcvtcggcttttt

SEQ ID NO: 261 - DNA, 30 bp, Artificial Sequence
  Probe sequence for SARS CoV-2 Clade variant
ttttttagtgtagytagtcaatccactttt

SEQ ID NO: 262 - DNA, 30 bp, Artificial Sequence
  Probe sequence for SARS CoV-2 Clade variant
ttttttcagtgtagctagtcaatttttttt

SEQ ID NO: 263 - DNA, 30 bp, Artificial Sequence
  Probe sequence for SARS CoV-2 Clade variant
ttttttcagtgtagttagtcaatttttttt

SEQ ID NO: 264 - DNA, 26 bp, Artificial Sequence
  Forward primer sequence for SARS CoV-2 Nucleocapsid gene
ttttgccaaaaggcttctacgcagaa

SEQ ID NO: 265 - DNA, 26 bp, Artificial Sequence
  Reverse primer sequence for SARS CoV-2 Nucleocapsid gene
ttgtttttgccgaggcttcttagaag

SEQ ID NO: 266 - DNA, 28 bp, Artificial Sequence
  Forward primer for Spike gene target sequence
ttacctttcttttccaatgttacttggt

SEQ ID NO: 267 - DNA, 26 bp, Artificial Sequence
  Forward primer for Spike gene target sequence
tttgtaattagaggtgatgaagtcag

SEQ ID NO: 268 - DNA, 23 bp, Artificial Sequence
  Forward primer for Spike gene target sequence
ttttgagagagatatttcactga

SEQ ID NO: 269 - DNA, 23 bp, Artificial Sequence
  Reverse primer for Spike gene target sequence
  modified_base 1: Thymidine at position 1 is modified with a fluorescent label
ggtatggcaatagagttattaga

SEQ ID NO: 270 - DNA, 25 bp, Artificial Sequence
  Reverse primer for Spike gene target sequence
  modified_base 1: Thymidine at position 1 is modified with a fluorescent label
aaggtgagcggctgtctccacaagt

SEQ ID NO: 271 - protein, 10 aa, Severe acute respiratory syndrome coronavirus-2
  Amino acid translation of Spike gene target sequence
LPFFSNVTWF

SEQ ID NO: 272 - protein, 9 aa, Severe acute respiratory syndrome coronavirus-2
  Amino acid translation of Spike gene target sequence
  SITE 2: Xaa at position 2 is Phe or Ser
KXNIIRGWI

SEQ ID NO: 273 - protein, 9 aa, Severe acute respiratory syndrome coronavirus-2
  Amino acid translation of Spike gene target sequence
LIVNNATNV

SEQ ID NO: 274 - protein, 9 aa, Severe acute respiratory syndrome coronavirus-2
  Amino acid translation of Spike gene target sequence
SSANNCTFE

SEQ ID NO: 275 - protein, 9 aa, Severe acute respiratory syndrome coronavirus-2
  Amino acid translation of Spike gene target sequence
FVIRGDEVR

SEQ ID NO: 276 - protein, 9 aa, Severe acute respiratory syndrome coronavirus-2
  Amino acid translation of Spike gene target sequence
FRKSNLKPF

SEQ ID NO: 277 - protein, 9 aa, Severe acute respiratory syndrome coronavirus-2
  Amino acid translation of Spike gene target sequence
PFFRDISTE

SEQ ID NO: 278 - protein, 9 aa, Severe acute respiratory syndrome coronavirus-2
  Amino acid translation of Spike gene target sequence
QPYRVVVLS

SEQ ID NO: 279 - protein, 8 aa, Severe acute respiratory syndrome coronavirus-2
  Amino acid translation of Spike gene target sequence
IGAGICAS

SEQ ID NO: 280 - protein, 8 aa, Severe acute respiratory syndrome coronavirus-2
  Amino acid translation of Spike gene target sequence
SNNSIAIP

SEQ ID NO: 281 - DNA, 30 bp, Artificial Sequence
  Probe for Spike gene target sequence
ttttttttaggtttgataaccccttttttt

SEQ ID NO: 282 - DNA, 30 bp, Artificial Sequence
  Probe for Spike gene target sequence
tttctggtgttacttctaaccacaattttt

SEQ ID NO: 283 - DNA, 30 bp, Artificial Sequence; identical to SEQ ID NO: 282
  Probe for Spike gene target sequence

SEQ ID NO: 284 - DNA, 32 bp, Artificial Sequence
  Probe for Spike gene target sequence
ttcgatccatttttggaccacaaaaacaattt

SEQ ID NO: 285 - DNA, 32 bp, Artificial Sequence
  Probe for Spike gene target sequence
tttatttttgggtgtttaccacaaaaacattt

SEQ ID NO: 286 - DNA, 32 bp, Artificial Sequence
  Probe for Spike gene target sequence
tttttaatgatccattttattaccacaatttt

SEQ ID NO: 287 - DNA, 32 bp, Artificial Sequence
  Probe for Spike gene target sequence
ttcttttggrtgtttattaccacaaaaacttt

SEQ ID NO: 288 - DNA, 30 bp, Artificial Sequence; identical to SEQ ID NO: 282
  Probe for Spike gene target sequence

SEQ ID NO: 289 - DNA, 32 bp, Artificial Sequence; identical to SEQ ID NO: 284
  Probe for Spike gene target sequence

SEQ ID NO: 290 - DNA, 30 bp, Artificial Sequence
  Probe for Spike gene target sequence
tttttagtwkkatggaaagtgagttctttt

SEQ ID NO: 291 - DNA, 30 bp, Artificial Sequence
  Probe for Spike gene target sequence
tttctttcaaaagtagcatggaatcttctt

SEQ ID NO: 292 - DNA, 30 bp, Artificial Sequence
  Probe for Spike gene target sequence
ttttcaaagtgagttcagagtttacctttt

SEQ ID NO: 293 - DNA, 30 bp, Artificial Sequence
  Probe for Spike gene target sequence
ttctttggaaagtggagtttattccttttt

SEQ ID NO: 294 - DNA, 30 bp, Artificial Sequence
  Probe for Spike gene target sequence
tttttcataattaccggtatagatcttttt

SEQ ID NO: 295 - DNA, 30 bp, Artificial Sequence
  Probe for Spike gene target sequence
ttttttttaattaccagtatagactttttt

SEQ ID NO: 296 - DNA, 30 bp, Artificial Sequence
  Probe for Spike gene target sequence
tttttttggtagcacaccttgttttttttt

SEQ ID NO: 297 - DNA, 32 bp, Artificial Sequence
  Probe for Spike gene target sequence
ttttttaatggtgytrmaggttttaatttttt

SEQ ID NO: 298 - DNA, 30 bp, Artificial Sequence
  Probe for Spike gene target sequence
ttttttcggtgttgcaggttttatcttttt

SEQ ID NO: 299 - DNA, 32 bp, Artificial Sequence
  Probe for Spike gene target sequence
tttttctcctttacaaycatatggtttttttt

SEQ ID NO: 300 - DNA, 31 bp, Artificial Sequence
  Probe for Spike gene target sequence
tttttctttacgatcatatagtttccttttt

SEQ ID NO: 301 - DNA, 30 bp, Artificial Sequence
  Probe for Spike gene target sequence
tttttcttacgatcatatagtttctttttt

SEQ ID NO: 302 - DNA, 33 bp, Artificial Sequence; identical to SEQ ID NO: 55
  Probe for Spike gene target sequence

SEQ ID NO: 303 - DNA, 30 bp, Artificial Sequence
  Probe for Spike gene target sequence
ttttttcagactaaktctcvtcggcttttt

SEQ ID NO: 304 - DNA, 30 bp, Artificial Sequence
  Probe for Spike gene target sequence
tttttttttaaktctcatcggcgttttttt

SEQ ID NO: 305 - DNA, 30 bp, Artificial Sequence
  Probe for Spike gene target sequence
tcttcttcttggtgtagaaaatcttctttt